(19) United States
(12) Patent Application Publication
(10) Pub. No.: US 2023/0383012 A1
(43) Pub. Date: Nov. 30, 2023

(54) ANTIBODIES THAT BIND PD-L1, PD-L2, AND/OR CD28

(71) Applicant: Xencor, Inc., Pasadena, CA (US)

(72) Inventors: Gregory Moore, Azusa, CA (US); John R. Desjarlais, Pasadena, CA (US); Michael Hedvat, Encino, CA (US); Veronica Gusti Zeng, Duarte, CA (US); Juan Diaz, Anaheim Hills, CA (US)

(21) Appl. No.: 18/300,302

(22) Filed: Apr. 13, 2023

Related U.S. Application Data
(60) Provisional application No. 63/330,769, filed on Apr. 13, 2022.
(60) Provisional application No. 63/379,100, filed on Oct. 11, 2022.
(60) Provisional application No. 63/382,592, filed on Nov. 7, 2022.
(60) Provisional application No. 63/478,881, filed on Jan. 6, 2023.
(60) Provisional application No. 63/480,478, filed on Jan. 18, 2023.

(51) Int. Cl. C07K 16/46; C12N 15/63

(52) U.S. Cl. CPC C07K 16/468; C12N 15/63; C07K 2317/31; C07K 2317/52; C07K 2317/622

(57) ABSTRACT

Provided herein are novel αPD-L1, αPD-L2, and αCD28 antibodies. In some embodiments, the antibodies are αPD-L1×αPD-L2×αCD28 antibodies. Such antibodies enhance anti-tumor activity by providing a costimulatory signal for T-cell activation against tumor cells while advantageously also blocking inhibitory PD-L1:PD1 and/or PD-L2:PD1 pathway interactions.

### PRIORITY CLAIM

This application claims priority to and benefit of U.S. Provisional Application No. 63/330,769, filed on Apr. 13, 2022; 63/379,100, filed on Oct. 11, 2022; 63/382,592, filed Nov. 7, 2022; 63/478,881, filed Jan. 6, 2023; and 63/480,478, filed on Jan. 18, 2023, the contents of which are hereby incorporated by reference in their entireties.

### SEQUENCE LISTING

The instant application contains a Sequence Listing which has been submitted electronically in XML file format and is hereby incorporated by reference in its entirety. Said XML copy, created on Apr. 11, 2023, is named 067461-5301-WO_SL.xml and is 3,420,050 bytes in size.

### BACKGROUND

The natural immune response against tumor dispatches immune effector cells such as natural killer (NK) cells and T cells to attack and destroy tumor cells. Tumor infiltrating lymphocytes (TILs) often express multiple immune checkpoint receptors (e.g., PD-1, CTLA-4) and costimulatory receptors (e.g., ICOS, 4-1BB, OX40, GITR, and CD28). TILs lose their cytotoxic ability over time due to upregulation of inhibitory immune checkpoints. While checkpoint blockade has demonstrated increased clinical response rates relative to other treatment options, many patients still fail to achieve a response to checkpoint blockade. Engagement of costimulatory receptors on TILs could provide a positive signal capable of overcoming negative signals of immune checkpoints. Preclinical and clinical studies of agonistic costimulatory receptor antibodies have indeed demonstrated that agonism of costimulatory receptors can result in impressive anti-tumor responses, activating T cells to attack tumor cells.

It is also important for cancer therapy to enhance anti-tumor activity by specifically destroying tumor cells while minimizing peripheral toxicity. In this context, it is crucial that only T cells in the presence of the target tumor cells are provided a costimulatory signal. However, agonism of costimulatory receptors with monospecific full-length antibodies is likely nondiscriminatory with regards to TILs vs. peripheral T cells vs. autoantigen-reactive T cells that contribute to autoimmune toxicities. For instance, urelumab, a monospecific, nondiscriminatory, pan-4-1BB agonist antibody, exhibited significant liver toxicity in early phase clinical trials (Segal et al., 2016). Thus, there remains a need for novel immune response enhancing compositions for the treatment of cancers.

### SUMMARY

Provided herein are novel αPD-L1, αPD-L2, and αCD28 antibodies. In some embodiments, the antibodies are αPD-L1×αPD-L2×αCD28 trispecific antibodies. Such antibodies enhance anti-tumor activity by providing a costimulatory signal for T-cell activation against tumor cells while advantageously also blocking inhibitory PD-L1:PD1 and/or PD-L2:PD1 pathway interactions (see FIG. 94). In some embodiments, such trispecific antibodies are useful for the treatment of cancers in conjunction with αCD3×α tumor target antigen (TTA) bispecific antibodies.

In one aspect, provided herein are multispecific antibodies that comprise a PD-L1 antigen binding domain, a PD-L2 antigen binding domain, and a CD28 binding domain, wherein each of the binding domains comprises a variable heavy domain and a variable light domain.

In one aspect, provided herein are novel 1+1+1 stackFab₂-Fab-Fc format antibodies (FIG. 83D). This antibody includes a) a first monomer, b) a second monomer, and c) a first, second, and third common light chain. The first monomer includes from N-terminal to C-terminal, VH1-CH1-hinge-CH2-CH3, wherein VH1 is a first variable heavy domain and CH2-CH3 is a first Fc domain. The second monomer includes from N-terminal to C-terminal, VH2-CH1-linker-VH3-CH1-hinge-CH2-CH3 wherein VH2 is a second variable heavy domain, VH3 is a third variable heavy domain, and CH2-CH3 is a second Fc domain. The common light chains each include from N-terminal to C-terminal, VL-CL, wherein VL is a common variable light domain and CL is a constant light domain. In this format, the first variable heavy domain and the common variable light domain of the first common light chain form a first antigen binding domain, the second variable heavy domain and the common variable light domain of the second common light chain form a second antigen binding domain, and the third variable heavy domain and common variable light domain form a third antigen binding domain of the third common light chain.

In some embodiments, the antibody is a trispecific antibody that binds PD-L1, PD-L2, and CD28, and each of the first antigen binding domain, second antigen binding domain, and third antigen binding domain is selected from the group consisting of a PD-L1 antigen binding domain, a PD-L2 antigen binding domain, and a CD 28 binding domain.

In some embodiments, the first antigen binding domain, the second antigen binding domain, and the third antigen binding domain are selected from the following: i) the first antigen binding domain is the PD-L1 binding domain, the second antigen binding domain is the PD-L2 binding domain, and the third antigen binding domain is the CD28 binding domain, ii) the first antigen binding domain is the PD-L1 binding domain, the second antigen binding domain is the CD28 binding domain, and the third antigen binding domain is the PD-L2 binding domain, iii) the first antigen binding domain is the PD-L2 binding domain, the second antigen binding domain is the PD-L1 binding domain, and the third antigen binding domain is the CD28 binding domain, iv) the first antigen binding domain is the PD-L2 binding domain, the second antigen binding domain is the CD28 binding domain, and the third antigen binding domain is the PD-L1 binding domain, v) the first antigen binding domain is the CD28 binding domain, the second antigen binding domain is the PD-L1 binding domain, and the third antigen binding domain is the PD-L2 binding domain, or vi) the first antigen binding domain is the CD28 binding domain, the second antigen binding domain is the PD-L2 binding domain, and the third antigen binding domain is the PD-L1 binding domain.

In some embodiments, the CD28 binding domain comprises a variable heavy domain having an amino acid selected from SEQ ID NOs:3354-3389 or a variant thereof, and a variable light domain having the amino acid sequence of SEQ ID NO:3239 or variant thereof.

In some embodiments, the PD-L1 antigen binding domain having an amino acid selected from SEQ ID NOs:3235 and 3243-3260 or a variant thereof, and a variable light domain having the amino acid sequence of SEQ ID NO:3239 or variant thereof.

In some embodiments, the PD-L2 antigen binding domain having an amino acid selected from SEQ ID NOs:3267 and 3275-3347 or a variant thereof, and a variable light domain having the amino acid sequence of SEQ ID NO:3239 or variant thereof.

In some embodiments, the first and second Fc domains are variant Fc domains. In certain embodiments, the first and second Fc domains comprise a set of heterodimerization skew variants selected from the group consisting of S364K/E357Q:L368D/K370S; S364K: L368D/K370S; S364K:L368E/K370S; D401K:T411E/K360E/Q362E; and T366W:T366S/L368A/Y407V, wherein numbering is according to EU numbering. In some embodiments, the first and second Fc domains comprise heterodimerization skew variants S364K/E357Q:L368D/K370S.

In some embodiments, the first and second Fc domains each comprise one or more ablation variants. In some embodiments, the one or more ablation variants are E233P/L234V/L235A/G236del/S267K, wherein numbering is according to EU numbering.

In certain embodiments, one of the first or second monomer comprises one or more pI variants. In some embodiments, the CH1-hinge-CH2-CH3 of the first monomer comprises pI variants N208D/Q295E/N384D/Q418E/N421D, wherein numbering is according to EU numbering.

In some embodiments, the first Fc domain comprises amino acid variants L368D/K370S/N208D/Q295E/N384D/Q418E/N421D/E233P/L234V/L235A/G236del/S267K, and the CH1-hinge-CH2-CH3 of the second monomer comprises amino acid variants S364K/E357Q/E233P/L234V/L235A/G236del/S267K, and wherein numbering is according to EU numbering.

In some embodiments, the first and second Fc domains each further comprise amino acid variants 428/434S.

In some embodiments, a) the PD-L1 binding domain comprises a variable heavy domain having the amino acid sequence of SEQ ID NO:3251, and a variable light domain having the amino acid sequence of SEQ ID NO:3239; b) the PD-L2 binding domain comprises a variable heavy domain having the amino acid sequence of SEQ ID NO:3319, and a variable light domain having the amino acid sequence of SEQ ID NO:3239; and c) the CD28 binding domain comprises a variable heavy domain having the amino acid sequence of SEQ ID NO:3380, and a variable light domain having the amino acid sequence of SEQ ID NO:3239.

In some embodiments, a) the first monomer has the amino acid sequence of SEQ ID NO:3201; b) the second monomer has the amino acid sequence of SEQ ID NO:3202; and c) the first, second, and third common light chains each have the amino acid sequence of SEQ ID NO:3203.

In one aspect, provided herein are novel 1+1+1 stackFab₂-scFv-Fc format antibodies (FIG. 83A). The 1+1+1 stackFab₂-scFv-Fc format includes a) a first monomer, b) second monomer, and c) a first and second common light chain. The first monomer includes from N-terminal to C-terminal, scFv-linker-CH2-CH3 wherein CH2-CH3 is a first Fc domain. The second monomer includes from N-terminal to C-terminal, VH1-CH1-linker-VH2-CH1-hinge-CH2-CH3 wherein VH1 is a first variable heavy domain, VH2 is a second variable heavy domain, and CH2-CH3 is a second Fc domain. The first and second common light chains each include from N-terminal to C-terminal, VL1-CL, wherein VL1 is a first variable light domain and CL is a constant light domain. Further, the scFv comprises a third VH domain (VH3), a scFv linker, and a second variable light domain (VL2). In this format, the first variable heavy domain and the first variable light domain of the first common light chain form a first antigen binding domain, the second variable heavy domain and the first variable light domain of the second common light chain form a second antigen binding domain, and the third variable heavy domain and second variable light domain form a third antigen binding domain.

In some embodiments, the antibody is a trispecific antibody that binds PD-L1, PD-L2, and CD28, and each of the first antigen binding domain, second antigen binding domain, and third antigen binding domain is selected from the group consisting of a PD-L1 antigen binding domain, a PD-L2 antigen binding domain, and a CD 28 binding domain.

In some embodiments, the first antigen binding domain, the second antigen binding domain, and the third antigen binding domain are selected from the following:

i) the first antigen binding domain is the PD-L1 binding domain, the second antigen binding domain is the PD-L2 binding domain, and the third antigen binding domain is the CD28 binding domain, ii) the first antigen binding domain is the PD-L1 binding domain, the second antigen binding domain is the CD28 binding domain, and the third antigen binding domain is the PD-L2 binding domain, iii) the first antigen binding domain is the PD-L2 binding domain, the second antigen binding domain is the PD-L1 binding domain, and the third antigen binding domain is the CD28 binding domain, iv) the first antigen binding domain is the PD-L2 binding domain, the second antigen binding domain is the CD28 binding domain, and the third antigen binding domain is the PD-L1 binding domain, v) the first antigen binding domain is the CD28 binding domain, the second antigen binding domain is the PD-L1 binding domain, and the third antigen binding domain is the PD-L2 binding domain, or vi) the first antigen binding domain is the CD28 binding domain, the second antigen binding domain is the PD-L2 binding domain, and the third antigen binding domain is the PD-L1 binding domain.

In some embodiments, the CD28 binding domain comprises a variable heavy domain and variable light domain selected from the following: i) a variable heavy domain selected from any of the variable heavy domains in FIGS. 35, 36, 38, and a variable light domain selected from any of the variable light domains in FIGS. 35, 37 and 38, ii) a variable heavy domain and a variable light domain of a CD28 binding domain in FIG. 38, iii) a variable heavy domain selected from any of the variable heavy domains in FIGS. 41-74, and a variable light domain selected from any of the variable light domains in FIGS. 41-74, iv) a variable heavy domain and variable light domain of a CD28 binding domain in FIGS. 41-74, v) a variable heavy domain selected from any of the variable light domains in FIGS. 41-74, and a variable light domain having the amino acid sequence of SEQ ID NO:20 (FIG. 24), vi) a variable heavy domain and variable light domain of a CD28 binding domains in FIG. 81, and vii) a variable heavy domain selected from any of the variable heavy domains in FIG. 163, and a variable light domain of selected from any of the variable light domains in FIGS. 35 and 37, or a variable light domain having the amino acid sequence of SEQ ID NO:3239.

In some embodiments, the PD-L1 antigen binding domain comprises a variable heavy domain and a variable light domain selected from the following: i) a variable heavy domain selected from any of the variable heavy domains in FIGS. 17 and 19, and a variable light domain selected from any of the variable light domains in FIGS. 17 and 20, ii) a variable heavy domain and variable light domain of a PD-L1 binding domain in FIGS. 17 and 21, iii) a variable heavy domain selected from any of the variable heavy domains in FIGS. 25 and 26, and a variable light domain selected from any of the variable light domains in FIGS. 25 and 26, iv) a variable heavy domain and variable light domain of a PD-L1 binding domain in FIGS. 25 and 26, v) a variable heavy domain selected from any of the variable heavy domains in FIGS. 25 and 26, and a variable light domain having the amino acid sequence of SEQ ID NO:20 (FIG. 24), vi) a variable heavy domain and variable light domain of a PD-L1 binding domain in FIG. 28; and vii) a variable heavy domain selected from any of the variable heavy domains in FIG. 161, and a variable light domain having the amino acid sequence of SEQ ID NO:3239.

In some embodiments, the PD-L2 antigen binding domain comprises a variable heavy domain and a variable light domain selected from the following: i) a variable heavy domain selected from any of the variable heavy domains in FIGS. 29-32, and a variable light domain selected from any of the variable light domains in in FIGS. 29-32, ii) a variable heavy domain and variable light domain of a PD-L2 binding domain in FIGS. 29-32, iii) a variable heavy domain selected from any of the variable heavy domains in FIGS. 29-32, and a variable light domain having the amino acid sequence of SEQ ID NO:20 (FIG. 24), iv) a variable heavy domain and variable light domain of a PD-L2 binding domain in FIG. 34, and v) a variable heavy domain selected from any of the variable heavy domains in FIG. 162, and a variable light domain having the amino acid sequence of SEQ ID NO:3239.

In some embodiments, the first and second Fc domains are variant Fc domains. In certain embodiments, the first and second Fc domains comprise a set of heterodimerization skew variants selected from the group consisting of S364K/E357Q:L368D/K370S; S364K: L368D/K370S; S364K:L368E/K370S; D401K:T411E/K360E/Q362E; and T366W:T366S/L368A/Y407V, wherein numbering is according to EU numbering. In some embodiments, the first and second Fc domains comprise heterodimerization skew variants S364K/E357Q:L368D/K370S.

In some embodiments, the first and second Fc domains each comprise one or more ablation variants. In some embodiments, the one or more ablation variants are E233P/L234V/L235A/G236del/S267K, wherein numbering is according to EU numbering.

In some embodiments, one of the first or second monomer comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the first Fc domain comprises amino acid variants S364K/E357Q/E233P/L234V/L235A/G236del/S267K, the CH1-hinge-CH2-CH3 of the second monomer comprises amino acid variants L368D/K370S/N208D/Q295E/N384D/Q418E/N421D/E233P/L234V/L235A/G236del/S267K, and numbering is according to EU numbering.

In some embodiments, the scFv linker is a charged scFv linker having the amino acid sequence (GKPGS)₄. In some embodiments, the first and second Fc domains each further comprise amino acid variants 428/434S.

In one aspect, provided herein are novel 1+1+1 Fab-(Fab-scFv)-Fc format antibodies (FIG. 83B). The 1+1+1 Fab-(Fab-scFv)-Fc format includes a) a first monomer, b) second monomer and c) a first and second common light chain. The first monomer includes from N-terminal to C-terminal, VH1-CH1-linker-scFv-linker-CH2-CH3 wherein VH1 is a first variable heavy domain, and CH2-CH3 is a first Fc domain. The second monomer includes from N-terminal to C-terminal, VH2-CH1-hinge-CH2-CH3 wherein VH2 is a second variable heavy domain, and CH2-CH3 is a second Fc domain. The first and second common light chains each include from N-terminal to C-terminal, VL1-CL, wherein VL1 is a first variable light domain and CL is a constant light domain. Further, the scFv comprises a third VH domain (VH3), a scFv linker, and a second variable light domain (VL2). In this format, the first variable heavy domain and the first variable light domain of the first common light chain form a first antigen binding domain, the second variable heavy domain and the first variable light domain of the second common light chain form a second antigen binding domain, and the third variable heavy domain and second variable light domain form a third antigen binding domain.

In some embodiments, the antibody is a trispecific antibody that binds PD-L1, PD-L2, and CD28, and each of the first antigen binding domain, second antigen binding domain, and third antigen binding domain is selected from the group consisting of a PD-L1 antigen binding domain, a PD-L2 antigen binding domain, and a CD 28 binding domain.

In some embodiments, the first antigen binding domain, the second antigen binding domain, and the third antigen binding domain are selected from the following:

i) the first antigen binding domain is the PD-L1 binding domain, the second antigen binding domain is the PD-L2 binding domain, and the third antigen binding domain is the CD28 binding domain, ii) the first antigen binding domain is the PD-L1 binding domain, the second antigen binding domain is the CD28 binding domain, and the third antigen binding domain is the PD-L2 binding domain, iii) the first antigen binding domain is the PD-L2 binding domain, the second antigen binding domain is the PD-L1 binding domain, and the third antigen binding domain is the CD28 binding domain, iv) the first antigen binding domain is the PD-L2 binding domain, the second antigen binding domain is the CD28 binding domain, and the third antigen binding domain is the PD-L1 binding domain, v) the first antigen binding domain is the CD28 binding domain, the second antigen binding domain is the PD-L1 binding domain, and the third antigen binding domain is the PD-L2 binding domain, or vi) the first antigen binding domain is the CD28 binding domain, the second antigen binding domain is the PD-L2 binding domain, and the third antigen binding domain is the PD-L1 binding domain.

In some embodiments, the CD28 binding domain comprises a variable heavy domain and variable light domain selected from the following: i) a variable heavy domain selected from any of the variable heavy domains in FIGS. 35, 36, 38, and a variable light domain selected from any of the variable light domains in FIGS. 35, 37 and 38, ii) a variable heavy domain and a variable light domain of a CD28 binding domain in FIG. 38, iii) a variable heavy domain selected from any of the variable heavy domains in FIGS. 41-74, and a variable light domain selected from any of the variable light domains in FIGS. 41-74, iv) a variable heavy domain and variable light domain of a CD28 binding domain in FIGS. 41-74, v) a variable heavy domain selected from any of the variable light domains in FIGS. 41-74, and a variable light domain having the amino acid sequence of SEQ ID NO:20 (FIG. 24), vi) a variable heavy domain and variable light domain of a CD28 binding domains in FIG. 81, and vii) a variable heavy domain selected from any of the variable heavy domains in FIG. 163, and a variable light domain of selected from any of the variable light domains in FIGS. 35 and 37, or a variable light domain having the amino acid sequence of SEQ ID NO:3239.

In some embodiments, the PD-L1 antigen binding domain comprises a variable heavy domain and a variable light domain selected from the following: i) a variable heavy domain selected from any of the variable heavy domains in FIGS. 17 and 19, and a variable light domain selected from any of the variable light domains in FIGS. 17 and 20, ii) a variable heavy domain and variable light domain of a PD-L1 binding domain in FIGS. 17 and 21, iii) a variable heavy domain selected from any of the variable heavy domains in FIGS. 25 and 26, and a variable light domain selected from any of the variable light domains in FIGS. 25 and 26, iv) a variable heavy domain and variable light domain of a PD-L1 binding domain in FIGS. 25 and 26, v) a variable heavy domain selected from any of the variable heavy domains in FIGS. 25 and 26, and a variable light domain having the amino acid sequence of SEQ ID NO:20 (FIG. 24), vi) a variable heavy domain and variable light domain of a PD-L1 binding domain in FIG. 28; and vii) a variable heavy domain selected from any of the variable heavy domains in FIG. 161, and a variable light domain having the amino acid sequence of SEQ ID NO:3239.

In some embodiments, the PD-L2 antigen binding domain comprises a variable heavy domain and a variable light domain selected from the following: i) a variable heavy domain selected from any of the variable heavy domains in FIGS. 29-32, and a variable light domain selected from any of the variable light domains in in FIGS. 29-32, ii) a variable heavy domain and variable light domain of a PD-L2 binding domain in FIGS. 29-32, iii) a variable heavy domain selected from any of the variable heavy domains in FIGS. 29-32, and a variable light domain having the amino acid sequence of SEQ ID NO:20 (FIG. 24), iv) a variable heavy domain and variable light domain of a PD-L2 binding domain in FIG. 34, and v) a variable heavy domain selected from any of the variable heavy domains in FIG. 162, and a variable light domain having the amino acid sequence of SEQ ID NO:3239.

In some embodiments, the first and second Fc domains are variant Fc domains. In certain embodiments, the first and second Fc domains comprise a set of heterodimerization skew variants selected from the group consisting of S364K/E357Q:L368D/K370S; S364K: L368D/K370S; S364K:L368E/K370S; D401K:T411E/K360E/Q362E; and T366W:T366S/L368A/Y407V, wherein numbering is according to EU numbering. In some embodiments, the first and second Fc domains comprise heterodimerization skew variants S364K/E357Q:L368D/K370S.

In some embodiments, the first and second Fc domains each comprise one or more ablation variants. In some embodiments, the one or more ablation variants are E233P/L234V/L235A/G236del/S267K, wherein numbering is according to EU numbering.

In some embodiments, one of the first or second monomer comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the first Fc domain comprises amino acid variants S364K/E357Q/E233P/L234V/L235A/G236del/S267K, the CH1-hinge-CH2-CH3 of the second monomer comprises amino acid variants L368D/K370S/N208D/Q295E/N384D/Q418E/N421D/E233P/L234V/L235A/G236del/S267K, and numbering is according to EU numbering.

In some embodiments, the scFv linker is a charged scFv linker having the amino acid sequence (GKPGS)₄. In some embodiments, the first and second Fc domains each further comprise amino acid variants 428/434S.

In one aspect, provided herein are novel 1+1+1 mAb-scFv format antibodies (FIG. 83C). The 1+1+1 mAb-scFv format antibody generally includes a) a first monomer, b) a second monomer, and c) first and second common light chain. The first monomer includes from N-terminal to C-terminal, VH1-CH1-hinge-CH2-CH3-domain linker-scFv, wherein VH1 is a first variable heavy domain, and CH2-CH3 is a first Fc domain. The second monomer includes from N-terminal to C-terminal, a VH2-CH1-hinge-CH2-CH3, wherein VH2 is a second variable heavy domain and CH2-CH3 is a second Fc domain. The first and second common light chains each include from N-terminal to C-terminal, VL1-CL, wherein VL1 is a first variable light domain and CL is a constant light domain. Further, the scFv comprises a third VH domain (VH3), a scFv linker, and a second variable light domain (VL2). In this format, the first variable heavy domain and the first variable light domain of the first common light chain form a first antigen binding domain, the second variable heavy domain and the first variable light domain of the second common light chain form a second antigen binding domain, and the third variable heavy domain and second variable light domain form a third antigen binding domain.

In some embodiments, the antibody is a trispecific antibody that binds PD-L1, PD-L2, and CD28, and each of the first antigen binding domain, second antigen binding domain, and third antigen binding domain is selected from the group consisting of a PD-L1 antigen binding domain, a PD-L2 antigen binding domain, and a CD 28 binding domain.

In some embodiments, the first antigen binding domain, the second antigen binding domain, and the third antigen binding domain are selected from the following:

i) the first antigen binding domain is the PD-L1 binding domain, the second antigen binding domain is the PD-L2 binding domain, and the third antigen binding domain is the CD28 binding domain, ii) the first antigen binding domain is the PD-L1 binding domain, the second antigen binding domain is the CD28 binding domain, and the third antigen binding domain is the PD-L2 binding domain, iii) the first antigen binding domain is the PD-L2 binding domain, the second antigen binding domain is the PD-L1 binding domain, and the third antigen binding domain is the CD28 binding domain, iv) the first antigen binding domain is the PD-L2 binding domain, the second antigen binding domain is the CD28 binding domain, and the third antigen binding domain is the PD-L1 binding domain, v) the first antigen binding domain is the CD28 binding domain, the second antigen binding domain is the PD-L1 binding domain, and the third antigen binding domain is the PD-L2 binding domain, or vi) the first antigen binding domain is the CD28 binding domain, the second antigen binding domain is the PD-L2 binding domain, and the third antigen binding domain is the PD-L1 binding domain.

In some embodiments, the CD28 binding domain comprises a variable heavy domain and variable light domain selected from the following: i) a variable heavy domain selected from any of the variable heavy domains in FIGS. 35, 36, 38, and a variable light domain selected from any of the variable light domains in FIGS. 35, 37 and 38, ii) a variable heavy domain and a variable light domain of a CD28 binding domain in FIG. 38, iii) a variable heavy domain selected from any of the variable heavy domains in FIGS. 41-74, and a variable light domain selected from any of the variable light domains in FIGS. 41-74, iv) a variable heavy domain and variable light domain of a CD28 binding domain in FIGS. 41-74, v) a variable heavy domain selected from any of the variable light domains in FIGS. 41-74, and a variable light domain having the amino acid sequence of SEQ ID NO:20 (FIG. 24), vi) a variable heavy domain and variable light domain of a CD28 binding domains in FIG. 81, and vii) a variable heavy domain selected from any of the variable heavy domains in FIG. 163, and a variable light domain of selected from any of the variable light domains in FIGS. 35 and 37, or a variable light domain having the amino acid sequence of SEQ ID NO:3239.

In some embodiments, the PD-L1 antigen binding domain comprises a variable heavy domain and a variable light domain selected from the following: i) a variable heavy domain selected from any of the variable heavy domains in FIGS. 17 and 19, and a variable light domain selected from any of the variable light domains in FIGS. 17 and 20, ii) a variable heavy domain and variable light domain of a PD-L1 binding domain in FIGS. 17 and 21, iii) a variable heavy domain selected from any of the variable heavy domains in FIGS. 25 and 26, and a variable light domain selected from any of the variable light domains in FIGS. 25 and 26, iv) a variable heavy domain and variable light domain of a PD-L1 binding domain in FIGS. 25 and 26, v) a variable heavy domain selected from any of the variable heavy domains in FIGS. 25 and 26, and a variable light domain having the amino acid sequence of SEQ ID NO:20 (FIG. 24), vi) a variable heavy domain and variable light domain of a PD-L1 binding domain in FIG. 28; and vii) a variable heavy domain selected from any of the variable heavy domains in FIG. 161, and a variable light domain having the amino acid sequence of SEQ ID NO:3239.

In some embodiments, the PD-L2 antigen binding domain comprises a variable heavy domain and a variable light domain selected from the following: i) a variable heavy domain selected from any of the variable heavy domains in FIGS. 29-32, and a variable light domain selected from any of the variable light domains in in FIGS. 29-32, ii) a variable heavy domain and variable light domain of a PD-L2 binding domain in FIGS. 29-32, iii) a variable heavy domain selected from any of the variable heavy domains in FIGS. 29-32, and a variable light domain having the amino acid sequence of SEQ ID NO:20 (FIG. 24), iv) a variable heavy domain and variable light domain of a PD-L2 binding domain in FIG. 34, and v) a variable heavy domain selected from any of the variable heavy domains in FIG. 162, and a variable light domain having the amino acid sequence of SEQ ID NO:3239.

In some embodiments, the first and second Fc domains are variant Fc domains. In certain embodiments, the first and second Fc domains comprise a set of heterodimerization skew variants selected from the group consisting of S364K/E357Q:L368D/K370S; S364K: L368D/K370S; S364K:L368E/K370S; D401K:T411E/K360E/Q362E; and T366W:T366S/L368A/Y407V, wherein numbering is according to EU numbering. In some embodiments, the first and second Fc domains comprise heterodimerization skew variants S364K/E357Q:L368D/K370S.

In some embodiments, the first and second Fc domains each comprise one or more ablation variants. In some embodiments, the one or more ablation variants are E233P/L234V/L235A/G236del/S267K, wherein numbering is according to EU numbering.

In some embodiments, one of the first or second monomer comprises one or more pI variants. In some embodiments, the CH1-hinge-CH2-CH3 of the second monomer comprises pI variants N208D/Q295E/N384D/Q418E/N421D, wherein numbering is according to EU numbering.

In some embodiments, the first Fe domain comprises amino acid variants S364K/E357Q/E233P/L234V/L235A/G236del/S267K, the CH1-hinge-CH2-CH3 of the second monomer comprises amino acid variants L368D/K370S/N208D/Q295E/N384D/Q418E/N421D/E233P/L234V/L235A/G236del/S267K, and numbering is according to EU numbering.

In some embodiments, the scFv linker is a charged scFv linker having the amino acid sequence (GKPGS)₄. In some embodiments, the first and second Fc domains each further comprise amino acid variants 428/434S.

In another aspect, provided herein is a method of treating a cancer comprising administering to a patient in need thereof a subject antibody disclosed herein or a pharmaceutical composition comprising the subject antibody and a pharmaceutically acceptable carrier. In some embodiments, the method further comprises administering an anti-CD3×tumor target antigen (TTA) bispecific antibody to the patient.

Also provided herein are nucleic acid compositions encoding the compositions and antibodies provided herein, expression vectors that include such nucleic acids, and host cells that include the nucleic acids and expression vectors.

In another aspect, provided herein is a method of treating a cancer that includes administering to a patient in need thereof one of the subject antibodies provided herein. In some embodiments, the method further includes administering an anti-CD3×tumor target antigen bispecific antibody to the patient.

In another aspect, provided herein is a composition comprising a CD28 antigen binding domain, wherein the CD28 binding domain comprises: a) a variable heavy domain having the vhCDR1-3 of a variable heavy domain in FIG. 163; and b) a variable light domain having the vlCDR1-3 of a variable light domain in FIGS. 35 and 37, or a variable light domain having the amino acid sequence of SEQ ID NO:3239.

In another aspect provided herein is a CD28 antigen binding domain, wherein the CD28 binding domain comprises: a) a variable heavy domain having at least 85%, 90%, 95%, or 99% sequence identity to a variable heavy domain in FIG. 163; and b) a variable light domain having at least 85%, 90%, 95%, or 99% sequence identity to a variable light domain in FIGS. 35 and 37, or a variable light domain having the amino acid sequence of SEQ ID NO:3239. In some embodiments, the variable heavy domain has an amino acid sequence of a variable heavy domain in FIG. 163; and the variable light domain has an amino acid sequence of a variable light domain in FIGS. 35 and 37, or a variable light domain having the amino acid sequence of SEQ ID NO:3239.

In another aspect, provided herein is a composition comprising a PD-L1 antigen binding domain, wherein the PD-L1 binding domain comprises: a) a variable heavy domain having the vhCDR1-3 of a variable heavy domain in FIG. 161; and b) a variable light domain having the vlCDR1-3 of a variable light domain having the amino acid sequence of SEQ ID NO:3239.

In another aspect, provided herein is a composition comprising a PD-L1 antigen binding domain, wherein the PD-L1 binding domain comprises: a) a variable heavy domain having at least 85%, 90%, 95%, or 99% sequence identity to a variable heavy domain in FIG. 161; and b) a variable light domain having at least 85%, 90%, 95%, or 99% sequence identity to a variable light domain having the amino acid sequence of SEQ ID NO:3239. In some embodiments, the variable heavy domain has an amino acid sequence of a variable heavy domain in FIG. 161; and the variable light domain has an amino acid sequence of a variable light domain having the amino acid sequence of SEQ ID NO:3239.

In another aspect, provided herein is a composition comprising a PD-L2 antigen binding domain, wherein the PD-L1 binding domain comprises: a) a variable heavy domain having the vhCDR1-3 of a variable heavy domain in FIG. 162; and b) a variable light domain having the vlCDR1-3 of a variable light domain having the amino acid sequence of SEQ ID NO:3239.

In another aspect, provided herein is a composition comprising a PD-L2 antigen binding domain, wherein the PD-L1 binding domain comprises: a) a variable heavy domain having at least 85% sequence identity to a variable heavy domain in FIG. 162; and b) a variable light domain having at least 85% sequence identity to a variable light domain having the amino acid sequence of SEQ ID NO:3239. In some embodiments, the variable heavy domain has an amino acid sequence of a variable heavy domain in FIG. 162; and the variable light domain has an amino acid sequence of a variable light domain having the amino acid sequence of SEQ ID NO:3239.

### BRIEF DESCRIPTION OF THE DRAWINGS

FIG. 1 depicts the sequences for human, mouse, and cynomolgus PDL1. Such PDL1 are useful for the development of cross-reactive PDL1 antigen binding domains for ease of clinical development.

FIG. 2 depicts the sequences for human, mouse, and cynomolgus PDL2. Such PDL2 are useful for the development of cross-reactive PDL2 antigen binding domains for ease of clinical development.

FIGS. 3A-3B depict the sequences for human, mouse, and cynomolgus CD28. Such CD28 are useful for the development of cross-reactive CD28 antigen binding domains for ease of clinical development.

FIGS. 4A-4F depict useful pairs of heterodimerization variant sets (including skew and pI variants). In FIG. 4F, there are variants for which there are no corresponding “monomer 2” variants. Such variants are pI variants that can be used alone on either monomer of a bispecific antibody (e.g., αPD-L1×αCD28 bsAb) or a trispecific antibody (e.g., PDL1×PDL2×CD28 triAb), or included, for example, on the non-scFv side of a format that utilizes an scFv having an appropriately charged scFv linker as a component on the second monomer (suitable charged linkers are shown in FIG. 7). Heterodimer yield (%) and CH3 Tₘ (° C.) of preferred Fc heterodimerization variants were previously described (see, e.g., FIG. 8 of U.S. Patent Application No. 2019/0248898).

FIG. 5 depicts a list of isosteric variant antibody constant regions and their respective substitutions. pI_(−) indicates lower pI variants, while pI_(+) indicates higher pI variants. These variants can be optionally and independently combined with other variants, including heterodimerization variants, outlined herein.

FIG. 6 depict useful ablation variants that ablate FcγR binding (also referred to as “knockouts” or “KO” variants). In some embodiments, such ablation variants are included in the Fc domain of both monomers of the subject antibody described herein. In other embodiments, the ablation variants are only included on only one variant Fc domain.

FIG. 7 depicts a number of charged scFv linkers that find use in increasing or decreasing the pI of the subject multimeric bispecific and trispecific antibodies that utilize one or more scFv as a component, as described herein (e.g., PDL1×CD28 bsAbs). The (+H) positive linker finds particular use herein, particularly with anti-CD28 VL and VH sequences shown herein. A single prior art scFv linker with a single charge is referenced as “Whitlow,” from Whitlow et al., Protein Engineering 6(8):989-995 (1993). It should be noted that this linker was used for reducing aggregation and enhancing proteolytic stability in scFvs. Such charged scFv linkers can be used in any of the subject antibody formats disclosed herein that include scFvs (e.g., 1+1 Fab-scFv-Fc, 2+1 Fab2-scFv-Fc formats, etc.).

FIGS. 8A-8B depicts a number of exemplary domain linkers. In some embodiments, these linkers find use linking a single-chain Fv to an Fc chain. In some embodiments, these linkers may be combined in any orientation. For example, a GGGGS linker may be combined with a “lower half hinge” linker at the N-terminus or at the C-terminus.

FIG. 9 shows a particularly useful embodiment of the heterodimeric Fc domains (i.e. CH2-CH3 in this embodiment) of the PDL1×CD28 and PDL2×CD28 bsAbs and PDL1×PDL2×CD28 triAbs of the invention.

FIG. 10 shows the sequences of several useful multimeric PDL1×CD28 and PDL2×CD28 bispecific antibodies (bsAbs) or PDL1×PDL2×CD28 trispecific antibodies (triAbs) backbones based on human IgG1, without the cytokine sequences. Heterodimeric Fc backbone 1 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 2 is based on human IgG1 (356E/358M allotype), and includes the L368D/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 3 is based on human IgG1 (356E/358M allotype), and includes the L368E/K370S skew variants and the Q295E/N384D/Q418E/N421D pI variants on a first heterodimeric Fc chain, the S364K skew variant on a second heterodimeric Fc chain, and the E233P/L234V/L235A/G236del/S267K ablation variants on both chains. Heterodimeric Fc backbone 4 is based on human IgG1 (356E/358M allotype), and includes the K360E/Q362E/T411E skew variants and the Q295E/N384D/Q418E/N421D pI variants on a first heterodimeric Fc chain, the D401K skew variant on a second heterodimeric Fc chain, and the E233P/L234V/L235A/G236del/S267K ablation variants on both chains. Heterodimeric Fc backbone 5 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants on both chains. Heterodimeric Fc backbone 6 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297A variant that removes glycosylation on both chains. Heterodimeric Fc backbone 7 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and N297S variant that removes glycosylation on both chains. Heterodimeric Fc backbone 8 is based on human IgG4, and includes the L368D/K370S skew variants and the Q295E/N384D/Q418E/N421D pI variants on a first heterodimeric Fc chain, the S364K/E357Q skew variants on a second heterodimeric Fc chain, and the S228P (according to EU numbering, S241P in Kabat) variant that ablates Fab arm exchange (as is known in the art) on both chains. Heterodimeric Fc backbone 9 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain. Heterodimeric Fc backbone 10 is based on human IgG2, and includes the L368D/K370S skew variants and the Q295E/N384D/Q418E/N421D pI variants on a first heterodimeric Fc chain, the S364K/E357Q skew variants on a second heterodimeric Fc chain, and the S267K ablation variant on both chains. Heterodimeric Fc backbone 11 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 12 is based on human IgG1 (356E/358M allotype), and includes the L368D/K370S skew variants on a first heterodimeric Fc chain, the S364K/E357Q skew variants and P217R/P229R/N276K pI variants on a second heterodimeric Fc chain, and the E233P/L234V/L235A/G236del/S267K ablation variants on both chains. Heterodimeric Fc backbone 13 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434S Xtend variants on both chains. Heterodimeric Fc backbone 14 is based on human IgG1 (356E/358M allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains. Heterodimeric Fc backbone 15 is based on human IgG1 (356D/358L allotype), and includes the L368D/K370S skew variants and the Q295E/N384D/Q418E/N421D pI variants on a first heterodimeric Fc chain, the S364K/E357Q skew variants on a second heterodimeric Fc chain, and the E233P/L234V/L235A/G236del/S267K ablation variants and M428L/N434A Xtend variants on both chains.

Included within each of these backbones are sequences that are 90, 95, 98 and 99% identical (as defined herein) to the recited sequences, and/or contain from 1, 2, 3, 4, 5, 6, 7, 8, 9 or 10 additional amino acid substitutions (as compared to the “parent” of the Figure, which, as will be appreciated by those in the art, already contain a number of amino acid modifications as compared to the parental human IgG1 (or IgG2 or IgG4, depending on the backbone). That is, the recited backbones may contain additional amino acid modifications (generally amino acid substitutions) in addition or as an alternative to the skew, pI and ablation variants contained within the backbones of this Figure. Additionally, the backbones depicted herein may include deletion of the C-terminal glycine (K446_) and/or lysine (K447_). The C-terminal glycine and/or lysine deletion may be intentionally engineered to reduce heterogeneity or in the context of certain bispecific formats, such as the mAb-scFv format. Additionally, C-terminal glycine and/or lysine deletion may occur naturally for example during production and storage. Additionally, these sequences may include the H435R/Y436F variant in either of monomer 1 or monomer 2 for facile purification.

FIG. 11 depicts illustrative sequences of backbone for use in the 2+1 mAb-scFv and 1+1+1 mAb-scFv formats. The format depicted here is based on heterodimeric Fc backbone 1 as depicted in Figure X, except further including G446_ on monomer 1 (−) and G446_/K447_ on monomer 2 (+). It should be noted that any of the additional backbones depicted in Figure X may be adapted for use in the 2+1 mAb-scFv format with or without including K447_ on one or both chains. It should be noted that these sequences may further include the M428L/N434S variants. Additionally, these sequences may include the H435R/Y436F variant in either of monomer 1 or monomer 2 for facile purification.

FIG. 12 depicts sequences for “CH1” that find use in embodiments of the bsAbs and triAbs of the invention.

FIG. 13 depicts sequences for “hinge” that find use in embodiments of bsAbs ant triAbs of the invention.

FIG. 14 depicts the constant domain of the cognate light chains that find use in the subject PDL1×CD28 or PDL2×CD28 bsAbs and PDL1×PDL2×CD28 triAbs that utilize a Fab binding domain.

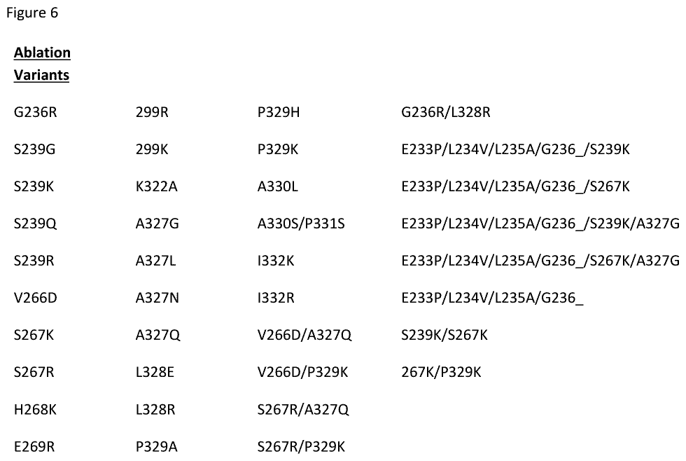

FIGS. 15A-15F depict sequences for exemplary anti-CD3 binding domains suitable for use in CD3 bispecific antibodies which may be combined with the CD28 bispecific or trispecific antibodies of the invention. The CDRs are underlined, the scFv linker is double underlined (in the sequences, the scFv linker is a positively charged scFv (GKPGS)4 linker (SEQ ID NO: 892), although as will be appreciated by those in the art, this linker can be replaced by other linkers, including uncharged or negatively charged linkers, some of which are depicted in FIG. 6), and the slashes indicate the border(s) of the variable domains. In addition, the naming convention illustrates the orientation of the scFv from N- to C-terminus. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 16 depict the sequences for illustrative αPSMA×αCD3 bsAbs in the 2+1 Fab2-scFv-Fc format and respectively comprising a H1.30_L1.47 anti-CD3 scFv (a.k.a. CD3 High [VHVL]) or a L1.47_H1.32 anti-CD3 scFv (a.k.a. CD3 High-Int #1 [VLVH]). CDRs are underlined and slashes indicate the border(s) between the variable regions and other chain components (e.g. constant region and domain linkers). It should be noted that the αPSMA×αCD3 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIG. 17 depicts the variable heavy and variable light chain sequences for 2G4, an exemplary humanized hybridoma-derived PDL1 binding domain, as well as the sequences for XENP25859, an anti-PDL1 mAb based on 2G4 and IgG1 backbone with E233P/L234V/L235A/G236del/S267K ablation variant, and XENP36627, a monovalent anti-PDL1 mAb based on 2G4. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 18 depicts PD1:PDL1 blockade (binding of PDL1-mFc fusion to PD1-transfected HEK293T cells) by anti-PDL1 clone 2G4 (XENP25859), a partial blocking anti-PDL1 (XENP25853), a non-blocking anti-PDL1 (XENP25858), and XENP24118 (a benchmark anti-PDL1 mAb based on avelumab).

FIG. 19 depicts the sequences for affinity-optimized variable heavy domains from anti-PDL1 clone 2G4. It should be noted that the variable heavy domains can be paired with any of the other 2G4 variable light domains depicted herein including SEQ ID NOs: 1467-1528 (e.g. 2G4_H1.12_L1.14 as utilized in XENP40706).

FIG. 20 depicts the sequences for affinity-optimized variable light domain from anti-PDL1 clone 2G4. It should be noted that the variable heavy domains can be paired with any of the other 2G4 variable heavy domains depicted herein including SEQ ID NOs: 1529-1599 (e.g. 2G4_H1.12_L1.14 as utilized in XENP40706).

FIG. 21 depicts the sequence for illustrative affinity-optimized 2G4 VH/VL pairs. It should be noted that these pairs may be formatted as Fabs or as scFvs.

FIG. 22 depicts consensus framework regions (FR) and complementarity determining regions (CDRs) (as in Kabat) for anti-PDL1 clone 2G4 variable heavy and variable light domain variants.

FIG. 23 depicts illustrative affinity-engineered 2G4 VH/VL pairs and their binding affinities in the context of scFvs (in the context of 1+1 Fab-scFv-Fc bsAb format).

FIG. 24 depicts A) sequence for the common variable light domain used in the humanized mice that were the basis of the PDL1, PDL2, and CD28 common light chain campaign (referred to as 6B1_L1). This variable light domain sequence may be paired with the variable heavy domains as depicted in FIGS. 25, 26, 29, 30, 31, 32, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, and 74.

FIG. 25 depicts novel PDL1 binding domain clone 13G1. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 26A-26D depicts novel PDL1 binding domain clone 13G7, alternative VH 13G7_H2, alternative VL, 13G7_L2, and illustrative affinity-engineered VH variants (additionally, it should be noted that the CDRs in the affinity-engineered VHs may be grafted onto alternative parental frameworks as described in Example 1B). It should be noted that both 13G7_H1 and 13G7_H2 and affinity-engineered 13G7 VH variants may additionally be paired with 6B1_L1 as depicted in FIG. 24. Additionally, consensus framework regions (FR) and complementarity determining regions (CDRs) for 13G7 VH are depicted. CDRs are underlined and slashes indicate the border(s) between the variable regions and constant domain. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 27 depicts dissociation constant (KD), association rate (ka), and dissociation rate (kd) of PDL1 antibodies generated by single-cell technology in mouse genetically engineered with complete human heavy chain variable domain combined with a human common light chain substitution.

FIG. 28 depicts the variable heavy and variable light chain sequences for additional PDL1 binding domains which find use in the PDL1×CD28 bsAbs and PDL1×PDL2×CD28 triAbs of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 29 depicts novel PDL2 binding domain clone 5C11, as well as alternative VH 5C11_H2. It should be noted that both 5C11_H1 and 5C11_H2 may additionally be paired with 6B1_L1 as depicted in FIG. 24. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 30 depicts novel PDL2 binding domain clone 8G2, as well as alternative VH 8G2_H2. It should be noted that both 8G2_H1 and 8G2_H2 may additionally be paired with 6B1_L1 as depicted in FIG. 24. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 31 depicts novel PDL2 binding domain clone 8G5, as well as alternative VH 8G5_H3. It should be noted that both 8G5_H1 and 8G5_H3 may additionally be paired with 6B1_L1 as depicted in FIG. 24. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 32A-32D depicts novel PDL2 binding domain clone 16G11, as well as alternative VH 16G11_H2 and 16G11_H3 and illustrative affinity-engineered VH variants (additionally, it should be noted that the CDRs in the affinity-engineered VHs may be grafted onto alternative parental frameworks as described in Example 1B). It should be noted that 16G11_H1, 16G11_H2, and 16G11_H3 and affinity-engineered 16G11 VH variants may additionally be paired with 6B1_L1 as depicted in FIG. 24. Additionally, consensus framework regions (FR) and complementarity determining regions (CDRs) for 16G11 VH are depicted. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 33 depicts dissociation constant (KD), association rate (ka), and dissociation rate (kd) of PDL2 antibodies generated by single-cell technology in mouse genetically engineered with complete human heavy chain variable domain combined with a human common light chain substitution.

FIG. 34 depicts the variable heavy and variable light chain sequences for additional PDL2 binding domains which find use in the PDL2×CD28 bsAbs and PDL1×PDL2×CD28 triAbs of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 35 depicts the variable heavy and variable light chain sequences for 1A7, an exemplary phage-derived CD28 binding domain that is not superagonistic. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 36 depicts the sequences for affinity-optimized variable heavy domains from anti-CD28 clone 1A7. It should be noted that the variable heavy domains can be paired with any of the other variable light domains depicted in FIGS. 35 and 37 including SEQ ID NOs: 2456-2524.

FIG. 37 depicts the sequences for affinity-optimized variable light domain from anti-CD28 clone 1A7. It should be noted that the variable light domains can be paired with any of the other variable light domains depicted in FIGS. 35 and 36 including SEQ ID NOs: 2525-2630.

FIGS. 38A-38C depicts the sequence for illustrative affinity-optimized 1A7 VH/VL pairs. It should be noted that these pairs may be formatted as Fabs or as scFvs. Additionally, in the scFv format, these pairs may be formatted in the VHVL orientation or the VLVH orientation.

FIGS. 39A-39B depicts consensus framework regions (FR) and complementarity determining regions (CDRs) (as in Kabat) for anti-CD28 clone 1A7 variable heavy and variable light domain variants.

FIG. 40 depicts illustrative affinity-engineered 1A7 VH/VL pairs and their binding affinities in the context of scFvs (in the context of 1+1 Fab-scFv-Fc bsAb format).

FIG. 41 depicts novel CD28 binding domain clone 1B1-5.88488, as well as the sequences for XENP41791, an anti-CD28 mAb based on 1B1-5.88488, and XENP42418, an anti-CD28 mAb utilizing the 1B1-5.88488 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 42 depicts novel CD28 binding domain clone 1B1-4.88488, as well as the sequences for XENP41792, an anti-CD28 mAb based on 1B1-4.88488, and XENP42419, an anti-CD28 mAb utilizing the 1B1-4.88488 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 43 depicts novel CD28 binding domain clone 1D8.88474, as well as the sequences for XENP41834, an anti-CD28 mAb based on 1D8.88474, and XENP42420, an anti-CD28 mAb utilizing the 1D8.88474 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 44 depicts novel CD28 binding domain clone 1G2-2.88474, as well as the sequences for XENP41846, an anti-CD28 mAb based on 1G2-2.88474, and XENP42421, an anti-CD28 mAb utilizing the 1G2-2.88474 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 45 depicts novel CD28 binding domain clone 2G5.88497, as well as the sequences for XENP41864, an anti-CD28 mAb based on 2G5.88497, and XENP42423, an anti-CD28 mAb utilizing the 2G5.88497 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 46 depicts novel CD28 binding domain clone 2A3.88497, as well as the sequences for XENP41882, an anti-CD28 mAb based on 2A3.88497, and XENP42425, an anti-CD28 mAb utilizing the 2A3.88497 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 47 depicts novel CD28 binding domain clone 1D9-3.83967, as well as the sequences for XENP41907, an anti-CD28 mAb based on 1D9-3.83967, and XENP42426, an anti-CD28 mAb utilizing the 1D9-3.83967 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 48 depicts novel CD28 binding domain clone 1C9-1.83967, as well as the sequences for XENP41927, an anti-CD28 mAb based on 1C9-1.83967, and XENP42429, an anti-CD28 mAb utilizing the 1C9-1.83967 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 49 depicts novel CD28 binding domain clone 1D10-4.83967, as well as the sequences for XENP41936, an anti-CD28 mAb based on 1D10-4.83967, and XENP42430, an anti-CD28 mAb utilizing the 1D10-4.83967 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 50 depicts novel CD28 binding domain clone 1A12.83967, as well as the sequences for XENP41957, an anti-CD28 mAb based on 1A12.83967. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 51 depicts novel CD28 binding domain clone 1B11.83967, as well as the sequences for XENP41949, an anti-CD28 mAb based on 1B11.83967. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 52 depicts novel CD28 binding domain clone 1D10-2.83967, as well as the sequences for XENP41935, an anti-CD28 mAb based on 1D10-2.83967. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 53 depicts novel CD28 binding domain clone 1D7.83967, as well as the sequences for XENP41904, an anti-CD28 mAb based on 1D7.83967. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 54 depicts novel CD28 binding domain clone 1D3.83967, as well as the sequences for XENP41901, an anti-CD28 mAb based on 1D3.83967. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 55 depicts novel CD28 binding domain clone 2B10.88497, as well as the sequences for XENP41891, an anti-CD28 mAb based on 2B10.88497. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 56 depicts novel CD28 binding domain clone 2B9.88497, as well as the sequences for XENP41890, an anti-CD28 mAb based on 2B9.88497. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 57 depicts novel CD28 binding domain clone 2B8.88497, as well as the sequences for XENP41889, an anti-CD28 mAb based on 2B8.88497. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 58 depicts novel CD28 binding domain clone 1G6-1.83967, as well as the sequences for XENP41877, an anti-CD28 mAb based on 1G6-1.83967. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 59 depicts novel CD28 binding domain clone 1C7.88474, as well as the sequences for XENP41874, an anti-CD28 mAb based on 1C7.88474. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 60 depicts novel CD28 binding domain clone 1A5-2.88474, as well as the sequences for XENP41869, an anti-CD28 mAb based on 1A5-2.88474. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 61 depicts novel CD28 binding domain clone 1A2.88474, as well as the sequences for XENP41868, an anti-CD28 mAb based on 1A2.88474. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 62 depicts novel CD28 binding domain clone 2F5.88497, as well as the sequences for XENP41860, an anti-CD28 mAb based on 2F5.88497. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 63 depicts novel CD28 binding domain clone 2E9.88497, as well as the sequences for XENP41858, an anti-CD28 mAb based on 2E9.88497. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 64A-64C depicts novel CD28 binding domain clone 1A3.88474, as well as the sequences for XENP41849, an anti-CD28 mAb based on 1A3.88474 and illustrative affinity-engineered VH variants. Additionally, consensus framework regions (FR) and complementarity determining regions (CDRs) for 1A3.88474 VH are depicted. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 65 depicts novel CD28 binding domain clone 1H11.88474, as well as the sequences for XENP41816, an anti-CD28 mAb based on 1H11.88474. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 66 depicts novel CD28 binding domain clone 1G1-1.88474, as well as the sequences for XENP41807, an anti-CD28 mAb based on 1G1-1.88474. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 67 depicts novel CD28 binding domain clone 1E6-1.88474, as well as the sequences for XENP41802, an anti-CD28 mAb based on 1E6-1.88474. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 68 depicts novel CD28 binding domain clone 1A1-5.88488, as well as the sequences for XENP41797, an anti-CD28 mAb based on 1A1-5.88488. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 69 depicts novel CD28 binding domain clone 1E2-5.88488, as well as the sequences for XENP41781, an anti-CD28 mAb based on 1E2-5.88488. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 70 depicts novel CD28 binding domain clone 2F6.88497, as well as the sequences for XENP41861, an anti-CD28 mAb based on 2F6.88497, and XENP42422, an anti-CD28 mAb utilizing the 2F6.88497 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 71 depicts novel CD28 binding domain clone 1H2.83967, as well as the sequences for XENP41880, an anti-CD28 mAb based on 1H2.83967, and XENP42424, an anti-CD28 mAb utilizing the 1H2.83967 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 72 depicts novel CD28 binding domain clone 1E4-3.83967, as well as the sequences for XENP41909, an anti-CD28 mAb based on 1E4-3.83967, and XENP42427, an anti-CD28 mAb utilizing the 1E4-3.83967 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 73 depicts novel CD28 binding domain clone 1C2-2.83967, as well as the sequences for XENP41918, an anti-CD28 mAb based on 1C2-2.83967, and XENP42428, an anti-CD28 mAb utilizing the 1C2-2.83967 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 74 depicts novel CD28 binding domain clone 1A11.83967, as well as the sequences for XENP41956, an anti-CD28 mAb based on 1A11.83967, and XENP42431, an anti-CD28 mAb utilizing the 1A11.83967 VH and the 6B1 common VL. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIG. 75 depicts maximum BLI-response of binding by CD28 mAbs based on single-cell technology derived binding domains (WT sequences in comparison to VH paired with 6B1_L1) to CD28 antigen. The data show that most of the binding domains retain CD28 binding when paired with 6B1_L1 VL; however, VH from several of the clones (e.g. 1B1-4.88488, 2G5.88497 and 1D10-4.83967) demonstrated diminished CD28 binding when paired with 6B1_L1.

FIG. 76 depicts maximum BLI-response of binding by CD28 mAbs based on additional single-cell technology derived binding domains to CD28 antigen.

FIG. 77 depicts binding to Jurkat cells by CD28 mAbs based on single-cell technology derived binding domains.

FIG. 78 depicts binding to Jurkat cells by CD28 mAbs based on single-cell technology derived binding domains (WT sequences in comparison to VH paired with 6B1_L1 common light chain).

FIG. 79 depicts induction of IL2 secretion from CD3 stimulated (100 ng/mL OKT3) purified T cells by CD28 mAbs based on single-cell technology derived binding domains.

FIG. 80 depicts induction of IL2 secretion from CD3 stimulated (100 ng/mL OKT3) purified T cells by CD28 mAbs based on single-cell technology derived binding domains (WT sequences in comparison to VH paired with 6B1_L1 common light chain).

FIG. 81 depicts the variable heavy and variable light chain sequences for additional CD28 binding domains which find use in the PDL1×CD28 and PDL2×CD28 bsAbs and PDL1×PDL2×CD28 triAbs of the invention. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 82A-82M depicts bispecific formats of the present invention. FIG. 82A depicts the “1+1 Fab-scFv-Fc” format, with a first Fab arm binding a first antigen and a second scFv arm binding second antigen. The 1+1 Fab-scFv-Fc format comprises a first monomer comprising a first heavy chain variable region (VH1) covalently attached to the N-terminus of a first heterodimeric Fc backbone (optionally via a linker), a second monomer comprising a single-chain Fv covalently attached to the N-terminus of a second corresponding heterodimeric Fc backbone (optionally via a linker), and a third monomer comprising a light chain variable region covalently to a light chain constant domain, wherein the light chain variable region is complementary to the VH1. FIG. 82B depicts the “2+1 Fab2-scFv-Fc” format, with a first Fab arm and a second Fab-scFv arm, wherein the Fab binds a first antigen and the scFv binds second antigen. The 2+1 Fab2-scFv-Fc format comprises a first monomer comprising a first heavy chain variable region (VH1) covalently attached to the N-terminus of a first heterodimeric Fc backbone (optionally via a linker), a second monomer comprising the VH1 covalently attached (optionally via a linker) to a single-chain Fv covalently attached (optionally via a linker) to the N-terminus of a second corresponding heterodimeric Fc backbone, and a third monomer comprising a light chain variable region covalently to a light chain constant domain, wherein the light chain variable region is complementary to the VH1. FIG. 82C depicts the “1+1 Common Light Chain” or “1+1 CLC” format, with a first Fc comprising a first Fab arm binding a first antigen and a second Fc comprising a second Fab arm binding second antigen. The 1+1 CLC format comprises a first monomer comprising VH1-CH1-hinge-CH2-CH3, a second monomer comprising VH2-CH1-hinge-CH2-CH3, and a third monomer comprising VL-CL. The VL pairs with the VH1 to form a binding domain with a first antigen binding specificity; and the VL pairs with the VH2 to form a binding domain with a second antigen binding specificity. FIG. 82D depicts the “2+1 Common Light Chain” or “2+1 CLC” format, with a first Fc comprising 2 Fab arms each binding a first antigen and a second Fc comprising 1 Fab arm binding a second antigen. The 2+1 CLC format comprises a first monomer comprising VH1-CH1-hinge-VH1-CH1-hinge-CH2-CH3, a second monomer comprising VH2-CH1-hinge-CH2-CH3, and a third monomer comprising VL-CL. The VL pairs with the first and second VH1 to form binding domains with a first antigen binding specificity; and the VL pairs with the VH2 to form a binding domain with a second antigen binding specificity. FIG. 82E depicts the “2+1 mAb-scFv” format, with a first Fc comprising an N-terminal Fab arm binding a first antigen and a second Fc comprising an N-terminal Fab arm binding the first antigen and a C-terminal scFv binding a second antigen. The 2+1 mAb-scFv format comprises a first monomer comprising VH1-CH1-hinge-CH2-CH3, a second monomer comprising VH1-CH1-hinge-CH2-CH3-scFv, and a third monomer comprising VL-CL. The VL pairs with the first and second VH1 to form binding domains with binding specificity for the first antigen. Additional bispecific formats include F) dual scFv, G) one-arm scFv-mAb, H) scFv-mAb, I) bispecific mAb, J) one-arm central-scFv, K) mAb-Fv, L) central-Fv, and M) trident.

FIGS. 83A-83D depicts trispecific formats of the present invention. FIG. 83A depicts the “1+1+1 stackFab2-scFv-Fc” format which comprises a first monomer comprising, from N-terminal to C-terminal VH1-CH1-linker-VH2-CH1-hinge-CH2-CH3 wherein CH2-CH3 is a first heterodimeric Fc domain; a second monomer comprising, from N-terminal to C-terminal scFv-linker-CH2-CH3 wherein CH2-CH3 is a second heterodimeric Fc domain complementary to the first heterodimeric Fc domain and wherein the scFv has a first antigen specificity; and a third monomer that is a common light chain comprising, from N-terminal to C-terminal VL-CL wherein the VL pairs with VH1 to form an antigen binding domain having a second antigen specificity and with VH2 to form an antigen binding domain having a third antigen specificity. FIG. 83D depicts the “1+1+1 Fab-(Fab-scFv)-Fc format) which comprises a first monomer that is a heavy chain comprising, from N-terminal to C-terminal VH1-CH1-hinge-CH2-CH3 wherein CH2-CH3 is a first heterodimeric Fc domain; a second monomer comprising, from N-terminal to C-terminal VH2-CH1-linker-scFv-linker-CH2-CH3 wherein CH2-CH3 is a second heterodimeric Fc domain complementary to the first heterodimeric Fc domain and wherein the scFv has a first antigen specificity; and a third monomer that is a common light chain comprising, from N-terminal to C-terminal VL-CL wherein the VL pairs with VH1 to form an antigen binding domain having a second antigen specificity and with VH2 to form an antigen binding domain having a third antigen specificity. FIG. 83C depicts the “1+1+1 mAb-scFv” format which comprises a first monomer that is a heavy chain comprising, from N-terminal to C-terminal VH1-CH1-hinge-CH2-CH3 wherein CH2-CH3 is a first heterodimeric Fc domain; a second monomer comprising, from N-terminal to C-terminal VH2-CH1-hinge-CH2-CH3-linker-scFv wherein CH2-CH3 is a second heterodimeric Fc domain complementary to the first heterodimeric Fc domain and wherein the scFv has a first antigen specificity; and a third monomer that is a common light chain comprising, from N-terminal to C-terminal VL-CL wherein the VL pairs with VH1 to form an antigen binding domain having a second antigen specificity and with VH2 to form an antigen binding domain having a third antigen specificity. FIG. 83D depicts the “1+1+1 stackFab2-Fab-Fc” format which comprises a first monomer that is a heavy chain comprising, from N-terminal to C-terminal VH1-CH1-hinge-CH2-CH3 wherein CH2-CH3 is a first heterodimeric Fe domain; a second monomer comprising, from N-terminal to C-terminal VH2-CH1-linker-VH3-CH1-hinge-CH2-CH3 wherein CH2-CH3 is a second heterodimeric Fc domain complementary to the first heterodimeric Fc domain; and a third monomer that is a common light chain comprising, from N-terminal to C-terminal VL-CL wherein the VL pairs with VH1 to form an antigen binding domain having a first antigen specificity, with VH2 to form an antigen binding domain having a second antigen specificity, and with VH3 to form an antigen binding domain having a third antigen specificity.

FIGS. 84A-84T depict the sequences for illustrative PDL1×CD28 bsAbs in the 1+1 Fab-scFv-Fc format. CDRs are underlined and slashes indicate the border(s) between the variable regions, Fc regions, and constant domains. It should be noted that the αPD-L1×αCD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

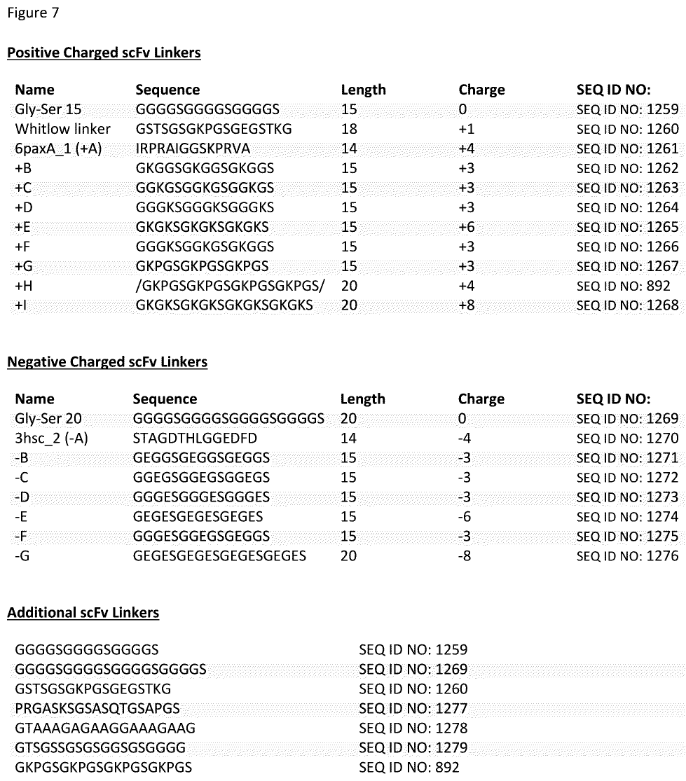

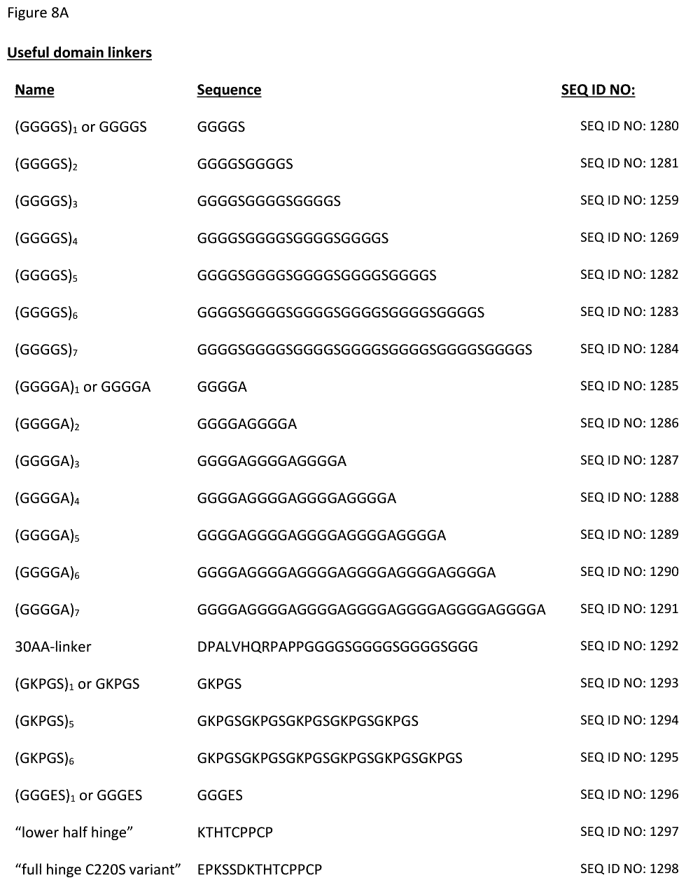

FIG. 85 depicts the sequences for illustrative αPD-L1×αCD28 bsAbs in the 2+1 Fab2-scFv-Fc format. CDRs are underlined and slashes indicate the border(s) between the variable regions, Fc regions, and constant domains. The scFv domain has orientation (N- to C-terminus) of VH-scFv linker-VL, although this can be reversed. It should be noted that the scFv domain sequences includes as the scFv linker between the variable heavy and variable light region the sequence GKPGSGKPGSGKPGSGKPGS (SEQ ID NO:XX); however, this linker can be replaced with any of the scFv linkers in FIG. 6. It should also be noted that the Chain 2 sequences include as the domain linker between the C-terminus of the scFv and the N-terminus of the CH2 domain the sequence GGGGSGGGGSKTHTCPPCP (SEQ ID NO:XX), which is a “flex half hinge” domain linker; however, this linker can be replaced with any of the “useful domain linkers” of FIG. 7. It should be noted that the αPD-L1×αCD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIG. 86 depicts the sequences for illustrative PDL1×CD28 bsAbs in the 2+1 mAb-scFv format. CDRs are underlined and slashes indicate the border(s) between the variable regions, linkers, Fc regions, and constant domains. The scFv domain has orientation (N- to C-terminus) of V_{H}-scFv linker-V_{L}, although this can be reversed. It should be noted that the Chain 2 sequences include as a domain linker the sequence GKPGSGKPGSGKPGSGKPGS (SEQ ID NO:XX); however, this linker can be replaced with any domain linker include any of the “useful domain linkers” of FIG. 6. It should be noted that the αPDL1×αCD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which results in longer half-life in serum.

FIGS. 87A-87B depict the sequences for illustrative PDL2×CD28 bsAbs in the 1+1 Fab-scFv-Fc format. CDRs are underlined and slashes indicate the border(s) between the variable regions, Fc regions, and constant domains. It should be noted that the PDL2×CD28 bsAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

FIGS. 88A-88M depict the sequences for illustrative PDL1×PDL2×CD28 triAbs in the 1+1+1 stackFab2-scFv-Fc format. CDRs are underlined and slashes indicate the border(s) between the variable regions and other domains (e.g. constant domains). It should be noted that the PDL1×PDL2×CD28 triAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

FIGS. 89A-89B depict the sequences for illustrative PDL1×PDL2×CD28 triAbs in the 1+1+1 Fab-(Fab-scFv)-Fc format. CDRs are underlined and slashes indicate the border(s) between the variable regions and other domains (e.g. constant domains). It should be noted that the PDL1×PDL2×CD28 triAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

FIG. 90 depict the sequences for illustrative PDL1×PDL2×CD28 triAbs in the 1+1+1 mAb-scFv format. CDRs are underlined and slashes indicate the border(s) between the variable regions and other domains (e.g. constant domains). It should be noted that the PDL1×PDL2×CD28 triAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

FIGS. 91A-91P depict the sequences for illustrative PDL1×PDL2×CD28 triAbs in the 1+1+1 stackFab2-Fab-Fc format. CDRs are underlined and slashes indicate the border(s) between the variable regions and other domains (e.g. constant domains). It should be noted that the PDL1×PDL2×CD28 triAbs can utilize variable region, Fc region, and constant domain sequences that are 90, 95, 98 and 99% identical (as defined herein), and/or contain from 1, 2, 3, 4, 5, 6, 7, 8, 9 or 10 amino acid substitutions. In addition, each sequence outlined herein can include or exclude the M428L/N434S variants in one or preferably both Fc domains, which including M428L/N434S results in longer half-life in serum.

FIG. 92 depicts A) classic T cell/APC interaction and B) replication of the classic T cell/APC interaction by combining CD3 bispecific antibodies with CD28 bispecific antibodies. In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells. In contrast in treatment with CD3 bispecifics, only the first signal is provided. The CD28 signal may be provided by a CD28 bispecific with the idea to promote activation and proliferation through CD28 costimulation.

FIG. 93 depicts the introduction of CD28 signaling by a CD28 bispecific antibody and mitigation of any checkpoint mediated repression of the added CD28 signal by checkpoint blockade (e.g. PD-1 blockade).

FIG. 94 depicts that PDL1×CD28 bispecific antibodies provide Signal 2 while advantageously further enabling blockade of PDL1:PD1 interaction. Although not depicted, PDL2×CD28 bispecific antibodies may similarly provide Signal 2 while advantageously further enabling blockade of PDL2:PD1 interaction.

FIG. 95 depicts the sequences for XENP24118, a PDL1 mAb based on avelumab and IgG1 backbone with E233P/L234V/L235A/G236del/S267K ablation variant. CDRs are underlined and slashes indicated border(s) between the variable region and constant domain.

FIG. 96 depicts expansion of CMV+ T cells following incubation of NLV-loaded MDA-MB-231 cancer cells with CD3+ T cells purified from a CMV+ donor and either αPD-L1 antibody XENP24118 or αPD-L1×αCD28 bsAb XENP34963. αPD-L1×αCD28 bsAb XENP34963 significantly enhanced T cell expansion in comparison to PD-L1 blockade alone.

FIG. 97 depicts induction of A) TL-2 secretion, B) IFNγ secretion, and C) CD3+ T cell expansion by 1 μg/ml αB7H3×αCD3 bsAb in combination with αPD-L1 mAb XENP24118 or in combination with αPD-L1×αCD28 bsAb XENP34963. αPD-L1×αCD28 bsAb enhances activity of a CD3 bsAb T cell engager.

FIG. 98 depicts cell kill over time following incubation of LNCaP cancer cells (PSMA+) with CD3+ T cells at a 1:1 effector:target ratio and illustrative CD3 bispecific (αPSMA×αCD3 XENP32220) alone or in combination with XENP36233. The data show that XENP32220 enhanced cell kill in comparison to incubation of cancer and T cells alone; however, addition of αPD-L1×αCD28 overcomes cancer cell resistance to the CD3 bispecific and further enhances cell kill.

FIG. 99 depicts group median change in tumor volume (as determined by caliper measurement; baseline corrected) A) over time (in days after first dose) and B) Day 20 (after first dose) in MC38 (engineered to stably expressing human PD-L1) and huPBMC-engrafted human CD28 knock-in mice dosed with 5 mg/kg αPD-L1 mAb XENP24118, 8.3 mg/kg αPD-L1×αCD28 bsAb XENP34963, 6 mg/kg αPD-L1×αCD28 XENP34961, or PBS control.

FIG. 100 depicts induction of A) IL-2 and B) IFNγ release by anti-PDL1 clone 2G4 (XENP25859), a partial blocking anti-PDL1 (XENP25853), a non-blocking anti-PDL1 (XENP25858), and XENP24118 (a benchmark anti-PDL1 mAb based on avelumab). The data show that the partial blocking and non-blocking anti-PDL1 clones induced less cytokine release in comparison to anti-PDL1 clone 2G4.

FIG. 101 depicts blockade of PD1:PDL1 interaction during T cell:cancer cell interaction (as modeled by Jurkat-PD1 cells incubated with CHO-PDL1-CD80-αCD3 and CHO-PDL1-αCD3 cells) by αPDL1×αCD28 bsAbs having anti-PDL1 clone 2G4 (XENP36233), a partial blocking anti-PDL1 (XENP36232), a non-blocking anti-PDL1 (XENP26783), and XENP34963 (a benchmark bsAb with an anti-PDL1 arm based on avelumab). The data show that bsAbs comprising partial blocking and non-blocking anti-PDL1 clones induced less activity in comparison to bsAb comprising anti-PDL1 clone 2G4.

FIG. 102 depicts binding of αPDL1×αCD28 (XENP36233) to parental PDL1^{null} MC38 or MC38 cells transfected to express PDL1 with low or medium high antigen densities.

FIG. 103 depicts induction of IL-2 release by αPDL1×αCD28 (XENP36233) in the presence of parental PDL1^{null} HEK293T cells or HEK293T transfected to express PDL1 with medium or high antigen densities.

FIG. 104 depicts induction of cell kill by αPSMA×αCD3 alone or in combination with αPDL1×αCD28 XENP36233 in the presence of CD3+ T cells and PDL1^{null} 22Rv1 cell line at A) 10:1 E:T ratio and B) 1:1 E:T ratio. The data show that αPDL1×αCD28 bsAbs do not synergize with CD3 bsAbs on PDL1 negative cell lines such as 22Rv1.

FIG. 105 depicts serum concentration of XENP36764 over time in cynomolgus monkeys. The αPDL1×αCD28 bsAb exhibited favorable pharmacokinetics.

FIG. 106 depicts diagram of assumptions used in a mechanism-based PK/PD computer model.

FIG. 107 depicts predictions from the mechanism-based modeling suggesting A) linear PK at dose levels consistent with typical checkpoint inhibitor regimens, B) trimer formation in the tumor indicating costimulation, and C) consistent blockade of PDL1.

FIG. 108 depicts induction of IL-2 release by αPDL1×αCD28 bsAbs having affinity-engineered CD28 binding domains in the presence of αB7H3×αCD3 bsAb, CD3+ T cells, and A) MDA-MB-231 or B) DU145-NLR cells. The data show that increasing CD28 affinity leads to more potent and efficacious TL-2 secretion by αPDL1×αCD28 bsAbs.

FIG. 109 depicts induction of cell kill by αPDL1×αCD28 bsAbs having affinity-engineered CD28 binding domains in the presence of αB7H3×αCD3 bsAb, CD3+ T cells, and PDL1^{low} LnCAP cancer cells. The data show that increasing the affinity of CD28 increases targeting of PDL1^{low} cancer cells at low E:T of 1:1.

FIG. 110 depicts induction of cell kill by αPDL1×αCD28 bsAbs having affinity-engineered CD28 binding domains in the presence of αB7H3×αCD3 bsAb, CD3+ T cells, and PDL1^{med} DU145 cells. The data show that increasing the affinity of CD28 increases targeting of PDL1^{med} cancer cells at even lower E:T of 0.1:1.

FIG. 111 depicts change in tumor volume (as determined by caliper measurements; baseline corrected) in individual mouse over time and D) on Day 28 in hPDL1-MC38-engrafted hCD28 knock-in mice dosed with A) PBS control, B) monovalent αPDL1 mAb XENP36627, and C) XENP37261 having enhanced CD28 binding affinity.

FIG. 112 depicts induction of IL-2 release by αPDL1×αCD28 bsAbs having affinity-engineered PDL1 binding domains in the presence of αB7H3×αCD3 bsAb, CD3+ T cells, and DU145-NLR cells. The data show that increasing PDL1 affinity promotes TL-2 secretion.

FIG. 113 depicts induction of A) IL2 and B) IFNγ release by αPDL1 mAb XENP24118 and αPDL1×αCD28 bsAb XENP38514 having enhanced PDL1 binding in a DC:T cell MLR. The data show that αPDL1×αCD28 enhanced T cell/APC interaction.

FIG. 114 depicts PD1:PDL1 blockade (binding of PDL1-mFc fusion to PD1-transfected HEK293T cells) by αPDL1×αCD28 bsAbs having affinity-engineered PDL1 binding domains. The data show the αPDL1×αCD28 bsAbs can block interaction between PD1 and PDL1.

FIG. 115 depicts induction of IL-2 release by αPDL1×αCD28 bsAbs having affinity-engineered CD28 binding domains and affinity-engineered PDL1 binding domains in the presence of SEB-stimulated PBMCs. The data show that increasing PDL1 affinity promotes TL-2 secretion.

FIG. 116 depicts induction of IL-2 release by αPDL1×αCD28 bsAbs having affinity-engineered CD28 binding domains and affinity-engineered PDL1 binding domains in the presence of CD3+ enriched T cells, MDA-MB0231 transfected to express αCD3 scFv (to act as Signal 1), and 1 μg/mL of an illustrative B7H3×CD3 bsAb. The data show that XENP40409 (non-Xtend analog of XENP40706) having 2G4_H1.12_L1.14 induced IL2 production most potently.

FIG. 117 depicts CD28 receptor occupancy on cynomolgus T cells (as indicated by decrease in binding by secondary CD28 mAb) after dosing with A) XENP36803 (1× dose, 4× dose, and 10× dose) or B) XENP36764 (4× dose, 10× dose, and 20× dose). The data show CD28 receptor occupancy up to day 14 on T cells.

FIG. 118 depicts PDL1 receptor occupancy on cynomolgus T cells (decrease in free receptor as indicated by decrease in binding by one-arm PDL1 mAb based on 2G4) after dosing with A) XENP36803 (1× dose, 4× dose, and 10× dose) or B) XENP36764 (4× dose, 10× dose, and 20× dose).

FIGS. 119A-119B depicts proliferation of cynomolgus T cells (as indicated by increased Ki67 expression) after dosing with A) XENP36803 (1× dose, 4× dose, and 10× dose) or B) XENP36764 (4× dose, 10× dose, and 20× dose). Notably, the PDL1×CD28 bsAbs selectively induce proliferation of effector CD4+ and CD8+ T cells (i.e. CD45RA−).

FIG. 120 depicts induction of A) IL-2 secretion and B) IFNγ secretion from T cells by 1 μg/ml illustrative B7H3×CD3 bsAb in combination with XENP37261, XENP40409, and additional PDL1×CD28 bsAbs utilizing additional PDL1 binding domains obtained from a common light chain campaign in the presence of MDA-MB-231 PDL1⁺PDL2⁺ cancer cells (1:1 E:T). XENP42047 and XENP42048 respectively utilizing 13G1 and 13G7 PDL1 CLC binding domains demonstrated similar potency and efficacy as XENP37261, while additional bsAbs utilizing other PDL1 CLC binding domains were less potent and/or efficacious.

FIG. 121 depicts induction of A) IL-2 secretion and B) IFNγ secretion from T cells by 1 μg/ml illustrative B7H3×CD3 bsAb in combination with XENP37261, XENP40409, and PDL2×CD28 bsAbs utilizing PDL2 binding domains obtained from a common light chain campaign in the presence of MDA-MB-231 PDL1⁺PDL2⁺ cancer cells (1:1 E:T). Most of the PDL2×CD28 bsAbs (XENP42051, XENP42052, XENP42053, and XENP42054 respectively utilizing 5C11, 8G2, 8G5, and 16G11 PDL2 CLC binding domains) but not all (i.e. XENP42050 using another high affinity PDL2 CLC binding domain) were equally or more potent and/or efficacious than the PDL1×CD28 bsAbs in inducing cytokine release

FIG. 122 depicts scenarios wherein tumor cells have different expression levels of PDL1 and PDL2 and relative binding capacities of a PDL1×CD28 bsAb, a combination of PDL1×CD28 and PDL2×CD28 bsAbs, and a PDL1×PDL2×CD28 triAb (in the 1+1+1 Fab-Fab-scFv format having PDL1 and PDL2 CLC binding domain and a CD28 binding domain) and illustrates that the triAb targets PDL1⁻PDL2⁺ tumors (assuming saturation, 100% avidity when possible (no switchover to monovalent interactions that may occur at high concentration)).

FIG. 123 depicts induction of IL-2 secretion by T cells by 1 μg/ml illustrative murine B7H3×CD3 bsAb in combination with XENP40409, PDL1×CD28 bsAbs utilizing CLC PDL1 binding domains (XENP42047 and XENP42049), PDL2×CD28 bsAbs utilizing CLC PDL2 binding domains (XENP42051, XENP42052, XENP42053, and XENP42054), PDL1×PDL2×CD28 triAbs in the 1+1+1 stackFab2-scFv-Fc format having PDL2 on the bottom (XENP42324, XENP42325, XENP42326, XENP42327, XENP42328, XENP42329, XENP42330, and XENP42331), PDL1×PDL2×CD28 triAbs in the 1+1+1 stackFab2-scFv-Fc format having PDL1 on the bottom (XENP42332, XENP42333, XENP42334, XENP42335, XENP42336, XENP42337, XENP42338, and XENP42339), and PDL1×PDL2×CD28 triAbs in the 1+1+1 mAb-scFv format (XENP42340, XENP42341, XENP42342, XENP42343, XENP42344, XENP42345, XENP42346, and XENP42347) in the presence of CHO-PDL1 cells (1:1 E:T).

FIG. 124 depicts induction of IL-2 secretion by T cells by 1 μg/ml illustrative murine B7H3×CD3 bsAb in combination with XENP40409, PDL1×CD28 bsAbs utilizing CLC PDL1 binding domains (XENP42047 and XENP42049), PDL2×CD28 bsAbs utilizing CLC PDL2 binding domains (XENP42051, XENP42052, XENP42053, and XENP42054), PDL1×PDL2×CD28 triAbs in the 1+1+1 stackFab2-scFv-Fc format having PDL2 on the bottom (XENP42324, XENP42325, XENP42326, XENP42327, XENP42328, XENP42329, XENP42330, and XENP42331), PDL1×PDL2×CD28 triAbs in the 1+1+1 stackFab2-scFv-Fc format having PDL1 on the bottom (XENP42332, XENP42333, XENP42334, XENP42335, XENP42336, XENP42337, XENP42338, and XENP42339), and PDL1×PDL2×CD28 triAbs in the 1+1+1 mAb-scFv format (XENP42340, XENP42341, XENP42342, XENP42343, XENP42344, XENP42345, XENP42346, and XENP42347) in the presence of CHO-PDL2 cells (1:1 E:T).

FIG. 125 depicts relative PDL1, PDL2, and B7H3 expression levels on LCLC-103H, SNU-423, and NCI-H460 cancer cells.

FIG. 126 depicts induction of IL-2 secretion from T cells by 1 μg/ml illustrative B7H3×CD3 bsAb in combination with XENP40409, PDL1×CD28 bsAbs utilizing PDL1 binding domains obtained from common light chain campaign (XENP42047 and XENP42049) and PDL2×CD28 bsAbs utilizing PDL2 binding domains obtained from common light chain campaign (XENP42051, XENP42052, XENP42053, and XENP42054) in the presence of A) LCLC-103H, B) SNU-423, and C) NCI-H460 cancer cells (1:1 E:T). Each of the bsAbs were active on LCLC-103H and SNU-423 with XPL1-13G1 as the most potent PDL1 binding domain (as in XENP42047) and 8G2 as the most potent PDL2 binding domain (as in XENP42052).

FIGS. 127A-127C depicts induction of IL-2 secretion by T cells by 1 μg/ml illustrative B7H3×CD3 bsAb in combination PDL1×PDL2×CD28 triAbs in the 1+1+1 stackFab2-scFv-Fc format having PDL2 on the bottom (XENP42324, XENP42325, XENP42326, XENP42327, XENP42328, XENP42329, XENP42330, and XENP42331) and PDL1×PDL2×CD28 triAbs in the 1+1+1 stackFab2-scFv-Fc format having PDL1 on the bottom (XENP42332, XENP42333, XENP42334, XENP42335, XENP42336, XENP42337, XENP42338, and XENP42339) in the presence of A) LCLC-103H, B) SNU-423, and C) NCI-H460 cancer cells (1:1 E:T). D) depicts the legend.

FIG. 128 depicts PD-1 blockade by PDL1×CD28 and PDL2×CD28 bsAbs based on various PDL1 and PDL2 binding domains on CHO-PDL1 cells.

FIG. 129 depicts PD-1 blockade by PDL1×CD28 and PDL2×CD28 bsAbs based on various PDL1 and PDL2 binding domains on CHO-PDL2 cells.

FIGS. 130A-130C depicts induction of IL-2 secretion by T cells by illustrative murine B7H3×CD3 bsAb in combination with A) XENP42049 PDL1×CD28, B) XENP42054 PDL2×CD28, and C) XENP43461 PDL1×PDL2×CD28 in the presence of LCLC103H-NLR cell line and PDL1 blockade or PDL2 blockade.

FIG. 131 depicts control antibodies utilizing an RSV binding domain.

FIG. 132 depicts PDL1 expression on PC3 cells cocultured with T cells after treatment with illustrative B7H3×CD3 bsAb alone or in combination with IFNγ neutralizing mAb. CD3 bsAb induces PDL1 and PDL2 expression by promoting IFNγ release.

FIGS. 133A-133C depicts IFNγ secretion by T cells cocultured with PC3 cells and then treated with the indicated antibodies (illustrative B7H3×CD3 bsAb alone or in combination with XENP40409) for A) 1 day, B) 2 days, and c) 5 days. CD3 bsAb synergizes with PDL1×CD28 over time as PDL1 expression increases.

FIG. 134 depicts induction of IFNγ release (5 days post-treatment) in mixed lymphocyte reactions (n=14) following incubation with PBS, XENP43456 (PDL1×PDL2×RSV), or anti-PD1 mAb. PDL1 and PDL2 blockade is functionally equivalent to PD1 blockade.

FIG. 135 depicts IFNγ release (1 day post-treatment) by PBMC treated with indicated air-dried αCD28 bivalent antibodies (TGN1412 or 1A7-derived). 1A7-derived anti-CD28 epitope lacks superagonistic properties.

FIGS. 136A-136B depicts induction of IL2 release by T cells cocultured with A) PDL1^{high} MDA-MB-231 cancer cells (1:1 effector:target ratio; ˜130,000 PDL1 antigens) and B) PDL1^{low} LNCaP (10:1 effector:target ratio; ˜13,000 PDL1 antigens) and treated with indicated concentrations of indicated PDL1×PDL2×CD28 triAb in combination with illustrative B7H3×CD3 bsAb. PDL1×PDL2×CD28 triAb enhanced IL-2 release on high PDL1 and low PDL1 cancer cells.

FIGS. 137A-137B depicts induction of redirected T cell cytotoxicity (RTCC; as determined by luminescence 5 days following treatment) by T cells cocultured with A) PDL1^{high} MDA-MB-231 cancer cells (1:50 effector:target ratio; ˜130,000 PDL1 antigens) and B) PDL1^{low} LNCaP (10:1 effector:target ratio; ˜13,000 PDL1 antigens) and treated with indicated concentrations of illustrative B7H3×CD3 bsAb alone or in combination with 1 μg/ml indicated PDL1×PDL2×CD28 triAb. PDL1×PDL2×CD28 triAb enhances redirected T cell cytotoxicity at low effector to target ratios as well as on low PDL1 cancer cells.

FIGS. 138A-138B depicts induction of IL2 release (24 hour post-treatment) by T cells from A) a first and B) a second CMV+ PBMC donor cocultured with A431-132M-null cells or A431-432M-null cells stably expression a fusion of HLA-A2, ß2M and NLV-peptide and treated with PDL1×PDL2×CD28, PDL1×PDL2×RSV, or RSV×CD28.

FIGS. 139A and 139B depict sequences for XENP43734 and XENP43735 which are respectively mouse surrogate PDL1×PDL2×CD28 and PDL1×PDL2×RSV triAbs. It should be noted that, XENP43735 can be considered as a mouse surrogate for XENP43461 (without Xtend).

FIGS. 140A-140D depict change in tumor volume (as determined by caliper measurements) in individual mouse over time in hPDL1(high)-MC38-engrafted hCD28 knock-in mice dosed with A) PBS control, B) anti-mouse PD1 mAb, and C) XENP43735 (mouse surrogate PDL1×PDL2×RSV triAb control), and D) XENP43734 (mouse surrogate PDL1×PDL2×CD28 triAb analogous to XENP43461 (without Xtend)). Arrows indicate days of dosing.

FIG. 141 depicts blockade of PD1:PDL1 and PD1:PDL2 on Jurkat-PD1 cells incubated with A431-αCD3 by bivalent PD-1 mAb or XENP43456 PDL1×PDL2×RSV triAb.

FIGS. 142A-142E depicts change in tumor volume (as determined by caliper measurements) in individual mouse over time in MDA-MB-231 and huPBMC-engrafted NSG-DKO mice dosed with A) PBS control, B) illustrative B7H3×CD3 bsAb (0.5 mg/kg), C) PDL1×PDL2×CD28 XENP44676 in combination with B7H3×CD3 bsAb (respectively 5 mg/kg and 0.5 mg/kg), D) control PDL1×PDL2×RSV XENP44796 in combination with B7H3×CD3 bsAb (respectively 5 mg/kg and 0.5 mg/kg), or E) control RSV×RSV×CD28 XENP44797 in combination with B7H3×CD3 bsAb (respectively 5 mg/kg and 0.5 mg/kg).

FIG. 143 depicts tumor volumes (as determined by caliper measurements) in individual MDA-MB-231 and huPBMC-engrafted NSG-DKO mice on Day 39 after dosing with A) PBS control, B) illustrative B7H3×CD3 bsAb (0.5 mg/kg), C) PDL1×PDL2×CD28 XENP44676 in combination with B7H3×CD3 bsAb (respectively 5 mg/kg and 0.5 mg/kg), D) control PDL1×PDL2×RSV XENP44796 in combination with B7H3×CD3 bsAb (respectively 5 mg/kg and 0.5 mg/kg), or E) control RSV×RSV×CD28 XENP44797 in combination with B7H3×CD3 bsAb (respectively 5 mg/kg and 0.5 mg/kg).

FIG. 144 depicts CD3+ cell counts in blood of MDA-MB-231 and huPBMC-engrafted NSG-DKO mice dosed with A) PBS control, B) illustrative B7H3×CD3 bsAb (0.5 mg/kg), C) PDL1×PDL2×CD28 XENP44676 in combination with B7H3×CD3 bsAb (respectively 5 mg/kg and 0.5 mg/kg), D) control PDL1×PDL2×RSV XENP44796 in combination with B7H3×CD3 bsAb (respectively 5 mg/kg and 0.5 mg/kg), or E) control RSV×RSV×CD28 XENP44797 in combination with B7H3×CD3 bsAb (respectively 5 mg/kg and 0.5 mg/kg) on Day 7 after first dose.

FIG. 145 depicts K_{D} binding constant, association constant (kₐ), and dissociation constant (k_{d}) for affinity-engineered anti-PDL1 clone 13G7 against human PDL1.

FIG. 146 depicts K_{D} binding constant, association constant (kₐ), and dissociation constant (k_{d}) for affinity-engineered anti-PDL1 clone 13G7 against cynomolgus PDL1.

FIG. 147 depicts K_{D} binding constant, association constant (kₐ), and dissociation constant (k_{d}) for affinity-engineered anti-PDL2 clone 16G11 against human PDL2.

FIG. 148 depicts K_{D} binding constant, association constant (kₐ), and dissociation constant (k_{d}) for affinity-engineered anti-PDL2 clone 16G11 against cynomolgus PDL2.

FIG. 149 depicts K_{D} binding constant, association constant (kₐ), and dissociation constant (k_{d}) for affinity-engineered anti-PDL1 clone 1A3A4.248 against human PDL1.

FIG. 150 depicts K_{D} binding constant, association constant (kₐ), and dissociation constant (k_{d}) for affinity-engineered anti-PDL1 clone 1A3A4.248 against cynomolgus PDL1.

FIG. 151 depicts K_{D} binding constant, association constant (kₐ), and dissociation constant (k_{d}) for affinity-engineered anti-PDL2 clone 1F12A4.249 (round 1) against human PDL2.

FIG. 152 depicts K_{D} binding constant, association constant (kₐ), and dissociation constant (k_{d}) for affinity-engineered anti-PDL2 clone 1F12A4.249 (round 1) against cynomolgus PDL2.

FIGS. 153A-153B depicts K_{D} binding constant, association constant (kₐ), and dissociation constant (k_{d}) for affinity-engineered anti-PDL2 clone 1F12A4.249 (round 2) against human PDL2.

FIGS. 154A-154B depicts K_{D} binding constant, association constant (kₐ), and dissociation constant (k_{d}) for affinity-engineered anti-PDL2 clone 1F12A4.249 (round 2) against cynomolgus PDL2.

FIG. 155 depicts induction of IL-2 secretion by 1 μg/mL illustration B7H3×CD3 bsAb in combination with PDL1×PDL2×CD28 in 1+1+1 Fab-(Fab-scFv)-Fc (with the CD28 scFv in the VHVL or VLVH orientations), 1+1+1 stackFab₂-scFv-Fc, and 1+1+1 mAb-scFv formats in the presence of A) LnCAP (PDL1^{low}PDL2^{null}), B) DU145 (PDL1^{med}PDL2^{null}), or C) LCLC103H (PDL1^{hi}PDL2^{med}) tumor cells.

FIG. 156 depicts induction of IL-2 secretion by 1 μg/mL illustration B7H3×CD3 bsAb in combination with PDL1×PDL2×CD28 in 1+1+1 stackFab₂-scFv-Fc with WT scFv (XENP43461) or with stapled scFv (XENP43462) in the presence of DU145 (PDL1^{med}PDL2^{null}) tumor cells (1:1 effector:target ratio).

FIG. 157 depicts induction of IL-2 secretion by 1 μg/mL illustrative B7H3×CD3 bsAb in combination with PDL1×PDL2×CD28 utilizing 1A7 or 1A3 CD28 binding domains in various formats and orientations in the presence of DU145 tumor cells. The data show that XENP43461 having the 1A7 binding domain outperformed triAbs in all formats having the 1A3 binding domain (PDL1 and PDL2 binding domains are matched).

FIG. 158 depicts induction of IL-2 secretion by 1 μg/mL illustrative B7H3×CD3 bsAb in combination with PDL1×PDL2×CD28 utilizing 1A7 having germline VL and affinity-optimized VHs in the presence of DU145 tumor cells. The data show that several of the full CLC triAbs achieved potency comparable to that of XENP43465.

FIG. 159 depicts EC50 of induction of IL-2 secretion by 1 μg/mL illustrative B7H3×CD3 bsAb in combination with PDL1×PDL2×CD28 utilizing 1A7 having germline VL and affinity-optimized VHs in the presence of DU145 tumor cells, as well as monovalent K_{D} for CD28 binding by the affinity-engineered 1A7 binding domains. The data show that several of the full CLC triAbs achieved potency comparable to that of XENP43465.

FIG. 160 depicts induction of IL-2 secretion by 1 μg/mL illustrative B7H3×CD3 bsAb in combination with PDL1×PDL2×CD28 utilizing 1A7 or 1A3-derived CD28 binding domains in the presence of Caki-1 tumor cells (1:1 effector:target). The data show that the 1A7-based full CLC triAb is more potent and efficacious that the 1A3-based full CLC triAb.

FIGS. 161A-161C depicts novel PDL1 binding domain clone 2A3A4.248, as well as illustrative affinity-engineered VH 2A3A4.248_H1.9 (additional variants depicted as SEQ ID NOs: 3235-3266) which may be paired with the L1 (it should be noted that the L1 is the IGKV1-39 germline sequence). Additionally, consensus framework regions (FR) and complementarity determining regions (CDRs) for 1F12A4.249 VH are depicted. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 162A-162G depicts novel PDL2 binding domain clone 1F12A4.249, as well as illustrative affinity-engineered VH 1F12A4.249_H1.45 (additional variants depicted as SEQ ID NOs: 3267-3353) which may be paired with the L1 (it should be noted that the L1 is the IGKV1-39 germline sequence). Additionally, consensus framework regions (FR) and complementarity determining regions (CDRs) for 1F12A4.249 VH are depicted. As noted herein and is true for every sequence herein containing CDRs, the exact identification of the CDR locations may be slightly different depending on the numbering used as is shown in Table 2, and thus included herein are not only the CDRs that are underlined but also CDRs included within the VH and VL domains using other numbering systems. Furthermore, as for all the sequences in the Figures, these VH and VL sequences can be used either in a scFv format or in a Fab format.

FIGS. 163A-163C depicts novel 1A7 VHs affinity-engineered to be paired with IGKV1-39 germline sequence, as well as consensus framework regions (FR) and complementarity determining regions (CDRs).

FIGS. 164A-164D depicts illustrative 1A7-based scFvs with engineered cysteines for disulfied stabilization.

FIG. 165 depicts CD28 affinity for additional 1A7 scFv variants, with or without disulfide stabilization (as denoted by [S—S]) obtained from different experiments in the context of different CD28 multispecific antibodies.

FIG. 166 depicts increase in thermostability of disulfide-stabilized 1A7 scFv.

### DETAILED DESCRIPTION

### I. Overview

Provided herein are novel anti-PD-L1, anti-PD-L2, and anti-CD28 antibodies, including novel anti-CD28×anti-PD-L1 and anti-CD28×anti-PD-L2 bispecific antibodies, and novel anti-CD28×anti-PD-L1×anti-PD-L2 trispecific antibodies. Also provided herein are methods for making and using such antibodies for the treatment of cancers. Subject bispecific and trispecific antibodies are capable of agonistically binding to CD28 costimulatory molecules on T cells and targeting to PD-L1 and/or PD-L2 on tumor cells. Thus, such antibodies selectively enhance anti-tumor activity at tumor sites while minimizing peripheral toxicity. The subject antibodies provided herein are particularly useful for enhancing anti-tumor activity when used in combination with other anti-cancer therapies.

Accordingly, in one aspect, provided herein are heterodimeric antibodies that bind to two or three different antigens. In some embodiments, the antibodies are “bispecific,” and bind two different target antigens, generally CD28 and PD-L1 or CD28 and PD-L2, as described below. In some embodiments, the antibodies are “trispecific,” and bind three different target antigens, generally CD28, PD-L1, and PD-L2, as described below. These heterodimeric antibodies can bind each of the target antigens either monovalently (e.g., there is a single antigen binding domain such as a variable heavy and variable light domain pair) or bivalently (there are two antigen binding domains that each independently bind the antigen). In some embodiments, the heterodimeric antibody provided herein includes 1) one CD28 binding domain, and 2) one PD-L1 or one PD-L2 binding domain (e.g., heterodimeric antibodies in the “1+1 Fab-scFv-Fc” format described herein). In other embodiments, the heterodimeric antibody provided herein includes 1) one CD28 binding domain, and 2) two PD-L1 or PD-L2 binding domains (e.g., heterodimeric antibodies in the “2+1 Fab₂-scFv-Fc” formats described herein). In some embodiments, the heterodimeric antibody provided herein is a trispecific antibody, that includes three different antigen binding domain that each bind a different target antigen, generally PD-L1, PD-L2, and CD28 (e.g., heterodimeric antibodies in the “1+1+1 stackFab₂-scFv-Fc,” “1+1+1 Fab-(Fab-scFv)-Fc,” “1+1+1 mAb-scFv,” and, “1+1+1 stackFab₂-Fab-Fc” formats, FIG. 83). The heterodimeric antibodies provided herein are based on the use of different monomers that contain amino acid substitutions (i.e., skew variants”) that “skew” formation of heterodimers over homodimers, as is more fully outlined below. In some embodiments, the heterodimer antibodies are also coupled with “pI variants” that allow simple purification of the heterodimers away from the homodimers, as is similarly outlined below. The heterodimeric bispecific antibodies provided generally rely on the use of engineered or variant Fc domains that can self-assemble in production cells to produce heterodimeric proteins, and methods to generate and purify such heterodimeric proteins.

### II. Nomenclature

The antibodies provided herein are listed in several different formats. In some instances, each monomer of a particular antibody is given a unique “XENP” number, although as will be appreciated in the art, a longer sequence might contain a shorter one. For example, a “scFv-Fc” monomer of a 1+1 Fab-scFv-Fc format antibody may have a first XENP number, while the scFv domain itself will have a different XENP number. Some molecules have three polypeptides, so the XENP number, with the components, is used as a name. Thus, the molecule XENP34961, which is in 2+1 Fab₂-scFv-Fc format, comprises three sequences (see FIG. 85A) a “Fab-Fc Heavy Chain” monomer (“Chain 1”); 2) a “Fab-scFv-Fc Heavy Chain” monomer (“Chain 2”); and 3) a “Light Chain” monomer or equivalents, although one of skill in the art would be able to identify these easily through sequence alignment. These XENP numbers are in the sequence listing as well as identifiers, and used in the Figures. In addition, one molecule, comprising the three components, gives rise to multiple sequence identifiers. For example, the listing of the Fab includes the full heavy chain sequence, the variable heavy domain sequence and the three CDRs of the variable heavy domain sequence, the full light chain sequence, a variable light domain sequence and the three CDRs of the variable light domain sequence. A Fab-scFv-Fc monomer includes a full-length sequence, a variable heavy domain sequence, 3 heavy CDR sequences, and an scFv sequence (include scFv variable heavy domain sequence, scFv variable light domain sequence and scFv linker). Note that some molecules herein with a scFv domain use a single charged scFv linker (+H), although others can be used. In addition, the naming nomenclature of particular antigen binding domains (e.g., PD-L1, PD-L2, and CD28 binding domains) use a “Hx.xx_Ly.yy” or “Hx.xxLy.yy” type of format, with the numbers being unique identifiers to particular variable chain sequences. Thus, the variable domain of the Fab side of PD-L1 binding domain 2G4[PDL1](e.g., FIG. 17) is “H1L1”, which indicates that the variable heavy domain, H1, was combined with the light domain L1. In the case that these sequences are used as scFvs, the designation “H1_L1” or “H1L1”, indicates that the variable heavy domain, H1 is combined with the light domain, L1, and is in VH-linker-VL orientation, from N- to C-terminus. This molecule with the identical sequences of the heavy and light variable domains but in the reverse order (VL-linker-VH orientation, from N- to C-terminus) would be designated “L1_H”. Similarly, different constructs may “mix and match” the heavy and light chains as will be evident from the sequence listing and the figures.

### III. Definitions

In order that the application may be more completely understood, several definitions are set forth below. Such definitions are meant to encompass grammatical equivalents.

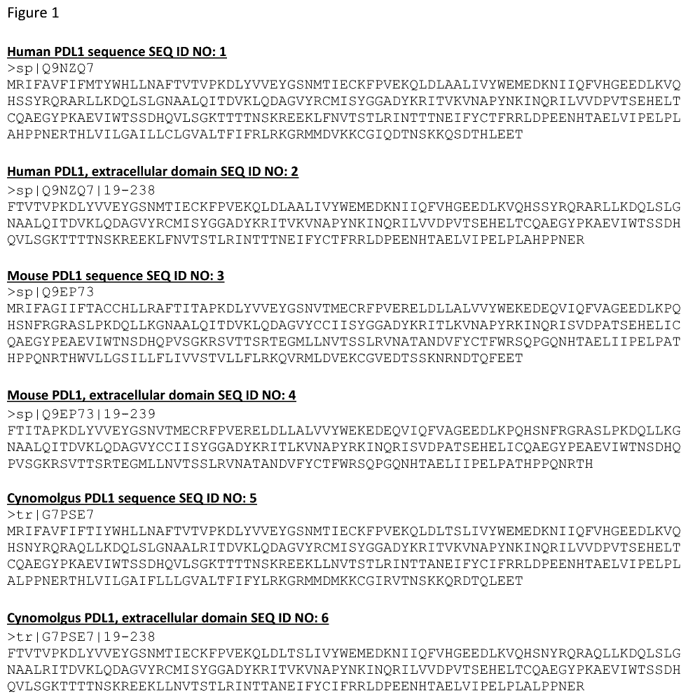

By “CD28,” “Cluster of Differentiation 28,” and “Tp44” (e.g., Genebank Accession Numbers NP_001230006 (human), NP_001230007 (human), NP_006130 (human), and NP_031668 (mouse)) herein is meant a B7 receptor expressed on T cells that provides co-stimulatory signals required for T cell activation and survival. T cell stimulation through CD28 in addition to the T cell receptor (TCR) provides a potent signal for the production of various interleukins. CD28 is the receptor for CD80 (B7.1) and CD86 (B7.2) proteins. CD28 includes an intercellular domain with a YMNM motif critical for the recruitment of SH2-domain containing proteins, particularly PI3K. CD28 also includes two proline-rich motifs that are able to bind SH3-containing proteins. Exemplary CD28 sequences are depicted in FIG. 1.

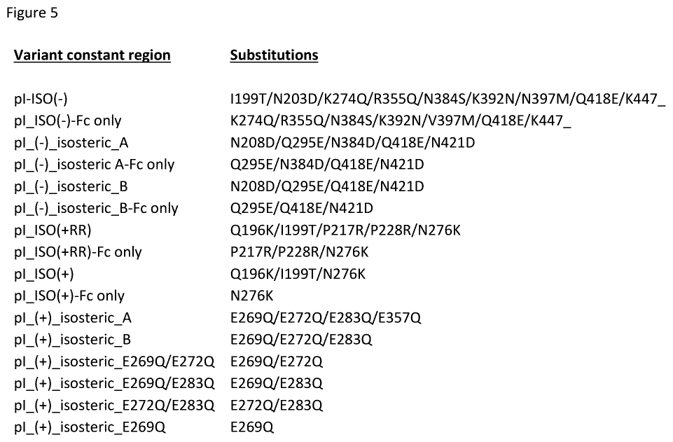

By “ablation” herein is meant a decrease or removal of activity. Thus, for example, “ablating FcγR binding” means the Fc region amino acid variant has less than 50% starting binding as compared to an Fc region not containing the specific variant, with more than 70-80-90-95-98% loss of activity being preferred, and in general, with the activity being below the level of detectable binding in a Biacore, SPR or BLI assay. Of particular use in the ablation of FcγR binding are those shown in FIG. 5, which generally are added to both monomers.

By “ADCC” or “antibody dependent cell-mediated cytotoxicity” as used herein is meant the cell-mediated reaction, wherein nonspecific cytotoxic cells that express FcγRs recognize bound antibody on a target cell and subsequently cause lysis of the target cell. ADCC is correlated with binding to FcγRIIIa; increased binding to FcγRIIIa leads to an increase in ADCC activity.

By “ADCP” or antibody dependent cell-mediated phagocytosis as used herein is meant the cell-mediated reaction wherein nonspecific phagocytic cells that express FcγRs recognize bound antibody on a target cell and subsequently cause phagocytosis of the target cell.

As used herein, the term “antibody” is used generally. Antibodies provided herein can take on a number of formats as described herein, including traditional antibodies as well as antibody derivatives, fragments and mimetics, described herein.

Traditional immunoglobulin (Ig) antibodies are “Y” shaped tetramers. Each tetramer is typically composed of two identical pairs of polypeptide chains, each pair having one “light chain” monomer (typically having a molecular weight of about 25 kDa) and one “heavy chain” monomer (typically having a molecular weight of about 50-70 kDa).

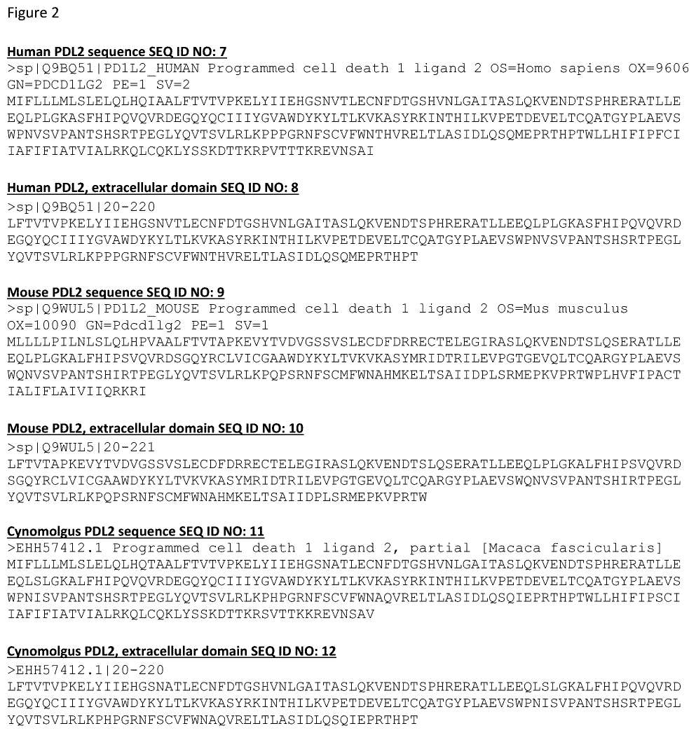

Useful bispecific antibody formats include, but are not limited to, the “1+1 Fab-scFv-Fc,” “2+1 Fab₂-scFv-Fc,” “1+1 common light chain,” and “2+1 common light chain” formats provided herein (see, e.g., FIG. 82). Useful trispecific antibody formats include, but are not limited to, 1+1+1 stackFab₂-scFv-Fc,” “1+1+1 Fab-(Fab-scFv)-Fc,” “1+1+1 mAb-scFv,” and, “1+1+1 stackFab₂-Fab-Fc” formats (see, e.g., FIG. 83). Additional useful antibody formats include, but are not limited to, “mAb-Fv,” “mAb-scFv,” “central-Fv”, “one armed scFv-mAb,” “scFv-mAb,” “dual scFv,” and “trident” format antibodies, as disclosed in US20180127501A1, which is incorporated by reference herein, particularly in pertinent part relating to antibody formats (see, e.g., FIG. 2).

Antibody heavy chains typically include a variable heavy (VH) domain, which includes vhCDR1-3, and an Fc domain, which includes a CH2-CH3 monomer. In some embodiments, antibody heavy chains include a hinge and CH1 domain. Traditional antibody heavy chains are monomers that are organized, from N- to C-terminus: VH-CH1-hinge-CH2-CH3. The CH1-hinge-CH2-CH3 is collectively referred to as the heavy chain “constant domain” or “constant region” of the antibody, of which there are five different categories or “isotypes”: IgA, IgD, IgG, IgE and IgM.

In some embodiments, the antibodies provided herein include IgG isotype constant domains, which has several subclasses, including, but not limited to IgG1, IgG2, IgG3, and IgG4. In the IgG subclass of immunoglobulins, there are several immunoglobulin domains in the heavy chain. By “immunoglobulin (Ig) domain” herein is meant a region of an immunoglobulin having a distinct tertiary structure. Of interest in the present invention are the heavy chain domains, including, the constant heavy (CH) domains and the hinge domains. In the context of IgG antibodies, the IgG isotypes each have three CH regions. Accordingly, “CH” domains in the context of IgG are as follows: “CH1” refers to positions 118-215 according to the EU index as in Kabat. “Hinge” refers to positions 216-230 according to the EU index as in Kabat. “CH2” refers to positions 231-340 according to the EU index as in Kabat, and “CH3” refers to positions 341-447 according to the EU index as in Kabat. As shown in Table 1, the exact numbering and placement of the heavy chain domains can be different among different numbering systems. As shown herein and described below, the pI variants can be in one or more of the CH regions, as well as the hinge region, discussed below.

It should be noted that IgG1 has different allotypes with polymorphisms at 356 (D or E) and 358 (L or M). The sequences depicted herein use the 356E/358M allotype, however the other allotype is included herein. That is, any sequence inclusive of an IgG1 Fc domain included herein can have 356D/358L replacing the 356E/358M allotype. It should be understood that therapeutic antibodies can also comprise hybrids of isotypes and/or subclasses. For example, as shown in US Publication 2009/0163699, incorporated by reference, the present antibodies, in some embodiments, include human IgG1/G2 hybrids.

By “Fc” or “Fc region” or “Fc domain” as used herein is meant the polypeptide comprising the constant region of an antibody, in some instances, excluding all of the first constant region immunoglobulin domain (e.g., CH1) or a portion thereof, and in some cases, optionally including all or part of the hinge. For IgG, the Fc domain comprises immunoglobulin domains CH2 and CH3 (Cγ2 and Cγ3), and optionally all or a portion of the hinge region between CH1 (Cγ1) and CH2 (Cγ2). Thus, in some cases, the Fc domain includes, from N- to C-terminal, CH2-CH3 and hinge-CH2-CH3. In some embodiments, the Fc domain is that from IgG1, IgG2, IgG3 or IgG4, with IgG1 hinge-CH2-CH3 and IgG4 hinge-CH2-CH3 finding particular use in many embodiments. Additionally, in the case of human IgG1 Fc domains, the hinge may include a C220S amino acid substitution. Furthermore, in the case of human IgG4 Fc domains, the hinge may include a S228P amino acid substitution. Although the boundaries of the Fc region may vary, the human IgG heavy chain Fc region is usually defined to include residues E216, C226, or A231 to its carboxyl-terminal, wherein the numbering is according to the EU index as in Kabat. In some embodiments, as is more fully described below, amino acid modifications are made to the Fc region, for example to alter binding to one or more FcγR or to the FcRn.

By “heavy chain constant region” herein is meant the CH1-hinge-CH2-CH3 portion of an antibody (or fragments thereof), excluding the variable heavy domain; in EU numbering of human IgG1 this is amino acids 118-447. By “heavy chain constant region fragment” herein is meant a heavy chain constant region that contains fewer amino acids from either or both of the N- and C-termini but still retains the ability to form a dimer with another heavy chain constant region.

Another type of domain of the heavy chain is the hinge region. By “hinge” or “hinge region” or “antibody hinge region” or “hinge domain” herein is meant the flexible polypeptide comprising the amino acids between the first and second constant domains of an antibody. Structurally, the IgG CH1 domain ends at EU position 215, and the IgG CH2 domain begins at residue EU position 231. Thus for IgG the antibody hinge is herein defined to include positions 216 (E216 in IgG1) to 230 (P230 in IgG1), wherein the numbering is according to the EU index as in Kabat. In some cases, a “hinge fragment” is used, which contains fewer amino acids at either or both of the N- and C-termini of the hinge domain. As noted herein, pI variants can be made in the hinge region as well. Many of the antibodies herein have at least one the cysteines at position 220 according to EU numbering (hinge region) replaced by a serine. Generally, this modification is on the “scFv monomer” side for most of the sequences depicted herein, although it can also be on the “Fab monomer” side, or both, to reduce disulfide formation. Specifically included within the sequences herein are one or both of these cysteines replaced (C220S).

As will be appreciated by those in the art, the exact numbering and placement of the heavy chain constant region domains (i.e., CH1, hinge, CH2 and CH3 domains) can be different among different numbering systems. A useful comparison of heavy constant region numbering according to EU and Kabat is as below, see Edelman et al., 1969, Proc Natl Acad Sci USA 63:78-85 and Kabat et al., 1991, Sequences of Proteins of Immunological Interest, 5th Ed., United States Public Health Service, National Institutes of Health, Bethesda, entirely incorporated by reference.

TABLE 1

| | EU Numbering | Kabat Numbering |
|---|---|---|
| CH1 | 118-215 | 114-223 |
| Hinge | 216-230 | 226-243 |
| CH2 | 231-340 | 244-360 |
| CH3 | 341-447 | 361-478 |

The antibody light chain generally comprises two domains: the variable light domain (VL), which includes light chain CDRs vlCDR1-3, and a constant light chain region (often referred to as CL or Cκ). The antibody light chain is typically organized from N- to C-terminus: VL-CL.

By “antigen binding domain” or “ABD” herein is meant a set of six Complementary Determining Regions (CDRs) that, when present as part of a polypeptide sequence, specifically binds a target antigen (e.g., PD-L1 or CD28) as discussed herein. As is known in the art, these CDRs are generally present as a first set of variable heavy CDRs (vhCDRs or VHCDRs) and a second set of variable light CDRs (vlCDRs or VLCDRs), each comprising three CDRs: vhCDR1, vhCDR2, vhCDR3 variable heavy CDRs and vlCDR1, vlCDR2 and vlCDR3 vhCDR3 variable light CDRs. The CDRs are present in the variable heavy domain (vhCDR1-3) and variable light domain (vlCDR1-3). The variable heavy domain and variable light domain from an Fv region.

The present invention provides a large number of different CDR sets. In this case, a “full CDR set” comprises the three variable light and three variable heavy CDRs, e.g., a vlCDR1, vlCDR2, vlCDR3, vhCDR1, vhCDR2 and vhCDR3. These can be part of a larger variable light or variable heavy domain, respectfully. In addition, as more fully outlined herein, the variable heavy and variable light domains can be on separate polypeptide chains, when a heavy and light chain is used (for example when Fabs are used), or on a single polypeptide chain in the case of scFv sequences.

As will be appreciated by those in the art, the exact numbering and placement of the CDRs can be different among different numbering systems. However, it should be understood that the disclosure of a variable heavy and/or variable light sequence includes the disclosure of the associated (inherent) CDRs. Accordingly, the disclosure of each variable heavy region is a disclosure of the vhCDRs (e.g., vhCDR1, vhCDR2 and vhCDR3) and the disclosure of each variable light region is a disclosure of the vlCDRs (e.g., vlCDR1, vlCDR2 and vlCDR3). A useful comparison of CDR numbering is as below, see Lafranc et al., Dev. Comp. Immunol. 27(1):55-77 (2003):

TABLE 2

| | Kabat+ | | | | | | |
|---|---|---|---|---|---|---|---|
| | Chothia | IMGT | Kabat | AbM | Chothia | Contact | Xencor |
| vhCDR1 | 26-35 | 27-38 | 31-35 | 26-35 | 26-32 | 30-35 | 27-35 |
| vhCDR2 | 50-65 | 56-65 | 50-65 | 50-58 | 52-56 | 47-58 | 54-61 |
| vhCDR3 | 95-102 | 105-117 | 95-102 | 95-102 | 95-102 | 93-101 | 103-116 |
| vlCDR1 | 24-34 | 27-38 | 24-34 | 24-34 | 24-34 | 30-36 | 27-38 |
| vlCDR2 | 50-56 | 56-65 | 50-56 | 50-56 | 50-56 | 46-55 | 56-62 |
| vlCDR3 | 89-97 | 105-117 | 89-97 | 89-97 | 89-97 | 89-96 | 97-105 |

Throughout the present specification, the Kabat numbering system is generally used when referring to a residue in the variable domain (approximately, residues 1-107 of the light chain variable region and residues 1-113 of the heavy chain variable region) and the EU numbering system for Fc regions (e.g., Kabat et al., supra (1991)).

The CDRs contribute to the formation of the antigen-binding, or more specifically, epitope binding site of the antigen binding domains and antibodies. “Epitope” refers to a determinant that interacts with a specific antigen binding site in the variable region of an antibody molecule known as a paratope. Epitopes are groupings of molecules such as amino acids or sugar side chains and usually have specific structural characteristics, as well as specific charge characteristics. A single antigen may have more than one epitope.

The epitope may comprise amino acid residues directly involved in the binding (also called immunodominant component of the epitope) and other amino acid residues, which are not directly involved in the binding, such as amino acid residues which are effectively blocked by the specifically antigen binding peptide; in other words, the amino acid residue is within the footprint of the specifically antigen binding peptide.

Epitopes may be either conformational or linear. A conformational epitope is produced by spatially juxtaposed amino acids from different segments of the linear polypeptide chain. A linear epitope is one produced by adjacent amino acid residues in a polypeptide chain. Conformational and nonconformational epitopes may be distinguished in that the binding to the former but not the latter is lost in the presence of denaturing solvents.

An epitope typically includes at least 3, and more usually, at least 5 or 8-10 amino acids in a unique spatial conformation. Antibodies that recognize the same epitope can be verified in a simple immunoassay showing the ability of one antibody to block the binding of another antibody to a target antigen, for example “binning.” As outlined below, the invention not only includes the enumerated antigen binding domains and antibodies herein, but those that compete for binding with the epitopes bound by the enumerated antigen binding domains.

In some embodiments, the six CDRs of the antigen binding domain are contributed by a variable heavy and a variable light domain. In a “Fab” format, the set of 6 CDRs are contributed by two different polypeptide sequences, the variable heavy domain (vh or VH; containing the vhCDR1, vhCDR2 and vhCDR3) and the variable light domain (vl or VL; containing the vlCDR1, vlCDR2 and vlCDR3), with the C-terminus of the vh domain being attached to the N-terminus of the CH1 domain of the heavy chain and the C-terminus of the vl domain being attached to the N-terminus of the constant light domain (and thus forming the light chain). In a scFv format, the vh and vl domains are covalently attached, generally through the use of a linker (a “scFv linker”) as outlined herein, into a single polypeptide sequence, which can be either (starting from the N-terminus) vh-linker-vl or vl-linker-vh, with the former being generally preferred (including optional domain linkers on each side, depending on the format used (e.g., from FIG. 26). In general, the C-terminus of the scFv domain is attached to the N-terminus of the hinge in the second monomer.

By “variable region” or “variable domain” as used herein is meant the region of an immunoglobulin that comprises one or more Ig domains substantially encoded by any of the Vx, VW, and/or VH genes that make up the kappa, lambda, and heavy chain immunoglobulin genetic loci respectively, and contains the CDRs that confer antigen specificity. Thus, a “variable heavy domain” pairs with a “variable light domain” to form an antigen binding domain (“ABD”). In addition, each variable domain comprises three hypervariable regions (“complementary determining regions,” “CDRs”) (vhCDR1, vhCDR2 and vhCDR3 for the variable heavy domain and vlCDR1, vlCDR2 and vlCDR3 for the variable light domain) and four framework (FR) regions, arranged from amino-terminus to carboxy-terminus in the following order: FR1-CDR1-FR2-CDR2-FR3-CDR3-FR4.

By “Fab” or “Fab region” as used herein is meant the antibody region that comprises the VH, CH1, VL, and CL immunoglobulin domains, generally on two different polypeptide chains (e.g., VH-CH1 on one chain and VL-CL on the other). Fab may refer to this region in isolation, or this region in the context of a bispecific antibody of the invention. In the context of a Fab, the Fab comprises an Fv region in addition to the CH1 and CL domains.

By “Fv” or “Fv fragment” or “Fv region” as used herein is meant the antibody region that comprises the VL and VH domains. Fv regions can be formatted as both Fabs (as discussed above, generally two different polypeptides that also include the constant regions as outlined above) and single chain Fvs (scFvs), where the vl and vh domains are included in a single peptide, attached generally with a linker as discussed herein.

By “single chain Fv” or “scFv” herein is meant a variable heavy domain covalently attached to a variable light domain, generally using a scFv linker as discussed herein, to form a scFv or scFv domain. A scFv domain can be in either orientation from N- to C-terminus (vh-linker-vl or vl-linker-vh). In the sequences depicted in the sequence listing and in the figures, the order of the vh and vl domain is indicated in the name, e.g., H.X_L.Y means N- to C-terminal is vh-linker-vl, and L.Y_H.X is vl-linker-vh.

Some embodiments of the subject antibodies provided herein comprise at least one scFv domain, which, while not naturally occurring, generally includes a variable heavy domain and a variable light domain, linked together by a scFv linker. As outlined herein, while the scFv domain is generally from N- to C-terminus oriented as VH-scFv linker-VL, this can be reversed for any of the scFv domains (or those constructed using vh and vl sequences from Fabs), to VL-scFv linker-VH, with optional linkers at one or both ends depending on the format.

By “modification” or “variant” herein is meant an amino acid substitution, insertion, and/or deletion in a polypeptide sequence or an alteration to a moiety chemically linked to a protein. For example, a modification may be an altered carbohydrate or PEG structure attached to a protein. By “amino acid modification” herein is meant an amino acid substitution, insertion, and/or deletion in a polypeptide sequence. For clarity, unless otherwise noted, the amino acid modification is always to an amino acid coded for by DNA, e.g., the 20 amino acids that have codons in DNA and RNA.

By “amino acid substitution” or “substitution” herein is meant the replacement of an amino acid at a particular position in a parent polypeptide sequence with a different amino acid. In particular, in some embodiments, the substitution is to an amino acid that is not naturally occurring at the particular position, either not naturally occurring within the organism or in any organism. For example, the substitution E272Y refers to a variant polypeptide, in this case an Fc variant, in which the glutamic acid at position 272 is replaced with tyrosine. For clarity, a protein which has been engineered to change the nucleic acid coding sequence but not change the starting amino acid (for example exchanging CGG (encoding arginine) to CGA (still encoding arginine) to increase host organism expression levels) is not an “amino acid substitution;” that is, despite the creation of a new gene encoding the same protein, if the protein has the same amino acid at the particular position that it started with, it is not an amino acid substitution.

By “amino acid insertion” or “insertion” as used herein is meant the addition of an amino acid sequence at a particular position in a parent polypeptide sequence. For example, −233E or 233E designates an insertion of glutamic acid after position 233 and before position 234. Additionally, −233ADE or A233ADE designates an insertion of AlaAspGlu after position 233 and before position 234.

By “amino acid deletion” or “deletion” as used herein is meant the removal of an amino acid sequence at a particular position in a parent polypeptide sequence. For example, E233- or E233 #, E233( ) or E233del designates a deletion of glutamic acid at position 233. Additionally, EDA233- or EDA233 # designates a deletion of the sequence GluAspAla that begins at position 233.

By “variant protein” or “protein variant”, or “variant” as used herein is meant a protein that differs from that of a parent protein by virtue of at least one amino acid modification. The protein variant has at least one amino acid modification compared to the parent protein, yet not so many that the variant protein will not align with the parental protein using an alignment program such as that described below. In general, variant proteins (such as variant Fc domains, etc., outlined herein, are generally at least 75, 80, 85, 90, 91, 92, 93, 94, 95, 96, 97, 98 or 99% identical to the parent protein, using the alignment programs described below, such as BLAST.

“Variant” as used herein also refers to particular amino acid modifications that confer particular function (e.g., a “heterodimerization variant,” “pI variant,” “ablation variant,” etc.).

As described below, in some embodiments the parent polypeptide, for example an Fc parent polypeptide, is a human wild-type sequence, such as the heavy constant domain or Fc region from IgG1, IgG2, IgG3 or IgG4, although human sequences with variants can also serve as “parent polypeptides”, for example the IgG1/2 hybrid of US Publication 2006/0134105 can be included. The protein variant sequence herein will preferably possess at least about 80% identity with a parent protein sequence, and most preferably at least about 90% identity, more preferably at least about 95-98-99% identity. Accordingly, by “antibody variant” or “variant antibody” as used herein is meant an antibody that differs from a parent antibody by virtue of at least one amino acid modification, “IgG variant” or “variant IgG” as used herein is meant an antibody that differs from a parent IgG (again, in many cases, from a human IgG sequence) by virtue of at least one amino acid modification, and “immunoglobulin variant” or “variant immunoglobulin” as used herein is meant an immunoglobulin sequence that differs from that of a parent immunoglobulin sequence by virtue of at least one amino acid modification. “Fc variant” or “variant Fc” as used herein is meant a protein comprising an amino acid modification in an Fc domain as compared to an Fc domain of human IgG1, IgG2 or IgG4.

“Fc variant” or “variant Fc” as used herein is meant a protein comprising an amino acid modification in an Fc domain. The modification can be an addition, deletion, or substitution. The Fc variants are defined according to the amino acid modifications that compose them. Thus, for example, N434S or 434S is an Fc variant with the substitution for serine at position 434 relative to the parent Fc polypeptide, wherein the numbering is according to the EU index. Likewise, M428L/N434S defines an Fc variant with the substitutions M428L and N434S relative to the parent Fc polypeptide. The identity of the WT amino acid may be unspecified, in which case the aforementioned variant is referred to as 428L/434S. It is noted that the order in which substitutions are provided is arbitrary, that is to say that, for example, 428L/434S is the same Fc variant as 434S/428L, and so on. For all positions discussed herein that relate to antibodies or derivatives and fragments thereof (e.g., Fe domains), unless otherwise noted, amino acid position numbering is according to the EU index. The “EU index” or “EU index as in Kabat” or “EU numbering” scheme refers to the numbering of the EU antibody (Edelman et al., 1969, Proc Natl Acad Sci USA 63:78-85, hereby entirely incorporated by reference). The modification can be an addition, deletion, or substitution.

In general, variant Fc domains have at least about 80, 85, 90, 95, 97, 98 or 99 percent identity to the corresponding parental human IgG Fc domain (using the identity algorithms discussed below, with one embodiment utilizing the BLAST algorithm as is known in the art, using default parameters). Alternatively, the variant Fc domains can have from 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Alternatively, the variant Fc domains can have up to 1, 2, 3, 4, 5, 6, 7, 8, 9, 10, 11, 12, 13, 14, 15, 16, 11, 12, 13, 14, 15, 16, 17, 18, 19 or 20 amino acid modifications as compared to the parental Fc domain. Additionally, as discussed herein, the variant Fc domains described herein still retain the ability to form a dimer with another Fc domain as measured using known techniques as described herein, such as non-denaturing gel electrophoresis.

By “protein” as used herein is meant at least two covalently attached amino acids, which includes proteins, polypeptides, oligopeptides and peptides. In addition, polypeptides that make up the antibodies of the invention may include synthetic derivatization of one or more side chains or termini, glycosylation, PEGylation, circular permutation, cyclization, linkers to other molecules, fusion to proteins or protein domains, and addition of peptide tags or labels.

By “residue” as used herein is meant a position in a protein and its associated amino acid identity. For example, Asparagine 297 (also referred to as Asn297 or N297) is a residue at position 297 in the human antibody IgG1.

By “IgG subclass modification” or “isotype modification” as used herein is meant an amino acid modification that converts one amino acid of one IgG isotype to the corresponding amino acid in a different, aligned IgG isotype. For example, because IgG1 comprises a tyrosine and IgG2 a phenylalanine at EU position 296, a F296Y substitution in IgG2 is considered an IgG subclass modification.

By “non-naturally occurring modification” as used herein is meant an amino acid modification that is not isotypic. For example, because none of the human IgGs comprise a serine at position 434, the substitution 434S in IgG1, IgG2, IgG3, or IgG4 (or hybrids thereof) is considered a non-naturally occurring modification.

By “amino acid” and “amino acid identity” as used herein is meant one of the 20 naturally occurring amino acids that are coded for by DNA and RNA.

By “effector function” as used herein is meant a biochemical event that results from the interaction of an antibody Fc region with an Fc receptor or ligand. Effector functions include but are not limited to ADCC, ADCP, and CDC.

By “IgG Fc ligand” as used herein is meant a molecule, preferably a polypeptide, from any organism that binds to the Fc region of an IgG antibody to form an Fc/Fc ligand complex. Fc ligands include but are not limited to FcγRIs, FcγRIIs, FcγRIIIs, FcRn, C1q, C3, mannan binding lectin, mannose receptor, staphylococcal protein A, streptococcal protein G, and viral FcγR. Fc ligands also include Fc receptor homologs (FcRH), which are a family of Fc receptors that are homologous to the FcγRs (Davis et al., 2002, Immunological Reviews 190:123-136, entirely incorporated by reference). Fc ligands may include undiscovered molecules that bind Fc. Particular IgG Fc ligands are FcRn and Fc gamma receptors. By “Fc ligand” as used herein is meant a molecule, preferably a polypeptide, from any organism that binds to the Fc region of an antibody to form an Fc/Fc ligand complex.

By “Fc gamma receptor”, “FcγR” or “FcgammaR” as used herein is meant any member of the family of proteins that bind the IgG antibody Fc region and is encoded by an FcγR gene. In humans this family includes but is not limited to FcγRI (CD64), including isoforms FcγRIa, FcγRIb, and FcγRIc; FcγRII (CD32), including isoforms FcγRIIa (including allotypes H131 and R131), FcγRIIb (including FcγRIIb-1 and FcγRIIb-2), and FcγRIIc; and FcγRIII (CD16), including isoforms FcγRIIIa (including allotypes V158 and F158) and FcγRIIIb (including allotypes FcγRIIb-NA1 and FcγRIIb-NA2) (Jefferis et al., 2002, Immunol Lett 82:57-65, entirely incorporated by reference), as well as any undiscovered human FcγRs or FcγR isoforms or allotypes. An FcγR may be from any organism, including but not limited to humans, mice, rats, rabbits, and monkeys. Mouse FcγRs include but are not limited to FcγRI (CD64), FcγRII (CD32), FcγRIII (CD16), and FcγRIII-2 (CD16-2), as well as any undiscovered mouse FcγRs or FcγR isoforms or allotypes.

By “FcRn” or “neonatal Fc Receptor” as used herein is meant a protein that binds the IgG antibody Fc region and is encoded at least in part by an FcRn gene. The FcRn may be from any organism, including but not limited to humans, mice, rats, rabbits, and monkeys. As is known in the art, the functional FcRn protein comprises two polypeptides, often referred to as the heavy chain and light chain. The light chain is beta-2-microglobulin and the heavy chain is encoded by the FcRn gene. Unless otherwise noted herein, FcRn or an FcRn protein refers to the complex of FcRn heavy chain with beta-2-microglobulin. A variety of FcRn variants used to increase binding to the FcRn receptor, and in some cases, to increase serum half-life. An “FcRn variant” is an amino acid modification that contributes to increased binding to the FcRn receptor, and suitable FcRn variants are shown below.

By “parent polypeptide” as used herein is meant a starting polypeptide that is subsequently modified to generate a variant. The parent polypeptide may be a naturally occurring polypeptide, or a variant or engineered version of a naturally occurring polypeptide. Accordingly, by “parent immunoglobulin” as used herein is meant an unmodified immunoglobulin polypeptide that is modified to generate a variant, and by “parent antibody” as used herein is meant an unmodified antibody that is modified to generate a variant antibody. It should be noted that “parent antibody” includes known commercial, recombinantly produced antibodies as outlined below. In this context, a “parent Fc domain” will be relative to the recited variant; thus, a “variant human IgG1 Fc domain” is compared to the parent Fc domain of human IgG1, a “variant human IgG4 Fc domain” is compared to the parent Fc domain human IgG4, etc.

By “position” as used herein is meant a location in the sequence of a protein. Positions may be numbered sequentially, or according to an established format, for example the EU index for numbering of antibody domains (e.g., a CH1, CH2, CH3 or hinge domain).

By “target antigen” as used herein is meant the molecule that is bound specifically by the antigen binding domain comprising the variable regions of a given antibody.

By “strandedness” in the context of the monomers of the heterodimeric antibodies of the invention herein is meant that, similar to the two strands of DNA that “match”, heterodimerization variants are incorporated into each monomer so as to preserve the ability to “match” to form heterodimers. For example, if some pI variants are engineered into monomer A (e.g., making the pI higher) then steric variants that are “charge pairs” that can be utilized as well do not interfere with the pI variants, e.g., the charge variants that make a pI higher are put on the same “strand” or “monomer” to preserve both functionalities. Similarly, for “skew” variants that come in pairs of a set as more fully outlined below, the skilled artisan will consider pI in deciding into which strand or monomer one set of the pair will go, such that pI separation is maximized using the pI of the skews as well.

By “target cell” as used herein is meant a cell that expresses a target antigen.

By “host cell” in the context of producing a bispecific antibody according to the invention herein is meant a cell that contains the exogeneous nucleic acids encoding the components of the bispecific antibody and is capable of expressing the bispecific antibody under suitable conditions. Suitable host cells are discussed below.

By “wild type” or “WT” herein is meant an amino acid sequence or a nucleotide sequence that is found in nature, including allelic variations. A WT protein has an amino acid sequence or a nucleotide sequence that has not been intentionally modified.

Provided herein are a number of antibody domains (e.g., Fc domains) that have sequence identity to human antibody domains. Sequence identity between two similar sequences (e.g., antibody variable domains) can be measured by algorithms such as that of Smith, T. F. & Waterman, M. S. (1981) “Comparison Of Biosequences,” Adv. Appl. Math. 2:482 [local homology algorithm]; Needleman, S. B. & Wunsch, C D. (1970) “A General Method Applicable To The Search For Similarities In The Amino Acid Sequence Of Two Proteins,” J. Mol. Biol. 48:443 [homology alignment algorithm], Pearson, W. R. & Lipman, D. J. (1988) “Improved Tools For Biological Sequence Comparison,” Proc. Natl. Acad. Sci. (U.S.A.) 85:2444 [search for similarity method]; or Altschul, S. F. et al, (1990) “Basic Local Alignment Search Tool,” J. Mol. Biol. 215:403-10, the “BLAST” algorithm, see https://blast.ncbi.nlm.nih.gov/Blast.cgi. When using any of the aforementioned algorithms, the default parameters (for Window length, gap penalty, etc.) are used. In one embodiment, sequence identity is done using the BLAST algorithm, using default parameters

The antibodies of the present invention are generally isolated or recombinant. “Isolated,” when used to describe the various polypeptides disclosed herein, means a polypeptide that has been identified and separated and/or recovered from a cell or cell culture from which it was expressed. Ordinarily, an isolated polypeptide will be prepared by at least one purification step. An “isolated antibody,” refers to an antibody which is substantially free of other antibodies having different antigenic specificities. “Recombinant” means the antibodies are generated using recombinant nucleic acid techniques in exogeneous host cells, and they can be isolated as well.

“Specific binding” or “specifically binds to” or is “specific for” a particular antigen or an epitope means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target.

Specific binding for a particular antigen or an epitope can be exhibited, for example, by an antibody having a KD for an antigen or epitope of at least about 10⁻⁴ M, at least about 10⁻⁵ M, at least about 10⁻⁶ M, at least about 10⁻⁷ M, at least about 10⁻⁸ M, at least about 10⁻⁹ M, alternatively at least about 10⁻¹⁰ M, at least about 10⁻¹¹ M, at least about 10⁻¹² M, or greater, where KD refers to a dissociation rate of a particular antibody-antigen interaction. Typically, an antibody that specifically binds an antigen will have a KD that is 20-, 50-, 100-, 500-, 1000-, 5,000-, 10,000- or more times greater for a control molecule relative to the antigen or epitope.

Also, specific binding for a particular antigen or an epitope can be exhibited, for example, by an antibody having a KA or Ka for an antigen or epitope of at least 20-, 50-, 100-, 500-, 1000-, 5,000-, 10,000- or more times greater for the epitope relative to a control, where KA or Ka refers to an association rate of a particular antibody-antigen interaction. Binding affinity is generally measured using a Biacore, SPR or BLI assay.

### IV. PD-L1, PD-L2, and CD28 Antigen Binding Domains

Provided herein are antigen binding domains (ABDs) and ABD compositions that bind either PD-L1, PD-L2, or CD28. In some embodiments, one or more of the ABDs are included in an antibody format described herein including, any of the bispecific and trispecific formats in FIGS. 82 and 83.

### A. PD-L1 Antigen Binding Domains

In one aspect, provided herein are PD-L1 antigen binding domains (ABDs) and compositions that include such PD-L1 antigen binding domains (ABDs), including anti-PD-L1 antibodies (e.g., anti-PD-L1×anti-CD28 bispecific antibodies, and anti-PD-L1×anti-PD-L2×anti-CD28 trispecific antibodies). Such PD-L1 binding domains and related antibodies (e.g., bispecific and trispecific antibodies disclosed herein) find use, for example, in the treatment of PD-L1 associated cancers. In some embodiments, the PD-L1 ABDs are capable of binding to human and cynomolgus PD-L1 (see FIG. 1 and Example 1).

As will be appreciated by those in the art, suitable PD-L1 binding domains can comprise a set of 6 CDRs as depicted in FIGS. 25 and 26 and the Sequence Listing. Suitable PD-L1 ABDs can also include the entire VH and VL sequences as depicted in these sequences and FIGS. 25 and 26, and the Sequence Listing, used as scFvs or as Fab domains.

In one embodiment, the PD-L1 antigen binding domain includes the 6 CDRs (i.e., vhCDR1-3 and vlCDR1-3) of a PD-L1 ABD described herein, including FIGS. 25 and 26, and the Sequence Listing, either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIGS. 25 and 26, and the Sequence Listing (see Table 2). Suitable PD-L1 ABDs can also include the entire VH and VL sequences as depicted in these sequences and figures, used as scFvs or as Fab domains.

In one embodiment, the PD-L1 antigen binding domain includes the 6 CDRs (i.e., vhCDR1-3 and vlCDR1-3) of an PD-L1 ABD described herein, including FIGS. 25 and 26, and the Sequence Listing. In exemplary embodiments, the PD-L1 antigen binding domain comprises the 6 CDRs of a PD-L1 comprising a variable heavy domain (VH) and a variable light domain (VL), wherein VH and VL selected from the following:
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:21; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:25; and
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:29; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:33;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:37; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:33;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:29; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:37; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20 (FIGS. 24-26).

In addition to the parental CDR sets disclosed in the figures and sequence listing that form an ABD to PD-L1, provided herein are variant PD-L1 ABDS having CDRs that include at least one modification of the PD-L1 ABD CDRs disclosed herein. In one embodiment, the PD-L1 ABD includes a set of 6 CDRs with 1, 2, 3, 4, 5, 6, 7, 8, 9, 10 amino acid modifications as compared to the 6 CDRs of an PD-L1 ABD described herein, including the figures and sequence listing. In exemplary embodiments, the PD-L1 ABD includes a set of 6 CDRs with 1, 2, 3, 4, 5, 6, 7, 8, 9, 10 amino acid modifications as compared to the 6 CDRs of an PD-L1 ABD comprising a variable heavy domain (VH) and a variable light domain (VL) selected from the following:
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:21; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:25; and
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:29; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:33;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:37; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:33;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:29; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:37; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20 (FIGS. 24-26).

In certain embodiments, the variant PD-L1 ABD is capable of binding PD-L1 antigen, as measured by at least one of a Biacore, surface plasmon resonance (SPR) and/or BLI (biolayer interferometry, e.g., Octet assay) assay, with the latter finding particular use in many embodiments. In particular embodiments, the PD-L1 ABD is capable of binding to human and cynomolgus PD-L1.

In one embodiment, the anti-PD-L1 ABD includes 6 CDRs that are at least 90, 95, 97, 98 or 99% identical to the 6 CDRs of an anti-PD-L1 ABD as described herein, including FIGS. 24-26, and the Sequence Listing. In exemplary embodiments, the anti-PD-L1 ABD includes 6 CDRs that are at least 90, 95, 97, 98 or 99% identical to the 6 CDRs of an PD-L1 ABD comprising a variable heavy domain (VH) and a variable light domain (VL) selected from the following:
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:21; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:25; and
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:29; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:33;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:37; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:33;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:29; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:37; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20 (FIGS. 24-26).

In certain embodiments, the anti-PD-L1 ABD is capable of binding to PD-L1 antigen, as measured by at least one of a Biacore, surface plasmon resonance (SPR) and/or BLI (biolayer interferometry, e.g., Octet assay) assay, with the latter finding particular use in many embodiments. In particular embodiments, the PD-L1 ABD is capable of binding to human and cynomolgus PD-L1.

In another exemplary embodiment, the anti-PD-L1 ABD include the variable heavy (VH) domain and/or variable light (VL) domain of any one of the PD-L1 ABDs described herein, including FIGS. 24-26 and the Sequence Listing. In exemplary embodiments, VH and VL are selected from the following:
(i) a VH having an amino acid sequence of SEQ ID NO:21; and (ii) a VL having an amino acid sequence of SEQ ID NO:25; and
(i) a VH having an amino acid sequence of SEQ ID NO:29; and (ii) a VL having an amino acid sequence of SEQ ID NO:33;
(i) a VH having an amino acid sequence of SEQ ID NO:37; and (ii) a VL having an amino acid sequence of SEQ ID NO:33;
(i) a VH having an amino acid sequence of SEQ ID NO:29; and (ii) a VL having an amino acid sequence of SEQ ID NO:20; and
(i) a VH having an amino acid sequence of SEQ ID NO:37; and (ii) a VL having an amino acid sequence of SEQ ID NO:20 (FIGS. 24-26).

In addition to the parental anti-PD-L1 binding domain variable heavy and variable light domains disclosed herein, provided herein are anti-PD-L1 ABDs that include a variable heavy domain and/or a variable light domain that are variants of an anti-PD-L1 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of an anti-PD-L1 ABD described herein, including FIGS. 28-31 and the Sequence Listing. In exemplary embodiments, the variant VH domain and/or VL domain has from 1, 2, 3, 4, 5, 6, 7, 8, 9 or 10 amino acid changes from a VH and/or VL are selected from the following:
(i) a VH having an amino acid sequence of SEQ ID NO:21; and (ii) a VL having an amino acid sequence of SEQ ID NO:25; and
(i) a VH having an amino acid sequence of SEQ ID NO:29; and (ii) a VL having an amino acid sequence of SEQ ID NO:33;
(i) a VH having an amino acid sequence of SEQ ID NO:37; and (ii) a VL having an amino acid sequence of SEQ ID NO:33;
(i) a VH having an amino acid sequence of SEQ ID NO:29; and (ii) a VL having an amino acid sequence of SEQ ID NO:20; and
(i) a VH having an amino acid sequence of SEQ ID NO:37; and (ii) a VL having an amino acid sequence of SEQ ID NO:20 (FIGS. 24-26).

In certain embodiments, the anti-PD-L1 ABD is capable of binding to PD-L1, as measured at least one of a Biacore, surface plasmon resonance (SPR) and/or BLI (biolayer interferometry, e.g., Octet assay) assay, with the latter finding particular use in many embodiments. In particular embodiments, the PD-L1 ABD is capable of binding to human and cynomolgus PD-L1.

In some embodiments, the PD-L1 ABD includes a variable heavy domain that has from 1, 2, 3, 4, 5, 6, 7, 8, 9 or 10 amino acid changes one of the following PD-L1 ABD variable heavy domains: SEQ ID NOs:21, 29 and 37. In some embodiments, the PD-L1 ABD includes any of the variable light domains provided herein or a variant thereof.

In some embodiments, the PD-L1 ABD includes a variable light domain that has from 1, 2, 3, 4, 5, 6, 7, 8, 9 or 10 amino acid changes one of the following PD-L1 ABD variable light domains: SEQ ID NOs: 20, 25, and 33. In some embodiments, the PD-L1 ABD includes any of the variable heavy domains provided herein or a variant thereof.

In one embodiment, the variant VH and/or VL domain is at least 90, 95, 97, 98 or 99% identical to the VH and/or VL of an anti-PD-L1 ABD as described herein, including FIGS. 25 and 26, and the Sequence Listing. In exemplary embodiments, the variant VH and/or VL domain is at least 90, 95, 97, 98 or 99% identical to the VH and/or VL of one of an anti-PD-L1 ABD comprising a VH and VL selected from the following:
(i) a VH having an amino acid sequence of SEQ ID NO:21; and (ii) a VL having an amino acid sequence of SEQ ID NO:25; and
(i) a VH having an amino acid sequence of SEQ ID NO:29; and (ii) a VL having an amino acid sequence of SEQ ID NO:33;
(i) a VH having an amino acid sequence of SEQ ID NO:37; and (ii) a VL having an amino acid sequence of SEQ ID NO:33;
(i) a VH having an amino acid sequence of SEQ ID NO:29; and (ii) a VL having an amino acid sequence of SEQ ID NO:20; and
(i) a VH having an amino acid sequence of SEQ ID NO:37; and (ii) a VL having an amino acid sequence of SEQ ID NO:20 (FIGS. 24-26).

In certain embodiments, the anti-PD-L1 ABD is capable of binding to PD-L1, as measured by at least one of a Biacore, surface plasmon resonance (SPR) and/or BLI (biolayer interferometry, e.g., Octet assay) assay, with the latter finding particular use in many embodiments. In particular embodiments, the PD-L1 ABD is capable of binding to human and cynomolgus PD-L1.

In some embodiments, the PD-L1 ABD includes a variable heavy domain that is at least 90, 95, 97, 98 or 99% identical to one of the following PD-L1 ABD variable heavy domains: SEQ ID NOs:21, 29 and 37. In some embodiments, the PD-L1 ABD includes any of the variable light domains provided herein or a variant thereof.

In some embodiments, the PD-L1 ABD includes a variable light domain that is at least 90, 95, 97, 98 or 99% identical to one of the following PD-L1 ABD variable light domains: SEQ ID NOs: 20, 25, and 33. In some embodiments, the PD-L1 ABD includes any of the variable heavy domains provided herein or a variant thereof.

In some embodiments, the PD-L1 ABDs have variable heavy and variable light domains that are 90, 95, 97, 98 or 99% identical to the VH and/or VL domains of a PD-L1 ABD as described herein, but the CDRs are identical. In some embodiments, the VH selected from the group consisting of: SEQ ID NOs:21, 29 and 37. In some embodiments, the VL is selected from the group consisting of: SEQ ID NOs: 20, 25, and 33.

In some embodiments, the PD-L1 ABDs have variable heavy and variable light domains that are 90, 95, 97, 98 or 99% identical to the VH and/or VL domains of a PD-L1 ABD as described herein, and there can be from 0 to 6 amino acid modifications in the CDRs. In some embodiments, the VH selected from the group consisting of: SEQ ID NOs:21, 29 and 37. In some embodiments, the VL is selected from the group consisting of: SEQ ID NOs: 20, 25, and 33.

In some embodiments, the PD-L1 ABDs have variable heavy and variable light domains that are 90, 95, 97, 98 or 99% identical to the VH and/or VL domains of a PD-L1 ABD as described herein, and there can be from 0 to 6 amino acid modifications in the CDRs, with no CDR having more than 1 amino acid modification. In some embodiments, the VH selected from the group consisting of: SEQ ID NOs:21, 29 and 37. In some embodiments, the VL is selected from the group consisting of: SEQ ID NOs: 20, 25, and 33.

In certain embodiments, the PD-L1 ABD is capable of binding to the PD-L1, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human PD-L1 (see FIG. 2) at detectable limits of the assay.

Such PD-L1 binding domains can be included in any of the antibodies provided herein including, for example, the bispecific and trispecific antibody formats provided in FIGS. 82 and 83.

1. Additional PD-L1 Binding Domains

In another aspect, provided herein are additional PD-L1 binding domains that can be used in anti-PD-L1 antibodies, including any of the anti-PD-L1 antibodies described herein. In some embodiments, the anti-PD-L1 binding domain includes a variable heavy domain is selected from any of those in FIG. 161 (SEQ ID NOs:3335, and 3243-3260) or a variant thereof, and a common light chain with a variable light domain referred to as “IGKV1-39_L1” (also referred to as, “2A3A4.248[PDL1]_L1” (SEQ ID NO:3239, see FIG. 161A) and “1F12A4.249[PDL2]_L1” (SEQ ID NO:3271, see FIG. 162A)) or variant thereof, wherein the common light chain can also be used as a light chain for a CD28 and/or PD-L2 binding domain. In some embodiments, the anti-PD-L1 antibodies provided herein (e.g., anti-CD28×anti-PD-L1, and anti-CD28×anti-PD-L1×anti-PDL2 antibodies) includes a PD-L1 binding domain that includes a common light chain with the IGKV1-39_L1 variable light domain. In some embodiments, the anti-PD-L1 antibody is an anti-CD28×anti-PD-L1 antibody having a CD28 binding domain and a PD-L1 binding domain that each include a common light chain with the IGKV1-39_L1 variable light domain (SEQ ID NO:3239). In some embodiments, the anti-PD-L1 antibody is an anti-CD28×anti-PD-L1×anti-PD-L2 antibody having a CD28 binding domain, a PD-L1 binding domain, and a PD-L2 binding domain that each include a common light chain with the IGKV1-39_L1 variable light domain (SEQ ID NO:3239). Such PD-L1 binding domains that utilize a common light chain with the IGKV1-39_L1 variable light domain (SEQ ID NO:3239) can be used, for example, in any of the antibody formats provided herein that utilize a common light chain (see, e.g., 1+1 CLC, 2+1 CLC, 1+1+1 stackFab2-scFv-Fc, 1+1+1 Fab-(Fab-scFv)-Fc, 1+1+1 mAb-scFv, and 1+1+1 stackFab2-Fab-Fc formats disclosed herein, FIGS. 82 and 83).

In one embodiment, the PD-L1 antigen binding domain includes the vhCDR1-3 of any of the PD-L1 variable heavy domains depicted in FIG. 161 (SEQ ID NOs:3335, and 3243-3260), and the vlCDR1-3 of the IGK1-39_L1 variable light domain (SEQ ID NO:3239).

In one embodiment, the PD-L1 ABD of the subject anti-PD-L1 antibodies described herein includes a) a vhCDR1, vhCDR2, and/or vhCDR3 with 1, 2, 3, 4, 5, 6, 7, 8, 9, 10 amino acid modifications as compared to the vhCDR1, vhCDR2, and/or vhCDR3 of one of the PD-L1 variable heavy domains depicted in FIG. 161 (SEQ ID NOs:3335, and 3243-3260), respectively, and/or b) a vlCDR1, vlCDR2, and/or vlCDR3 with 1, 2, 3, 4, 5, 6, 7, 8, 9, 10 amino acid modifications as compared to the vlCDR1, vlCDR2, and/or vlCDR3 of the IGK1-39_L1 variable light domain (SEQ ID NO:3239), respectively. In certain embodiments, the PD-L1 ABD of the subject anti-PD-L1 antibody is capable of binding PD-L1 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the PD-L1 ABD is capable of binding human PD-L1 antigen (see FIG. 1).

In one embodiment, the PD-L1 ABD of the subject anti-PD-L1 antibody includes a vhCDR1, vhCDR2, and/or vhCDR3 that is at least 90, 95, 97, 98 or 99% identical to the vhCDR1, vhCDR2, and/or vhCDR3 of one of the PD-L1 variable heavy domains depicted in FIG. 161 (SEQ ID NOs:3335, and 3243-3260), respectively, and/or a vlCDR1, vlCDR2, and/or vlCDR3 that is at least 90, 95, 97, 98 or 99% identical to the vlCDR1, vlCDR2, and/or vlCDR3 of the IGK1-39_L1 variable light domain (SEQ ID NO:3239), respectively. In certain embodiments, the PD-L1 ABD is capable of binding to the PD-L1, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the PD-L1 ABD is capable of binding human PD-L1 antigen (see FIG. 1).

In another exemplary embodiment, the PD-L1 ABD of the subject anti-PD-L1 antibody includes the variable heavy (VH) domain of one of the PD-L1 variable heavy domains depicted in FIG. 161 (SEQ ID NOs:3335, and 3243-3260), and the IGK1-39_L1 variable light domain (SEQ ID NO:3239).

In some embodiments, the anti-PD-L1 antibody includes a PD-L1 ABD that includes a variable heavy domain that is a variant of one of the PD-L1 variable heavy domains depicted in FIG. 161 (SEQ ID NOs:3335, and 3243-3260), and/or a variable light domain that is a variant of the IGK1-39_L1 variable light domain (SEQ ID NO:3239). In some embodiments, the variant VH domain has from 1, 2, 3, 4, 5, 6, 7, 8, 9 or 10 amino acid changes compared to one of the PD-L1 variable heavy domains depicted in FIG. 161 (SEQ ID NOs:3335, and 3243-3260) and/or the variable light domain has from 1, 2, 3, 4, 5, 6, 7, 8, 9 or 10 amino acid changes from the IGK1-39_L1 variable light domain (SEQ ID NO:3239). In some embodiments, the one or more amino acid changes are in the VH and/or VL framework regions (FR1, FR2, FR3, and/or FR4). In some embodiments, the one or more amino acid change(s) are in one or more CDRs. In certain embodiments, the PD-L1 ABD is capable of binding to PD-L1, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the PD-L1 ABD is capable of binding human PD-L1 antigen (see FIG. 1).

In one embodiment, the variant VH domain is at least 90, 95, 97, 98 or 99% identical to one of the PD-L1 variable heavy domains depicted in FIG. 161 (SEQ ID NOs:3335, and 3243-3260) and/or the variable light domain is at least 90, 95, 97, 98 or 99% identical to the IGK1-39_L1 variable light domain (SEQ ID NO:3239). In certain embodiments, the PD-L1 ABD is capable of binding to PD-L1, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the PD-L1 ABD is capable of binding human PD-L1 antigen (see FIG. 1).

In some embodiments, the PD-L1 binding domain includes a VH that includes any one of the VHCDR1-3 and/or HFR1-4 sequences depicted in FIG. 161C and the IGK1-39_L1 variable light domain (SEQ ID NO:3239) or a variant thereof.

In another aspect, provided herein is a PD-L1 binding domain that competes with any of the PD-L1 binding domains disclosed herein for binding to human PD-L1.

### B. PD-L2 Antigen Binding Domains

In one aspect, provided herein are PD-L2 antigen binding domains (ABDs) and compositions that include such PD-L2 antigen binding domains (ABDs), including anti-PD-L2 antibodies (e.g., anti-PD-L1×anti-CD28 bispecific antibodies, and anti-PD-L1×anti-PD-L2×anti-CD28 trispecific antibodies). Such PD-L2 binding domains and related antibodies (e.g., bispecific and trispecific antibodies disclosed herein) find use, for example, in the treatment of PD-L2 associated cancers. In some embodiments, the PD-L2 ABDs are capable of binding to human and cynomolgus PD-L2 (see FIG. 2 and Example 1).

As will be appreciated by those in the art, suitable PD-L2 binding domains can comprise a set of 6 CDRs as depicted in FIGS. 29-32 and the Sequence Listing. Suitable PD-L2 ABDs can also include the entire VH and VL sequences as depicted in these sequences and FIGS. 29-32, and the Sequence Listing, used as scFvs or as Fab domains.

In one embodiment, the PD-L2 antigen binding domain includes the 6 CDRs (i.e., vhCDR1-3 and vlCDR1-3) of a PD-L2 ABD described herein, including FIGS. 29-32, and the Sequence Listing, either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIGS. 29-32, and the Sequence Listing (see Table 2). Suitable PD-L2 ABDs can also include the entire VH and VL sequences as depicted in these sequences and figures, used as scFvs or as Fab domains.

In one embodiment, the PD-L2 antigen binding domain includes the 6 CDRs (i.e., vhCDR1-3 and vlCDR1-3) of an PD-L2 ABD described herein, including FIGS. 29-32, and the Sequence Listing. In exemplary embodiments, the PD-L2 antigen binding domain comprises the 6 CDRs of a PD-L2 comprising a variable heavy domain (VH) and a variable light domain (VL), wherein VH and VL selected from the following:
((i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:41; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:45;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:49; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:45;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:41; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:49; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:53; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:57;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:57;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:53; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:69;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:73; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:69;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:73; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:77; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:81;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:85; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:81;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:77; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20; and
(i) a VH having an amino acid sequence of SEQ ID NO:85; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20 (FIG. 29-32). (FIGS. 24, and 29-32).

In addition to the parental CDR sets disclosed in the figures and sequence listing that form an ABD to PD-L2, provided herein are variant PD-L2 ABDS having CDRs that include at least one modification of the PD-L2 ABD CDRs disclosed herein. In one embodiment, the PD-L2 ABD includes a set of 6 CDRs with 1, 2, 3, 4, 5, 6, 7, 8, 9, 10 amino acid modifications as compared to the 6 CDRs of an PD-L2 ABD described herein, including the figures and sequence listing. In exemplary embodiments, the PD-L2 ABD includes a set of 6 CDRs with 1, 2, 3, 4, 5, 6, 7, 8, 9, 10 amino acid modifications as compared to the 6 CDRs of an PD-L2 ABD comprising a variable heavy domain (VH) and a variable light domain (VL) selected from the following:
((i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:41; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:45;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:49; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:45;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:41; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:49; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:53; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:57;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:57;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:53; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:69;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:73; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:69;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:73; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:77; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:81;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:85; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:81;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:77; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20; and
(i) a VH having an amino acid sequence of SEQ ID NO:85; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20 (FIG. 29-32). (FIGS. 24, and 29-32).

In certain embodiments, the variant PD-L2 ABD is capable of binding PD-L2 antigen, as measured by at least one of a Biacore, surface plasmon resonance (SPR) and/or BLI (biolayer interferometry, e.g., Octet assay) assay, with the latter finding particular use in many embodiments. In particular embodiments, the PD-L2 ABD is capable of binding to human and cynomolgus PD-L2.

In one embodiment, the anti-PD-L2 ABD includes 6 CDRs that are at least 90, 95, 97, 98 or 99% identical to the 6 CDRs of an anti-PD-L2 ABD as described herein, including FIGS. 24, and 29-32, and the Sequence Listing. In exemplary embodiments, the anti-PD-L2 ABD includes 6 CDRs that are at least 90, 95, 97, 98 or 99% identical to the 6 CDRs of an PD-L2 ABD comprising a variable heavy domain (VH) and a variable light domain (VL) selected from the following:
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:41; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:45;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:49; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:45;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:41; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:49; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:53; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:57;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:57;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:53; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:61; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:69;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:73; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:69;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:65; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:73; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:77; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:81;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:85; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:81;
(i) a VH comprising a VH CDR1, a VH CDR2, and a VH CDR3 having an amino acid sequence of a VH CDR1, a VH CDR2, and a VH CDR3, respectively, of a VH having an amino acid sequence of SEQ ID NO:77; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20; and
(i) a VH having an amino acid sequence of SEQ ID NO:85; and (ii) a VL comprising a VL CDR1, a VL CDR2, and a VL CDR3 having an amino acid sequence of a VL CDR1, a VL CDR2, and a VL CDR3, respectively, of a VL having an amino acid sequence of SEQ ID NO:20 (FIG. 29-32). (FIGS. 24, and 29-32).

In certain embodiments, the anti-PD-L2 ABD is capable of binding to PD-L2 antigen, as measured by at least one of a Biacore, surface plasmon resonance (SPR) and/or BLI (biolayer interferometry, e.g., Octet assay) assay, with the latter finding particular use in many embodiments. In particular embodiments, the PD-L2 ABD is capable of binding to human and cynomolgus PD-L2.

In another exemplary embodiment, the anti-PD-L2 ABD include the variable heavy (VH) domain and/or variable light (VL) domain of any one of the PD-L2 ABDs described herein, including FIGS. 24, and 29-32 and the Sequence Listing. In exemplary embodiments, VH and VL are selected from the following:
((i) a VH having an amino acid sequence of SEQ ID NO:41; and (ii) a VL having an amino acid sequence of SEQ ID NO:45;
(i) a VH having an amino acid sequence of SEQ ID NO:49; and (ii) a VL having an amino acid sequence of SEQ ID NO:45;
(i) a VH having an amino acid sequence of SEQ ID NO:41; and (ii) a VL having an amino acid sequence of SEQ ID NO:20; and
(i) a VH having an amino acid sequence of SEQ ID NO:49; and (ii) a VL having an amino acid sequence of SEQ ID NO:20;
(i) a VH having an amino acid sequence of SEQ ID NO:53; and (ii) a VL having an amino acid sequence of SEQ ID NO:57;
(i) a VH having an amino acid sequence of SEQ ID NO:61; and (ii) a VL having an amino acid sequence of SEQ ID NO:57;
(i) a VH having an amino acid sequence of SEQ ID NO:53; and (ii) a VL having an amino acid sequence of SEQ ID NO:20;
(i) a VH having an amino acid sequence of SEQ ID NO:61; and (ii) a VL having an amino acid sequence of SEQ ID NO:20;
(i) a VH having an amino acid sequence of SEQ ID NO:65; and (ii) a VL having an amino acid sequence of SEQ ID NO:69;
(i) a VH having an amino acid sequence of SEQ ID NO:73; and (ii) a VL having an amino acid sequence of SEQ ID NO:69;
(i) a VH having an amino acid sequence of SEQ ID NO:65; and (ii) a VL having an amino acid sequence of SEQ ID NO:20;
(i) a VH having an amino acid sequence of SEQ ID NO:73; and (ii) a VL having an amino acid sequence of SEQ ID NO:20;
(i) a VH having an amino acid sequence of SEQ ID NO:77; and (ii) a VL having an amino acid sequence of SEQ ID NO:81;
(i) a VH having an amino acid sequence of SEQ ID NO:85; and (ii) a VL having an amino acid sequence of SEQ ID NO:81;
(i) a VH having an amino acid sequence of SEQ ID NO:77; and (ii) a VL having an amino acid sequence of SEQ ID NO:20; and
(i) a VH having an amino acid sequence of SEQ ID NO:85; and (ii) a VL having an amino acid sequence of SEQ ID NO:20 (FIGS. 24, and 29-32).

In addition to the parental anti-PD-L2 binding domain variable heavy and variable light domains disclosed herein, provided herein are anti-PD-L2 ABDs that include a variable heavy domain and/or a variable light domain that are variants of an anti-PD-L2 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of an anti-PD-L2 ABD described herein, including FIGS. 28-31 and the Sequence Listing. In exemplary embodiments, the variant VH domain and/or VL domain has from 1, 2, 3, 4, 5, 6, 7, 8, 9 or 10 amino acid changes from a VH and/or VL are selected from the following:
(i) a VH having an amino acid sequence of SEQ ID NO:41; and (ii) a VL having an amino acid sequence of SEQ ID NO:45;
(i) a VH having an amino acid sequence of SEQ ID NO:49; and (ii) a VL having an amino acid sequence of SEQ ID NO:45;
(i) a VH having an amino acid sequence of SEQ ID NO:41; and (ii) a VL having an amino acid sequence of SEQ ID NO:20; and
(i) a VH having an amino acid sequence of SEQ ID NO:49; and (ii) a VL having an amino acid sequence of SEQ ID NO:20;
(i) a VH having an amino acid sequence of SEQ ID NO:53; and (ii) a VL having an amino acid sequence of SEQ ID NO:57;
(i) a VH having an amino acid sequence of SEQ ID NO:61; and (ii) a VL having an amino acid sequence of SEQ ID NO:57;
(i) a VH having an amino acid sequence of SEQ ID NO:53; and (ii) a VL having an amino acid sequence of SEQ ID NO:20;
(i) a VH having an amino acid sequence of SEQ ID NO:61; and (ii) a VL having an amino acid sequence of SEQ ID NO:20;
(i) a VH having an amino acid sequence of SEQ ID NO:65; and (ii) a VL having an amino acid sequence of SEQ ID NO:69;
(i) a VH having an amino acid sequence of SEQ ID NO:73; and (ii) a VL having an amino acid sequence of SEQ ID NO:69;
(i) a VH having an amino acid sequence of SEQ ID NO:65; and (ii) a VL having an amino acid sequence of SEQ ID NO:20;
(i) a VH having an amino acid sequence of SEQ ID NO:73; and (ii) a VL having an amino acid sequence of SEQ ID NO:20;
(i) a VH having an amino acid sequence of SEQ ID NO:77; and (ii) a VL having an amino acid sequence of SEQ ID NO:81;
(i) a VH having an amino acid sequence of SEQ ID NO:85; and (ii) a VL having an amino acid sequence of SEQ ID NO:81;
(i) a VH having an amino acid sequence of SEQ ID NO:77; and (ii) a VL having an amino acid sequence of SEQ ID NO:20; and
(i) a VH having an amino acid sequence of SEQ ID NO:85; and (ii) a VL having an amino acid sequence of SEQ ID NO:20 (FIGS. 24, and 29-32).

In certain embodiments, the anti-PD-L2 ABD is capable of binding to PD-L2, as measured at least one of a Biacore, surface plasmon resonance (SPR) and/or BLI (biolayer interferometry, e.g., Octet assay) assay, with the latter finding particular use in many embodiments. In particular embodiments, the PD-L2 ABD is capable of binding to human and cynomolgus PD-L2.

In some embodiments, the PD-L2 ABD includes a variable heavy domain that has from 1, 2, 3, 4, 5, 6, 7, 8, 9 or 10 amino acid changes one of the following PD-L2 ABD variable heavy domains: SEQ ID NOs:41, 49, 53, 61, 65, 73, 77 and 85. In some embodiments, the PD-L2 ABD includes any of the variable light domains provided herein or a variant thereof.

In some embodiments, the PD-L2 ABD includes a variable light domain that has from 1, 2, 3, 4, 5, 6, 7, 8, 9 or 10 amino acid changes one of the following PD-L2 ABD variable light domains: SEQ ID NOs: 20, 45, 57, 69, and 81. In some embodiments, the PD-L2 ABD includes any of the variable heavy domains provided herein or a variant thereof.

In one embodiment, the variant VH and/or VL domain is at least 90, 95, 97, 98 or 99% identical to the VH and/or VL of an anti-PD-L2 ABD as described herein, including FIGS. 29-32, and the Sequence Listing. In exemplary embodiments, the variant VH and/or VL domain is at least 90, 95, 97, 98 or 99% identical to the VH and/or VL of one of an anti-PD-L2 ABD comprising a VH and VL selected from the following:
((i) a VH having an amino acid sequence of SEQ ID NO:41; and (ii) a VL having an amino acid sequence of SEQ ID NO:45;
(i) a VH having an amino acid sequence of SEQ ID NO:49; and (ii) a VL having an amino acid sequence of SEQ ID NO:45;
(i) a VH having an amino acid sequence of SEQ ID NO:41; and (ii) a VL having an amino acid sequence of SEQ ID NO:20; and
(i) a VH having an amino acid sequence of SEQ ID NO:49; and (ii) a VL having an amino acid sequence of SEQ ID NO:20;
(i) a VH having an amino acid sequence of SEQ ID NO:53; and (ii) a VL having an amino acid sequence of SEQ ID NO:57;
(i) a VH having an amino acid sequence of SEQ ID NO:61; and (ii) a VL having an amino acid sequence of SEQ ID NO:57;
(i) a VH having an amino acid sequence of SEQ ID NO:53; and (ii) a VL having an amino acid sequence of SEQ ID NO:20;
(i) a VH having an amino acid sequence of SEQ ID NO:61; and (ii) a VL having an amino acid sequence of SEQ ID NO:20;
(i) a VH having an amino acid sequence of SEQ ID NO:65; and (ii) a VL having an amino acid sequence of SEQ ID NO:69;
(i) a VH having an amino acid sequence of SEQ ID NO:73; and (ii) a VL having an amino acid sequence of SEQ ID NO:69;
(i) a VH having an amino acid sequence of SEQ ID NO:65; and (ii) a VL having an amino acid sequence of SEQ ID NO:20;
(i) a VH having an amino acid sequence of SEQ ID NO:73; and (ii) a VL having an amino acid sequence of SEQ ID NO:20;
(i) a VH having an amino acid sequence of SEQ ID NO:77; and (ii) a VL having an amino acid sequence of SEQ ID NO:81;
(i) a VH having an amino acid sequence of SEQ ID NO:85; and (ii) a VL having an amino acid sequence of SEQ ID NO:81;
(i) a VH having an amino acid sequence of SEQ ID NO:77; and (ii) a VL having an amino acid sequence of SEQ ID NO:20; and
(i) a VH having an amino acid sequence of SEQ ID NO:85; and (ii) a VL having an amino acid sequence of SEQ ID NO:20 (FIGS. 24, and 29-32).

In certain embodiments, the anti-PD-L2 ABD is capable of binding to PD-L2, as measured by at least one of a Biacore, surface plasmon resonance (SPR) and/or BLI (biolayer interferometry, e.g., Octet assay) assay, with the latter finding particular use in many embodiments. In particular embodiments, the PD-L2 ABD is capable of binding to human and cynomolgus PD-L2.

In some embodiments, the PD-L2 ABD includes a variable heavy domain that is at least 90, 95, 97, 98 or 99% identical to one of the following PD-L2 ABD variable heavy domains: SEQ ID NOs:41, 49, 53, 61, 65, 73, 77 and 85. In some embodiments, the PD-L2 ABD includes any of the variable light domains provided herein or a variant thereof.

In some embodiments, the PD-L2 ABD includes a variable light domain that is at least 90, 95, 97, 98 or 99% identical to one of the following PD-L2 ABD variable light domains: SEQ ID NOs: 20, 45, 57, 69, and 81. In some embodiments, the PD-L2 ABD includes any of the variable heavy domains provided herein or a variant thereof.

In some embodiments, the PD-L2 ABDs have variable heavy and variable light domains that are 90, 95, 97, 98 or 99% identical to the VH and/or VL domains of a PD-L2 ABD as described herein, but the CDRs are identical. In some embodiments, the VH selected from the group consisting of: SEQ ID NOs:41, 49, 53, 61, 65, 73, 77 and 85. In some embodiments, the VL is selected from the group consisting of: SEQ ID NOs: 20, 45, 57, 69, and 81.

In some embodiments, the PD-L2 ABDs have variable heavy and variable light domains that are 90, 95, 97, 98 or 99% identical to the VH and/or VL domains of a PD-L2 ABD as described herein, and there can be from 0 to 6 amino acid modifications in the CDRs. In some embodiments, the VH selected from the group consisting of: SEQ ID NOs:41, 49, 53, 61, 65, 73, 77 and 85. In some embodiments, the VL is selected from the group consisting of: SEQ ID NOs: 20, 45, 57, 69, and 81.

In some embodiments, the PD-L2 ABDs have variable heavy and variable light domains that are 90, 95, 97, 98 or 99% identical to the VH and/or VL domains of a PD-L2 ABD as described herein, and there can be from 0 to 6 amino acid modifications in the CDRs, with no CDR having more than 1 amino acid modification. In some embodiments, the VH selected from the group consisting of: SEQ ID NOs:41, 49, 53, 61, 65, 73, 77 and 85. In some embodiments, the VL is selected from the group consisting of: SEQ ID NOs: 20, 45, 57, 69, and 81.

In certain embodiments, the PD-L2 ABD is capable of binding to the PD-L2, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human PD-L2 (see FIG. 2) at detectable limits of the assay.

Such PD-L2 binding domains can be included in any of the antibodies provided herein including, for example, the bispecific and trispecific antibody formats provided in FIGS. 82 and 83.

1. Additional PD-L2 Binding Domains

In another aspect, provided herein are additional PD-L2 binding domains that can be used in anti-PD-L2 antibodies, including any of the anti-PD-L1 antibodies described herein. In some embodiments, the anti-PD-L1 binding domain includes a variable heavy domain selected from any of those in FIG. 162 (SEQ ID NOs:3267, and 3275-3347) or a variant thereof, and a common light chain with a variable light domain referred to as “IGKV1-39_L1” (also referred to as, “2A3A4.248[PDL1]_L1” (SEQ ID NO:3239, see FIG. 161A) and “1F12A4.249[PDL2]_L1” (SEQ ID NO:3271, see FIG. 162A)) or variant thereof, wherein the common light chain can also be used as a light chain for a CD28 and/or PD-L1 binding domain. In some embodiments, the anti-PD-L1 antibodies provided herein (e.g., anti-CD28×anti-PD-L2, and anti-CD28×anti-PD-L1×anti-PDL2 antibodies) includes a PD-L2 binding domain that includes a common light chain with the IGKV1-39_L1 variable light domain. In some embodiments, the anti-PD-L2 antibody is an anti-CD28×anti-PD-L2 antibody having a CD28 binding domain and a PD-L2 binding domain that each include a common light chain with the IGKV1-39_L1 variable light domain (SEQ ID NO:3239). In some embodiments, the anti-PD-L2 antibody is an anti-CD28×anti-PD-L1×anti-PD-L2 antibody having a CD28 binding domain, a PD-L1 binding domain, and a PD-L2 binding domain that each include a common light chain with the IGKV1-39_L1 variable light domain (SEQ ID NO:3239). Such PD-L2 binding domains that utilize a common light chain with the IGKV1-39_L1 variable light domain (SEQ ID NO:3239) can be used, for example, in any of the antibody formats provided herein that utilize a common light chain (see, e.g., 1+1 CLC, 2+1 CLC, 1+1+1 stackFab2-scFv-Fc, 1+1+1 Fab-(Fab-scFv)-Fc, 1+1+1 mAb-scFv, and 1+1+1 stackFab2-Fab-Fc formats disclosed herein, FIGS. 82 and 83).

In one embodiment, the PD-L2 antigen binding domain includes the vhCDR1-3 of any of the PD-L2 variable heavy domains depicted in FIG. 161 (SEQ ID NOs:3335, and 3243-3260), and the vlCDR1-3 of the IGK1-39_L1 variable light domain (SEQ ID NO:3239).

In one embodiment, the PD-L2 ABD of the subject anti-PD-L2 antibodies described herein includes a) a vhCDR1, vhCDR2, and/or vhCDR3 with 1, 2, 3, 4, 5, 6, 7, 8, 9, 10 amino acid modifications as compared to the vhCDR1, vhCDR2, and/or vhCDR3 of one of the PD-L2 variable heavy domains depicted in FIG. 162 (SEQ ID NOs:3267, and 3275-3347), respectively, and/or b) a vlCDR1, vlCDR2, and/or vlCDR3 with 1, 2, 3, 4, 5, 6, 7, 8, 9, 10 amino acid modifications as compared to the vlCDR1, vlCDR2, and/or vlCDR3 of the IGK1-39_L1 variable light domain (SEQ ID NO:3239), respectively. In certain embodiments, the PD-L2 ABD of the subject anti-PD-L2 antibody is capable of binding PD-L2 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the PD-L2 ABD is capable of binding human PD-L2 antigen (see FIG. 1).

In one embodiment, the PD-L2 ABD of the subject anti-PD-L2 antibody includes a vhCDR1, vhCDR2, and/or vhCDR3 that is at least 90, 95, 97, 98 or 99% identical to the vhCDR1, vhCDR2, and/or vhCDR3 of one of the PD-L2 variable heavy domains depicted in FIG. 162 (SEQ ID NOs:3267, and 3275-3347), respectively, and/or a vlCDR1, vlCDR2, and/or vlCDR3 that is at least 90, 95, 97, 98 or 99% identical to the vlCDR1, vlCDR2, and/or vlCDR3 of the IGK1-39_L1 variable light domain (SEQ ID NO:3239), respectively. In certain embodiments, the PD-L2 ABD is capable of binding to the PD-L2, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the PD-L2 ABD is capable of binding human PD-L2 antigen (see FIG. 1).

In another exemplary embodiment, the PD-L2 ABD of the subject anti-PD-L2 antibody includes the variable heavy (VH) domain of one of the PD-L2 variable heavy domains depicted in FIG. 162 (SEQ ID NOs:3267, and 3275-3347), and the IGK1-39_L1 variable light domain (SEQ ID NO:3239).

In some embodiments, the anti-PD-L2 antibody includes a PD-L2 ABD that includes a variable heavy domain that is a variant of one of the PD-L2 variable heavy domains depicted in FIG. 162 (SEQ ID NOs:3267, and 3275-3347), and/or a variable light domain that is a variant of the IGK1-39_L1 variable light domain (SEQ ID NO:3239). In some embodiments, the variant VH domain has from 1, 2, 3, 4, 5, 6, 7, 8, 9 or 10 amino acid changes compared to one of the PD-L2 variable heavy domains depicted in FIG. 162 (SEQ ID NOs:3267, and 3275-3347) and/or the variable light domain has from 1, 2, 3, 4, 5, 6, 7, 8, 9 or 10 amino acid changes from the IGK1-39_L1 variable light domain (SEQ ID NO:3239). In some embodiments, the one or more amino acid changes are in the VH and/or VL framework regions (FR1, FR2, FR3, and/or FR4). In some embodiments, the one or more amino acid change(s) are in one or more CDRs. In certain embodiments, the PD-L2 ABD is capable of binding to PD-L2, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the PD-L2 ABD is capable of binding human PD-L2 antigen (see FIG. 1).

In one embodiment, the variant VH domain is at least 90, 95, 97, 98 or 99% identical to one of the PD-L2 variable heavy domains depicted in FIG. 162 (SEQ ID NOs:3267, and 3275-3347) and/or the variable light domain is at least 90, 95, 97, 98 or 99% identical to the IGK1-39_L1 variable light domain (SEQ ID NO:3239). In certain embodiments, the PD-L2 ABD is capable of binding to PD-L2, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the PD-L2 ABD is capable of binding human PD-L2 antigen (see FIG. 1).

In some embodiments, the PD-L2 binding domain includes a VH that includes any one of the VHCDR1-3 and/or HFR1-4 sequences depicted in FIG. 162G and the IGK1-39_L1 variable light domain (SEQ ID NO:3239) or a variant thereof.

In another aspect, provided herein is a PD-L2 binding domain that competes with any of the PD-L1 binding domains disclosed herein for binding to human PD-L2.

### C. CD28 Antigen Binding Domains

In one aspect, provided herein are CD28 antigen binding domains (ABDs) and compositions that include such CD28 antigen binding domains (ABDs), including anti-CD28 antibodies (e.g., anti-PD-L1×anti-CD28 bispecific antibodies, and anti-PD-L1×anti-PD-L2×anti-CD28 trispecific antibodies). Such CD28 binding domains and related antibodies (e.g., bispecific and trispecific antibodies disclosed herein) find use, for example, in the treatment of CD28 associated cancers. In some embodiments, the CD28 ABDs are capable of binding to human and cynomolgus CD28 (see FIG. 2 and Example 2).

As will be appreciated by those in the art, suitable CD28 binding domains can comprise a set of 6 CDRs as depicted in FIGS. 41-74 and the Sequence Listing. Suitable CD28 ABDs can also include the entire VH and VL sequences as depicted in these sequences and FIGS. 41-74, and the Sequence Listing, used as scFvs or as Fab domains.

In one embodiment, the CD28 antigen binding domain includes the 6 CDRs (i.e., vhCDR1-3 and vlCDR1-3) of a CD28 ABD described herein, including FIGS. 41-74, and the Sequence Listing, either as the CDRs are underlined or, in the case where a different numbering scheme is used as described herein and as shown in Table 2, as the CDRs that are identified using other alignments within the variable heavy (VH) domain and variable light domain (VL) sequences of those depicted in FIGS. 41-74, and the Sequence Listing (see Table 2). Suitable CD28 ABDs can also include the entire VH and VL sequences as depicted in these sequences and figures, used as scFvs or as Fab domains.

In one embodiment, the CD28 antigen binding domain includes the 6 CDRs (i.e., vhCDR1-3 and vlCDR1-3) of an CD28 ABD described herein, including FIGS. 41-74, and the Sequence Listing. In exemplary embodiments, the CD28 antigen binding domain comprises the 6 CDRs of a CD28 comprising a variable heavy domain (VH) and a variable light domain (VL), wherein VH and VL selected from the following:
(i) a VH having an amino acid sequence of SEQ ID NO:89; and (ii) a VL having an amino acid sequence of SEQ ID NO:93;
(i) a VH having an amino acid sequence of SEQ ID NO:97; and (ii) a VL having an amino acid sequence of SEQ ID NO:101;
(i) a VH having an amino acid sequence of SEQ ID NO:105; and (ii) a VL having an amino acid sequence of SEQ ID NO:109;
(i) a VH having an amino acid sequence of SEQ ID NO:113; and (ii) a VL having an amino acid sequence of SEQ ID NO:117;
(i) a VH having an amino acid sequence of SEQ ID NO:121; and (ii) a VL having an amino acid sequence of SEQ ID NO:125;
(i) a VH having an amino acid sequence of SEQ ID NO:129; and (ii) a VL having an amino acid sequence of SEQ ID NO:133;
(i) a VH having an amino acid sequence of SEQ ID NO:137; and (ii) a VL having an amino acid sequence of SEQ ID NO:141;
(i) a VH having an amino acid sequence of SEQ ID NO:145; and (ii) a VL having an amino acid sequence of SEQ ID NO:149;
(i) a VH having an amino acid sequence of SEQ ID NO:153; and (ii) a VL having an amino acid sequence of SEQ ID NO:157;
(i) a VH having an amino acid sequence of SEQ ID NO:161; and (ii) a VL having an amino acid sequence of SEQ ID NO:165;
(i) a VH having an amino acid sequence of SEQ ID NO:169; and (ii) a VL having an amino acid sequence of SEQ ID NO:173;
(i) a VH having an amino acid sequence of SEQ ID NO:177; and (ii) a VL having an amino acid sequence of SEQ ID NO:181;
(i) a VH having an amino acid sequence of SEQ ID NO:185; and (ii) a VL having an amino acid sequence of SEQ ID NO:189;
(i) a VH having an amino acid sequence of SEQ ID NO:193; and (ii) a VL having an amino acid sequence of SEQ ID NO:197;
(i) a VH having an amino acid sequence of SEQ ID NO:201; and (ii) a VL having an amino acid sequence of SEQ ID NO:205;
(i) a VH having an amino acid sequence of SEQ ID NO:209; and (ii) a VL having an amino acid sequence of SEQ ID NO:213;
(i) a VH having an amino acid sequence of SEQ ID NO:217; and (ii) a VL having an amino acid sequence of SEQ ID NO:221;
(i) a VH having an amino acid sequence of SEQ ID NO:225; and (ii) a VL having an amino acid sequence of SEQ ID NO:229;
(i) a VH having an amino acid sequence of SEQ ID NO:233; and (ii) a VL having an amino acid sequence of SEQ ID NO:237; and
(i) a VH having an amino acid sequence of SEQ ID NO:241; and (ii) a VL having an amino acid sequence of SEQ ID NO:245;
(i) a VH having an amino acid sequence of SEQ ID NO:249; and (ii) a VL having an amino acid sequence of SEQ ID NO:253;
(i) a VH having an amino acid sequence of SEQ ID NO:257; and (ii) a VL having an amino acid sequence of SEQ ID NO:261;
(i) a VH having an amino acid sequence of SEQ ID NO:265; and (ii) a VL having an amino acid sequence of SEQ ID NO:269;
(i) a VH having an amino acid sequence of SEQ ID NO:273; and (ii) a VL having an amino acid sequence of SEQ ID NO:277;
(i) a VH having an amino acid sequence of SEQ ID NO:281; and (ii) a VL having an amino acid sequence of SEQ ID NO:285;
(i) a VH having an amino acid sequence of SEQ ID NO:289; and (ii) a VL having an amino acid sequence of SEQ ID NO:293;
(i) a VH having an amino acid sequence of SEQ ID NO:297; and (ii) a VL having an amino acid sequence of SEQ ID NO:301;
(i) a VH having an amino acid sequence of SEQ ID NO:305; and (ii) a VL having an amino acid sequence of SEQ ID NO:309;
(i) a VH having an amino acid sequence of SEQ ID NO:313; and (ii) a VL having an amino acid sequence of SEQ ID NO:317;
(i) a VH having an amino acid sequence of SEQ ID NO:321; and (ii) a VL having an amino acid sequence of SEQ ID NO:325;
(i) a VH having an amino acid sequence of SEQ ID NO:329; and (ii) a VL having an amino acid sequence of SEQ ID NO:333;
(i) a VH having an amino acid sequence of SEQ ID NO:337; and (ii) a VL having an amino acid sequence of SEQ ID NO:341;
(i) a VH having an amino acid sequence of SEQ ID NO:89; and (ii) a VL having an amino acid sequence of SEQ ID NO:20;
(i) a VH having an amino acid sequence of SEQ ID NO:97; and (ii) a VL having an amino acid sequence of SEQ ID NO:20;
(i) a VH having an amino acid sequence of SEQ ID NO:105; and (ii) a VL having an amino acid sequence of SEQ ID NO:20;
(i) a VH having an amino acid sequence of SEQ ID NO:113; and (ii) a VL having an amino acid sequence of SEQ ID NO:20;
(i) a VH having an amino acid sequence of SEQ ID NO:121; and (ii) a VL having an amino acid sequence of SEQ ID NO:20;
(i) a VH having an amino acid sequence of SEQ ID NO:129; and (ii) a VL having an amino acid sequence of SEQ ID NO:20;
(i) a VH having an amino acid sequence of SEQ ID NO:137; and (ii) a VL having an amino acid sequence of SEQ ID NO:20;
(i) a VH having an amino acid sequence of SEQ ID NO:145; and (ii) a VL having an amino acid sequence of SEQ ID NO:20;
(i) a VH having an amino acid sequence of SEQ ID NO:153; and (ii) a VL having an amino acid sequence of SEQ ID NO:20;
(i) a VH having an amino acid sequence of SEQ ID NO:161; and (ii) a VL having an amino acid sequence of SEQ ID NO:20;
(i) a VH having an amino acid sequence of SEQ ID NO:169; and (ii) a VL having an amino acid sequence of SEQ ID NO:20;
(i) a VH having an amino acid sequence of SEQ ID NO:177; and (ii) a VL having an amino acid sequence of SEQ ID NO:20;
(i) a VH having an amino acid sequence of SEQ ID NO:185; and (ii) a VL having an amino acid sequence of SEQ ID NO:20;
(i) a VH having an amino acid sequence of SEQ ID NO:193; and (ii) a VL having an amino acid sequence of SEQ ID NO:20;
(i) a VH having an amino acid sequence of SEQ ID NO:201; and (ii) a VL having an amino acid sequence of SEQ ID NO:20;
(i) a VH having an amino acid sequence of SEQ ID NO:209; and (ii) a VL having an amino acid sequence of SEQ ID NO:20;
(i) a VH having an amino acid sequence of SEQ ID NO:217; and (ii) a VL having an amino acid sequence of SEQ ID NO:20;
(i) a VH having an amino acid sequence of SEQ ID NO:225; and (ii) a VL having an amino acid sequence of SEQ ID NO:20;
(i) a VH having an amino acid sequence of SEQ ID NO:233; and (ii) a VL having an amino acid sequence of SEQ ID NO:20; and
(i) a VH having an amino acid sequence of SEQ ID NO:241; and (ii) a VL having an amino acid sequence of SEQ ID NO:20;
(i) a VH having an amino acid sequence of SEQ ID NO:249; and (ii) a VL having an amino acid sequence of SEQ ID NO:20;
(i) a VH having an amino acid sequence of SEQ ID NO:257; and (ii) a VL having an amino acid sequence of SEQ ID NO:20;
(i) a VH having an amino acid sequence of SEQ ID NO:265; and (ii) a VL having an amino acid sequence of SEQ ID NO:20;
(i) a VH having an amino acid sequence of SEQ ID NO:273; and (ii) a VL having an amino acid sequence of SEQ ID NO:20;
(i) a VH having an amino acid sequence of SEQ ID NO:281; and (ii) a VL having an amino acid sequence of SEQ ID NO:20;
(i) a VH having an amino acid sequence of SEQ ID NO:289; and (ii) a VL having an amino acid sequence of SEQ ID NO:20;
(i) a VH having an amino acid sequence of SEQ ID NO:297; and (ii) a VL having an amino acid sequence of SEQ ID NO:20;
(i) a VH having an amino acid sequence of SEQ ID NO:305; and (ii) a VL having an amino acid sequence of SEQ ID NO:20;
(i) a VH having an amino acid sequence of SEQ ID NO:313; and (ii) a VL having an amino acid sequence of SEQ ID NO:20;
(i) a VH having an amino acid sequence of SEQ ID NO:321; and (ii) a VL having an amino acid sequence of SEQ ID NO:20;
(i) a VH having an amino acid sequence of SEQ ID NO:329; and (ii) a VL having an amino acid sequence of SEQ ID NO:20; and
(i) a VH having an amino acid sequence of SEQ ID NO:337; and (ii) a VL having an amino acid sequence of SEQ ID NO:20. (FIGS. 24, and 41-74).

In addition to the parental CDR sets disclosed in the figures and sequence listing that form an ABD to CD28, provided herein are variant CD28 ABDS having CDRs that include at least one modification of the CD28 ABD CDRs disclosed herein. In one embodiment, the CD28 ABD includes a set of 6 CDRs with 1, 2, 3, 4, 5, 6, 7, 8, 9, 10 amino acid modifications as compared to the 6 CDRs of an CD28 ABD described herein, including the figures and sequence listing. In exemplary embodiments, the CD28 ABD includes a set of 6 CDRs with 1, 2, 3, 4, 5, 6, 7, 8, 9, 10 amino acid modifications as compared to the 6 CDRs of an CD28 ABD comprising a variable heavy domain (VH) and a variable light domain (VL) selected from the following:
(i) a VH having an amino acid sequence of SEQ ID NO:89; and (ii) a VL having an amino acid sequence of SEQ ID NO:93;
(i) a VH having an amino acid sequence of SEQ ID NO:97; and (ii) a VL having an amino acid sequence of SEQ ID NO:101;
(i) a VH having an amino acid sequence of SEQ ID NO:105; and (ii) a VL having an amino acid sequence of SEQ ID NO:109;
(i) a VH having an amino acid sequence of SEQ ID NO:113; and (ii) a VL having an amino acid sequence of SEQ ID NO:117;
(i) a VH having an amino acid sequence of SEQ ID NO:121; and (ii) a VL having an amino acid sequence of SEQ ID NO:125;
(i) a VH having an amino acid sequence of SEQ ID NO:129; and (ii) a VL having an amino acid sequence of SEQ ID NO:133;
(i) a VH having an amino acid sequence of SEQ ID NO:137; and (ii) a VL having an amino acid sequence of SEQ ID NO:141;
(i) a VH having an amino acid sequence of SEQ ID NO:145; and (ii) a VL having an amino acid sequence of SEQ ID NO:149;
(i) a VH having an amino acid sequence of SEQ ID NO:153; and (ii) a VL having an amino acid sequence of SEQ ID NO:157;
(i) a VH having an amino acid sequence of SEQ ID NO:161; and (ii) a VL having an amino acid sequence of SEQ ID NO:165;
(i) a VH having an amino acid sequence of SEQ ID NO:169; and (ii) a VL having an amino acid sequence of SEQ ID NO:173;
(i) a VH having an amino acid sequence of SEQ ID NO:177; and (ii) a VL having an amino acid sequence of SEQ ID NO:181;
(i) a VH having an amino acid sequence of SEQ ID NO:185; and (ii) a VL having an amino acid sequence of SEQ ID NO:189;
(i) a VH having an amino acid sequence of SEQ ID NO:193; and (ii) a VL having an amino acid sequence of SEQ ID NO:197;
(i) a VH having an amino acid sequence of SEQ ID NO:201; and (ii) a VL having an amino acid sequence of SEQ ID NO:205;
(i) a VH having an amino acid sequence of SEQ ID NO:209; and (ii) a VL having an amino acid sequence of SEQ ID NO:213;
(i) a VH having an amino acid sequence of SEQ ID NO:217; and (ii) a VL having an amino acid sequence of SEQ ID NO:221;
(i) a VH having an amino acid sequence of SEQ ID NO:225; and (ii) a VL having an amino acid sequence of SEQ ID NO:229;
(i) a VH having an amino acid sequence of SEQ ID NO:233; and (ii) a VL having an amino acid sequence of SEQ ID NO:237; and
(i) a VH having an amino acid sequence of SEQ ID NO:241; and (ii) a VL having an amino acid sequence of SEQ ID NO:245;
(i) a VH having an amino acid sequence of SEQ ID NO:249; and (ii) a VL having an amino acid sequence of SEQ ID NO:253;
(i) a VH having an amino acid sequence of SEQ ID NO:257; and (ii) a VL having an amino acid sequence of SEQ ID NO:261;
(i) a VH having an amino acid sequence of SEQ ID NO:265; and (ii) a VL having an amino acid sequence of SEQ ID NO:269;
(i) a VH having an amino acid sequence of SEQ ID NO:273; and (ii) a VL having an amino acid sequence of SEQ ID NO:277;
(i) a VH having an amino acid sequence of SEQ ID NO:281; and (ii) a VL having an amino acid sequence of SEQ ID NO:285;
(i) a VH having an amino acid sequence of SEQ ID NO:289; and (ii) a VL having an amino acid sequence of SEQ ID NO:293;
(i) a VH having an amino acid sequence of SEQ ID NO:297; and (ii) a VL having an amino acid sequence of SEQ ID NO:301;
(i) a VH having an amino acid sequence of SEQ ID NO:305; and (ii) a VL having an amino acid sequence of SEQ ID NO:309;
(i) a VH having an amino acid sequence of SEQ ID NO:313; and (ii) a VL having an amino acid sequence of SEQ ID NO:317;
(i) a VH having an amino acid sequence of SEQ ID NO:321; and (ii) a VL having an amino acid sequence of SEQ ID NO:325;
(i) a VH having an amino acid sequence of SEQ ID NO:329; and (ii) a VL having an amino acid sequence of SEQ ID NO:333;
(i) a VH having an amino acid sequence of SEQ ID NO:337; and (ii) a VL having an amino acid sequence of SEQ ID NO:341;
(i) a VH having an amino acid sequence of SEQ ID NO:89; and (ii) a VL having an amino acid sequence of SEQ ID NO:20;
(i) a VH having an amino acid sequence of SEQ ID NO:97; and (ii) a VL having an amino acid sequence of SEQ ID NO:20;
(i) a VH having an amino acid sequence of SEQ ID NO:105; and (ii) a VL having an amino acid sequence of SEQ ID NO:20;
(i) a VH having an amino acid sequence of SEQ ID NO:113; and (ii) a VL having an amino acid sequence of SEQ ID NO:20;
(i) a VH having an amino acid sequence of SEQ ID NO:121; and (ii) a VL having an amino acid sequence of SEQ ID NO:20;
(i) a VH having an amino acid sequence of SEQ ID NO:129; and (ii) a VL having an amino acid sequence of SEQ ID NO:20;
(i) a VH having an amino acid sequence of SEQ ID NO:137; and (ii) a VL having an amino acid sequence of SEQ ID NO:20;
(i) a VH having an amino acid sequence of SEQ ID NO:145; and (ii) a VL having an amino acid sequence of SEQ ID NO:20;
(i) a VH having an amino acid sequence of SEQ ID NO:153; and (ii) a VL having an amino acid sequence of SEQ ID NO:20;
(i) a VH having an amino acid sequence of SEQ ID NO:161; and (ii) a VL having an amino acid sequence of SEQ ID NO:20;
(i) a VH having an amino acid sequence of SEQ ID NO:169; and (ii) a VL having an amino acid sequence of SEQ ID NO:20;
(i) a VH having an amino acid sequence of SEQ ID NO:177; and (ii) a VL having an amino acid sequence of SEQ ID NO:20;
(i) a VH having an amino acid sequence of SEQ ID NO:185; and (ii) a VL having an amino acid sequence of SEQ ID NO:20;
(i) a VH having an amino acid sequence of SEQ ID NO:193; and (ii) a VL having an amino acid sequence of SEQ ID NO:20;
(i) a VH having an amino acid sequence of SEQ ID NO:201; and (ii) a VL having an amino acid sequence of SEQ ID NO:20;
(i) a VH having an amino acid sequence of SEQ ID NO:209; and (ii) a VL having an amino acid sequence of SEQ ID NO:20;
(i) a VH having an amino acid sequence of SEQ ID NO:217; and (ii) a VL having an amino acid sequence of SEQ ID NO:20;
(i) a VH having an amino acid sequence of SEQ ID NO:225; and (ii) a VL having an amino acid sequence of SEQ ID NO:20;
(i) a VH having an amino acid sequence of SEQ ID NO:233; and (ii) a VL having an amino acid sequence of SEQ ID NO:20; and
(i) a VH having an amino acid sequence of SEQ ID NO:241; and (ii) a VL having an amino acid sequence of SEQ ID NO:20;
(i) a VH having an amino acid sequence of SEQ ID NO:249; and (ii) a VL having an amino acid sequence of SEQ ID NO:20;
(i) a VH having an amino acid sequence of SEQ ID NO:257; and (ii) a VL having an amino acid sequence of SEQ ID NO:20;
(i) a VH having an amino acid sequence of SEQ ID NO:265; and (ii) a VL having an amino acid sequence of SEQ ID NO:20;
(i) a VH having an amino acid sequence of SEQ ID NO:273; and (ii) a VL having an amino acid sequence of SEQ ID NO:20;
(i) a VH having an amino acid sequence of SEQ ID NO:281; and (ii) a VL having an amino acid sequence of SEQ ID NO:20;
(i) a VH having an amino acid sequence of SEQ ID NO:289; and (ii) a VL having an amino acid sequence of SEQ ID NO:20;
(i) a VH having an amino acid sequence of SEQ ID NO:297; and (ii) a VL having an amino acid sequence of SEQ ID NO:20;
(i) a VH having an amino acid sequence of SEQ ID NO:305; and (ii) a VL having an amino acid sequence of SEQ ID NO:20;
(i) a VH having an amino acid sequence of SEQ ID NO:313; and (ii) a VL having an amino acid sequence of SEQ ID NO:20;
(i) a VH having an amino acid sequence of SEQ ID NO:321; and (ii) a VL having an amino acid sequence of SEQ ID NO:20;
(i) a VH having an amino acid sequence of SEQ ID NO:329; and (ii) a VL having an amino acid sequence of SEQ ID NO:20; and
(i) a VH having an amino acid sequence of SEQ ID NO:337; and (ii) a VL having an amino acid sequence of SEQ ID NO:20. (FIGS. 24, and 29-32).

In certain embodiments, the variant CD28 ABD is capable of binding CD28 antigen, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding to human and cynomolgus CD28.

In one embodiment, the anti-CD28 ABD includes 6 CDRs that are at least 90, 95, 97, 98 or 99% identical to the 6 CDRs of an anti-CD28 ABD as described herein, including FIGS. 24, and 41-74, and the Sequence Listing. In exemplary embodiments, the anti-CD28 ABD includes 6 CDRs that are at least 90, 95, 97, 98 or 99% identical to the 6 CDRs of an CD28 ABD comprising a variable heavy domain (VH) and a variable light domain (VL) selected from the following:
(i) a VH having an amino acid sequence of SEQ ID NO:89; and (ii) a VL having an amino acid sequence of SEQ ID NO:93;
(i) a VH having an amino acid sequence of SEQ ID NO:97; and (ii) a VL having an amino acid sequence of SEQ ID NO:101;
(i) a VH having an amino acid sequence of SEQ ID NO:105; and (ii) a VL having an amino acid sequence of SEQ ID NO:109;
(i) a VH having an amino acid sequence of SEQ ID NO:113; and (ii) a VL having an amino acid sequence of SEQ ID NO:117;
(i) a VH having an amino acid sequence of SEQ ID NO:121; and (ii) a VL having an amino acid sequence of SEQ ID NO:125;
(i) a VH having an amino acid sequence of SEQ ID NO:129; and (ii) a VL having an amino acid sequence of SEQ ID NO:133;
(i) a VH having an amino acid sequence of SEQ ID NO:137; and (ii) a VL having an amino acid sequence of SEQ ID NO:141;
(i) a VH having an amino acid sequence of SEQ ID NO:145; and (ii) a VL having an amino acid sequence of SEQ ID NO:149;
(i) a VH having an amino acid sequence of SEQ ID NO:153; and (ii) a VL having an amino acid sequence of SEQ ID NO:157;
(i) a VH having an amino acid sequence of SEQ ID NO:161; and (ii) a VL having an amino acid sequence of SEQ ID NO:165;
(i) a VH having an amino acid sequence of SEQ ID NO:169; and (ii) a VL having an amino acid sequence of SEQ ID NO:173;
(i) a VH having an amino acid sequence of SEQ ID NO:177; and (ii) a VL having an amino acid sequence of SEQ ID NO:181;
(i) a VH having an amino acid sequence of SEQ ID NO:185; and (ii) a VL having an amino acid sequence of SEQ ID NO:189;
(i) a VH having an amino acid sequence of SEQ ID NO:193; and (ii) a VL having an amino acid sequence of SEQ ID NO:197;
(i) a VH having an amino acid sequence of SEQ ID NO:201; and (ii) a VL having an amino acid sequence of SEQ ID NO:205;
(i) a VH having an amino acid sequence of SEQ ID NO:209; and (ii) a VL having an amino acid sequence of SEQ ID NO:213;
(i) a VH having an amino acid sequence of SEQ ID NO:217; and (ii) a VL having an amino acid sequence of SEQ ID NO:221;
(i) a VH having an amino acid sequence of SEQ ID NO:225; and (ii) a VL having an amino acid sequence of SEQ ID NO:229;
(i) a VH having an amino acid sequence of SEQ ID NO:233; and (ii) a VL having an amino acid sequence of SEQ ID NO:237; and
(i) a VH having an amino acid sequence of SEQ ID NO:241; and (ii) a VL having an amino acid sequence of SEQ ID NO:245;
(i) a VH having an amino acid sequence of SEQ ID NO:249; and (ii) a VL having an amino acid sequence of SEQ ID NO:253;
(i) a VH having an amino acid sequence of SEQ ID NO:257; and (ii) a VL having an amino acid sequence of SEQ ID NO:261;
(i) a VH having an amino acid sequence of SEQ ID NO:265; and (ii) a VL having an amino acid sequence of SEQ ID NO:269;
(i) a VH having an amino acid sequence of SEQ ID NO:273; and (ii) a VL having an amino acid sequence of SEQ ID NO:277;
(i) a VH having an amino acid sequence of SEQ ID NO:281; and (ii) a VL having an amino acid sequence of SEQ ID NO:285;
(i) a VH having an amino acid sequence of SEQ ID NO:289; and (ii) a VL having an amino acid sequence of SEQ ID NO:293;
(i) a VH having an amino acid sequence of SEQ ID NO:297; and (ii) a VL having an amino acid sequence of SEQ ID NO:301;
(i) a VH having an amino acid sequence of SEQ ID NO:305; and (ii) a VL having an amino acid sequence of SEQ ID NO:309;
(i) a VH having an amino acid sequence of SEQ ID NO:313; and (ii) a VL having an amino acid sequence of SEQ ID NO:317;
(i) a VH having an amino acid sequence of SEQ ID NO:321; and (ii) a VL having an amino acid sequence of SEQ ID NO:325;
(i) a VH having an amino acid sequence of SEQ ID NO:329; and (ii) a VL having an amino acid sequence of SEQ ID NO:333;
(i) a VH having an amino acid sequence of SEQ ID NO:337; and (ii) a VL having an amino acid sequence of SEQ ID NO:341;
(i) a VH having an amino acid sequence of SEQ ID NO:89; and (ii) a VL having an amino acid sequence of SEQ ID NO:20;
(i) a VH having an amino acid sequence of SEQ ID NO:97; and (ii) a VL having an amino acid sequence of SEQ ID NO:20;
(i) a VH having an amino acid sequence of SEQ ID NO:105; and (ii) a VL having an amino acid sequence of SEQ ID NO:20;
(i) a VH having an amino acid sequence of SEQ ID NO:113; and (ii) a VL having an amino acid sequence of SEQ ID NO:20;
(i) a VH having an amino acid sequence of SEQ ID NO:121; and (ii) a VL having an amino acid sequence of SEQ ID NO:20;
(i) a VH having an amino acid sequence of SEQ ID NO:129; and (ii) a VL having an amino acid sequence of SEQ ID NO:20;
(i) a VH having an amino acid sequence of SEQ ID NO:137; and (ii) a VL having an amino acid sequence of SEQ ID NO:20;
(i) a VH having an amino acid sequence of SEQ ID NO:145; and (ii) a VL having an amino acid sequence of SEQ ID NO:20;
(i) a VH having an amino acid sequence of SEQ ID NO:153; and (ii) a VL having an amino acid sequence of SEQ ID NO:20;
(i) a VH having an amino acid sequence of SEQ ID NO:161; and (ii) a VL having an amino acid sequence of SEQ ID NO:20;
(i) a VH having an amino acid sequence of SEQ ID NO:169; and (ii) a VL having an amino acid sequence of SEQ ID NO:20;
(i) a VH having an amino acid sequence of SEQ ID NO:177; and (ii) a VL having an amino acid sequence of SEQ ID NO:20;
(i) a VH having an amino acid sequence of SEQ ID NO:185; and (ii) a VL having an amino acid sequence of SEQ ID NO:20;
(i) a VH having an amino acid sequence of SEQ ID NO:193; and (ii) a VL having an amino acid sequence of SEQ ID NO:20;
(i) a VH having an amino acid sequence of SEQ ID NO:201; and (ii) a VL having an amino acid sequence of SEQ ID NO:20;
(i) a VH having an amino acid sequence of SEQ ID NO:209; and (ii) a VL having an amino acid sequence of SEQ ID NO:20;
(i) a VH having an amino acid sequence of SEQ ID NO:217; and (ii) a VL having an amino acid sequence of SEQ ID NO:20;
(i) a VH having an amino acid sequence of SEQ ID NO:225; and (ii) a VL having an amino acid sequence of SEQ ID NO:20;
(i) a VH having an amino acid sequence of SEQ ID NO:233; and (ii) a VL having an amino acid sequence of SEQ ID NO:20; and
(i) a VH having an amino acid sequence of SEQ ID NO:241; and (ii) a VL having an amino acid sequence of SEQ ID NO:20;
(i) a VH having an amino acid sequence of SEQ ID NO:249; and (ii) a VL having an amino acid sequence of SEQ ID NO:20;
(i) a VH having an amino acid sequence of SEQ ID NO:257; and (ii) a VL having an amino acid sequence of SEQ ID NO:20;
(i) a VH having an amino acid sequence of SEQ ID NO:265; and (ii) a VL having an amino acid sequence of SEQ ID NO:20;
(i) a VH having an amino acid sequence of SEQ ID NO:273; and (ii) a VL having an amino acid sequence of SEQ ID NO:20;
(i) a VH having an amino acid sequence of SEQ ID NO:281; and (ii) a VL having an amino acid sequence of SEQ ID NO:20;
(i) a VH having an amino acid sequence of SEQ ID NO:289; and (ii) a VL having an amino acid sequence of SEQ ID NO:20;
(i) a VH having an amino acid sequence of SEQ ID NO:297; and (ii) a VL having an amino acid sequence of SEQ ID NO:20;
(i) a VH having an amino acid sequence of SEQ ID NO:305; and (ii) a VL having an amino acid sequence of SEQ ID NO:20;
(i) a VH having an amino acid sequence of SEQ ID NO:313; and (ii) a VL having an amino acid sequence of SEQ ID NO:20;
(i) a VH having an amino acid sequence of SEQ ID NO:321; and (ii) a VL having an amino acid sequence of SEQ ID NO:20;
(i) a VH having an amino acid sequence of SEQ ID NO:329; and (ii) a VL having an amino acid sequence of SEQ ID NO:20; and
(i) a VH having an amino acid sequence of SEQ ID NO:337; and (ii) a VL having an amino acid sequence of SEQ ID NO:20. (FIGS. 24, and 41-74).

In certain embodiments, the anti-CD28 ABD is capable of binding to CD28 antigen, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding to human and cynomolgus CD28.

In another exemplary embodiment, the anti-CD28 ABD include the variable heavy (VH) domain and/or variable light (VL) domain of any one of the CD28 ABDs described herein, including FIGS. 24, and 41-74 and the Sequence Listing. In exemplary embodiments, VH and VL are selected from the following:
(i) a VH having an amino acid sequence of SEQ ID NO:89; and (ii) a VL having an amino acid sequence of SEQ ID NO:93;
(i) a VH having an amino acid sequence of SEQ ID NO:97; and (ii) a VL having an amino acid sequence of SEQ ID NO:101;
(i) a VH having an amino acid sequence of SEQ ID NO:105; and (ii) a VL having an amino acid sequence of SEQ ID NO:109;
(i) a VH having an amino acid sequence of SEQ ID NO:113; and (ii) a VL having an amino acid sequence of SEQ ID NO:117;
(i) a VH having an amino acid sequence of SEQ ID NO:121; and (ii) a VL having an amino acid sequence of SEQ ID NO:125;
(i) a VH having an amino acid sequence of SEQ ID NO:129; and (ii) a VL having an amino acid sequence of SEQ ID NO:133;
(i) a VH having an amino acid sequence of SEQ ID NO:137; and (ii) a VL having an amino acid sequence of SEQ ID NO:141;
(i) a VH having an amino acid sequence of SEQ ID NO:145; and (ii) a VL having an amino acid sequence of SEQ ID NO:149;
(i) a VH having an amino acid sequence of SEQ ID NO:153; and (ii) a VL having an amino acid sequence of SEQ ID NO:157;
(i) a VH having an amino acid sequence of SEQ ID NO:161; and (ii) a VL having an amino acid sequence of SEQ ID NO:165;
(i) a VH having an amino acid sequence of SEQ ID NO:169; and (ii) a VL having an amino acid sequence of SEQ ID NO:173;
(i) a VH having an amino acid sequence of SEQ ID NO:177; and (ii) a VL having an amino acid sequence of SEQ ID NO:181;
(i) a VH having an amino acid sequence of SEQ ID NO:185; and (ii) a VL having an amino acid sequence of SEQ ID NO:189;
(i) a VH having an amino acid sequence of SEQ ID NO:193; and (ii) a VL having an amino acid sequence of SEQ ID NO:197;
(i) a VH having an amino acid sequence of SEQ ID NO:201; and (ii) a VL having an amino acid sequence of SEQ ID NO:205;
(i) a VH having an amino acid sequence of SEQ ID NO:209; and (ii) a VL having an amino acid sequence of SEQ ID NO:213;
(i) a VH having an amino acid sequence of SEQ ID NO:217; and (ii) a VL having an amino acid sequence of SEQ ID NO:221;
(i) a VH having an amino acid sequence of SEQ ID NO:225; and (ii) a VL having an amino acid sequence of SEQ ID NO:229;
(i) a VH having an amino acid sequence of SEQ ID NO:233; and (ii) a VL having an amino acid sequence of SEQ ID NO:237; and
(i) a VH having an amino acid sequence of SEQ ID NO:241; and (ii) a VL having an amino acid sequence of SEQ ID NO:245;
(i) a VH having an amino acid sequence of SEQ ID NO:249; and (ii) a VL having an amino acid sequence of SEQ ID NO:253;
(i) a VH having an amino acid sequence of SEQ ID NO:257; and (ii) a VL having an amino acid sequence of SEQ ID NO:261;
(i) a VH having an amino acid sequence of SEQ ID NO:265; and (ii) a VL having an amino acid sequence of SEQ ID NO:269;
(i) a VH having an amino acid sequence of SEQ ID NO:273; and (ii) a VL having an amino acid sequence of SEQ ID NO:277;
(i) a VH having an amino acid sequence of SEQ ID NO:281; and (ii) a VL having an amino acid sequence of SEQ ID NO:285;
(i) a VH having an amino acid sequence of SEQ ID NO:289; and (ii) a VL having an amino acid sequence of SEQ ID NO:293;
(i) a VH having an amino acid sequence of SEQ ID NO:297; and (ii) a VL having an amino acid sequence of SEQ ID NO:301;
(i) a VH having an amino acid sequence of SEQ ID NO:305; and (ii) a VL having an amino acid sequence of SEQ ID NO:309;
(i) a VH having an amino acid sequence of SEQ ID NO:313; and (ii) a VL having an amino acid sequence of SEQ ID NO:317;
(i) a VH having an amino acid sequence of SEQ ID NO:321; and (ii) a VL having an amino acid sequence of SEQ ID NO:325;
(i) a VH having an amino acid sequence of SEQ ID NO:329; and (ii) a VL having an amino acid sequence of SEQ ID NO:333;
(i) a VH having an amino acid sequence of SEQ ID NO:337; and (ii) a VL having an amino acid sequence of SEQ ID NO:341;
(i) a VH having an amino acid sequence of SEQ ID NO:89; and (ii) a VL having an amino acid sequence of SEQ ID NO:20;
(i) a VH having an amino acid sequence of SEQ ID NO:97; and (ii) a VL having an amino acid sequence of SEQ ID NO:20;
(i) a VH having an amino acid sequence of SEQ ID NO:105; and (ii) a VL having an amino acid sequence of SEQ ID NO:20;
(i) a VH having an amino acid sequence of SEQ ID NO:113; and (ii) a VL having an amino acid sequence of SEQ ID NO:20;
(i) a VH having an amino acid sequence of SEQ ID NO:121; and (ii) a VL having an amino acid sequence of SEQ ID NO:20;
(i) a VH having an amino acid sequence of SEQ ID NO:129; and (ii) a VL having an amino acid sequence of SEQ ID NO:20;
(i) a VH having an amino acid sequence of SEQ ID NO:137; and (ii) a VL having an amino acid sequence of SEQ ID NO:20;
(i) a VH having an amino acid sequence of SEQ ID NO:145; and (ii) a VL having an amino acid sequence of SEQ ID NO:20;
(i) a VH having an amino acid sequence of SEQ ID NO:153; and (ii) a VL having an amino acid sequence of SEQ ID NO:20;
(i) a VH having an amino acid sequence of SEQ ID NO:161; and (ii) a VL having an amino acid sequence of SEQ ID NO:20;
(i) a VH having an amino acid sequence of SEQ ID NO:169; and (ii) a VL having an amino acid sequence of SEQ ID NO:20;
(i) a VH having an amino acid sequence of SEQ ID NO:177; and (ii) a VL having an amino acid sequence of SEQ ID NO:20;
(i) a VH having an amino acid sequence of SEQ ID NO:185; and (ii) a VL having an amino acid sequence of SEQ ID NO:20;
(i) a VH having an amino acid sequence of SEQ ID NO:193; and (ii) a VL having an amino acid sequence of SEQ ID NO:20;
(i) a VH having an amino acid sequence of SEQ ID NO:201; and (ii) a VL having an amino acid sequence of SEQ ID NO:20;
(i) a VH having an amino acid sequence of SEQ ID NO:209; and (ii) a VL having an amino acid sequence of SEQ ID NO:20;
(i) a VH having an amino acid sequence of SEQ ID NO:217; and (ii) a VL having an amino acid sequence of SEQ ID NO:20;
(i) a VH having an amino acid sequence of SEQ ID NO:225; and (ii) a VL having an amino acid sequence of SEQ ID NO:20;
(i) a VH having an amino acid sequence of SEQ ID NO:233; and (ii) a VL having an amino acid sequence of SEQ ID NO:20; and
(i) a VH having an amino acid sequence of SEQ ID NO:241; and (ii) a VL having an amino acid sequence of SEQ ID NO:20;
(i) a VH having an amino acid sequence of SEQ ID NO:249; and (ii) a VL having an amino acid sequence of SEQ ID NO:20;
(i) a VH having an amino acid sequence of SEQ ID NO:257; and (ii) a VL having an amino acid sequence of SEQ ID NO:20;
(i) a VH having an amino acid sequence of SEQ ID NO:265; and (ii) a VL having an amino acid sequence of SEQ ID NO:20;
(i) a VH having an amino acid sequence of SEQ ID NO:273; and (ii) a VL having an amino acid sequence of SEQ ID NO:20;
(i) a VH having an amino acid sequence of SEQ ID NO:281; and (ii) a VL having an amino acid sequence of SEQ ID NO:20;
(i) a VH having an amino acid sequence of SEQ ID NO:289; and (ii) a VL having an amino acid sequence of SEQ ID NO:20;
(i) a VH having an amino acid sequence of SEQ ID NO:297; and (ii) a VL having an amino acid sequence of SEQ ID NO:20;
(i) a VH having an amino acid sequence of SEQ ID NO:305; and (ii) a VL having an amino acid sequence of SEQ ID NO:20;
(i) a VH having an amino acid sequence of SEQ ID NO:313; and (ii) a VL having an amino acid sequence of SEQ ID NO:20;
(i) a VH having an amino acid sequence of SEQ ID NO:321; and (ii) a VL having an amino acid sequence of SEQ ID NO:20;
(i) a VH having an amino acid sequence of SEQ ID NO:329; and (ii) a VL having an amino acid sequence of SEQ ID NO:20; and
(i) a VH having an amino acid sequence of SEQ ID NO:337; and (ii) a VL having an amino acid sequence of SEQ ID NO:20. (FIGS. 24, and 41-74).

In addition to the parental anti-CD28 binding domain variable heavy and variable light domains disclosed herein, provided herein are anti-CD28 ABDs that include a variable heavy domain and/or a variable light domain that are variants of an anti-CD28 ABD VH and VL domain disclosed herein. In one embodiment, the variant VH domain and/or VL domain has from 1, 2, 3, 4, 5, 6, 7, 8, 9 or 10 amino acid changes from a VH and/or VL domain of an anti-CD28 ABD described herein, including FIGS. 28-31 and the Sequence Listing. In exemplary embodiments, the variant VH domain and/or VL domain has from 1, 2, 3, 4, 5, 6, 7, 8, 9 or 10 amino acid changes from a VH and/or VL are selected from the following:
(i) a VH having an amino acid sequence of SEQ ID NO:89; and (ii) a VL having an amino acid sequence of SEQ ID NO:93;
(i) a VH having an amino acid sequence of SEQ ID NO:97; and (ii) a VL having an amino acid sequence of SEQ ID NO:101;
(i) a VH having an amino acid sequence of SEQ ID NO:105; and (ii) a VL having an amino acid sequence of SEQ ID NO:109;
(i) a VH having an amino acid sequence of SEQ ID NO:113; and (ii) a VL having an amino acid sequence of SEQ ID NO:117;
(i) a VH having an amino acid sequence of SEQ ID NO:121; and (ii) a VL having an amino acid sequence of SEQ ID NO:125;
(i) a VH having an amino acid sequence of SEQ ID NO:129; and (ii) a VL having an amino acid sequence of SEQ ID NO:133;
(i) a VH having an amino acid sequence of SEQ ID NO:137; and (ii) a VL having an amino acid sequence of SEQ ID NO:141;
(i) a VH having an amino acid sequence of SEQ ID NO:145; and (ii) a VL having an amino acid sequence of SEQ ID NO:149;
(i) a VH having an amino acid sequence of SEQ ID NO:153; and (ii) a VL having an amino acid sequence of SEQ ID NO:157;
(i) a VH having an amino acid sequence of SEQ ID NO:161; and (ii) a VL having an amino acid sequence of SEQ ID NO:165;
(i) a VH having an amino acid sequence of SEQ ID NO:169; and (ii) a VL having an amino acid sequence of SEQ ID NO:173;
(i) a VH having an amino acid sequence of SEQ ID NO:177; and (ii) a VL having an amino acid sequence of SEQ ID NO:181;
(i) a VH having an amino acid sequence of SEQ ID NO:185; and (ii) a VL having an amino acid sequence of SEQ ID NO:189;
(i) a VH having an amino acid sequence of SEQ ID NO:193; and (ii) a VL having an amino acid sequence of SEQ ID NO:197;
(i) a VH having an amino acid sequence of SEQ ID NO:201; and (ii) a VL having an amino acid sequence of SEQ ID NO:205;
(i) a VH having an amino acid sequence of SEQ ID NO:209; and (ii) a VL having an amino acid sequence of SEQ ID NO:213;
(i) a VH having an amino acid sequence of SEQ ID NO:217; and (ii) a VL having an amino acid sequence of SEQ ID NO:221;
(i) a VH having an amino acid sequence of SEQ ID NO:225; and (ii) a VL having an amino acid sequence of SEQ ID NO:229;
(i) a VH having an amino acid sequence of SEQ ID NO:233; and (ii) a VL having an amino acid sequence of SEQ ID NO:237; and
(i) a VH having an amino acid sequence of SEQ ID NO:241; and (ii) a VL having an amino acid sequence of SEQ ID NO:245;
(i) a VH having an amino acid sequence of SEQ ID NO:249; and (ii) a VL having an amino acid sequence of SEQ ID NO:253;
(i) a VH having an amino acid sequence of SEQ ID NO:257; and (ii) a VL having an amino acid sequence of SEQ ID NO:261;
(i) a VH having an amino acid sequence of SEQ ID NO:265; and (ii) a VL having an amino acid sequence of SEQ ID NO:269;
(i) a VH having an amino acid sequence of SEQ ID NO:273; and (ii) a VL having an amino acid sequence of SEQ ID NO:277;
(i) a VH having an amino acid sequence of SEQ ID NO:281; and (ii) a VL having an amino acid sequence of SEQ ID NO:285;
(i) a VH having an amino acid sequence of SEQ ID NO:289; and (ii) a VL having an amino acid sequence of SEQ ID NO:293;
(i) a VH having an amino acid sequence of SEQ ID NO:297; and (ii) a VL having an amino acid sequence of SEQ ID NO:301;
(i) a VH having an amino acid sequence of SEQ ID NO:305; and (ii) a VL having an amino acid sequence of SEQ ID NO:309;
(i) a VH having an amino acid sequence of SEQ ID NO:313; and (ii) a VL having an amino acid sequence of SEQ ID NO:317;
(i) a VH having an amino acid sequence of SEQ ID NO:321; and (ii) a VL having an amino acid sequence of SEQ ID NO:325;
(i) a VH having an amino acid sequence of SEQ ID NO:329; and (ii) a VL having an amino acid sequence of SEQ ID NO:333;
(i) a VH having an amino acid sequence of SEQ ID NO:337; and (ii) a VL having an amino acid sequence of SEQ ID NO:341;
(i) a VH having an amino acid sequence of SEQ ID NO:89; and (ii) a VL having an amino acid sequence of SEQ ID NO:20;
(i) a VH having an amino acid sequence of SEQ ID NO:97; and (ii) a VL having an amino acid sequence of SEQ ID NO:20;
(i) a VH having an amino acid sequence of SEQ ID NO:105; and (ii) a VL having an amino acid sequence of SEQ ID NO:20;
(i) a VH having an amino acid sequence of SEQ ID NO:113; and (ii) a VL having an amino acid sequence of SEQ ID NO:20;
(i) a VH having an amino acid sequence of SEQ ID NO:121; and (ii) a VL having an amino acid sequence of SEQ ID NO:20;
(i) a VH having an amino acid sequence of SEQ ID NO:129; and (ii) a VL having an amino acid sequence of SEQ ID NO:20;
(i) a VH having an amino acid sequence of SEQ ID NO:137; and (ii) a VL having an amino acid sequence of SEQ ID NO:20;
(i) a VH having an amino acid sequence of SEQ ID NO:145; and (ii) a VL having an amino acid sequence of SEQ ID NO:20;
(i) a VH having an amino acid sequence of SEQ ID NO:153; and (ii) a VL having an amino acid sequence of SEQ ID NO:20;
(i) a VH having an amino acid sequence of SEQ ID NO:161; and (ii) a VL having an amino acid sequence of SEQ ID NO:20;
(i) a VH having an amino acid sequence of SEQ ID NO:169; and (ii) a VL having an amino acid sequence of SEQ ID NO:20;
(i) a VH having an amino acid sequence of SEQ ID NO:177; and (ii) a VL having an amino acid sequence of SEQ ID NO:20;
(i) a VH having an amino acid sequence of SEQ ID NO:185; and (ii) a VL having an amino acid sequence of SEQ ID NO:20;
(i) a VH having an amino acid sequence of SEQ ID NO:193; and (ii) a VL having an amino acid sequence of SEQ ID NO:20;
(i) a VH having an amino acid sequence of SEQ ID NO:201; and (ii) a VL having an amino acid sequence of SEQ ID NO:20;
(i) a VH having an amino acid sequence of SEQ ID NO:209; and (ii) a VL having an amino acid sequence of SEQ ID NO:20;
(i) a VH having an amino acid sequence of SEQ ID NO:217; and (ii) a VL having an amino acid sequence of SEQ ID NO:20;
(i) a VH having an amino acid sequence of SEQ ID NO:225; and (ii) a VL having an amino acid sequence of SEQ ID NO:20;
(i) a VH having an amino acid sequence of SEQ ID NO:233; and (ii) a VL having an amino acid sequence of SEQ ID NO:20; and
(i) a VH having an amino acid sequence of SEQ ID NO:241; and (ii) a VL having an amino acid sequence of SEQ ID NO:20;
(i) a VH having an amino acid sequence of SEQ ID NO:249; and (ii) a VL having an amino acid sequence of SEQ ID NO:20;
(i) a VH having an amino acid sequence of SEQ ID NO:257; and (ii) a VL having an amino acid sequence of SEQ ID NO:20;
(i) a VH having an amino acid sequence of SEQ ID NO:265; and (ii) a VL having an amino acid sequence of SEQ ID NO:20;
(i) a VH having an amino acid sequence of SEQ ID NO:273; and (ii) a VL having an amino acid sequence of SEQ ID NO:20;
(i) a VH having an amino acid sequence of SEQ ID NO:281; and (ii) a VL having an amino acid sequence of SEQ ID NO:20;
(i) a VH having an amino acid sequence of SEQ ID NO:289; and (ii) a VL having an amino acid sequence of SEQ ID NO:20;
(i) a VH having an amino acid sequence of SEQ ID NO:297; and (ii) a VL having an amino acid sequence of SEQ ID NO:20;
(i) a VH having an amino acid sequence of SEQ ID NO:305; and (ii) a VL having an amino acid sequence of SEQ ID NO:20;
(i) a VH having an amino acid sequence of SEQ ID NO:313; and (ii) a VL having an amino acid sequence of SEQ ID NO:20;
(i) a VH having an amino acid sequence of SEQ ID NO:321; and (ii) a VL having an amino acid sequence of SEQ ID NO:20;
(i) a VH having an amino acid sequence of SEQ ID NO:329; and (ii) a VL having an amino acid sequence of SEQ ID NO:20; and
(i) a VH having an amino acid sequence of SEQ ID NO:337; and (ii) a VL having an amino acid sequence of SEQ ID NO:20. (FIGS. 24, and 41-74).

In certain embodiments, the anti-CD28 ABD is capable of binding to CD28, as measured at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding to human and cynomolgus CD28.

In some embodiments, the CD28 ABD includes a variable heavy domain that has from 1, 2, 3, 4, 5, 6, 7, 8, 9 or 10 amino acid changes one of the following CD28 ABD variable heavy domains: SEQ ID NOs:89, 97, 105, 113, 121, 129, 137, 145, 153, 161, 169, 177, 185, 193, 201, 209, 217, 225, 233, 241, 249, 257, 265, 273, 281, 289, 297, 305, 313, 321, 329, and 337. In some embodiments, the CD28 ABD includes any of the variable light domains provided herein or a variant thereof.

In some embodiments, the CD28 ABD includes a variable light domain that has from 1, 2, 3, 4, 5, 6, 7, 8, 9 or 10 amino acid changes one of the following CD28 ABD variable light domains: SEQ ID NOs: 20, 93, 101, 109, 117, 125, 133, 141, 149, 157, 165, 173, 181, 189, 197, 205, 213, 221, 229, 237, 245, 253, 261, 269, 277, 285, 293, 301, 309, 317, 325, 333 and, 341. In some embodiments, the CD28 ABD includes any of the variable heavy domains provided herein or a variant thereof.

In one embodiment, the variant VH and/or VL domain is at least 90, 95, 97, 98 or 99% identical to the VH and/or VL of an anti-CD28 ABD as described herein, including FIGS. 41-74, and the Sequence Listing. In exemplary embodiments, the variant VH and/or VL domain is at least 90, 95, 97, 98 or 99% identical to the VH and/or VL of one of an anti-CD28 ABD comprising a VH and VL selected from the following:
(i) a VH having an amino acid sequence of SEQ ID NO: 89; and (ii) a VL having an amino acid sequence of SEQ ID NO:93;
(i) a VH having an amino acid sequence of SEQ ID NO:97; and (ii) a VL having an amino acid sequence of SEQ ID NO:101;
(i) a VH having an amino acid sequence of SEQ ID NO:105; and (ii) a VL having an amino acid sequence of SEQ ID NO:109;
(i) a VH having an amino acid sequence of SEQ ID NO:113; and (ii) a VL having an amino acid sequence of SEQ ID NO:117;
(i) a VH having an amino acid sequence of SEQ ID NO:121; and (ii) a VL having an amino acid sequence of SEQ ID NO:125;
(i) a VH having an amino acid sequence of SEQ ID NO:129; and (ii) a VL having an amino acid sequence of SEQ ID NO:133;
(i) a VH having an amino acid sequence of SEQ ID NO:137; and (ii) a VL having an amino acid sequence of SEQ ID NO:141;
(i) a VH having an amino acid sequence of SEQ ID NO:145; and (ii) a VL having an amino acid sequence of SEQ ID NO:149;
(i) a VH having an amino acid sequence of SEQ ID NO:153; and (ii) a VL having an amino acid sequence of SEQ ID NO:157;
(i) a VH having an amino acid sequence of SEQ ID NO:161; and (ii) a VL having an amino acid sequence of SEQ ID NO:165;
(i) a VH having an amino acid sequence of SEQ ID NO:169; and (ii) a VL having an amino acid sequence of SEQ ID NO:173;
(i) a VH having an amino acid sequence of SEQ ID NO:177; and (ii) a VL having an amino acid sequence of SEQ ID NO:181;
(i) a VH having an amino acid sequence of SEQ ID NO:185; and (ii) a VL having an amino acid sequence of SEQ ID NO:189;
(i) a VH having an amino acid sequence of SEQ ID NO:193; and (ii) a VL having an amino acid sequence of SEQ ID NO:197;
(i) a VH having an amino acid sequence of SEQ ID NO:201; and (ii) a VL having an amino acid sequence of SEQ ID NO:205;
(i) a VH having an amino acid sequence of SEQ ID NO:209; and (ii) a VL having an amino acid sequence of SEQ ID NO:213;
(i) a VH having an amino acid sequence of SEQ ID NO:217; and (ii) a VL having an amino acid sequence of SEQ ID NO:221;
(i) a VH having an amino acid sequence of SEQ ID NO:225; and (ii) a VL having an amino acid sequence of SEQ ID NO:229;
(i) a VH having an amino acid sequence of SEQ ID NO:233; and (ii) a VL having an amino acid sequence of SEQ ID NO:237; and
(i) a VH having an amino acid sequence of SEQ ID NO:241; and (ii) a VL having an amino acid sequence of SEQ ID NO:245;
(i) a VH having an amino acid sequence of SEQ ID NO:249; and (ii) a VL having an amino acid sequence of SEQ ID NO:253;
(i) a VH having an amino acid sequence of SEQ ID NO:257; and (ii) a VL having an amino acid sequence of SEQ ID NO:261;
(i) a VH having an amino acid sequence of SEQ ID NO:265; and (ii) a VL having an amino acid sequence of SEQ ID NO:269;
(i) a VH having an amino acid sequence of SEQ ID NO:273; and (ii) a VL having an amino acid sequence of SEQ ID NO:277;
(i) a VH having an amino acid sequence of SEQ ID NO:281; and (ii) a VL having an amino acid sequence of SEQ ID NO:285;
(i) a VH having an amino acid sequence of SEQ ID NO:289; and (ii) a VL having an amino acid sequence of SEQ ID NO:293;
(i) a VH having an amino acid sequence of SEQ ID NO:297; and (ii) a VL having an amino acid sequence of SEQ ID NO:301;
(i) a VH having an amino acid sequence of SEQ ID NO:305; and (ii) a VL having an amino acid sequence of SEQ ID NO:309;
(i) a VH having an amino acid sequence of SEQ ID NO:313; and (ii) a VL having an amino acid sequence of SEQ ID NO:317;
(i) a VH having an amino acid sequence of SEQ ID NO:321; and (ii) a VL having an amino acid sequence of SEQ ID NO:325;
(i) a VH having an amino acid sequence of SEQ ID NO:329; and (ii) a VL having an amino acid sequence of SEQ ID NO:333;
(i) a VH having an amino acid sequence of SEQ ID NO:337; and (ii) a VL having an amino acid sequence of SEQ ID NO:341;
(i) a VH having an amino acid sequence of SEQ ID NO:89; and (ii) a VL having an amino acid sequence of SEQ ID NO:20;
(i) a VH having an amino acid sequence of SEQ ID NO:97; and (ii) a VL having an amino acid sequence of SEQ ID NO:20;
(i) a VH having an amino acid sequence of SEQ ID NO:105; and (ii) a VL having an amino acid sequence of SEQ ID NO:20;
(i) a VH having an amino acid sequence of SEQ ID NO:113; and (ii) a VL having an amino acid sequence of SEQ ID NO:20;
(i) a VH having an amino acid sequence of SEQ ID NO:121; and (ii) a VL having an amino acid sequence of SEQ ID NO:20;
(i) a VH having an amino acid sequence of SEQ ID NO:129; and (ii) a VL having an amino acid sequence of SEQ ID NO:20;
(i) a VH having an amino acid sequence of SEQ ID NO:137; and (ii) a VL having an amino acid sequence of SEQ ID NO:20;
(i) a VH having an amino acid sequence of SEQ ID NO:145; and (ii) a VL having an amino acid sequence of SEQ ID NO:20;
(i) a VH having an amino acid sequence of SEQ ID NO:153; and (ii) a VL having an amino acid sequence of SEQ ID NO:20;
(i) a VH having an amino acid sequence of SEQ ID NO:161; and (ii) a VL having an amino acid sequence of SEQ ID NO:20;
(i) a VH having an amino acid sequence of SEQ ID NO:169; and (ii) a VL having an amino acid sequence of SEQ ID NO:20;
(i) a VH having an amino acid sequence of SEQ ID NO:177; and (ii) a VL having an amino acid sequence of SEQ ID NO:20;
(i) a VH having an amino acid sequence of SEQ ID NO:185; and (ii) a VL having an amino acid sequence of SEQ ID NO:20;
(i) a VH having an amino acid sequence of SEQ ID NO:193; and (ii) a VL having an amino acid sequence of SEQ ID NO:20;
(i) a VH having an amino acid sequence of SEQ ID NO:201; and (ii) a VL having an amino acid sequence of SEQ ID NO:20;
(i) a VH having an amino acid sequence of SEQ ID NO:209; and (ii) a VL having an amino acid sequence of SEQ ID NO:20;
(i) a VH having an amino acid sequence of SEQ ID NO:217; and (ii) a VL having an amino acid sequence of SEQ ID NO:20;
(i) a VH having an amino acid sequence of SEQ ID NO:225; and (ii) a VL having an amino acid sequence of SEQ ID NO:20;
(i) a VH having an amino acid sequence of SEQ ID NO:233; and (ii) a VL having an amino acid sequence of SEQ ID NO:20; and
(i) a VH having an amino acid sequence of SEQ ID NO:241; and (ii) a VL having an amino acid sequence of SEQ ID NO:20;
(i) a VH having an amino acid sequence of SEQ ID NO:249; and (ii) a VL having an amino acid sequence of SEQ ID NO:20;
(i) a VH having an amino acid sequence of SEQ ID NO:257; and (ii) a VL having an amino acid sequence of SEQ ID NO:20;
(i) a VH having an amino acid sequence of SEQ ID NO:265; and (ii) a VL having an amino acid sequence of SEQ ID NO:20;
(i) a VH having an amino acid sequence of SEQ ID NO:273; and (ii) a VL having an amino acid sequence of SEQ ID NO:20;
(i) a VH having an amino acid sequence of SEQ ID NO:281; and (ii) a VL having an amino acid sequence of SEQ ID NO:20;
(i) a VH having an amino acid sequence of SEQ ID NO:289; and (ii) a VL having an amino acid sequence of SEQ ID NO:20;
(i) a VH having an amino acid sequence of SEQ ID NO:297; and (ii) a VL having an amino acid sequence of SEQ ID NO:20;
(i) a VH having an amino acid sequence of SEQ ID NO:305; and (ii) a VL having an amino acid sequence of SEQ ID NO:20;
(i) a VH having an amino acid sequence of SEQ ID NO:313; and (ii) a VL having an amino acid sequence of SEQ ID NO:20;
(i) a VH having an amino acid sequence of SEQ ID NO:321; and (ii) a VL having an amino acid sequence of SEQ ID NO:20;
(i) a VH having an amino acid sequence of SEQ ID NO:329; and (ii) a VL having an amino acid sequence of SEQ ID NO:20; and
(i) a VH having an amino acid sequence of SEQ ID NO:337; and (ii) a VL having an amino acid sequence of SEQ ID NO:20. (FIGS. 24, and 41-74).

In certain embodiments, the anti-CD28 ABD is capable of binding to CD28, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding to human and cynomolgus CD28.

In some embodiments, the CD28 ABD includes a variable heavy domain that is at least 90, 95, 97, 98 or 99% identical to one of the following CD28 ABD variable heavy domains: SEQ ID NOs:89, 97, 105, 113, 121, 129, 137, 145, 153, 161, 169, 177, 185, 193, 201, 209, 217, 225, 233, 241, 249, 257, 265, 273, 281, 289, 297, 305, 313, 321, 329, and 337. In some embodiments, the CD28 ABD includes any of the variable light domains provided herein or a variant thereof.

In some embodiments, the CD28 ABD includes a variable light domain that is at least 90, 95, 97, 98 or 99% identical to one of the following CD28 ABD variable light domains: SEQ ID NOs: 20, 93, 101, 109, 117, 125, 133, 141, 149, 157, 165, 173, 181, 189, 197, 205, 213, 221, 229, 237, 245, 253, 261, 269, 277, 285, 293, 301, 309, 317, 325, 333 and, 341. In some embodiments, the CD28 ABD includes any of the variable heavy domains provided herein or a variant thereof.

In some embodiments, the CD28 ABDs have variable heavy and variable light domains that are 90, 95, 97, 98 or 99% identical to the VH and/or VL domains of a CD28 ABD as described herein, but the CDRs are identical. In some embodiments, the VH selected from the group consisting of: SEQ ID NOs:89, 97, 105, 113, 121, 129, 137, 145, 153, 161, 169, 177, 185, 193, 201, 209, 217, 225, 233, 241, 249, 257, 265, 273, 281, 289, 297, 305, 313, 321, 329, and 337. In some embodiments, the VL is selected from the group consisting of: SEQ ID NOs: 20, 93, 101, 109, 117, 125, 133, 141, 149, 157, 165, 173, 181, 189, 197, 205, 213, 221, 229, 237, 245, 253, 261, 269, 277, 285, 293, 301, 309, 317, 325, 333 and, 341.

In some embodiments, the CD28 ABDs have variable heavy and variable light domains that are 90, 95, 97, 98 or 99% identical to the VH and/or VL domains of a CD28 ABD as described herein, and there can be from 0 to 6 amino acid modifications in the CDRs. In some embodiments, the VH selected from the group consisting of: SEQ ID NOs:89, 97, 105, 113, 121, 129, 137, 145, 153, 161, 169, 177, 185, 193, 201, 209, 217, 225, 233, 241, 249, 257, 265, 273, 281, 289, 297, 305, 313, 321, 329, and 337. In some embodiments, the VL is selected from the group consisting of: SEQ ID NOs: 20, 93, 101, 109, 117, 125, 133, 141, 149, 157, 165, 173, 181, 189, 197, 205, 213, 221, 229, 237, 245, 253, 261, 269, 277, 285, 293, 301, 309, 317, 325, 333 and, 341.

In some embodiments, the CD28 ABDs have variable heavy and variable light domains that are 90, 95, 97, 98 or 99% identical to the VH and/or VL domains of a CD28 ABD as described herein, and there can be from 0 to 6 amino acid modifications in the CDRs, with no CDR having more than 1 amino acid modification. In some embodiments, the VH selected from the group consisting of: SEQ ID NOs:89, 97, 105, 113, 121, 129, 137, 145, 153, 161, 169, 177, 185, 193, 201, 209, 217, 225, 233, 241, 249, 257, 265, 273, 281, 289, 297, 305, 313, 321, 329, and 337. In some embodiments, the VL is selected from the group consisting of: SEQ ID NOs: 20, 93, 101, 109, 117, 125, 133, 141, 149, 157, 165, 173, 181, 189, 197, 205, 213, 221, 229, 237, 245, 253, 261, 269, 277, 285, 293, 301, 309, 317, 325, 333 and, 341.

In certain embodiments, the CD28 ABD is capable of binding to the CD28, as measured by at least one of a Biacore, surface plasmon resonance (SPR) and/or BLI (biolayer interferometry, e.g., Octet assay) assay, with the latter finding particular use in many embodiments. In particular embodiments, the CD28 ABD is capable of binding human CD28 (see FIG. 2) at detectable limits of the assay.

Such CD28 binding domains can be included in any of the antibodies provided herein including, for example, the bispecific and trispecific antibody formats provided in FIGS. 82 and 83.

1. Additional CD28 Binding Domains

In another aspect, provided herein are additional CD28 binding domains that can be used in anti-CD28 antibodies including any of the anti-CD28 antibodies described herein. In some embodiments, the anti-CD28 binding domain includes a variable heavy domain selected from any of those in FIG. 163 (SEQ ID NOs:3354-3389) or a variant thereof, and a common light chain with a variable light domain referred to as “IGKV1-39_L1” (also referred to as, “2A3A4.248[PDL1]_L1” (SEQ ID NO:3239, see FIG. 161A) and “1F12A4.249[PDL2]_L1” (SEQ ID NO:3271, see FIG. 162A)) or variant thereof, wherein common light chain can also be used as a light chain for a PD-L1 and/or PD-L2 binding domain. In some embodiments, the anti-CD28 antibodies provided herein (e.g., anti-CD28×anti-PD-L1, and anti-CD28×anti-PD-L2 antibodies, and anti-CD28×anti-PD-L1×anti-PDL2 trispecific) includes a CD28 binding domain that includes a common light chain with the IGKV1-39_L1 variable light domain. In some embodiments, the anti-CD28 antibody is an anti-CD28×anti-PD-L1 antibody having a CD28 binding domain and a PD-L1 binding domain that each include a common light chain with the IGKV1-39_L1 variable light domain (SEQ ID NO:3239). In some embodiments, the anti-CD28 antibody is an anti-CD28×anti-PD-L2 antibody having a CD28 binding domain and a PD-L2 binding domain that each include a common light chain with the IGKV1-39_L1 variable light domain (SEQ ID NO:3239). In some embodiments, the anti-CD28 antibody is an anti-CD28×anti-PD-L1×anti-PD-L2 antibody having a CD28 binding domain, a PD-L1 binding domain, and a PD-L2 binding domain that each include a common light chain with the IGKV1-39_L1 variable light domain (SEQ ID NO:3239). Such CD28 binding domains that utilize a common light chain with the IGKV1-39_L1 variable light domain (SEQ ID NO:3239) can be used, for example, in any of the antibody formats provided herein that utilize a common light chain (see, e.g., 1+1 CLC, 2+1 CLC, 1+1+1 stackFab2-scFv-Fc, 1+1+1 Fab-(Fab-scFv)-Fc, 1+1+1 mAb-scFv, and 1+1+1 stackFab2-Fab-Fc formats disclosed herein, FIGS. 82 and 83).

In one embodiment, the CD28 antigen binding domain includes the vhCDR1-3 of any of the CD28 variable heavy domains depicted in FIG. 163 (SEQ ID NOs:3354-3389), and the vlCDR1-3 of the IGK1-39_L1 variable light domain (SEQ ID NO:3239).

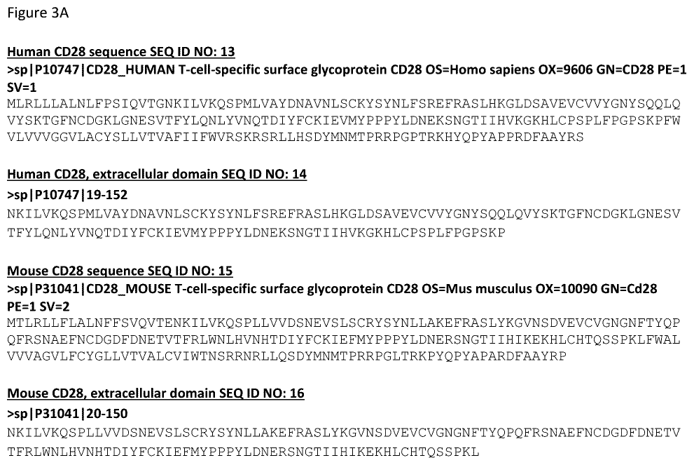

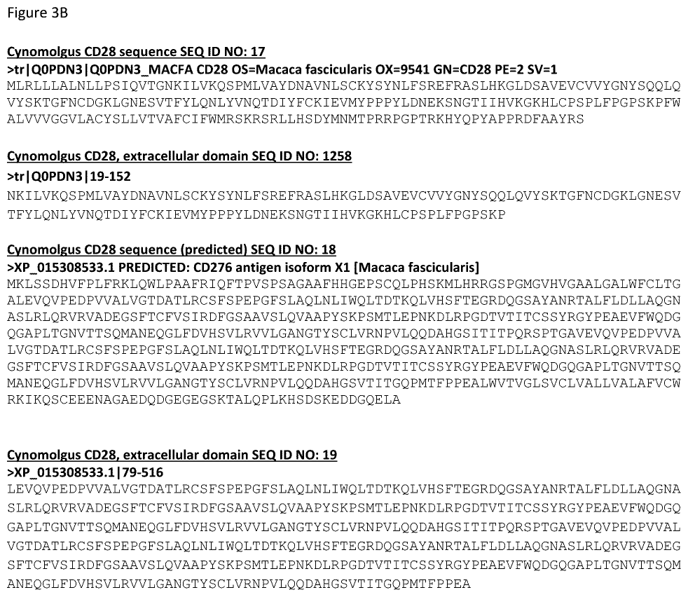

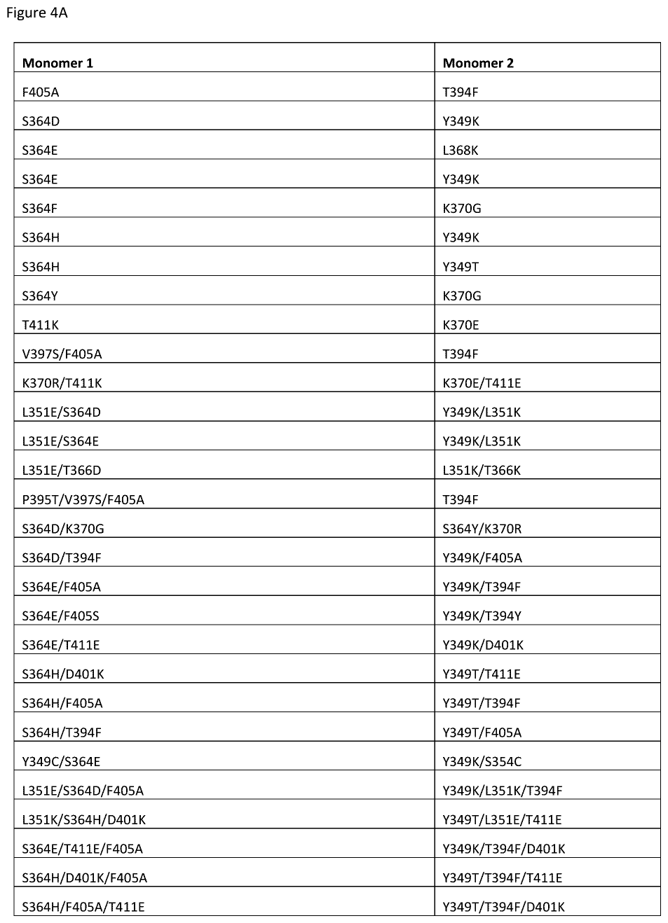

In one embodiment, the CD28 ABD of the subject anti-CD28 antibodies described herein includes a) a vhCDR1, vhCDR2, and/or vhCDR3 with 1, 2, 3, 4, 5, 6, 7, 8, 9, 10 amino acid modifications as compared to the vhCDR1, vhCDR2, and/or vhCDR3 of one of the CD28 variable heavy domains depicted in FIG. 163 (SEQ ID NOs:3354-3389), respectively, and/or b) a vlCDR1, vlCDR2, and/or vlCDR3 with 1, 2, 3, 4, 5, 6, 7, 8, 9, 10 amino acid modifications as compared to the vlCDR1, vlCDR2, and/or vlCDR3 of the IGK1-39_L1 variable light domain (SEQ ID NO:3239), respectively. In certain embodiments, the CD28 ABD of the subject anti-CD28 antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 3).

In one embodiment, the CD28 ABD of the subject anti-CD28 antibody includes a vhCDR1, vhCDR2, and/or vhCDR3 that is at least 90, 95, 97, 98 or 99% identical to the vhCDR1, vhCDR2, and/or vhCDR3 of one of the CD28 variable heavy domains depicted in FIG. 163 (SEQ ID NOs:3354-3389), respectively, and/or a vlCDR1, vlCDR2, and/or vlCDR3 that is at least 90, 95, 97, 98 or 99% identical to the vlCDR1, vlCDR2, and/or vlCDR3 of the IGK1-39_L1 variable light domain (SEQ ID NO:3239), respectively. In certain embodiments, the CD28 ABD is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 3).

In another exemplary embodiment, the CD28 ABD of the subject anti-CD28 antibody includes the variable heavy (VH) domain of one of the CD28 variable heavy domains depicted in FIG. 163 (SEQ ID NOs:3354-3389), and the IGK1-39_L1 variable light domain (SEQ ID NO:3239).

In some embodiments, the anti-CD28 antibody includes a CD28 ABD that includes a variable heavy domain that is a variant of one of the CD28 variable heavy domains depicted in FIG. 163 (SEQ ID NOs:3354-3389), and/or a variable light domain that is a variant of the IGK1-39_L1 variable light domain (SEQ ID NO:3239). In some embodiments, the variant VH domain has from 1, 2, 3, 4, 5, 6, 7, 8, 9 or 10 amino acid changes compared to one of the CD28 variable heavy domains depicted in FIG. 163 (SEQ ID NOs:3354-3389) and/or the variable light domain has from 1, 2, 3, 4, 5, 6, 7, 8, 9 or 10 amino acid changes from the IGK1-39_L1 variable light domain (SEQ ID NO:3239). In some embodiments, the one or more amino acid changes are in the VH and/or VL framework regions (FR1, FR2, FR3, and/or FR4). In some embodiments, the one or more amino acid change(s) are in one or more CDRs. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 3).

In one embodiment, the variant VH domain is at least 90, 95, 97, 98 or 99% identical to one of the CD28 variable heavy domains depicted in FIG. 163 (SEQ ID NOs:3354-3389) and/or the variable light domain is at least 90, 95, 97, 98 or 99% identical to the IGK1-39_L1 variable light domain (SEQ ID NO:3239). In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 3).

In some embodiments, the CD28 binding domain includes a VH that includes any one of the VHCDR1-3 and/or HFR1-4 sequences depicted in FIG. 163C (SEQ ID NOs:3390-3393 and 25674-2676) and the IGK1-39_L1 variable light domain (SEQ ID NO:3239) or a variant thereof.

In another embodiments, CD28 binding domains includes a variable heavy domain selected from any of those in FIG. 163 (SEQ ID NOs:3354-3389) or a variant thereof, and a variable light domain selected from those in FIG. 35 or 37 or a variant thereof.

In one embodiment, the CD28 ABD of the subject anti-CD28 antibodies described herein includes a) a vhCDR1, vhCDR2, and/or vhCDR3 with 1, 2, 3, 4, 5, 6, 7, 8, 9, 10 amino acid modifications as compared to the vhCDR1, vhCDR2, and/or vhCDR3 of one of the CD28 variable heavy domains depicted in FIG. 163 (SEQ ID NOs:3354-3389), respectively, and/or b) a vlCDR1, vlCDR2, and/or vlCDR3 with 1, 2, 3, 4, 5, 6, 7, 8, 9, 10 amino acid modifications as compared to the vlCDR1, vlCDR2, and/or vlCDR3 of a variable light domain selected from those in FIG. 35 or 37, respectively. In certain embodiments, the CD28 ABD of the subject anti-CD28 antibody is capable of binding CD28 antigen, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 3).

In one embodiment, the CD28 ABD of the subject anti-CD28 antibody includes a vhCDR1, vhCDR2, and/or vhCDR3 that is at least 90, 95, 97, 98 or 99% identical to the vhCDR1, vhCDR2, and/or vhCDR3 of one of the CD28 variable heavy domains depicted in FIG. 163 (SEQ ID NOs:3354-3389), respectively, and/or a vlCDR1, vlCDR2, and/or vlCDR3 that is at least 90, 95, 97, 98 or 99% identical to the vlCDR1, vlCDR2, and/or vlCDR3 of a variable light domain selected from those in FIG. 35 or 37, respectively. In certain embodiments, the CD28 ABD is capable of binding to the CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 3).

In another exemplary embodiment, the CD28 ABD of the subject anti-CD28 antibody includes the variable heavy (VH) domain of one of the CD28 variable heavy domains depicted in FIG. 163 (SEQ ID NOs:3354-3389), and a variable light domain selected from those in FIG. 35 or 37.

In some embodiments, the anti-CD28 antibody includes a CD28 ABD that includes a variable heavy domain that is a variant of one of the CD28 variable heavy domains depicted in FIG. 163 (SEQ ID NOs:3354-3389), and/or a variable light domain that is a variant of a variable light domain selected from those in FIG. 35 or 37. In some embodiments, the variant VH domain has from 1, 2, 3, 4, 5, 6, 7, 8, 9 or 10 amino acid changes compared to one of the CD28 variable heavy domains depicted in FIG. 163 (SEQ ID NOs:3354-3389) and/or the variable light domain has from 1, 2, 3, 4, 5, 6, 7, 8, 9 or 10 amino acid changes from a variable light domain selected from those in FIG. 35 or 37. In some embodiments, the one or more amino acid changes are in the VH and/or VL framework regions (FR1, FR2, FR3, and/or FR4). In some embodiments, the one or more amino acid change(s) are in one or more CDRs. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 3).

In one embodiment, the variant VH domain is at least 90, 95, 97, 98 or 99% identical to one of the CD28 variable heavy domains depicted in FIG. 163 (SEQ ID NOs:3354-3389) and/or the variable light domain is at least 90, 95, 97, 98 or 99% identical to a variable light domain selected from those in FIG. 35 or 37. In certain embodiments, the CD28 ABD is capable of binding to CD28, as measured by at least one of a Biacore, surface plasmon resonance (SPR), flow cytometry, and/or BLI (biolayer interferometry, e.g., Octet assay) assay, with the latter finding particular use in many embodiments. In particular embodiments, the CD28 ABD is capable of binding human CD28 antigen (see FIG. 1).

In another aspect, provided herein is a CD28 binding domain that competes with any of the PD-L1 binding domains disclosed herein for binding to human CD28.

### V. Antibodies

In one aspect provided herein are anti-CD28 antibodies and anti-PD-L1 antibodies. Antibodies provided herein can include any of the PD-L1, PD-L2, and/or CD28 binding domains provided herein (e.g., the antibody formats described in Section V.F. and FIGS. 82 and 83).

The antibodies provided herein include different antibody domains. As described herein and known in the art, the antibodies described herein include different domains within the heavy and light chains, which can be overlapping as well. These domains include, but are not limited to, the Fc domain, the CH1 domain, the CH2 domain, the CH3 domain, the hinge domain, the heavy constant domain (CH1-hinge-Fc domain or CH1-hinge-CH2-CH3), the variable heavy domain, the variable light domain, the light constant domain, Fab domains and scFv domains.

As shown herein, there are a number of suitable linkers (for use as either domain linkers or scFv linkers) that can be used to covalently attach the recited domains (e.g., scFvs, Fabs, Fc domains, etc.), including traditional peptide bonds, generated by recombinant techniques. Exemplary linkers to attach domains of the subject antibody to each other are depicted in FIG. 7. In some embodiments, the linker peptide may predominantly include the following amino acid residues: Gly, Ser, Ala, or Thr. The linker peptide should have a length that is adequate to link two molecules in such a way that they assume the correct conformation relative to one another so that they retain the desired activity. In one embodiment, the linker is from about 1 to 50 amino acids in length, preferably about 1 to 30 amino acids in length. In one embodiment, linkers of 1 to 20 amino acids in length may be used, with from about 5 to about 10 amino acids finding use in some embodiments. Useful linkers include glycine-serine polymers, including for example (GS)n, (GSGGS)n, (GGGGS)n, and (GGGS)n, where n is an integer of at least one (and generally from 3 to 4), glycine-alanine polymers, alanine-serine polymers, and other flexible linkers. Alternatively, a variety of nonproteinaceous polymers, including but not limited to polyethylene glycol (PEG), polypropylene glycol, polyoxyalkylenes, or copolymers of polyethylene glycol and polypropylene glycol, may find use as linkers.

Other linker sequences may include any sequence of any length of CL/CH1 domain but not all residues of CL/CH1 domain; for example the first 5-12 amino acid residues of the CL/CH1 domains. Linkers can be derived from immunoglobulin light chain, for example Cκ or Cλ. Linkers can be derived from immunoglobulin heavy chains of any isotype, including for example Cγ1, Cγ2, Cγ3, Cγ4, Cα1, Cα2, Cδ, Cε, and Cμ. Linker sequences may also be derived from other proteins such as Ig-like proteins (e.g., TCR, FcR, KIR), hinge region-derived sequences, and other natural sequences from other proteins.

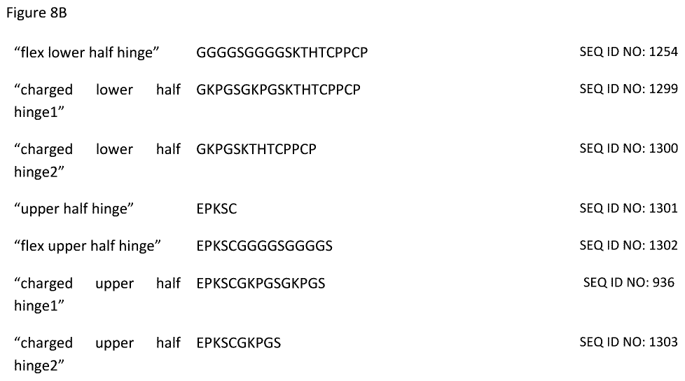

In some embodiments, the linker is a “domain linker”, used to link any two domains as outlined herein together. For example, in the 2+1 Fab₂-scFv-Fc format, there may be a domain linker that attaches the C-terminus of the CH1 domain of the Fab to the N-terminus of the scFv, with another optional domain linker attaching the C-terminus of the scFv to the CH2 domain (although in many embodiments the hinge is used as this domain linker). While any suitable linker can be used, many embodiments utilize a glycine-serine polymer as the domain linker, including for example (GS)n, (GSGGS)n, (GGGGS)n, and (GGGS)n, where n is an integer of at least one (and generally from 3 to 4 to 5) as well as any peptide sequence that allows for recombinant attachment of the two domains with sufficient length and flexibility to allow each domain to retain its biological function. In some cases, and with attention being paid to “strandedness”, as outlined below, charged domain linkers, as used in some embodiments of scFv linkers can be used. Exemplary useful domain linkers are depicted in FIG. 8.

In some embodiments, the linker is a scFv linker that is used to covalently attach the VH and VL domains as discussed herein. In many cases, the scFv linker is a charged scFv linker, a number of which are shown in FIG. 7. Accordingly, provided herein are charged scFv linkers, to facilitate the separation in pI between a first and a second monomer. That is, by incorporating a charged scFv linker, either positive or negative (or both, in the case of scaffolds that use scFvs on different monomers), this allows the monomer comprising the charged linker to alter the pI without making further changes in the Fc domains. These charged linkers can be substituted into any scFv containing standard linkers. Again, as will be appreciated by those in the art, charged scFv linkers are used on the correct “strand” or monomer, according to the desired changes in pI. For example, as discussed herein, to make 1+1 Fab-scFv-Fc format heterodimeric antibody, the original pI of the Fv region for each of the desired antigen binding domains are calculated, and one is chosen to make an scFv, and depending on the pI, either positive or negative linkers are chosen.

Charged domain linkers can also be used to increase the pI separation of the monomers of the invention as well, and thus those included in FIG. 6 can be used in any embodiment herein where a linker is utilized.

The PD-L1 binding domains, PD-L2 binding domains, and CD28 binding domains provided can be included in any useful antibody format including, for example, canonical immunoglobulin, as well as the formats provided herein (see, e.g., Section V.F. and FIGS. 82 and 83).

In some embodiments, the subject antibody includes one or more of the PD-L1 and/or PD-L2 ABDs provided herein. In some embodiments, the antibody includes one PD-L1 ABD. In other embodiments, the antibody includes two PD-L1 ABDs. In some embodiments, the antibody includes one PD-L2 ABD. In other embodiments, the antibody includes two PD-L2 ABDs. In exemplary embodiments, the antibody includes one PD-L1 ABD and one PD-L2 ABD.

In an exemplary embodiment, the antibody is a bispecific or trispecific antibody that includes one or two PD-L1 ABDs, including any of the PD-L1 ABDs provided herein. In an exemplary embodiment, the antibody is a bispecific or trispecific antibody that includes one or two PD-L2 ABDs, including any of the PD-L2 ABDs provided herein. Bispecific and trispecific antibody that include such PD-L1 and PD-L2 ABDs include, for example, “1+1 Fab-scFv-Fc,” “2+1 Fab₂-scFv-Fc,” “2+1 mAb-scFv,” “1+1 common light chain,” “2+1 common light chain,” “1+1+1 stackFab₂-scFv-Fc,” “1+1+1 Fab-(Fab-scFv)-Fc,” “1+1+1 mAb-scFv,” and, “1+1+1 stackFab₂-Fab-Fc” format antibodies (See FIGS. 82 and 83).

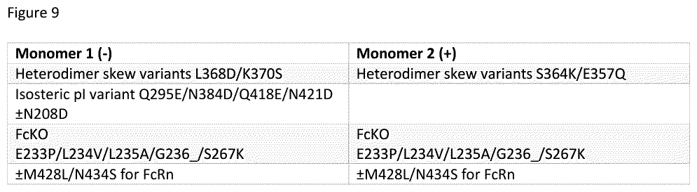

In exemplary embodiments, the PD-L1 ABD includes a VH and VL selected from those in FIGS. 17, 19, 20, 21, 24-26, and 28 or a variant thereof. In some embodiments, such bispecific and trispecific antibodies are heterodimeric bispecific antibodies that include any of the heterodimerization skew variants, pI variants and/or ablation variants described herein. See FIG. 9. In some embodiments, the PD-L1 ABD includes a VH and VL selected from the following:
i) a variable heavy domain of any of the PD-L1 variable heavy domains in FIGS. 17 and 19, and a variable light domain of any of the PD-L1 variable light domains in FIGS. 17 and 20,
ii) a variable heavy domain and variable light domain of any of the PD-L1 binding domains in FIGS. 17 and 21,
iii) a variable heavy domain of any of the PD-L1 binding domains in FIGS. 25 and 26, and a variable light domain of any of the PD-L1 binding domains in FIGS. 25 and 26,
iv) a variable heavy domain and variable light domain of any of the PD-L1 binding domains in FIGS. 25 and 26,
v) a variable heavy domain of any of the PD-L1 binding domains in FIGS. 25 and 26, and a variable light domain having the amino acid sequence of SEQ ID NO:20 (FIG. 24), and
vi) a variable heavy domain and variable light domain of any of the PD-L1 binding domains in FIG. 28.

In exemplary embodiments, the PD-L2 ABD includes a VH and VL selected from those in FIGS. 24, 29-32 and 34 or a variant thereof. In some embodiments, such bispecific and trispecific antibodies are heterodimeric bispecific antibodies that include any of the heterodimerization skew variants, pI variants and/or ablation variants described herein. See FIG. 9. In some embodiments, the PD-L2 ABD includes a VH and VL selected from the following:
i) a variable heavy domain of any of the PD-L2 binding domains in FIGS. 29-32, and a variable light domain of any of the PD-L2 binding domains in FIGS. 29-32,
ii) a variable heavy domain and variable light domain of any of the PD-L2 binding domains in FIGS. 29-32,
iii) a variable heavy domain of any of the PD-L2 binding domains in FIGS. 29-32, and a variable light domain having the amino acid sequence of SEQ ID NO:20 (FIG. 24), and
iv) a variable heavy domain and variable light domain of any of the PD-L2 binding domains in FIG. 34.

In some embodiments, the subject antibody includes one or more of the CD28 ABDs provided herein. In some embodiments, the antibody includes one CD28 ABD. In other embodiments, the antibody includes two CD28 ABDs. In exemplary embodiments, the antibody includes a CD28 ABD having a VH and VL selected from those depicted in FIGS. 35, 37, 38, 41-74 and 81 or a variant thereof. In exemplary embodiments, the CD28 ABD includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38,
ii) a variable heavy domain and a variable light domain of any of the CD28 binding domains in FIG. 38,
iii) a variable heavy domain of any of the CD28 binding domains in FIGS. 41-74, and a variable light domain of any of the CD28 binding domains in FIGS. 41-74,
iv) a variable heavy domain and variable light domain of any of the CD28 binding domains in FIGS. 41-74,
v) a variable heavy domain of any of the CD28 binding domains in FIGS. 41-74, and a variable light domain having the amino acid sequence of SEQ ID NO:20 (FIG. 24), and
vi) a variable heavy domain and variable light domain of any of the CD28 binding domains in FIG. 81.

### A. Chimeric and Humanized Antibodies

In certain embodiments, the subject antibodies provided herein include a heavy chain variable region from a particular germline heavy chain immunoglobulin gene and/or a light chain variable region from a particular germline light chain immunoglobulin gene. For example, such antibodies may comprise or consist of a human antibody comprising heavy or light chain variable regions that are “the product of” or “derived from” a particular germline sequence. A human antibody that is “the product of” or “derived from” a human germline immunoglobulin sequence can be identified as such by comparing the amino acid sequence of the human antibody to the amino acid sequences of human germline immunoglobulins and selecting the human germline immunoglobulin sequence that is closest in sequence (i.e., greatest % identity) to the sequence of the human antibody (using the methods outlined herein). A human antibody that is “the product of” or “derived from” a particular human germline immunoglobulin sequence may contain amino acid differences as compared to the germline sequence, due to, for example, naturally-occurring somatic mutations or intentional introduction of site-directed mutation. However, a humanized antibody typically is at least 90% identical in amino acids sequence to an amino acid sequence encoded by a human germline immunoglobulin gene and contains amino acid residues that identify the antibody as being derived from human sequences when compared to the germline immunoglobulin amino acid sequences of other species (e.g., murine germline sequences). In certain cases, a humanized antibody may be at least 95, 96, 97, 98 or 99%, or even at least 96%, 97%, 98%, or 99% identical in amino acid sequence to the amino acid sequence encoded by the germline immunoglobulin gene. Typically, a humanized antibody derived from a particular human germline sequence will display no more than 10-20 amino acid differences from the amino acid sequence encoded by the human germline immunoglobulin gene (prior to the introduction of any skew, pI and ablation variants herein; that is, the number of variants is generally low, prior to the introduction of the variants of the invention). In certain cases, the humanized antibody may display no more than 5, or even no more than 4, 3, 2, or 1 amino acid difference from the amino acid sequence encoded by the germline immunoglobulin gene (again, prior to the introduction of any skew, pI and ablation variants herein; that is, the number of variants is generally low, prior to the introduction of the variants of the invention).

In one embodiment, the parent antibody has been affinity matured, as is known in the art. Structure-based methods may be employed for humanization and affinity maturation, for example as described in U.S. Ser. No. 11/004,590. Selection based methods may be employed to humanize and/or affinity mature antibody variable regions, including but not limited to methods described in Wu et al., 1999, J. Mol. Biol. 294:151-162; Baca et al., 1997, J. Biol. Chem. 272(16):10678-10684; Rosok et al., 1996, J. Biol. Chem. 271(37): 22611-22618; Rader et al., 1998, Proc. Natl. Acad. Sci. USA 95: 8910-8915; Krauss et al., 2003, Protein Engineering 16(10):753-759, all entirely incorporated by reference. Other humanization methods may involve the grafting of only parts of the CDRs, including but not limited to methods described in U.S. Ser. No. 09/810,510; Tan et al., 2002, J. Immunol. 169:1119-1125; De Pascalis et al., 2002, J. Immunol. 169:3076-3084, all entirely incorporated by reference.

### B. Anti-CD28, Anti-PD-L1, and Anti-PD-L2 Bispecific and Trispecific Antibodies

In another aspect, provided herein are anti-CD28×anti-PD-L1, and anti-CD28×anti-PD-L2 antibodies, and anti-CD28×anti-PD-L1×anti-PDL2 trispecific. In some embodiments, the bispecific and trispecific antibodies provided herein includes an agonistic CD28 binding domain that provides co-stimulatory function by binding to CD28 on T cells. As such, the bispecific and trispecific antibodies provided herein enhance immune responses selectively at tumor sites that express PD-L1 and/or PD-L2. In some embodiments, the anti-CD28×anti-PD-L1 and CD28×anti-PD-L2 antibodies are bispecific antibodies. In some embodiments, the anti-CD28×anti-PD-L1×anti-PD-L2 antibody is a trispecific antibody. In some embodiments, the anti-CD28×anti-PD-L1 and CD28×anti-PD-L2 antibodies are bivalent antibodies. In some embodiments, the anti-CD28×anti-PD-L1 and CD28×anti-PD-L2 antibodies are trivalent antibodies. In some embodiments, the anti-CD28×anti-PD-L1 and CD28×anti-PD-L2 antibodies are bispecific, bivalent antibody. In exemplary embodiments, the anti-CD28×anti-PD-L1 and CD28×anti-PD-L2 antibodies are bispecific, trivalent antibodies. In some embodiments, the anti-CD28×anti-PD-L1×anti-PD-L2 antibody is a trispecific, trivalent antibody.

As is more fully outlined herein, the anti-CD28×anti-PD-L1 antibody can be in a variety of formats, as outlined below. Exemplary formats include the “1+1 Fab-scFv-Fc,” “2+1 Fab₂-scFv-Fc,” “2+1 mAb-scFv,” “1+1 common light chain,” “2+1 common light chain,” “1+1+1 stackFab₂-scFv-Fc,” “1+1+1 Fab-(Fab-scFv)-Fc,” “1+1+1 mAb-scFv,” and, “1+1+1 stackFab₂-Fab-Fc” format antibodies (See FIGS. 82 and 83). Other useful antibody formats include, but are not limited to, “mAb-Fv,” “mAb-scFv,” “central-Fv”, “one armed scFv-mAb,” “scFv-mAb,” “dual scFv,” and “trident” format antibodies, as disclosed in US20180127501A1, which is incorporated by reference herein, particularly in pertinent part relating to antibody formats (see, e.g., FIG. 2).

The can include any suitable CD28 ABD, including those described herein. In some embodiments, the CD28 ABD is an agonistic ABD that provides co-stimulatory function upon binding to CD28. In some embodiments, the subject antibody includes a CD28 binding domain that includes one of the following variable heavy domain and variable light domains or a variant thereof:
i) a variable heavy domain of any of the PD-L1 variable heavy domains in FIGS. 17 and 19, and a variable light domain of any of the PD-L1 variable light domains in FIGS. 17 and 20,
ii) a variable heavy domain and variable light domain of any of the PD-L1 binding domains in FIGS. 17 and 21,
iii) a variable heavy domain of any of the PD-L1 binding domains in FIGS. 25 and 26, and a variable light domain of any of the PD-L1 binding domains in FIGS. 25 and 26,
iv) a variable heavy domain and variable light domain of any of the PD-L1 binding domains in FIGS. 25 and 26,
v) a variable heavy domain of any of the PD-L1 binding domains in FIGS. 25 and 26, and a variable light domain having the amino acid sequence of SEQ ID NO:20 (FIG. 24),
vi) a variable heavy domain and variable light domain of any of the PD-L1 binding domains in FIG. 28,
vii) a variable heavy domain of any of the PD-L2 binding domains in FIGS. 29-32, and a variable light domain of any of the PD-L2 binding domains in FIGS. 29-32,
viii) a variable heavy domain and variable light domain of any of the PD-L2 binding domains in FIGS. 29-32,
ix) a variable heavy domain of any of the PD-L2 binding domains in FIGS. 29-32, and a variable light domain having the amino acid sequence of SEQ ID NO:20 (FIG. 24), and
x) a variable heavy domain and variable light domain of any of the PD-L2 binding domains in FIG. 34.

The anti-CD28×anti-PD-L1 antibody provided herein can include one or more PD-L1 binding domains and/or one or more PD-L2 binding domains. In some embodiments, the subject antibody includes one PD-L1 binding domain. In certain embodiments, the subject antibody includes two PD-L1 binding domains. In some embodiments, the subject antibody includes one PD-L2 binding domain. In certain embodiments, the subject antibody includes two PD-L2 binding domains.

Note that unless specified herein, the order of the antigen list in the name does not confer structure; that is an anti-PD-L1×anti-CD28 1+1 Fab-scFv-Fc antibody can have the scFv bind to PD-L1 or CD28, although in some cases, the order specifies structure as indicated.

In addition, in embodiments wherein the subject antibody includes an scFv, the scFv can be in an orientation from N- to C-terminus of VH-scFv linker-VL or VL-scFv linker-VH. In some formats, one or more of the ABDs generally is a Fab that includes a VH domain on one protein chain (generally as a component of a heavy chain) and a VL on another protein chain (generally as a component of a light chain).

As will be appreciated by those in the art, any set of 6 CDRs or VH and VL domains can be in the scFv format or in the Fab format, which is then added to the heavy and light constant domains, where the heavy constant domains comprise variants (including within the CH1 domain as well as the Fc domain). The scFv sequences contained in the sequence listing utilize a particular charged linker, but as outlined herein, uncharged or other charged linkers can be used, including those depicted in FIG. 6.

In addition, as discussed above, the numbering used in the Sequence Listing for the identification of the CDRs is Kabat, however, different numbering can be used, which will change the amino acid sequences of the CDRs as shown in Table 2.

For all of the variable heavy and light domains listed herein, further variants can be made. As outlined herein, in some embodiments the set of 6 CDRs can have from 0, 1, 2, 3, 4 or 5 amino acid modifications (with amino acid substitutions finding particular use), as well as changes in the framework regions of the variable heavy and light domains, as long as the frameworks (excluding the CDRs) retain at least about 80, 85 or 90% identity to a human germline sequence selected from those listed in FIG. 1 of U.S. Pat. No. 7,657,380, which Figure and Legend is incorporated by reference in its entirety herein. Thus, for example, the identical CDRs as described herein can be combined with different framework sequences from human germline sequences, as long as the framework regions retain at least 80, 85 or 90% identity to a human germline sequence selected from those listed in FIG. 1 of U.S. Pat. No. 7,657,380. Alternatively, the CDRs can have amino acid modifications (e.g., from 1, 2, 3, 4 or 5 amino acid modifications in the set of CDRs (that is, the CDRs can be modified as long as the total number of changes in the set of 6 CDRs is less than 6 amino acid modifications, with any combination of CDRs being changed; e.g., there may be one change in vlCDR1, two in vhCDR2, none in vhCDR3, etc.)), as well as having framework region changes, as long as the framework regions retain at least 80, 85 or 90% identity to a human germline sequence selected from those listed in FIG. 1 of U.S. Pat. No. 7,657,380.

### C. Heterodimeric Antibodies

In exemplary embodiments, the bispecific and trispecific antibodies provided herein are heterodimeric antibodies that include two variant Fc domain sequences. Such variant Fc domains include amino acid modifications to facilitate the self-assembly and/or purification of the heterodimeric antibodies.

An ongoing problem in antibody technologies is the desire for multispecific antibodies that bind to two or more different antigens simultaneously, thus allowing the different antigens to be brought into proximity and resulting in new functionalities and new therapies. In general, these antibodies are made by including genes for each heavy and light chain into the host cells. This generally results in the formation of the desired heterodimer (A-B), as well as the two homodimers (A-A and B-B (not including the light chain heterodimeric issues)). However, a major obstacle in the formation of bispecific antibodies is the difficulty in biasing the formation of the desired heterodimeric antibody over the formation of the homodimers and/or purifying the heterodimeric antibody away from the homodimers.

There are a number of mechanisms that can be used to generate the subject heterodimeric antibodies. In addition, as will be appreciated by those in the art, these different mechanisms can be combined to ensure high heterodimerization. Amino acid modifications that facilitate the production and purification of heterodimers are collectively referred to generally as “heterodimerization variants.” As discussed below, heterodimerization variants include “skew” variants (e.g., the “knobs and holes” and the “charge pairs” variants described below) as well as “pI variants,” which allow purification of heterodimers from homodimers. As is generally described in U.S. Pat. No. 9,605,084, hereby incorporated by reference in its entirety and specifically as below for the discussion of heterodimerization variants, useful mechanisms for heterodimerization include “knobs and holes” (“KIH”) as described in U.S. Pat. No. 9,605,084, “electrostatic steering” or “charge pairs” as described in U.S. Pat. No. 9,605,084, pI variants as described in U.S. Pat. No. 9,605,084, and general additional Fc variants as outlined in U.S. Pat. No. 9,605,084 and below.

Heterodimerization variants that are useful for the formation and purification of the subject heterodimeric antibody (e.g., bispecific antibodies) are further discussed in detailed below.

1. Skew Variants

In some embodiments, the αPD-L1 heterodimeric antibody (e.g., αPD-L1×αCD28 heterodimeric antibody) includes skew variants which are one or more amino acid modifications in a first Fc domain (A) and/or a second Fc domain (B) that favor the formation of Fc heterodimers (Fc dimers that include the first and the second Fc domain; (A-B) over Fc homodimers (Fc dimers that include two of the first Fc domain or two of the second Fc domain; A-A or B-B). Suitable skew variants are included in the FIG. 29 of US Publ. App. No. 2016/0355608, hereby incorporated by reference in its entirety and specifically for its disclosure of skew variants, as well as in FIGS. 3, 8 and 9.

One particular type of skew variants is generally referred to in the art as “knobs and holes,” referring to amino acid engineering that creates steric influences to favor heterodimeric formation and disfavor homodimeric formation, as described in U.S. Ser. No. 61/596,846, Ridgway et al., Protein Engineering 9(7):617 (1996); Atwell et al., J. Mol. Biol. 1997 270:26; U.S. Pat. No. 8,216,805, all of which are hereby incorporated by reference in their entirety and specifically for the disclosure of “knobs and holes” mutations. This is sometime referred to herein as “steric variants.” The figures identify a number of “monomer A-monomer B” pairs that rely on “knobs and holes”. In addition, as described in Merchant et al., Nature Biotech. 16:677 (1998), these “knobs and holes” mutations can be combined with disulfide bonds to further favor formation of Fc heterodimers.

Another method that finds use in the generation of heterodimers is sometimes referred to as “electrostatic steering” as described in Gunasekaran et al., J. Biol. Chem. 285(25):19637 (2010), hereby incorporated by reference in its entirety. This is sometimes referred to herein as “charge pairs”. In this embodiment, electrostatics are used to skew the formation towards heterodimerization. As those in the art will appreciate, these may also have an effect on pI, and thus on purification, and thus could in some cases also be considered pI variants. However, as these were generated to force heterodimerization and were not used as purification tools, they are classified as “skew variants”. These include, but are not limited to, D221E/P228E/L368E paired with D221R/P228R/K409R (e.g., these are “monomer corresponding sets) and C220E/P228E/368E paired with C220R/E224R/P228R/K409R.

In some embodiments, the skew variants advantageously and simultaneously favor heterodimerization based on both the “knobs and holes” mechanism as well as the “electrostatic steering” mechanism. In some embodiments, the heterodimeric antibody includes one or more sets of such heterodimerization skew variants. These variants come in “pairs” of “sets”. That is, one set of the pair is incorporated into the first monomer and the other set of the pair is incorporated into the second monomer. It should be noted that these sets do not necessarily behave as “knobs in holes” variants, with a one-to-one correspondence between a residue on one monomer and a residue on the other. That is, these pairs of sets may instead form an interface between the two monomers that encourages heterodimer formation and discourages homodimer formation, allowing the percentage of heterodimers that spontaneously form under biological conditions to be over 90%, rather than the expected 50% (25% homodimer A/A:50% heterodimer A/B:25% homodimer B/B). Exemplary heterodimerization “skew” variants are depicted in FIGS. 3, 8, and 9. In exemplary embodiments, the heterodimeric antibody includes a S364K/E357Q:L368D/K370S; L368D/K370S:S364K; L368E/K370S:S364K; T411T/E360E/Q362E:D401K; L368D/K370S:S364K/E357L; K370S:S364K/E357Q; or a T366S/L368A/Y407V:T366W (optionally including a bridging disulfide, T366S/L368A/Y407V/Y349C:T366W/S354C) “skew” variant amino acid substitution set. In an exemplary embodiment, the heterodimeric antibody includes a “S364K/E357Q:L368D/K370S” amino acid substitution set. In terms of nomenclature, the pair “S364K/E357Q:L368D/K370S” means that one of the monomers includes an Fc domain that includes the amino acid substitutions S364K and E357Q and the other monomer includes an Fc domain that includes the amino acid substitutions L368D and K370S; as above, the “strandedness” of these pairs depends on the starting pI.

In some embodiments, the skew variants provided herein can be optionally and independently incorporated with any other modifications, including, but not limited to, other skew variants (see, e.g., in FIG. 37 of US Publ. App. No. 2012/0149876, herein incorporated by reference, particularly for its disclosure of skew variants), pI variants, isotpypic variants, FcRn variants, ablation variants, etc. into one or both of the first and second Fc domains of the heterodimeric antibody. Further, individual modifications can also independently and optionally be included or excluded from the subject the heterodimeric antibody.

In some embodiments, the skew variants outlined herein can be optionally and independently incorporated with any pI variant (or other variants such as Fc variants, FcRn variants, etc.) into one or both heavy chain monomers, and can be independently and optionally included or excluded from the subject heterodimeric antibodies.

2. pI (Isoelectric Point) Variants for Heterodimers

In some embodiments, the αPD-L1 heterodimeric antibody (e.g., αPD-L1×αCD28 heterodimeric antibody) includes purification variants that advantageously allow for the separation of heterodimeric antibody from homodimeric proteins.

There are several basic mechanisms that can lead to ease of purifying heterodimeric antibodies. For example, modifications to one or both of the antibody heavy chain monomers A and B such that each monomer has a different pI allows for the isoelectric purification of heterodimeric A-B antibody from monomeric A-A and B-B proteins. Alternatively, some scaffold formats, such as the “1+1 Fab-scFv-Fc” format, the “2+1 Fab₂-scFv-Fc” format, and the “2+1 CLC” format allows separation on the basis of size. As described above, it is also possible to “skew” the formation of heterodimers over homodimers using skew variants. Thus, a combination of heterodimerization skew variants and pI variants find particular use in the heterodimeric antibodies provided herein.

Additionally, as more fully outlined below, depending on the format of the heterodimeric antibody, pI variants either contained within the constant region and/or Fc domains of a monomer, and/or domain linkers can be used. In some embodiments, the heterodimeric antibody includes additional modifications for alternative functionalities that can also create pI changes, such as Fc, FcRn and KO variants.

In some embodiments, the subject heterodimeric antibodies provided herein include at least one monomer with one or more modifications that alter the pI of the monomer (i.e., a “pI variant”). In general, as will be appreciated by those in the art, there are two general categories of pI variants: those that increase the pI of the protein (basic changes) and those that decrease the pI of the protein (acidic changes). As described herein, all combinations of these variants can be done: one monomer may be wild type, or a variant that does not display a significantly different pI from wild-type, and the other can be either more basic or more acidic. Alternatively, each monomer is changed, one to more basic and one to more acidic.

Depending on the format of the heterodimer antibody, pI variants can be either contained within the constant and/or Fc domains of a monomer, or charged linkers, either domain linkers or scFv linkers, can be used. That is, antibody formats that utilize scFv(s) such as “1+1 Fab-scFv-Fc”, format can include charged scFv linkers (either positive or negative), that give a further pI boost for purification purposes. As will be appreciated by those in the art, some 1+1 Fab-scFv-Fc and 2+1 Fab₂-scFv-Fc formats are useful with just charged scFv linkers and no additional pI adjustments, although the invention does provide pI variants that are on one or both of the monomers, and/or charged domain linkers as well. In addition, additional amino acid engineering for alternative functionalities may also confer pI changes, such as Fc, FcRn and KO variants.

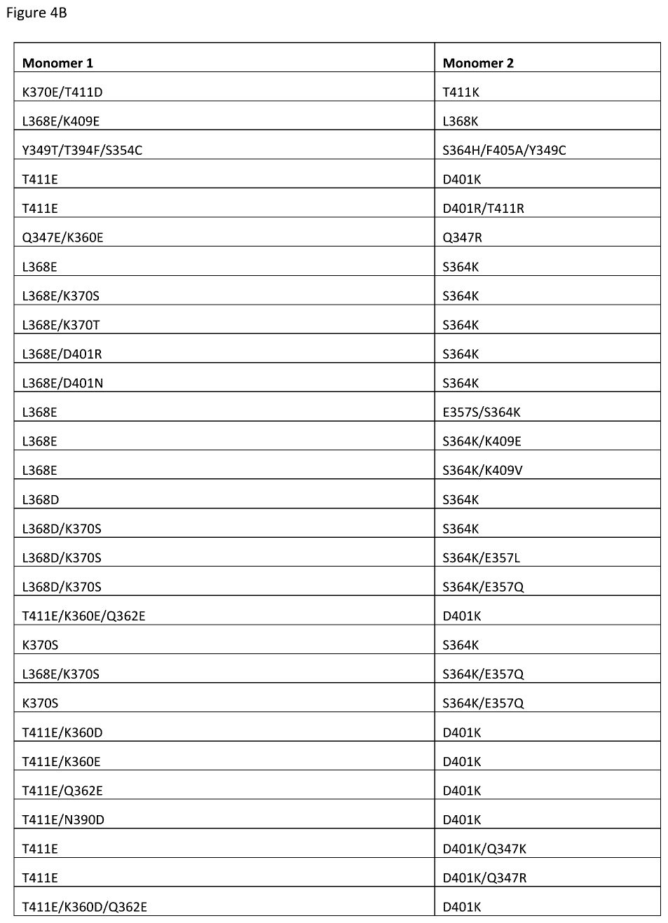

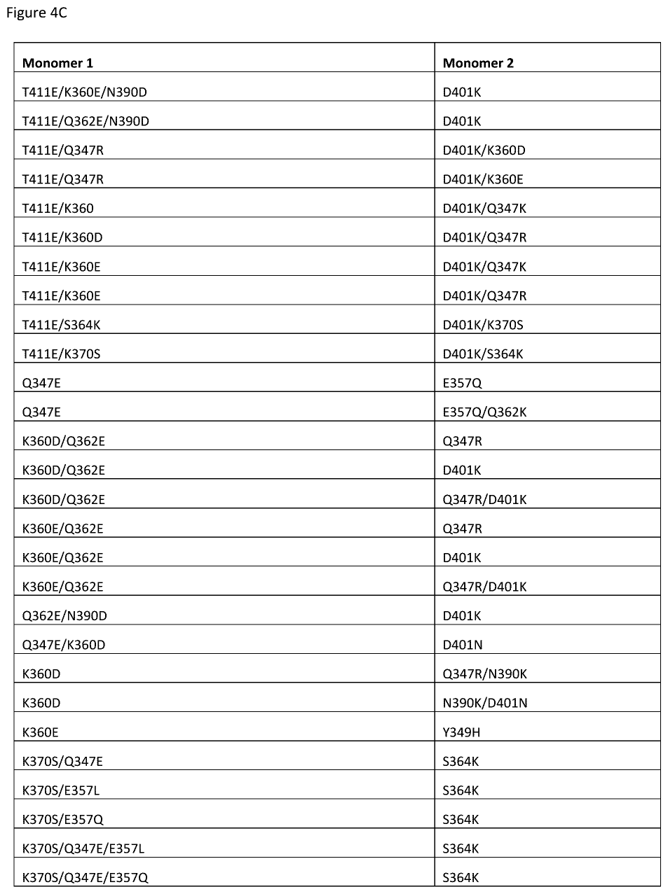

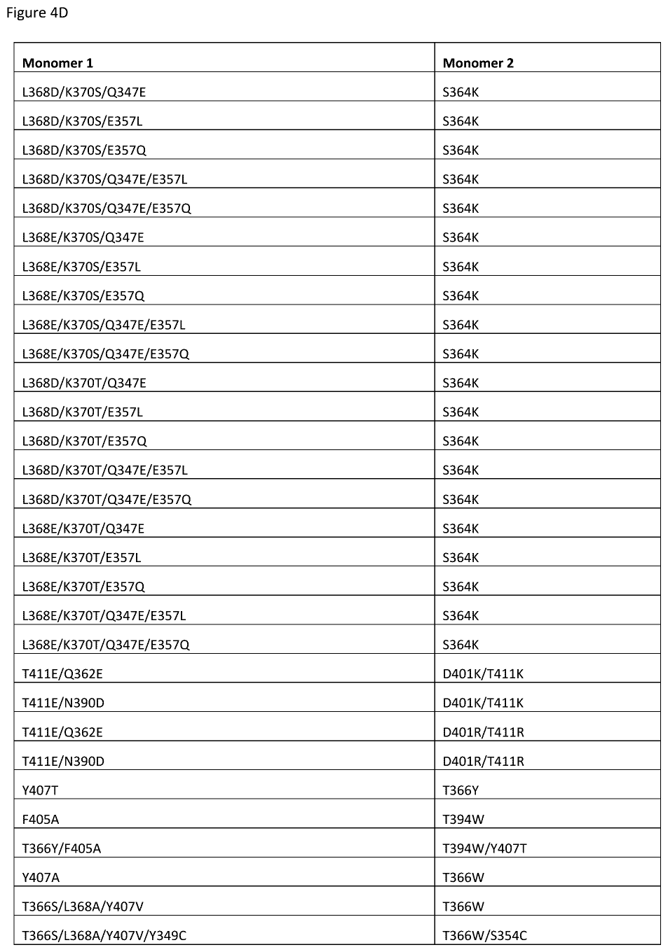

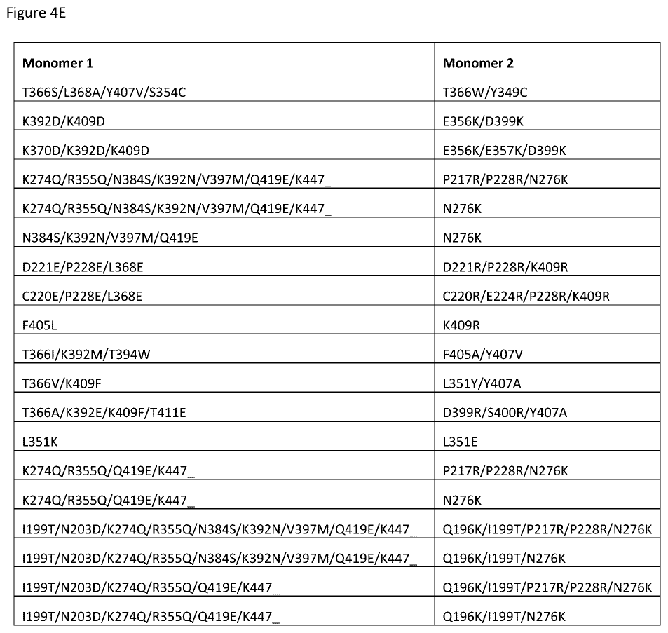

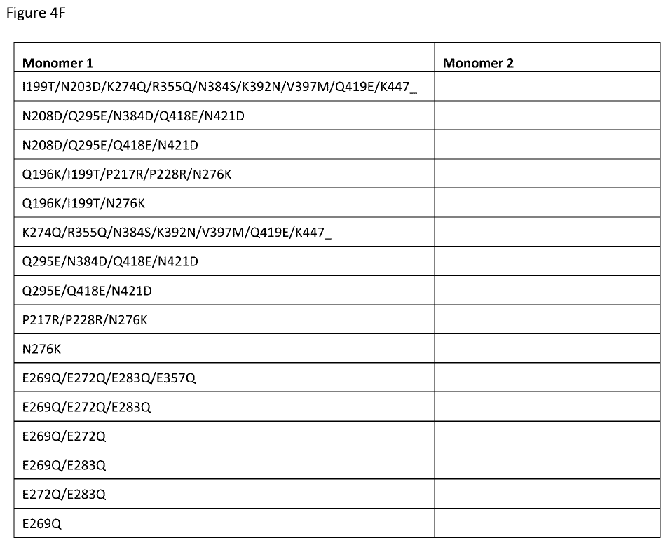

In subject heterodimeric antibodies that utilizes pI as a separation mechanism to allow the purification of heterodimeric proteins, amino acid variants are introduced into one or both of the monomer polypeptides. That is, the pI of one of the monomers (referred to herein for simplicity as “monomer A”) can be engineered away from monomer B, or both monomer A and B change be changed, with the pI of monomer A increasing and the pI of monomer B decreasing. As is outlined more fully below, the pI changes of either or both monomers can be done by removing or adding a charged residue (e.g., a neutral amino acid is replaced by a positively or negatively charged amino acid residue, e.g., glycine to glutamic acid), changing a charged residue from positive or negative to the opposite charge (aspartic acid to lysine) or changing a charged residue to a neutral residue (e.g., loss of a charge; lysine to serine). A number of these variants are shown in the FIGS. 3 and 4.

Thus, in some embodiments, the subject heterodimeric antibody includes amino acid modifications in the constant regions that alter the isoelectric point (pI) of at least one, if not both, of the monomers of a dimeric protein to form “pI antibodies”) by incorporating amino acid substitutions (“pI variants” or “pI substitutions”) into one or both of the monomers. As shown herein, the separation of the heterodimers from the two homodimers can be accomplished if the pIs of the two monomers differ by as little as 0.1 pH unit, with 0.2, 0.3, 0.4 and 0.5 or greater all finding use in the present invention.

As will be appreciated by those in the art, the number of pI variants to be included on each or both monomer(s) to get good separation will depend in part on the starting pI of the components, for example in the 1+1 Fab-scFv-Fc, 2+1 Fab₂-scFv-Fc, 1+1 CLC and 2+1 CLC formats, the starting pI of the scFv (1+1 Fab-scFv-Fc, 2+1 Fab₂-scFv-Fc) and Fab(s) of interest. That is, to determine which monomer to engineer or in which “direction” (e.g., more positive or more negative), the Fv sequences of the two target antigens are calculated and a decision is made from there. As is known in the art, different Fvs will have different starting pIs which are exploited in the present invention. In general, as outlined herein, the pIs are engineered to result in a total pI difference of each monomer of at least about 0.1 logs, with 0.2 to 0.5 being preferred as outlined herein.

In the case where pI variants are used to achieve heterodimerization, by using the constant region(s) of the heavy chain(s), a more modular approach to designing and purifying bispecific proteins, including antibodies, is provided. Thus, in some embodiments, heterodimerization variants (including skew and pI heterodimerization variants) are not included in the variable regions, such that each individual antibody must be engineered. In addition, in some embodiments, the possibility of immunogenicity resulting from the pI variants is significantly reduced by importing pI variants from different IgG isotypes such that pI is changed without introducing significant immunogenicity. Thus, an additional problem to be solved is the elucidation of low pI constant domains with high human sequence content, e.g., the minimization or avoidance of non-human residues at any particular position. Alternatively or in addition to isotypic substitutions, the possibility of immunogenicity resulting from the pI variants is significantly reduced by utilizing isosteric substitutions (e.g. Asn to Asp; and Gln to Glu).

As discussed below, a side benefit that can occur with this pI engineering is also the extension of serum half-life and increased FcRn binding. That is, as described in US Publ. App. No. US 2012/0028304 (incorporated by reference in its entirety), lowering the pI of antibody constant domains (including those found in antibodies and Fc fusions) can lead to longer serum retention in vivo. These pI variants for increased serum half-life also facilitate pI changes for purification.

In addition, it should be noted that the pI variants give an additional benefit for the analytics and quality control process of bispecific antibodies, as the ability to either eliminate, minimize and distinguish when homodimers are present is significant. Similarly, the ability to reliably test the reproducibility of the heterodimeric antibody production is important.

In general, embodiments of particular use rely on sets of variants that include skew variants, which encourage heterodimerization formation over homodimerization formation, coupled with pI variants, which increase the pI difference between the two monomers to facilitate purification of heterodimers away from homodimers.

Exemplary combinations of pI variants are shown in FIGS. 4 and 5, and FIG. 30 of US Publ. App. No. 2016/0355608, all of which are herein incorporated by reference in its entirety and specifically for the disclosure of pI variants. Preferred combinations of pI variants are shown in FIGS. 4 and 5. As outlined herein and shown in the figures, these changes are shown relative to IgG1, but all isotypes can be altered this way, as well as isotype hybrids. In the case where the heavy chain constant domain is from IgG2-4, R133E and R133Q can also be used.

In one embodiment, a preferred combination of pI variants has one monomer (the negative Fab side) comprising 208D/295E/384D/418E/421D variants (N208D/Q295E/N384D/Q418E/N421D when relative to human IgG1) and a second monomer (the positive scFv side) comprising a positively charged scFv linker, including (GKPGS)₄ (SEQ ID NO:892). However, as will be appreciated by those in the art, the first monomer includes a CH1 domain, including position 208. Accordingly, in constructs that do not include a CH1 domain (for example for antibodies that do not utilize a CH1 domain on one of the domains), a preferred negative pI variant Fc set includes 295E/384D/418E/421D variants (Q295E/N384D/Q418E/N421D when relative to human IgG1).

Accordingly, in some embodiments, one monomer has a set of substitutions from FIG. 4 and the other monomer has a charged linker (either in the form of a charged scFv linker because that monomer comprises an scFv or a charged domain linker, as the format dictates, which can be selected from those depicted in FIG. 6).

In some embodiments, modifications are made in the hinge of the Fc domain, including positions 216, 217, 218, 219, 220, 221, 222, 223, 224, 225, 226, 227, 228, 229, and 230 based on EU numbering. Thus, pI mutations and particularly substitutions can be made in one or more of positions 216-230, with 1, 2, 3, 4 or 5 mutations finding use. Again, all possible combinations are contemplated, alone or with other pI variants in other domains.

Specific substitutions that find use in lowering the pI of hinge domains include, but are not limited to, a deletion at position 221, a non-native valine or threonine at position 222, a deletion at position 223, a non-native glutamic acid at position 224, a deletion at position 225, a deletion at position 235 and a deletion or a non-native alanine at position 236. In some cases, only pI substitutions are done in the hinge domain, and in others, these substitution(s) are added to other pI variants in other domains in any combination.

In some embodiments, mutations can be made in the CH2 region, including positions 233, 234, 235, 236, 274, 296, 300, 309, 320, 322, 326, 327, 334 and 339, based on EU numbering. It should be noted that changes in 233-236 can be made to increase effector function (along with 327A) in the IgG2 backbone. Again, all possible combinations of these 14 positions can be made; e.g., an anti-CD28 or anti-PD-L1 antibody provided herein may include a variant Fc domain with 1, 2, 3, 4, 5, 6, 7, 8, 9 or 10 CH2 pI substitutions.

Specific substitutions that find use in lowering the pI of CH2 domains include, but are not limited to, a non-native glutamine or glutamic acid at position 274, a non-native phenylalanine at position 296, a non-native phenylalanine at position 300, a non-native valine at position 309, a non-native glutamic acid at position 320, a non-native glutamic acid at position 322, a non-native glutamic acid at position 326, a non-native glycine at position 327, a non-native glutamic acid at position 334, a non-native threonine at position 339, and all possible combinations within CH2 and with other domains.

In this embodiment, the modifications can be independently and optionally selected from position 355, 359, 362, 384, 389,392, 397, 418, 419, 444 and 447 (EU numbering) of the CH3 region. Specific substitutions that find use in lowering the pI of CH3 domains include, but are not limited to, a non-native glutamine or glutamic acid at position 355, a non-native serine at position 384, a non-native asparagine or glutamic acid at position 392, a non-native methionine at position 397, a non-native glutamic acid at position 419, a non-native glutamic acid at position 359, a non-native glutamic acid at position 362, a non-native glutamic acid at position 389, a non-native glutamic acid at position 418, a non-native glutamic acid at position 444, and a deletion or non-native aspartic acid at position 447.

3. Isotypic Variants

In addition, many embodiments of the subject heterodimeric antibodies rely on the “importation” of pI amino acids at particular positions from one IgG isotype into another, thus reducing or eliminating the possibility of unwanted immunogenicity being introduced into the variants. A number of these are shown in FIG. 21 of US Publ. 2014/0370013, hereby incorporated by reference. That is, IgG1 is a common isotype for therapeutic antibodies for a variety of reasons, including high effector function. However, the heavy constant region of IgG1 has a higher pI than that of IgG2 (8.10 versus 7.31). By introducing IgG2 residues at particular positions into the IgG1 backbone, the pI of the resulting monomer is lowered (or increased) and additionally exhibits longer serum half-life. For example, IgG1 has a glycine (pI 5.97) at position 137, and IgG2 has a glutamic acid (pI 3.22); importing the glutamic acid will affect the pI of the resulting protein. As is described below, a number of amino acid substitutions are generally required to significant affect the pI of the variant antibody. However, it should be noted as discussed below that even changes in IgG2 molecules allow for increased serum half-life.

In other embodiments, non-isotypic amino acid changes are made, either to reduce the overall charge state of the resulting protein (e.g., by changing a higher pI amino acid to a lower pI amino acid), or to allow accommodations in structure for stability, etc. as is more further described below.

In addition, by pI engineering both the heavy and light constant domains, significant changes in each monomer of the heterodimer can be seen. As discussed herein, having the pIs of the two monomers differ by at least 0.5 can allow separation by ion exchange chromatography or isoelectric focusing, or other methods sensitive to isoelectric point.

4. Calculating pI

The pI of each monomer of the antibodies provided herein can depend on the pI of the variant heavy chain constant domain and the pI of the total monomer, including the variant heavy chain constant domain and the fusion partner. Thus, in some embodiments, the change in pI is calculated on the basis of the variant heavy chain constant domain, using the chart in the FIG. 19 of US Pub. 2014/0370013. As discussed herein, which monomer to engineer is generally decided by the inherent pI of the Fv and scaffold regions. Alternatively, the pI of each monomer can be compared.

5. pI Variants that Also Confer Better FcRn In Vivo Binding

In the case where the pI variant decreases the pI of the monomer, the pI variant can have the added benefit of improving serum retention in vivo.

Although still under examination, Fc regions are believed to have longer half-lives in vivo, because binding to FcRn at pH 6 in an endosome sequesters the Fc (Ghetie and Ward, 1997 Immunol Today. 18(12): 592-598, entirely incorporated by reference). The endosomal compartment then recycles the Fc to the cell surface. Once the compartment opens to the extracellular space, the higher pH, ˜7.4, induces the release of Fc back into the blood. In mice, Dall' Acqua et al. showed that Fc mutants with increased FcRn binding at pH 6 and pH 7.4 actually had reduced serum concentrations and the same half-life as wild-type Fc (Dall' Acqua et al. 2002, J. Immunol. 169:5171-5180, entirely incorporated by reference). The increased affinity of Fc for FcRn at pH 7.4 is thought to forbid the release of the Fc back into the blood. Therefore, the Fc mutations that will increase Fc's half-life in vivo will ideally increase FcRn binding at the lower pH while still allowing release of Fc at higher pH. The amino acid histidine changes its charge state in the pH range of 6.0 to 7.4. Therefore, it is not surprising to find His residues at important positions in the Fc/FcRn complex.

Recently it has been suggested that antibodies with variable regions that have lower isoelectric points may also have longer serum half-lives (Igawa et al., 2010 PEDS. 23(5): 385-392, entirely incorporated by reference). However, the mechanism of this is still poorly understood. Moreover, variable regions differ from antibody to antibody. Constant region variants with reduced pI and extended half-life would provide a more modular approach to improving the pharmacokinetic properties of antibodies, as described herein.

### D. Additional Fc Variants for Additional Functionality

In addition to the heterodimerization variants discussed above, there are a number of useful Fc amino acid modification that can be made for a variety of reasons, including, but not limited to, altering binding to one or more FcγR receptors, altered binding to FcRn receptors, etc., as discussed below.

Accordingly, the antibodies provided herein (heterodimeric, as well as homodimeric) can include such amino acid modifications with or without the heterodimerization variants outlined herein (e.g., the pI variants and steric variants). Each set of variants can be independently and optionally included or excluded from any particular heterodimeric protein.

1. FcγR Variants

Accordingly, there are a number of useful Fc substitutions that can be made to alter binding to one or more of the FcγR receptors. In certain embodiments, the subject antibody includes modifications that alter the binding to one or more FcγR receptors (i.e., “FcγR variants”). Substitutions that result in increased binding as well as decreased binding can be useful. For example, it is known that increased binding to FcγRIIIa generally results in increased ADCC (antibody dependent cell-mediated cytotoxicity; the cell-mediated reaction wherein nonspecific cytotoxic cells that express FcγRs recognize bound antibody on a target cell and subsequently cause lysis of the target cell). Similarly, decreased binding to FcγRIIb (an inhibitory receptor) can be beneficial as well in some circumstances. Amino acid substitutions that find use in the subject antibodies include those listed in U.S. Pat. No. 8,188,321 (particularly FIG. 41) and U.S. Pat. No. 8,084,582, and US Publ. App. Nos. 20060235208 and 20070148170, all of which are expressly incorporated herein by reference in their entirety and specifically for the variants disclosed therein that affect Fcγ receptor binding. Particular variants that find use include, but are not limited to, 236A, 239D, 239E, 332E, 332D, 239D/332E, 267D, 267E, 328F, 267E/328F, 236A/332E, 239D/332E/330Y, 239D, 332E/330L, 243A, 243L, 264A, 264V and 299T. Such modification may be included in one or both Fc domains of the subject antibody.

In some embodiments, the subject antibody includes one or more Fc modifications that increase serum half-life. Fc substitutions that find use in increased binding to the FcRn receptor and increased serum half-life, as specifically disclosed in U.S. Ser. No. 12/341,769, hereby incorporated by reference in its entirety, including, but not limited to, 434S, 434A, 428L, 308F, 259I, 428L/434S, 259I/308F, 436I/428L, 436I or V/434S, 436V/428L and 259I/308F/428L. Such modification may be included in one or both Fc domains of the subject antibody.

2. Ablation Variants

In some embodiments, the heterodimeric antibody (e.g., anti-PD-L1×anti-CD28 bispecific antibody) includes one or more modifications that reduce or remove the normal binding of the Fc domain to one or more or all of the Fcγ receptors (e.g., FcγR1, FcγRIIa, FcγRIIb, FcγRIIIa, etc.) to avoid additional mechanisms of action. Such modifications are referred to as “FcγR ablation variants” or “Fc knock out (FcKO or KO)” variants. In these embodiments, for some therapeutic applications, it is desirable to reduce or remove the normal binding of the Fc domain to one or more or all of the Fcγ receptors (e.g., FcγR1, FcγRIIa, FcγRIIb, FcγRIIIa, etc.) to avoid additional mechanisms of action. That is, for example, in many embodiments, particularly in the use of bispecific antibodies that bind CD28 monovalently, it is generally desirable to ablate FcγRIIIa binding to eliminate or significantly reduce ADCC activity. In some embodiments, of the subject antibodies described herein, at least one of the Fc domains comprises one or more Fcγ receptor ablation variants. In some embodiments, of the subject antibodies described herein, both of the Fc domains comprises one or more Fcγ receptor ablation variants. These ablation variants are depicted in FIG. 5, and each can be independently and optionally included or excluded, with preferred aspects utilizing ablation variants selected from the group consisting of G236R/L328R, E233P/L234V/L235A/G236del/S239K, E233P/L234V/L235A/G236del/S267K, E233P/L234V/L235A/G236del/S239K/A327G, E233P/L234V/L235A/G236del/S267K/A327G and E233P/L234V/L235A/G236del. It should be noted that the ablation variants referenced herein ablate FcγR binding but generally not FcRn binding.

As is known in the art, the Fc domain of human IgG1 has the highest binding to the Fcγ receptors, and thus ablation variants can be used when the constant domain (or Fc domain) in the backbone of the heterodimeric antibody is IgG1. Alternatively, or in addition to ablation variants in an IgG1 background, mutations at the glycosylation position 297 (generally to A or S) can significantly ablate binding to FcγRIIIa, for example. Human IgG2 and IgG4 have naturally reduced binding to the Fcγ receptors, and thus those backbones can be used with or without the ablation variants.

### E. Combination of Heterodimeric and Fc Variants

As will be appreciated by those in the art, all of the recited heterodimerization variants (including skew and/or pI variants) can be optionally and independently combined in any way, as long as they retain their “strandedness” or “monomer partition”. In addition, all of these variants can be combined into any of the heterodimerization formats.

In the case of pI variants, while embodiments finding particular use are shown in the figures, other combinations can be generated, following the basic rule of altering the pI difference between two monomers to facilitate purification.

In addition, any of the heterodimerization variants, skew and pI, are also independently and optionally combined with Fc ablation variants, Fc variants, FcRn variants, as generally outlined herein.

Exemplary combination of variants that are included in some embodiments of the subject heterodimeric antibodies are included in FIG. 9. In some embodiments, the heterodimeric antibody includes a combination of variants as depicted in FIG. 9. In certain embodiments, the antibody is a heterodimeric “1+1 Fab-scFv-Fc,” “2+1 Fab₂-scFv-Fc,” “2+1 mAb-scFv,” “1+1 common light chain,” “2+1 common light chain,” “1+1+1 stackFab₂-scFv-Fc,” “1+1+1 Fab-(Fab-scFv)-Fc,” “1+1+1 mAb-scFv,” and, “1+1+1 stackFab₂-Fab-Fc” format antibody (see FIGS. 82 and 83).

### F. Useful Antibody Formats

As will be appreciated by those in the art and discussed more fully below, the heterodimeric antibodies provided herein can take on several different configurations as generally depicted in FIGS. 82 and 83.

As will be appreciated by those in the art, the heterodimeric formats of the invention can have different valencies as well as be bispecific. That is, heterodimeric antibodies of the invention can be bivalent and bispecific, or trivalent and bispecific, wherein the first antigen is bound by two binding domains and the second antigen by a second binding domain.

In some embodiments, the present invention utilizes PD-L1 antigen binding domains. Any of the anti-PD-L1 antigen binding domains can be used, whether CDRs, variable light and variable heavy domains, Fabs and scFvs as depicted herein, including in any of the Figures (e.g., FIGS. 17, 19, 20, 21, 24-26, and 28 or a variant thereof) and the Sequence Listing, can be used, optionally and independently combined in any combination. In some embodiments, the antibody includes a PD-L1 binding domain having a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20,
ii) a variable heavy domain and variable light domain of any of the PD-L1 binding domains in FIGS. 17 and 21,
iii) a variable heavy domain of any of the PD-L1 binding domains in FIGS. 25 and 26, and a variable light domain of any of the PD-L1 binding domains in FIGS. 25 and 26,
iv) a variable heavy domain and variable light domain of any of the PD-L1 binding domains in FIGS. 25 and 26,
v) a variable heavy domain of any of the PD-L1 binding domains in FIGS. 25 and 26, and a variable light domain having the amino acid sequence of SEQ ID NO:20 (FIG. 24), and
vi) a variable heavy domain and variable light domain of any of the PD-L1 binding domains in FIG. 28.

In some embodiments, the present invention utilizes PD-L2 antigen binding domains. Any of the anti-PD-L2 antigen binding domains can be used, whether CDRs, variable light and variable heavy domains, Fabs and scFvs as depicted herein, including in any of the Figures (e.g., FIGS. 24, 29-32 and 34 or a variant thereof) and the Sequence Listing, can be used, optionally and independently combined in any combination. In some embodiments, the antibody includes a PD-L2 binding domain having a variable heavy domain and variable light domain selected from the following:
i) a variable heavy domain of any of the PD-L2 binding domains in FIGS. 29-32, and a variable light domain of any of the PD-L2 binding domains in FIGS. 29-32,
ii) a variable heavy domain and variable light domain of any of the PD-L2 binding domains in FIGS. 29-32,
iii) a variable heavy domain of any of the PD-L2 binding domains in FIGS. 29-32, and a variable light domain having the amino acid sequence of SEQ ID NO:20 (FIG. 24), and
iv) a variable heavy domain and variable light domain of any of the PD-L2 binding domains in FIG. 34.

In some embodiments, the present invention utilizes CD28 antigen binding domains in combination with PD-L1 and/or PD-L2 binding domains. As is outlined herein, when CD28 is one of the target antigens, it is preferable that the CD28 is bound only monovalently. As will be appreciated by those in the art, any collection of anti-CD28 CDRs, anti-CD28 variable light and variable heavy domains, Fabs and scFvs as depicted herein, including in any of the Figures (see particularly FIGS. 35, 37, 38, 41-74 and 81) and the Sequence Listing, can be used. In some embodiments, the antibody includes a CD28 binding domain having a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38,
ii) a variable heavy domain and a variable light domain of any of the CD28 binding domains in FIG. 38,
iii) a variable heavy domain of any of the CD28 binding domains in FIGS. 41-74, and a variable light domain of any of the CD28 binding domains in FIGS. 41-74,
iv) a variable heavy domain and variable light domain of any of the CD28 binding domains in FIGS. 41-74,
v) a variable heavy domain of any of the CD28 binding domains in FIGS. 41-74, and a variable light domain having the amino acid sequence of SEQ ID NO:20 (FIG. 24), and
vi) a variable heavy domain and variable light domain of any of the CD28 binding domains in FIG. 81.

1. 1+1 Fab-scFv-Fc Format (“Bottle Opener”)

One heterodimeric antibody format that finds particular use in subject bispecific antibodies provided herein (e.g., anti-PD-L1×anti-CD28 and anti-PD-L1×anti-CD28 antibodies) is the “1+1 Fab-scFv-Fc” or “bottle opener” format as shown in FIG. 82A. The 1+1 Fab-scFv-Fc format antibody includes a first monomer that is a “regular” heavy chain (VH1-CH1-hinge-CH2-CH3), wherein VH1 is a first variable heavy domain and CH2-CH3 is a first Fc domain. The 1+1 Fab-scFv-Fc also includes a light chain that includes a first variable light domain VL1 and a constant light domain CL. The light chain interacts with the VH1-CH1 of the first monomer to form a first antigen binding domain that is a Fab. The second monomer of the antibody includes a second binding domain that is a single chain Fv (“scFv”, as defined below) and a second Fc domain. The scFv includes a second variable heavy domain (VH2) and a second variable light domain (VL2), wherein the VH2 is attached to the VL2 using an scFv linker that can be charged (see, e.g., FIG. 7). The scFv is attached to the heavy chain using a domain linker (see, e.g., FIG. 8). The two monomers are brought together by the use of amino acid variants (e.g., heterodimerization variants, discussed above) in the constant regions (e.g., the Fc domain, the CH1 domain and/or the hinge region) that promote the formation of heterodimeric antibodies as is described more fully below. This structure is sometimes referred to herein as the “bottle-opener”format, due to a rough visual similarity to a bottle-opener. In some embodiments, the 1+1 Fab-scFv-Fc format antibody is a bivalent antibody.

There are several distinct advantages to the present “1+1 Fab-scFv-Fc” format. As is known in the art, antibody analogs relying on two scFv constructs often have stability and aggregation problems, which can be alleviated in the present invention by the addition of a “regular” heavy and light chain pairing. In addition, as opposed to formats that rely on two heavy chains and two light chains, there is no issue with the incorrect pairing of heavy and light chains (e.g., heavy 1 pairing with light 2, etc.).

In some embodiments of the 1+1 Fab-scFv-Fc format antibody, one of the first or second antigen binding domain is a PD-L1 binding domain. In some embodiments, the first binding domain (i.e., the Fab) is a PD-L1 binding domain. In certain embodiments, the second binding domain (i.e., the scFv) is the PD-L1 binding domain. Any suitable PD-L1 binding domain can be included in the subject antibody including those provided herein or a variant thereof (see, e.g., FIGS. 17, 19, 20, 21, 24-26, and 28 and the Sequence Listing). In some embodiments, the PD-L1 binding domain includes a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20,
ii) a variable heavy domain and variable light domain of any of the PD-L1 binding domains in FIGS. 17 and 21,
iii) a variable heavy domain of any of the PD-L1 binding domains in FIGS. 25 and 26, and a variable light domain of any of the PD-L1 binding domains in FIGS. 25 and 26,
iv) a variable heavy domain and variable light domain of any of the PD-L1 binding domains in FIGS. 25 and 26.
v) a variable heavy domain of any of the PD-L1 binding domains in FIGS. 25 and 26, and a variable light domain having the amino acid sequence of SEQ ID NO:20 (FIG. 24), and
vi) a variable heavy domain and variable light domain of any of the PD-L1 binding domains in FIG. 28.

In some embodiments of the 1+1 Fab-scFv-Fc format antibody, one of the first or second antigen binding domain is a PD-L2 binding domain. In some embodiments, the first binding domain (i.e., the Fab) is a PD-L2 binding domain. In certain embodiments, the second binding domain (i.e., the scFv) is the PD-L2 binding domain. Any suitable PD-L2 binding domain can be included in the subject antibody including those provided herein or a variant thereof (see, e.g., FIGS. 24, 29-32 and 34 and the Sequence Listing). In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32,
ii) a variable heavy domain and variable light domain of any of the PD-L2 binding domains in FIGS. 29-32,
iii) a variable heavy domain of any of the PD-L2 binding domains in FIGS. 29-32, and a variable light domain having the amino acid sequence of SEQ ID NO:20 (FIG. 24), and
iv) a variable heavy domain and variable light domain of any of the PD-L2 binding domains in FIG. 34.

In some embodiments of the 1+1 Fab-scFv-Fc format antibody, one of the first or second antigen binding domain is a CD28 binding domain and the other binding domain is a PD-L1 or PD-L2 binding domain. In some embodiments where the 1+1 Fab-scFv-Fc includes a CD28 binding domain and a PD-L1 or PD-L2 binding domain, it is the scFv that binds to the CD28, and the Fab that binds PD-L1 or PD-L2. Exemplary anti-PD-L1×anti-CD28 bispecific antibody in the 1+1 Fab-scFv-Fc format is depicted in FIG. 84. In some embodiments, the anti-PD-L1×anti-CD28 bispecific antibody is an antibody depicted in FIG. 84. Exemplary anti-PD-L2×anti-CD28 bispecific antibody in the 1+1 Fab-scFv-Fc format is depicted in FIG. 87. In some embodiments, the anti-PD-L2×anti-CD28 bispecific antibody is an antibody depicted in FIG. 87.

In some embodiments, the first and second Fc domains of the 1+1 Fab-scFv-Fc format antibody are variant Fc domains that include heterodimerization skew variants (e.g., a set of amino acid substitutions as shown in FIGS. 4 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 6). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 5). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the CH1-hinge-CH2-CH3 of the first monomer comprises amino acid variants L368D/K370S/N208D/Q295E/N384D/Q418E/N421D/E233P/L234V/L235A/G236del/S267K, the second Fc domain comprises amino acid variants S364K/E357Q/E233P/L234V/L235A/G236del/S267K, wherein numbering is according to EU numbering.

In some embodiments, the scFv of the 1+1 Fab-scFv-Fc format antibody provided herein includes a charged scFv linker (including those shown in FIG. 7). In some embodiments, the 1+1 Fab-scFv-Fc format antibody provided herein includes FcRn variants M428L/N434S, wherein numbering is according to EU numbering.

In exemplary embodiments, the first variant Fe domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 1+1 Fab-scFv-Fc format antibody provided herein includes a (GKPGS)₄ charged scFv linker. In some embodiments, the 1+1 Fab-scFv-Fc format antibody provided herein includes FcRn variants M428L/N434S, wherein numbering is according to EU numbering.

In some embodiments, one of the first binding domain or the second binding domain binds CD28 and the other binding domain binds PD-L1 or PD-L2. In some embodiments of the 1+1 Fab-scFv-Fc format antibody, the first antigen binding domain (i.e., the Fab binding domain) binds PD-L1 or PD-L2 and the second antigen binding domain (i.e., the scFv) binds CD28. In some embodiments of the 1+1 Fab-scFv-Fc format antibody, the first antigen binding domain binds CD28 (i.e., the Fab binding domain) and the second antigen binding domain (i.e., the scFv) binds PD-L1 or PD-L2.

Any suitable CD28 binding domain can be included in subject 1+1 Fab-scFv-Fc format antibody, including any of the CD28 binding domains provided herein (see FIGS. 35, 37, 38, 41-74 and 81). In some embodiments, the CD28 binding domain a VH and VL selected from those depicted in FIGS. 35, 37, 38, 41-74 and 81. In some embodiments, the CD28 binding domain includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38,
ii) a variable heavy domain and a variable light domain of any of the CD28 binding domains in FIG. 38,
iii) a variable heavy domain of any of the CD28 binding domains in FIGS. 41-74, and a variable light domain of any of the CD28 binding domains in FIGS. 41-74,
iv) a variable heavy domain and variable light domain of any of the CD28 binding domains in FIGS. 41-74,
v) a variable heavy domain of any of the CD28 binding domains in FIGS. 41-74, and a variable light domain having the amino acid sequence of SEQ ID NO:20 (FIG. 24), and

vi) a variable heavy domain and variable light domain of any of the CD28 binding domains in FIG. 81.

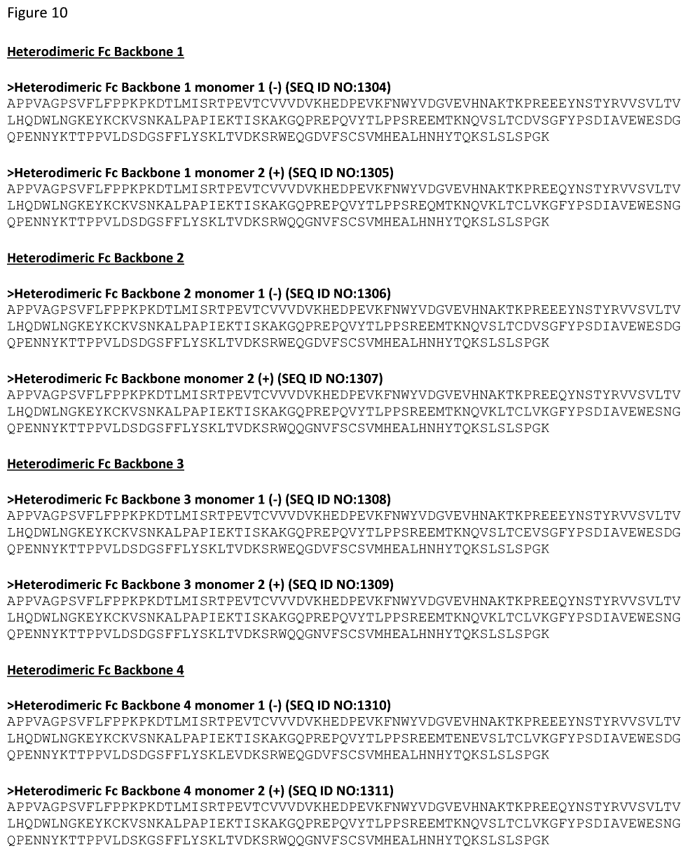

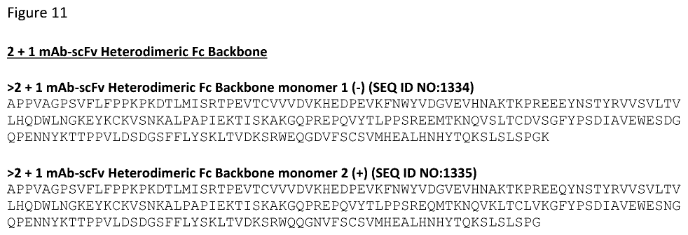

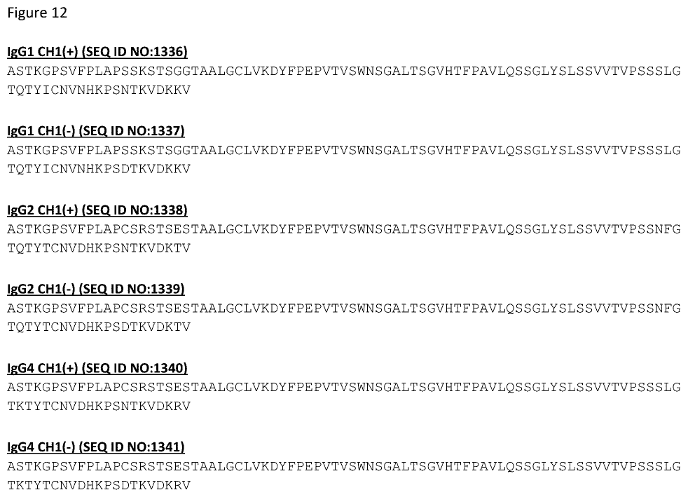

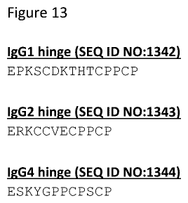

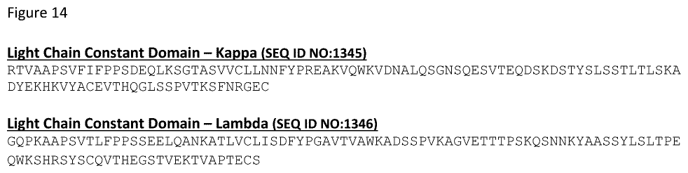

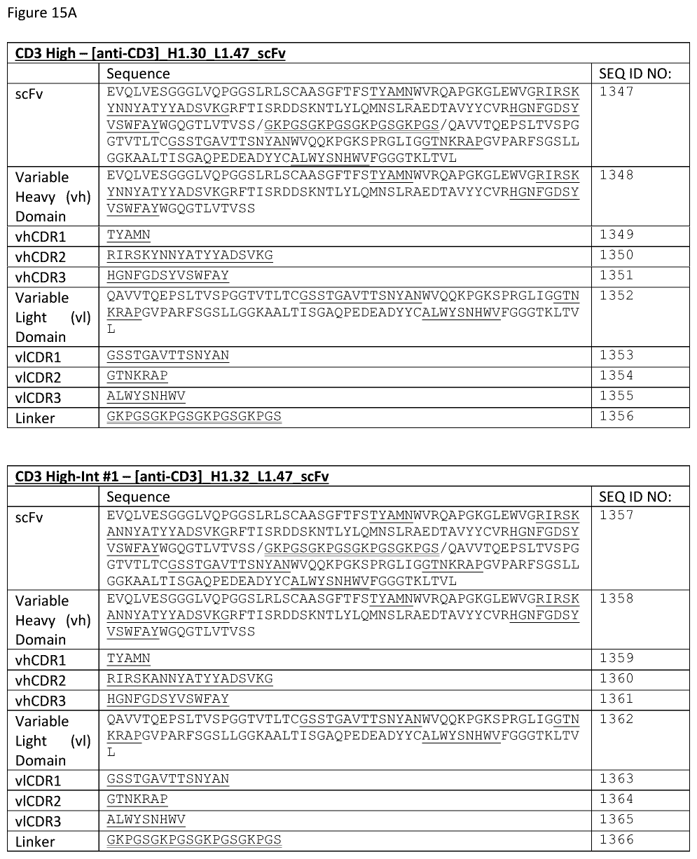

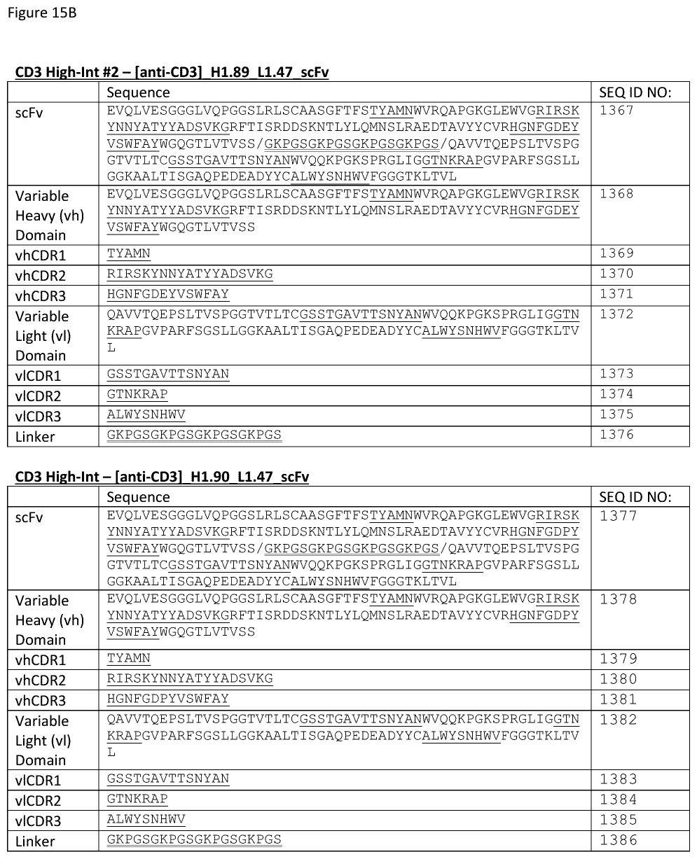

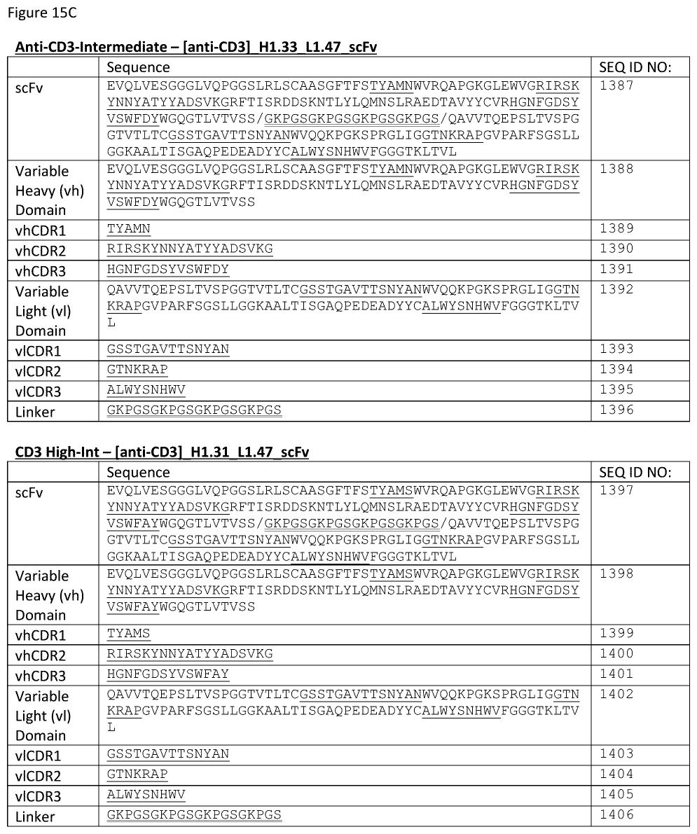

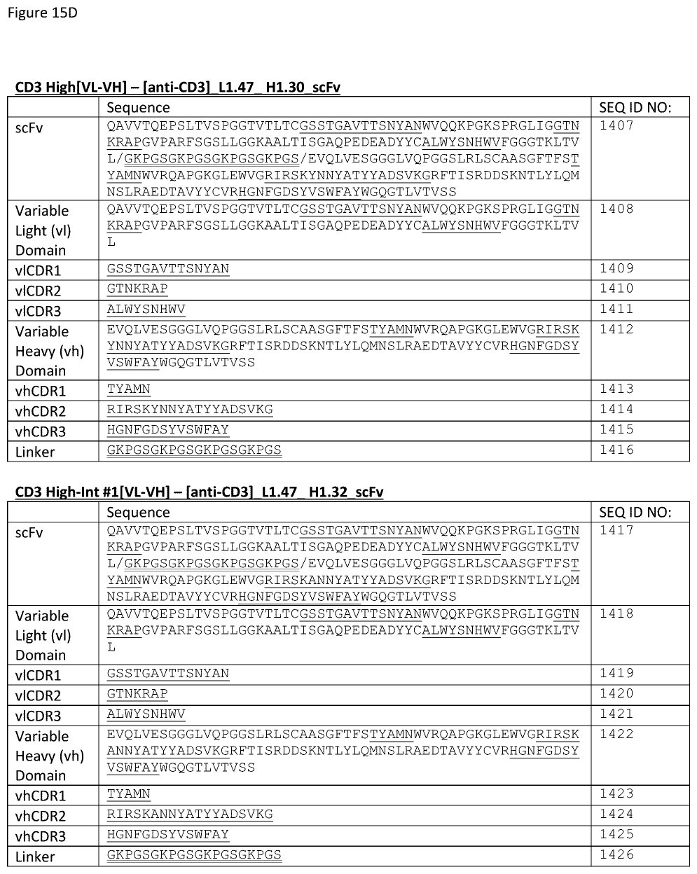

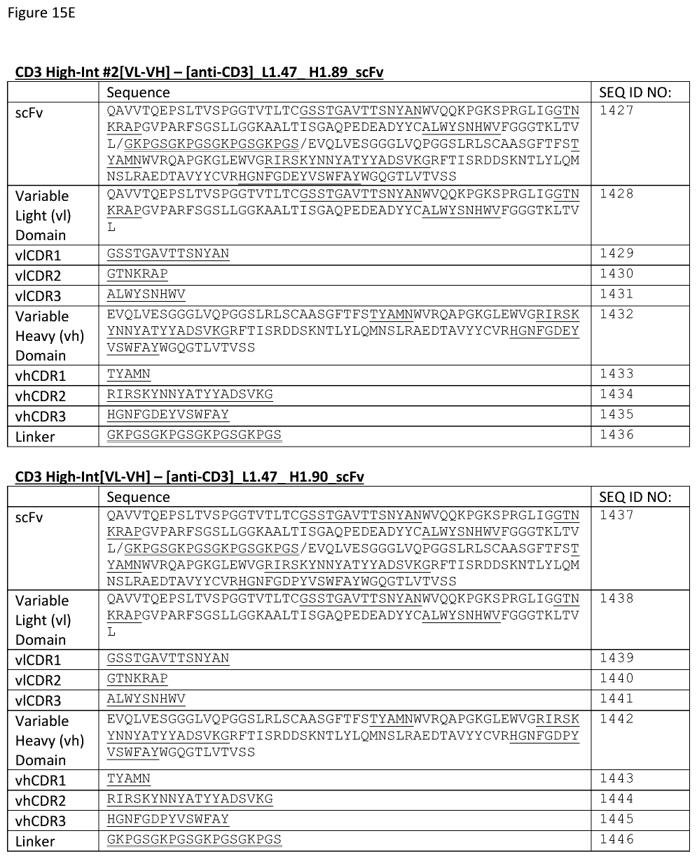

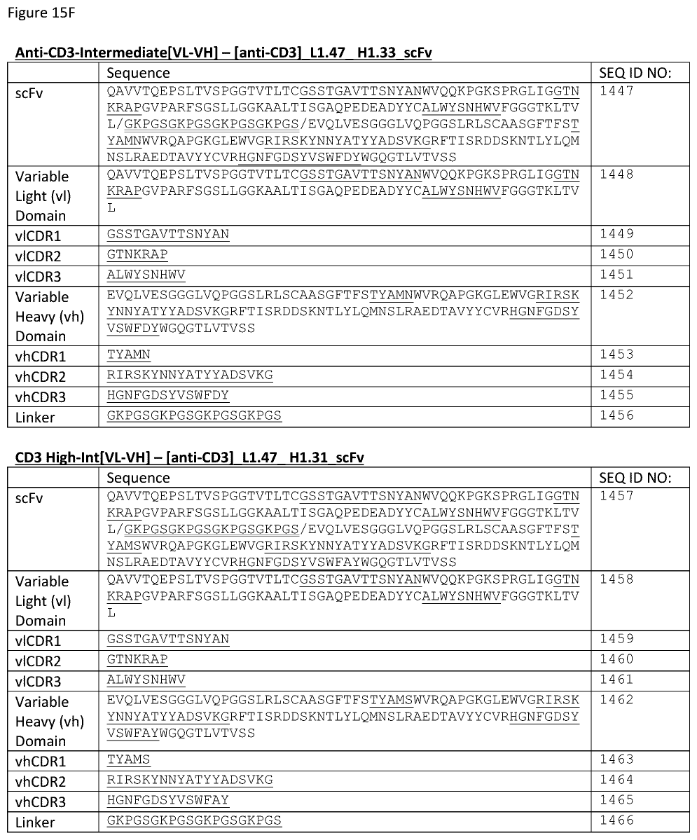

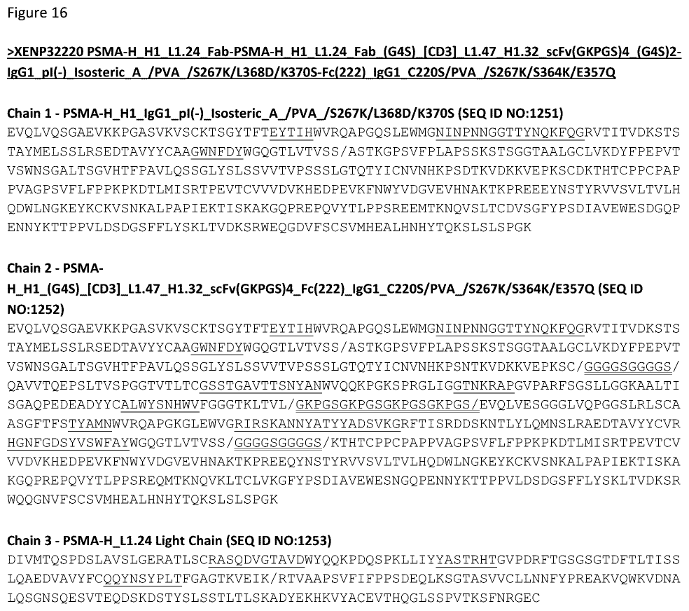

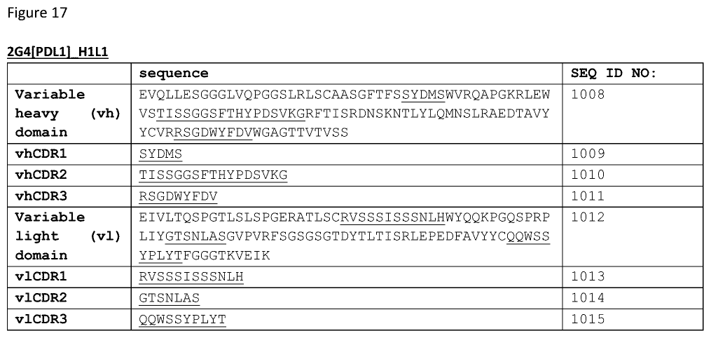

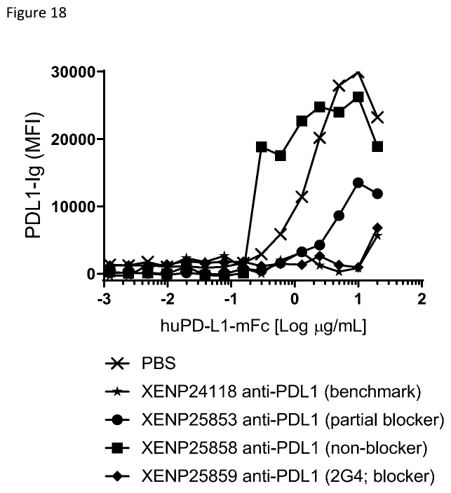

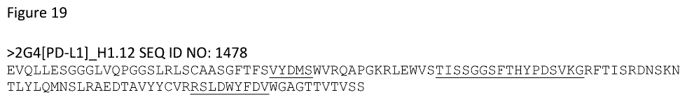

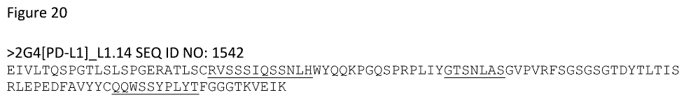

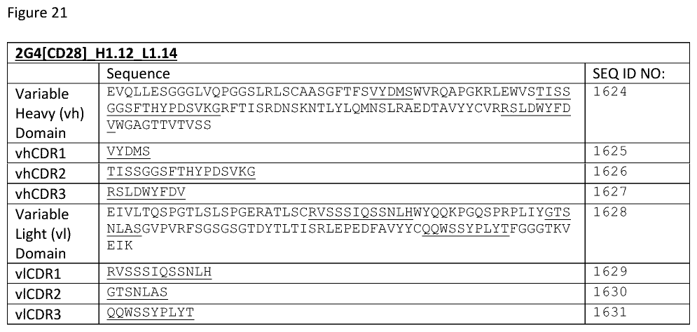

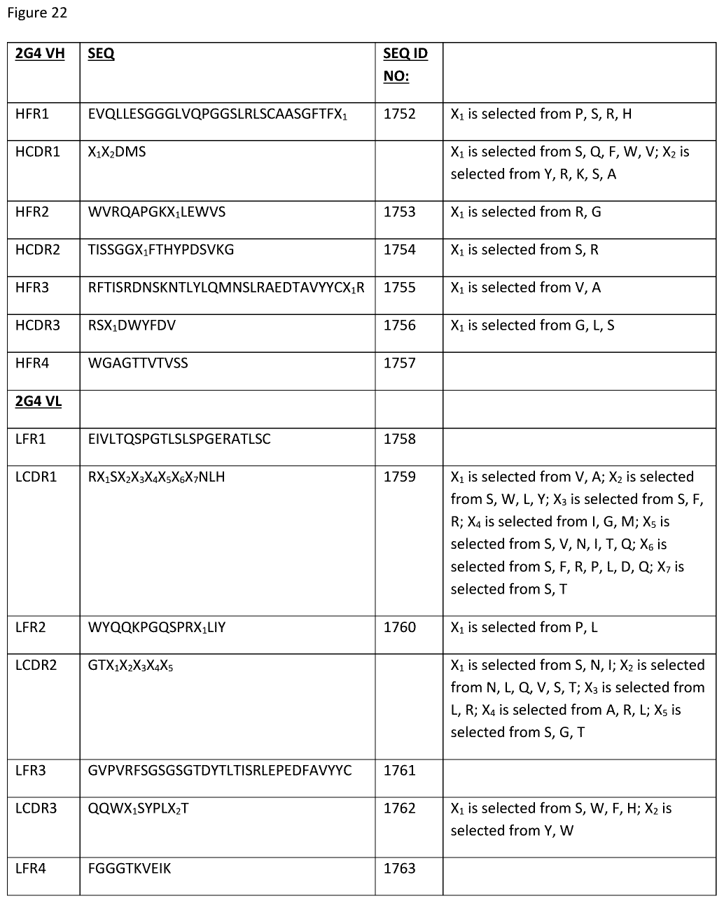

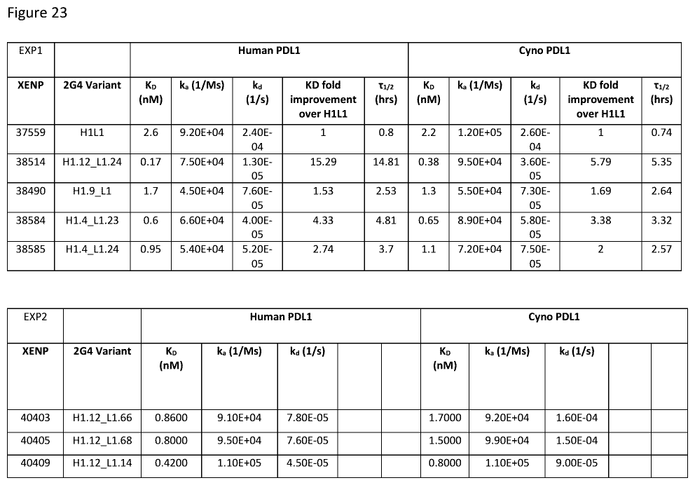

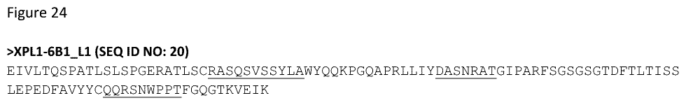

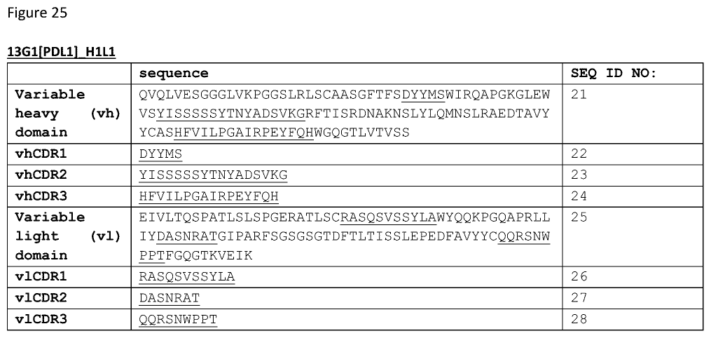

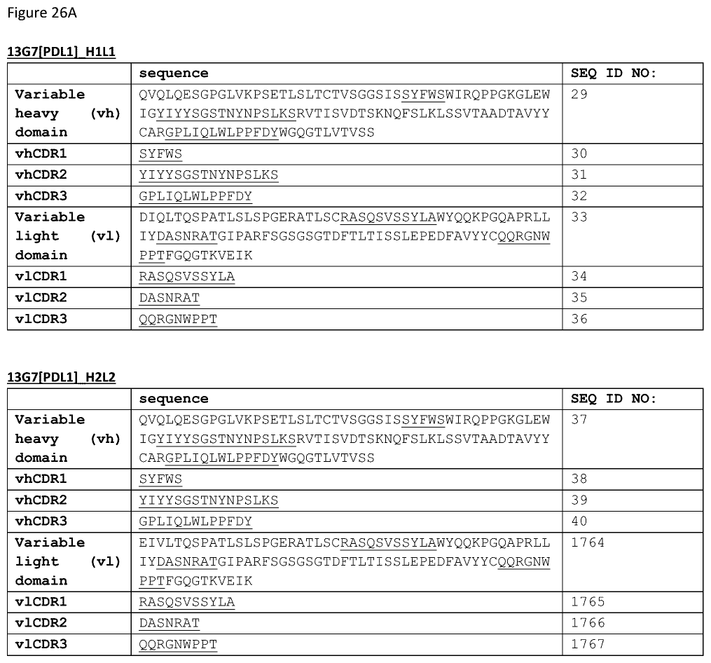

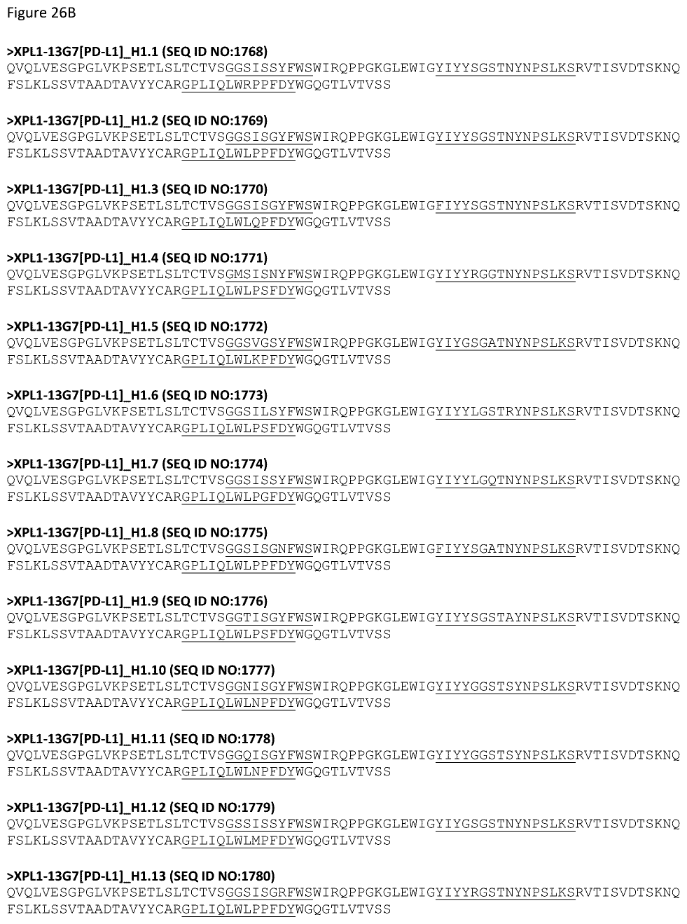

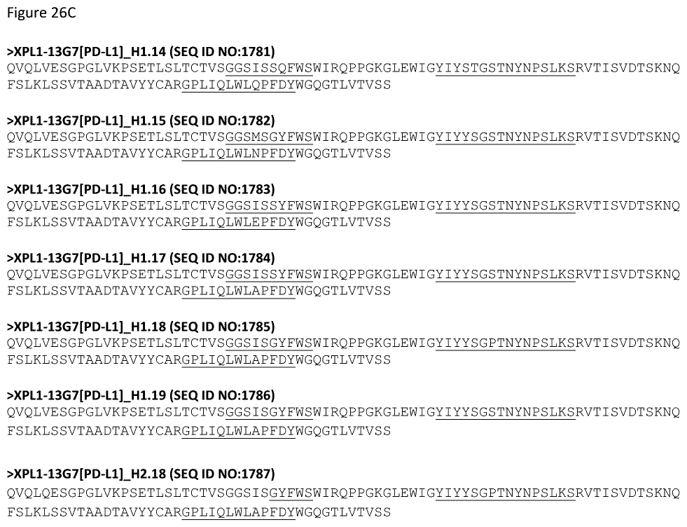

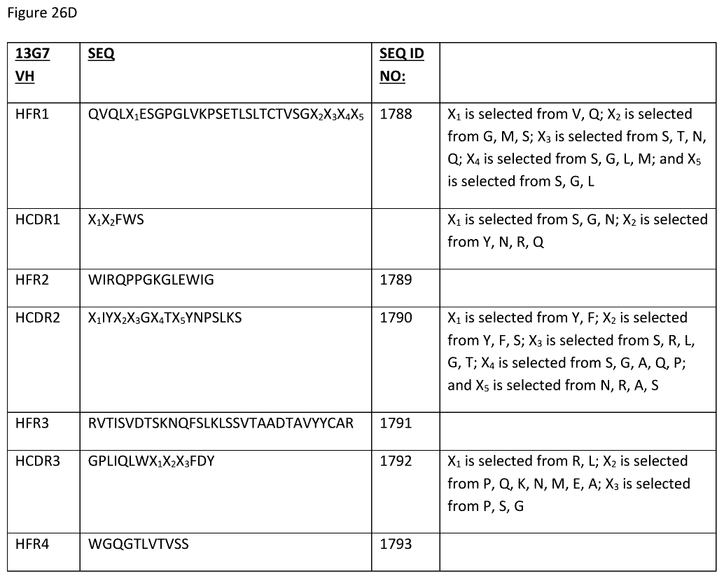

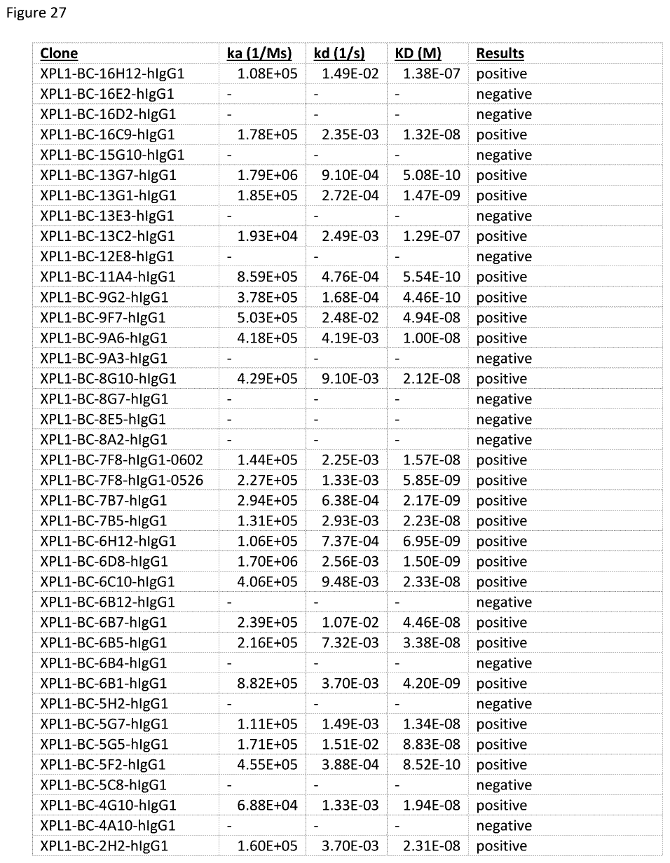

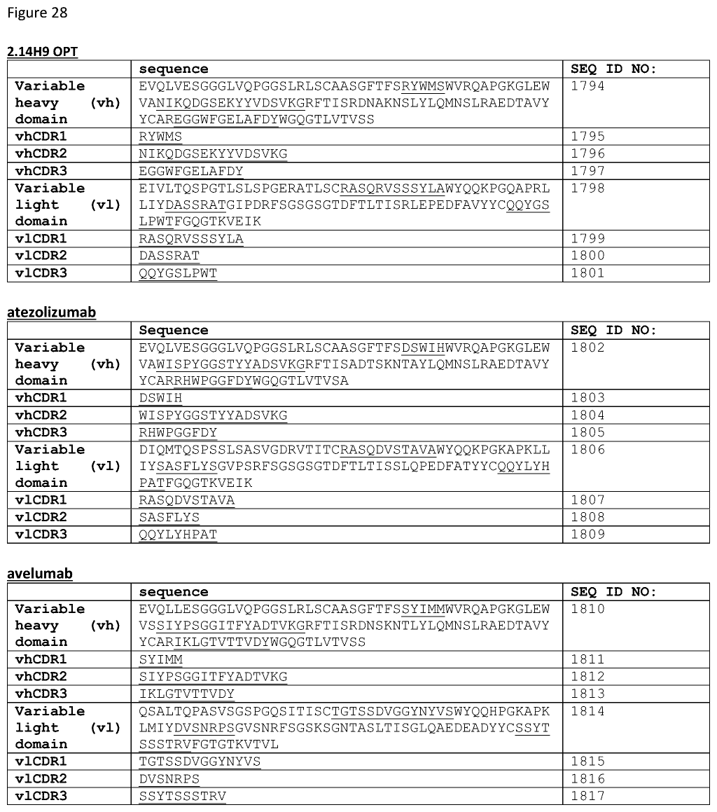

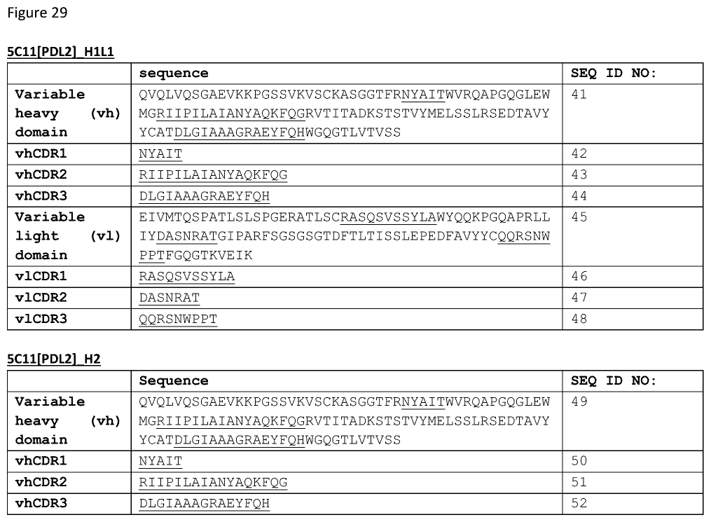

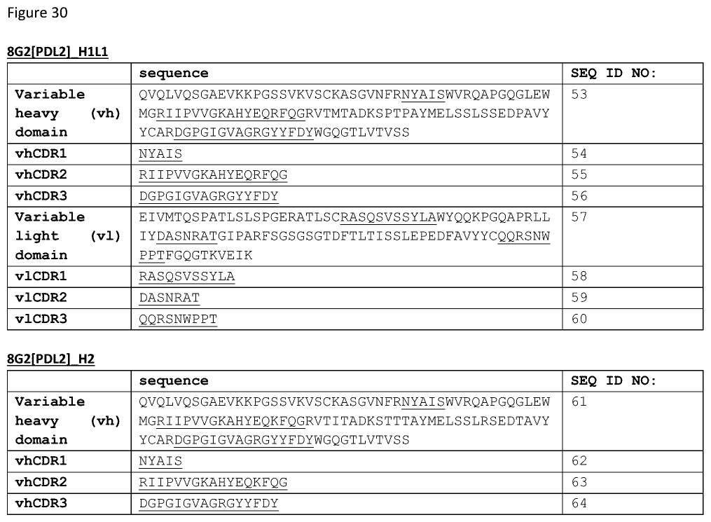

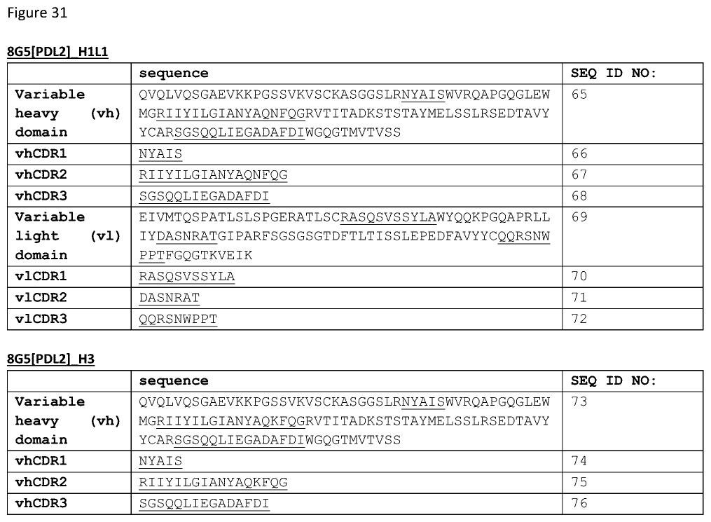

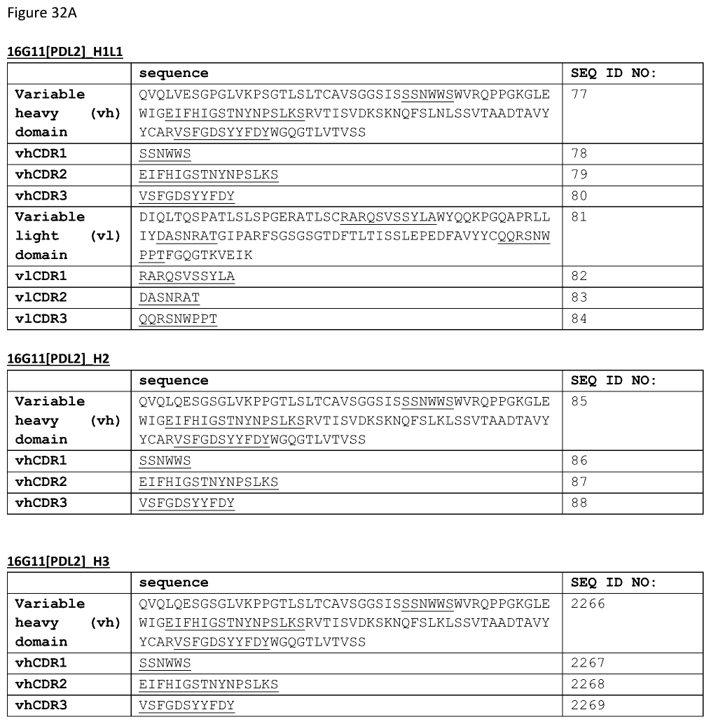

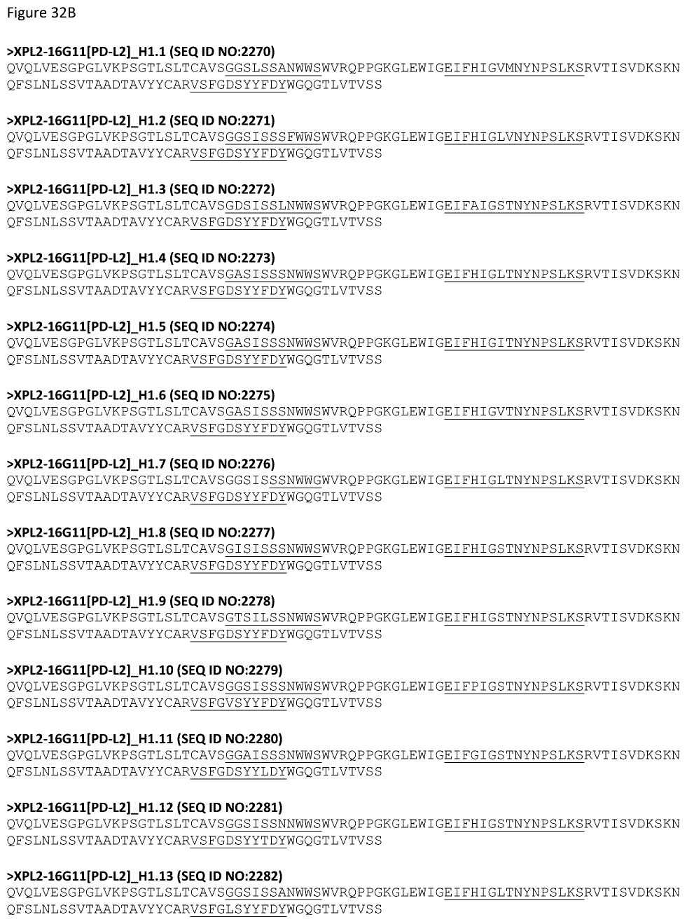

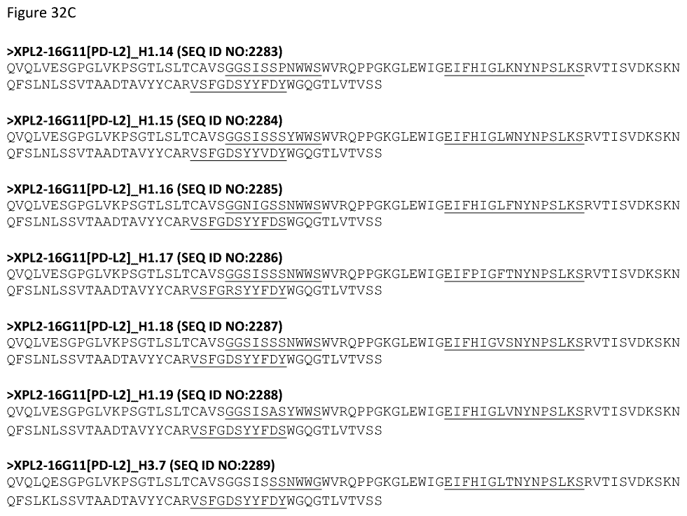

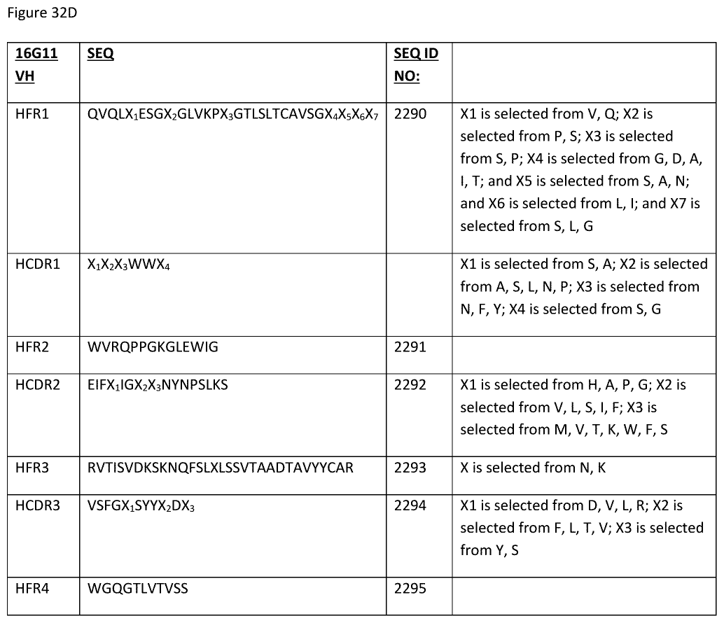

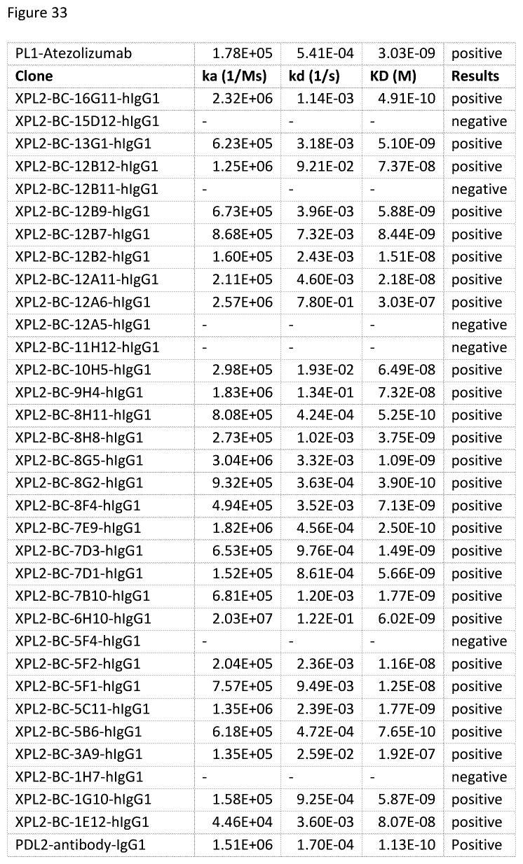

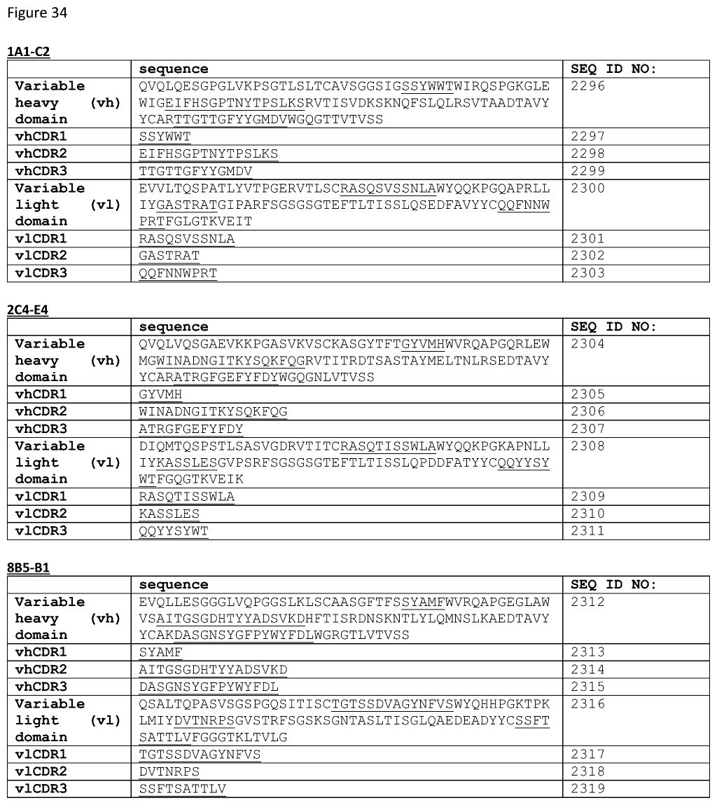

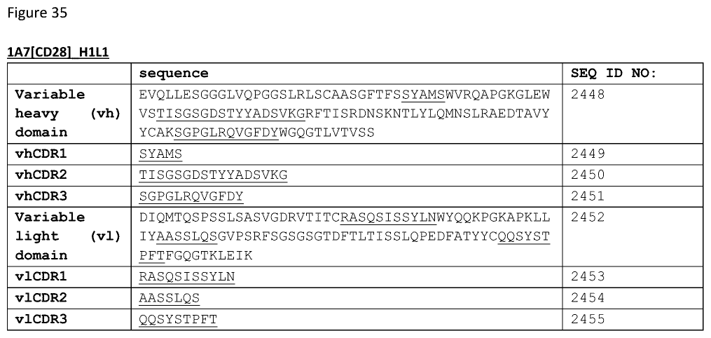

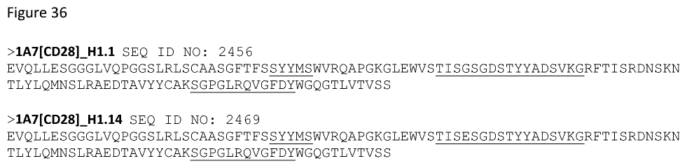

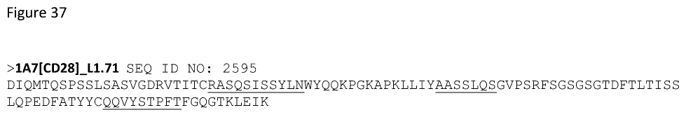

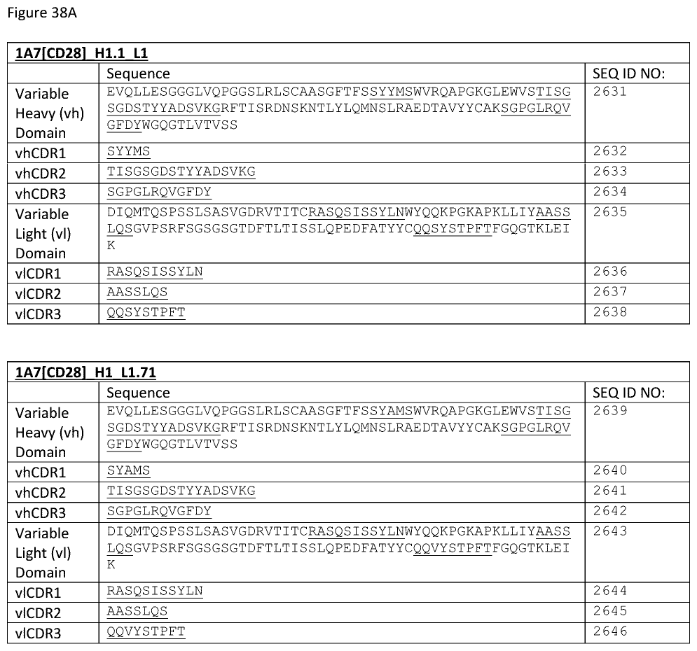

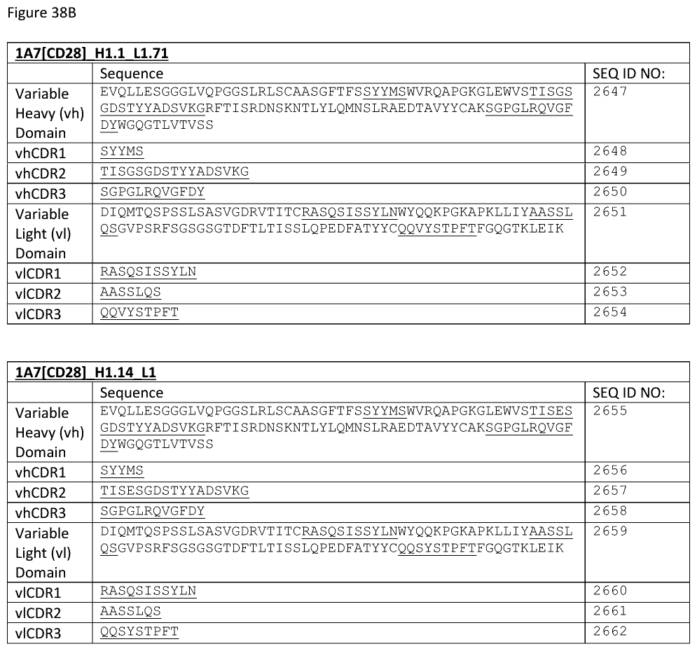

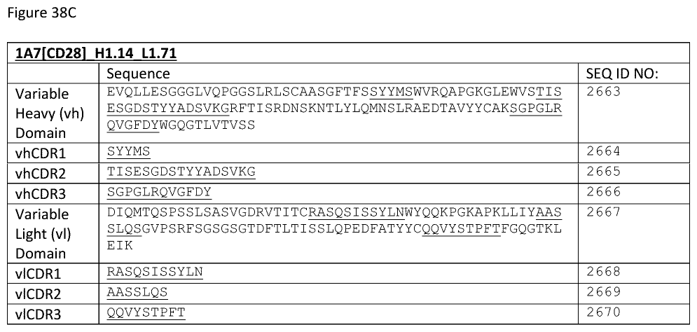

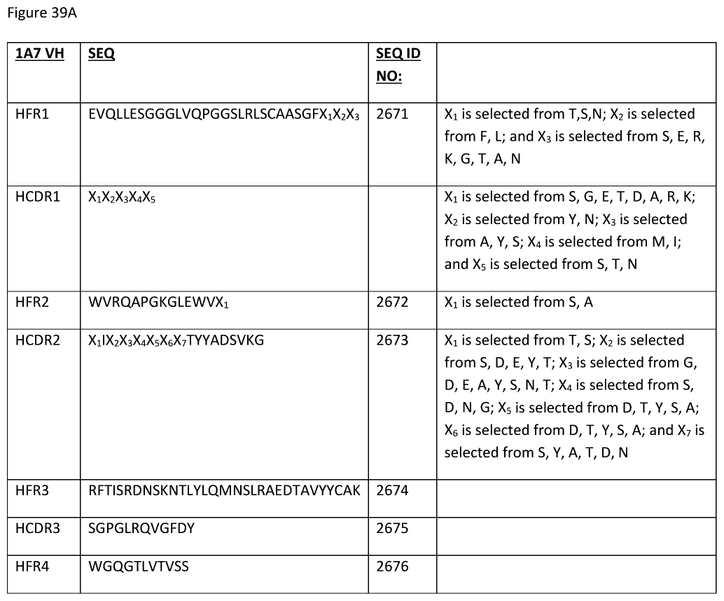

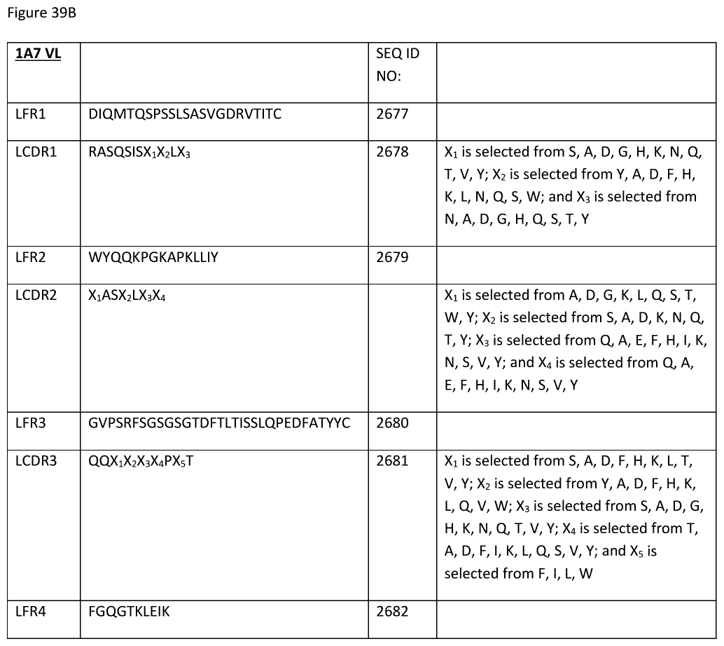

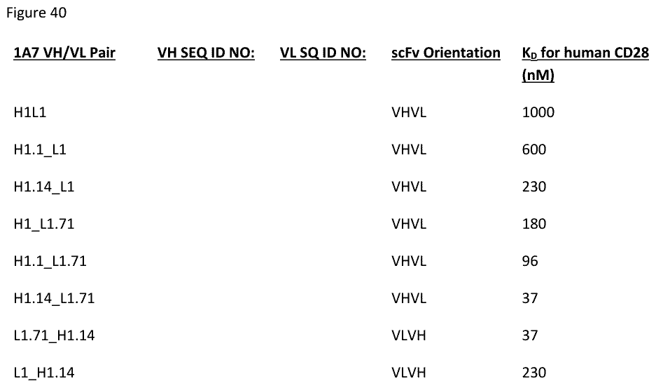

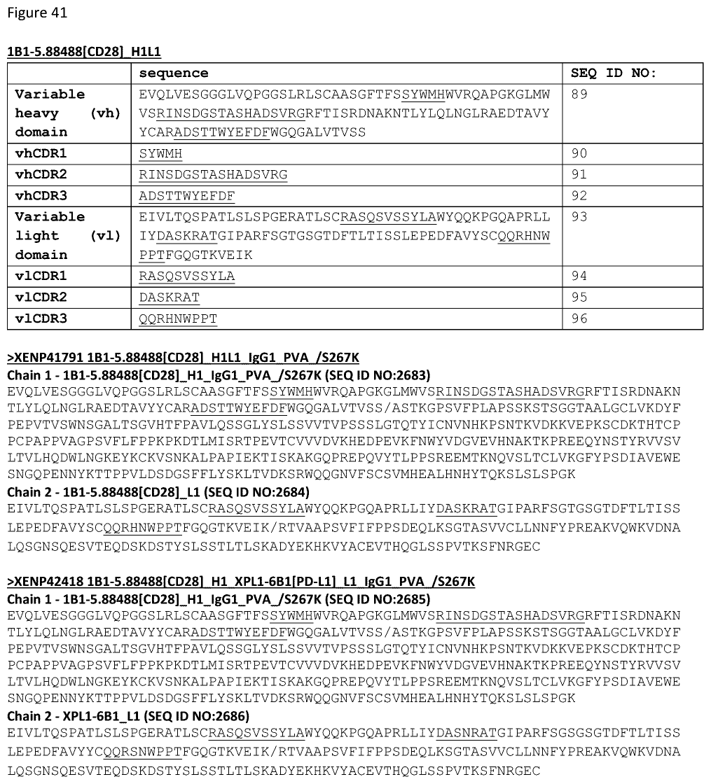

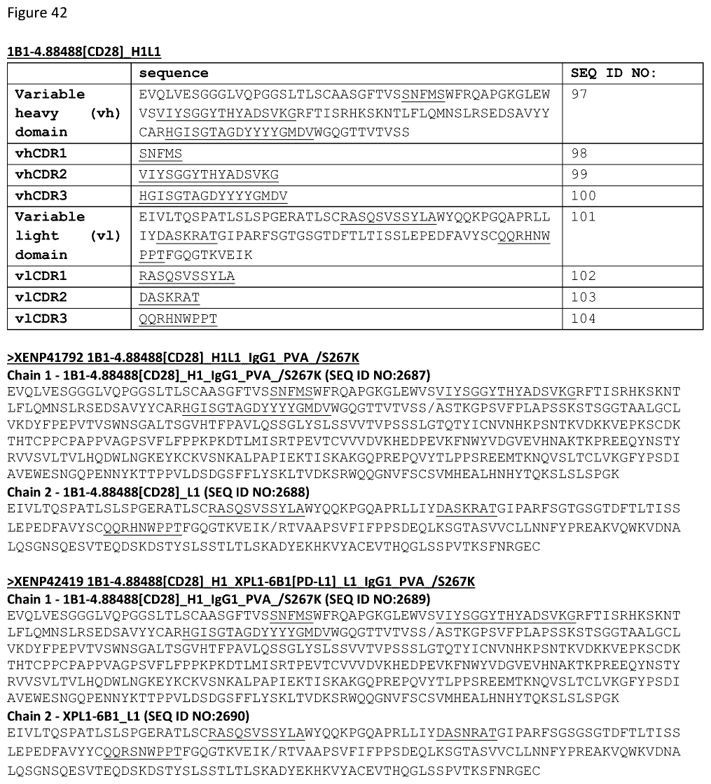

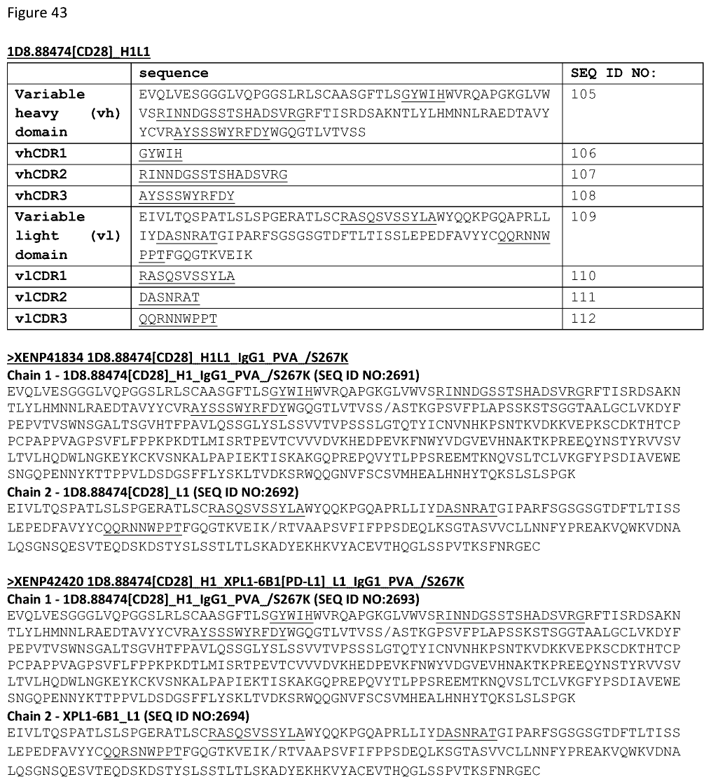

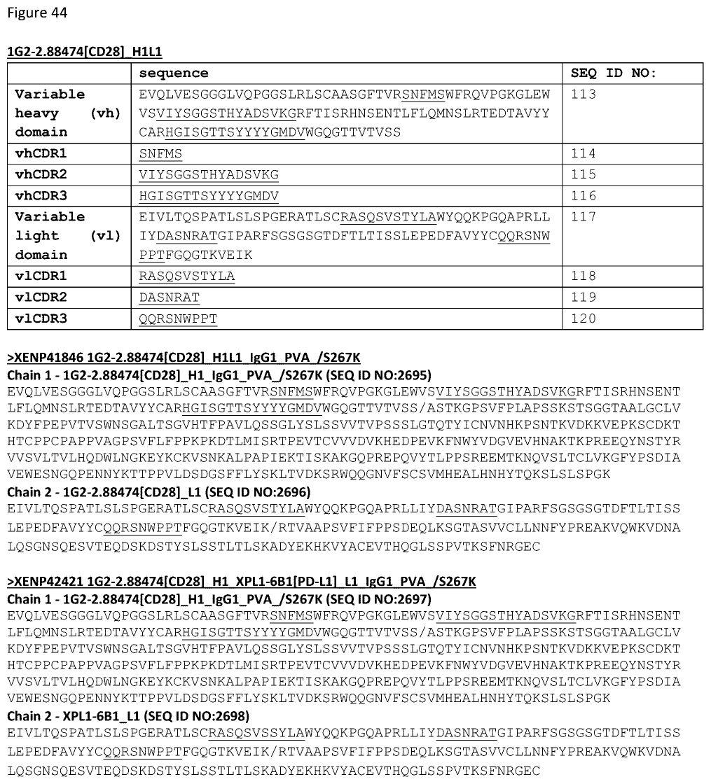

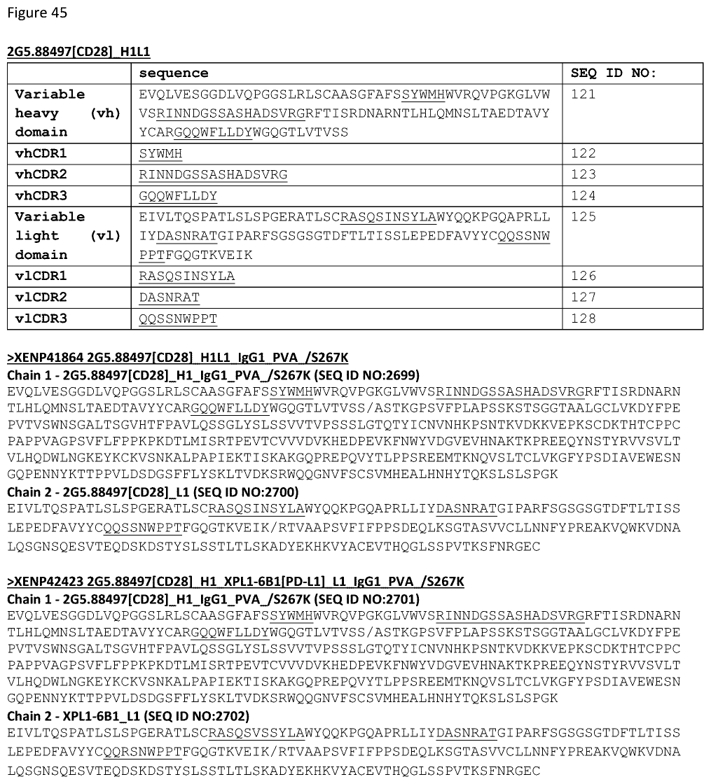

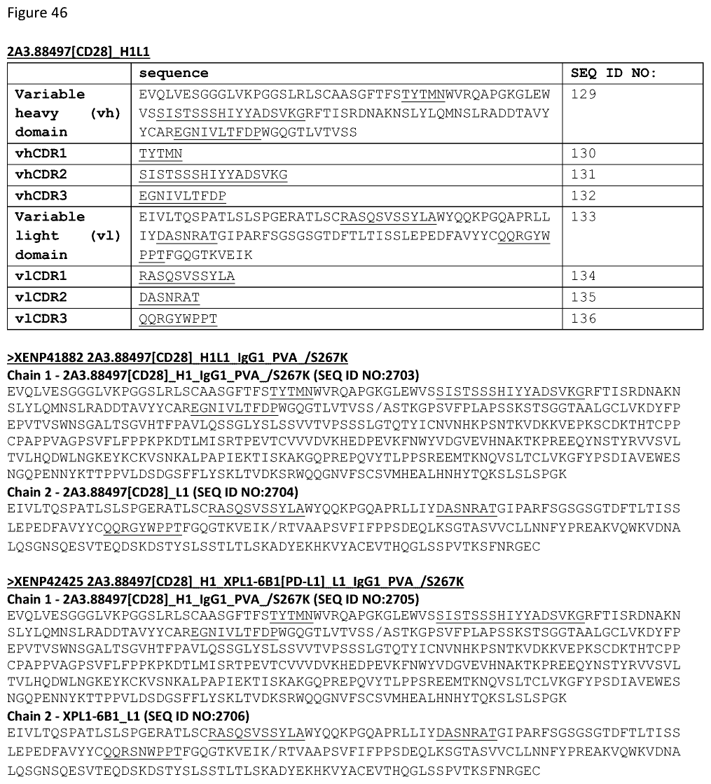

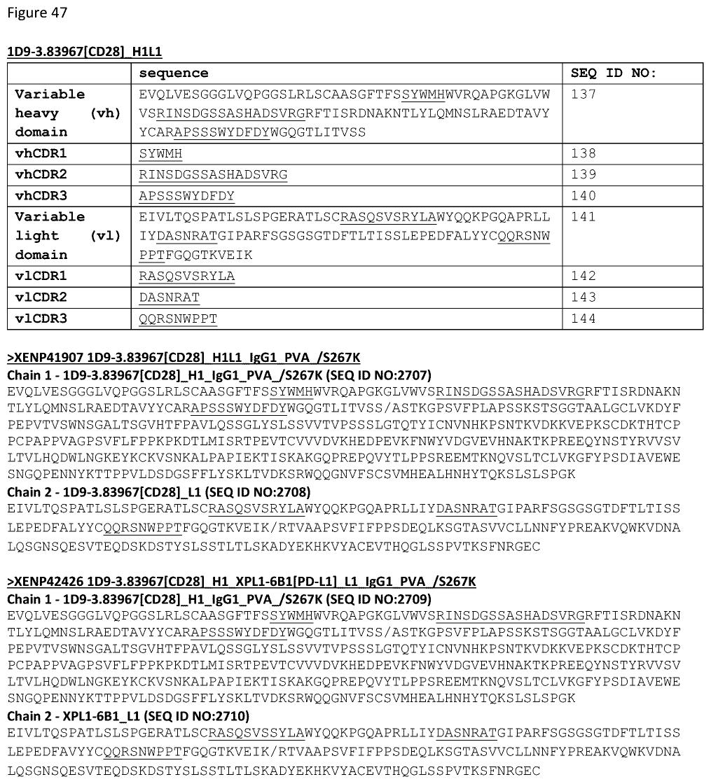

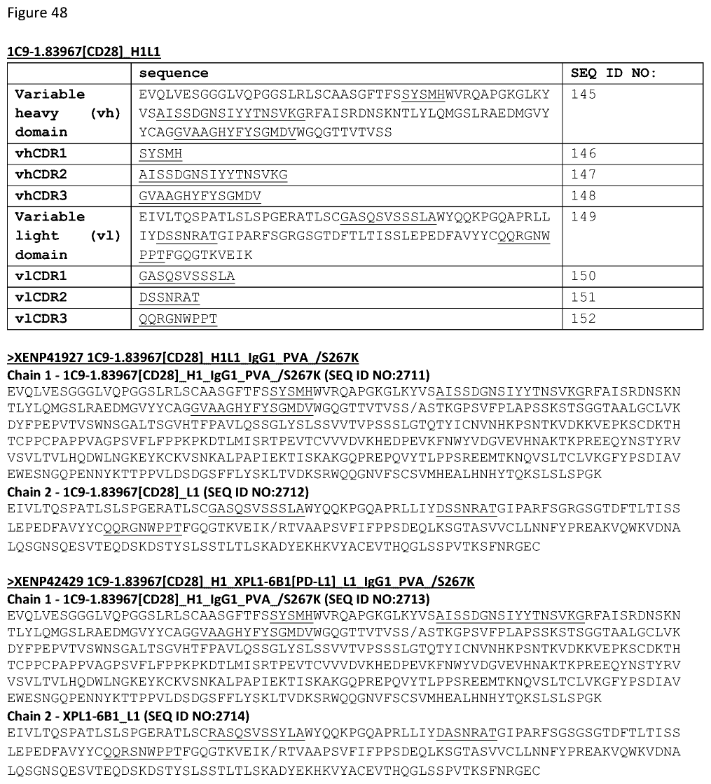

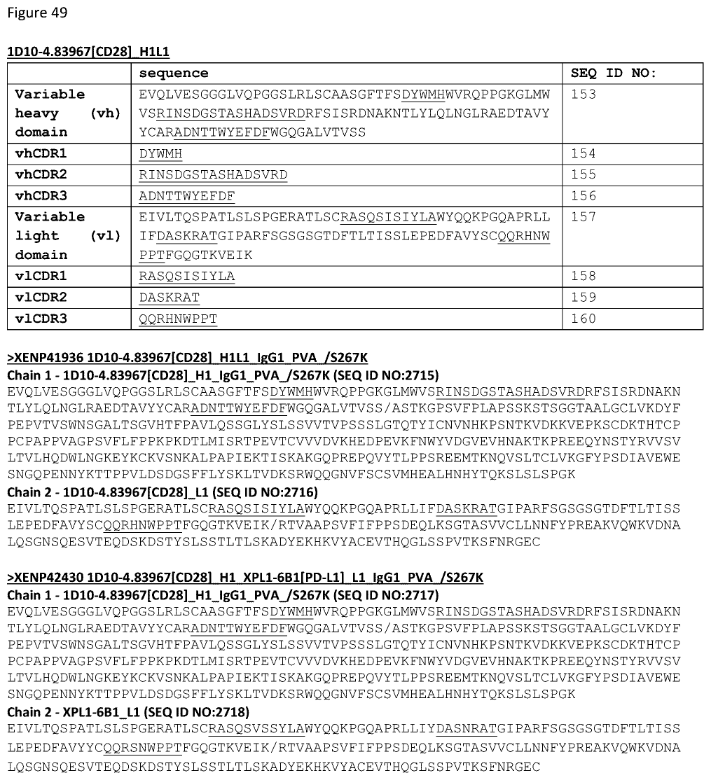

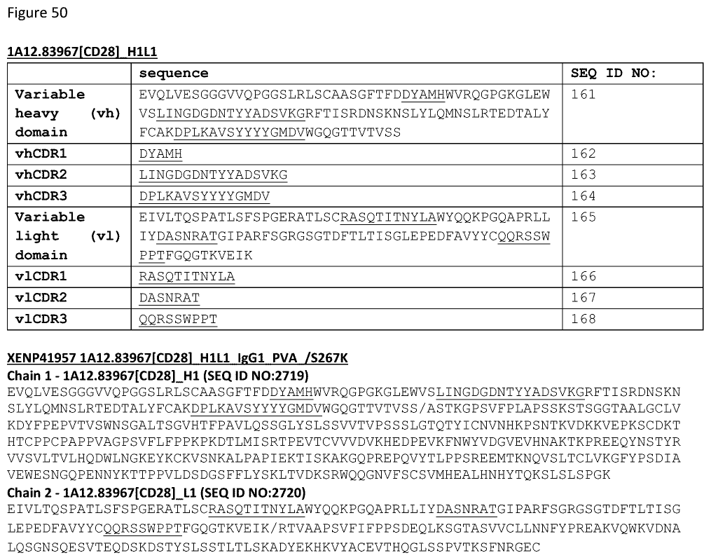

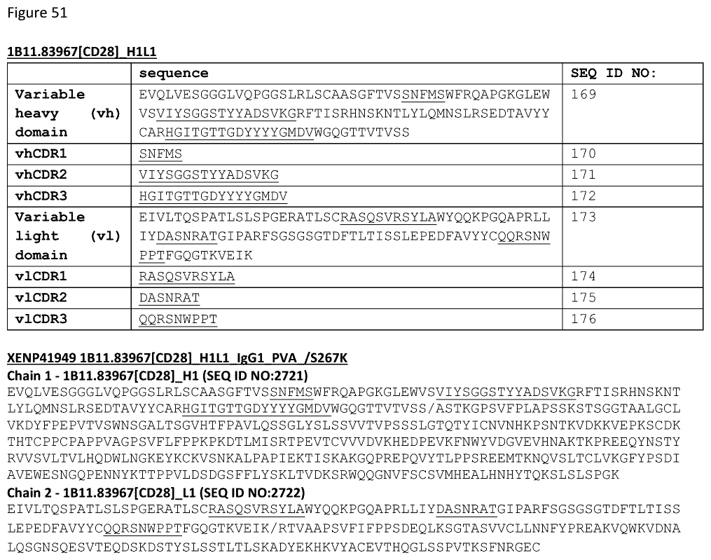

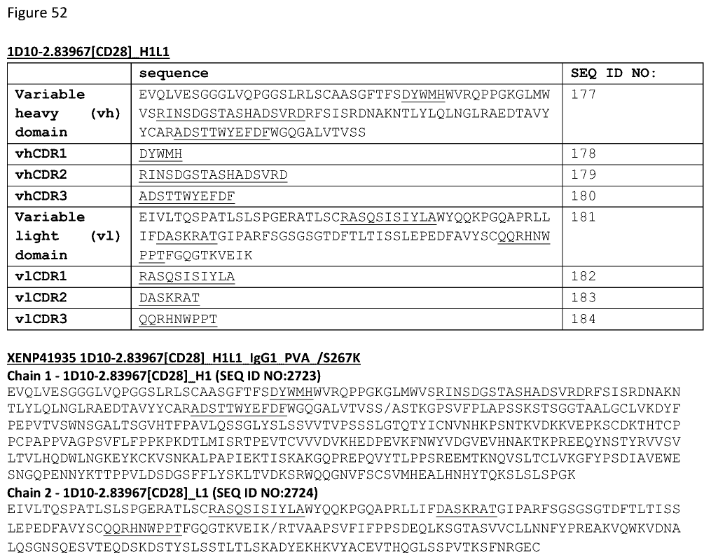

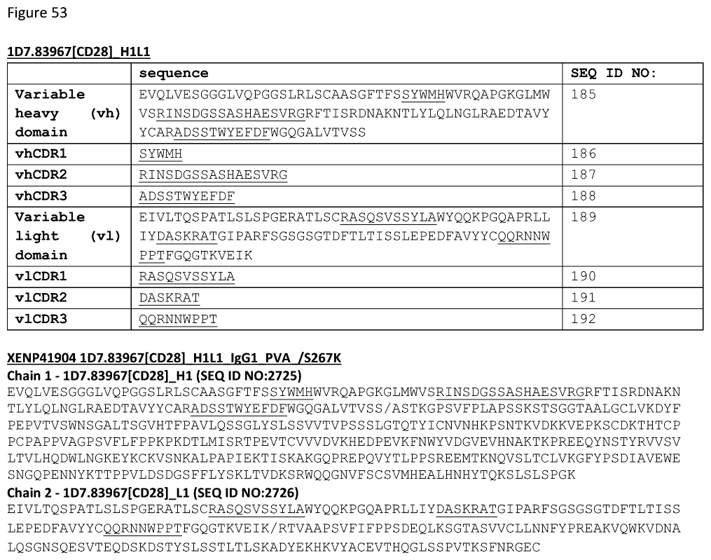

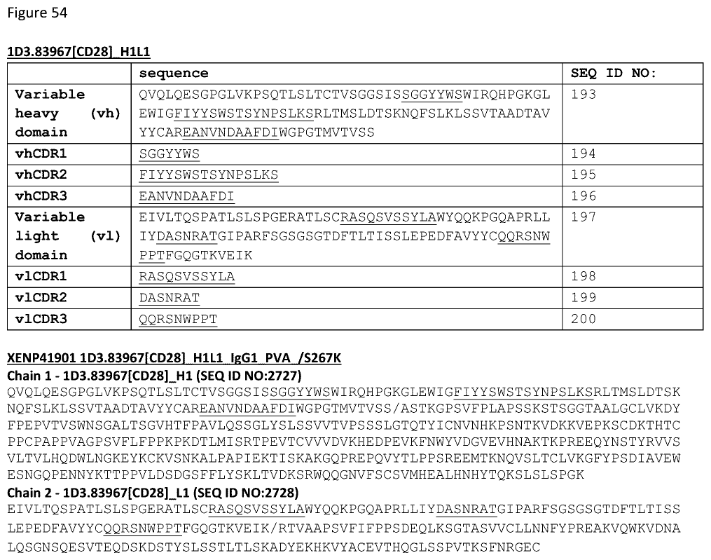

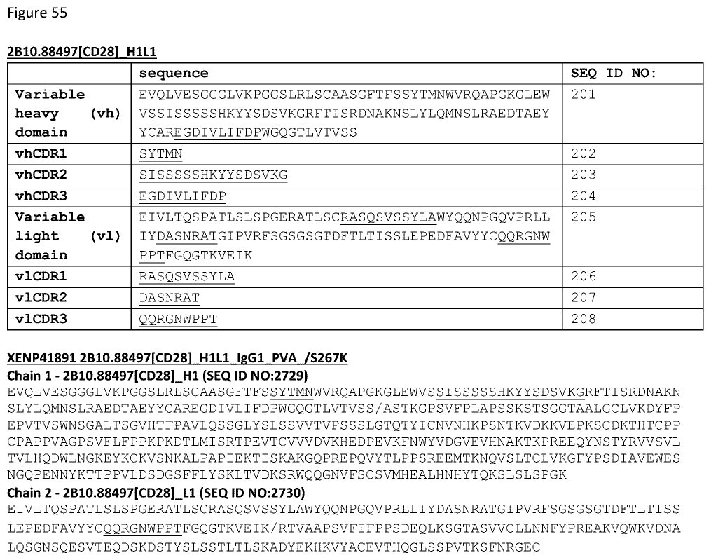

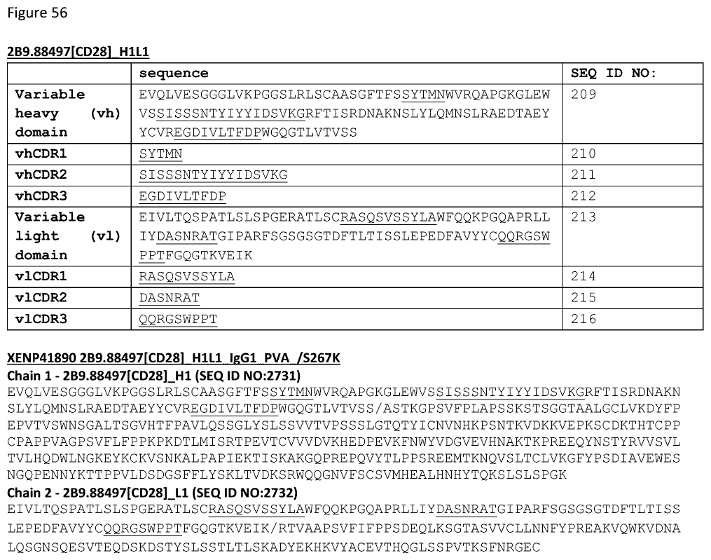

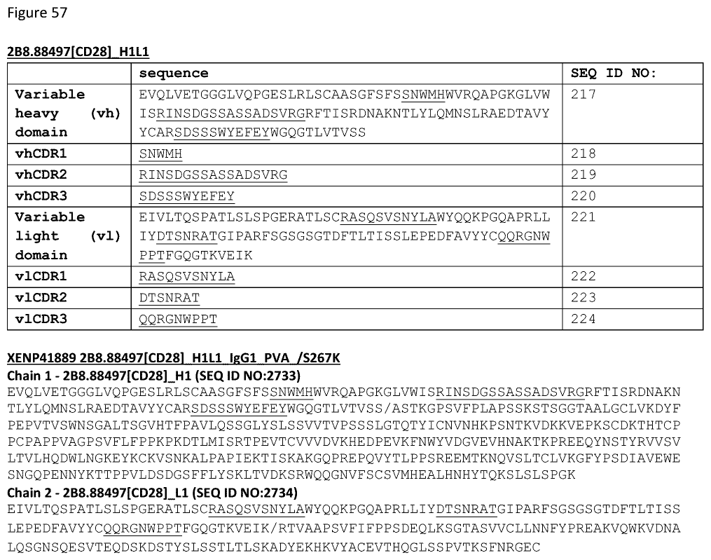

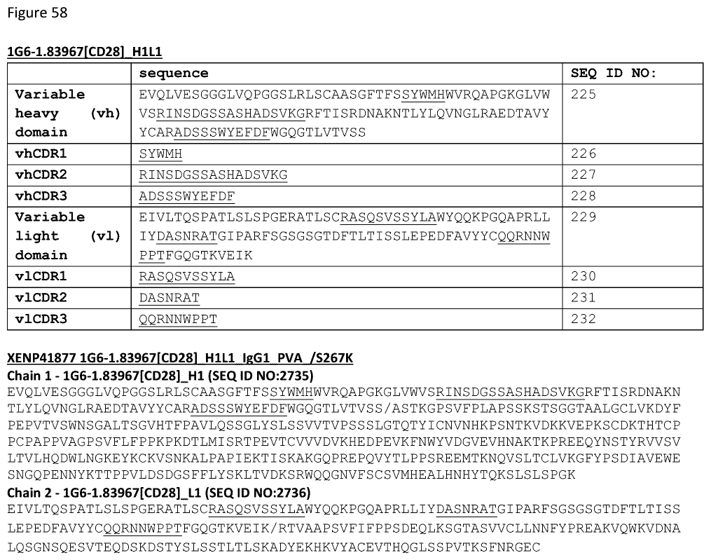

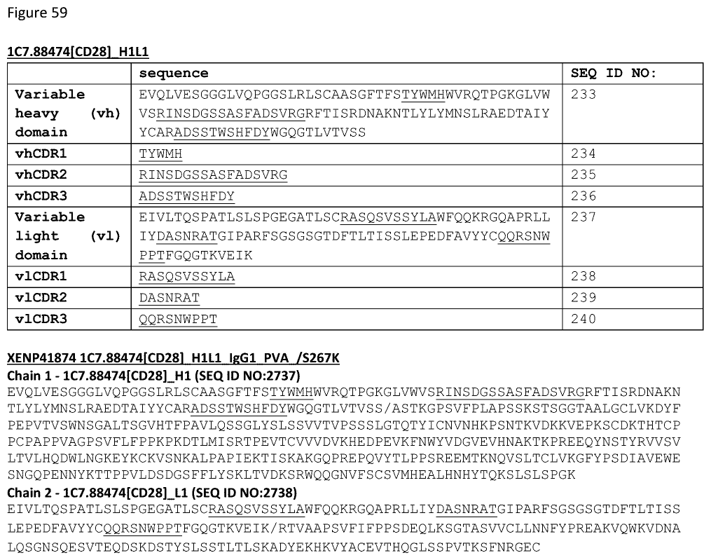

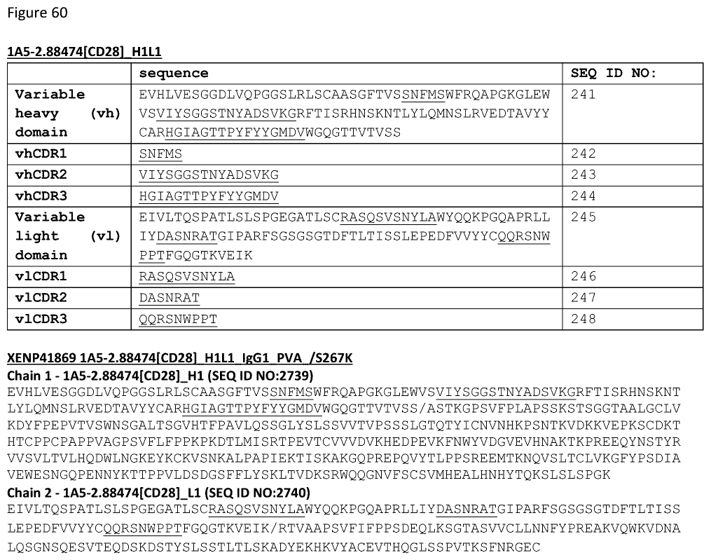

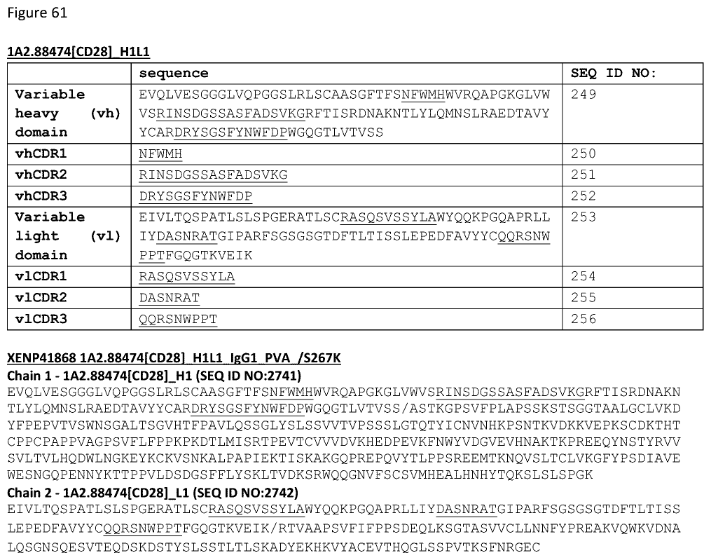

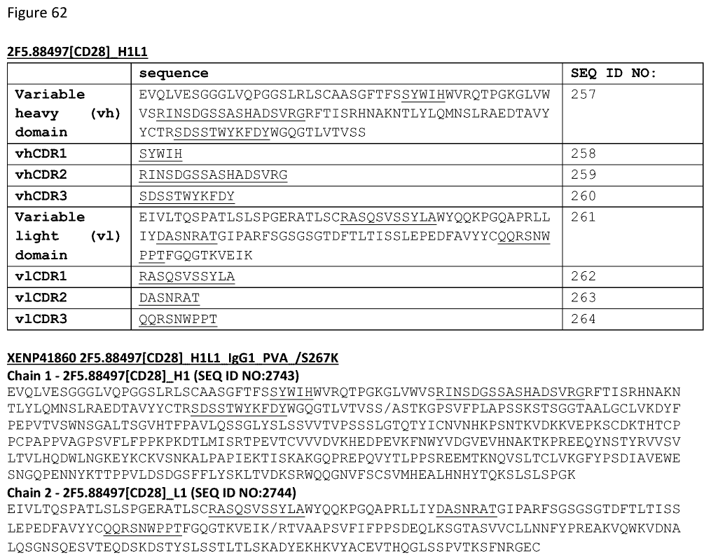

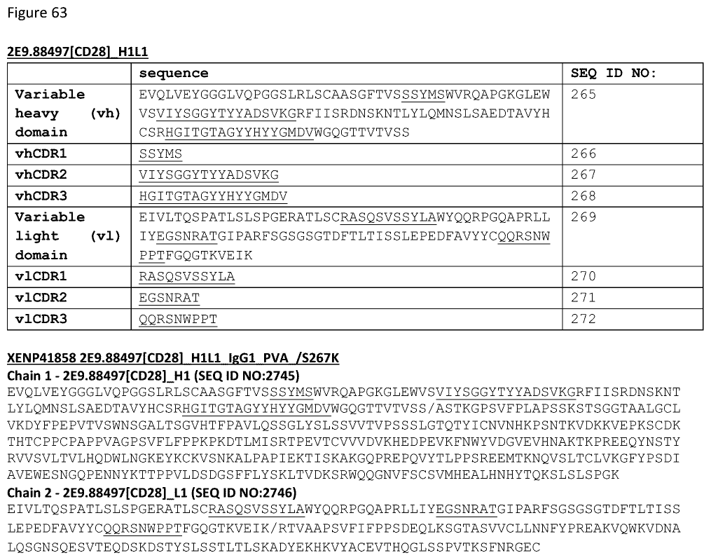

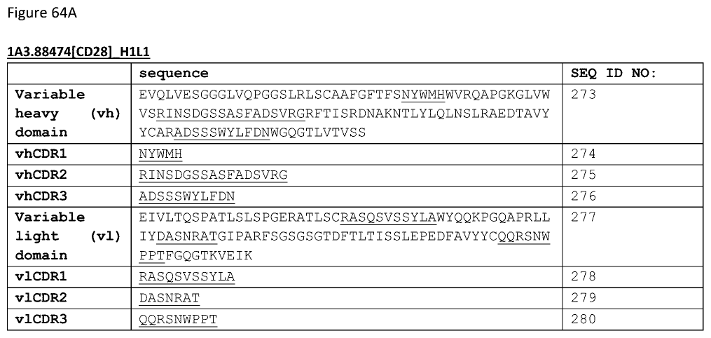

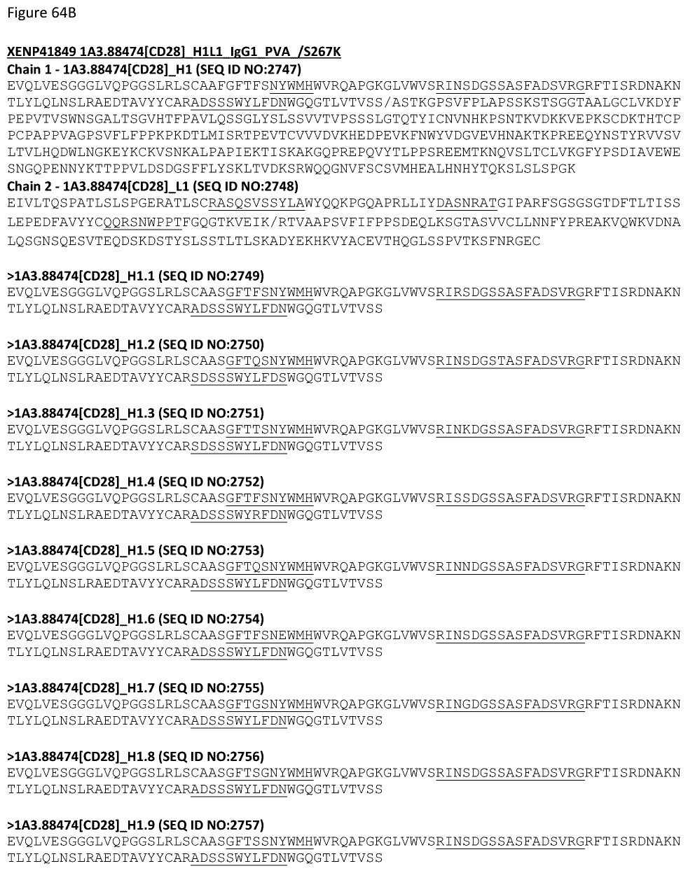

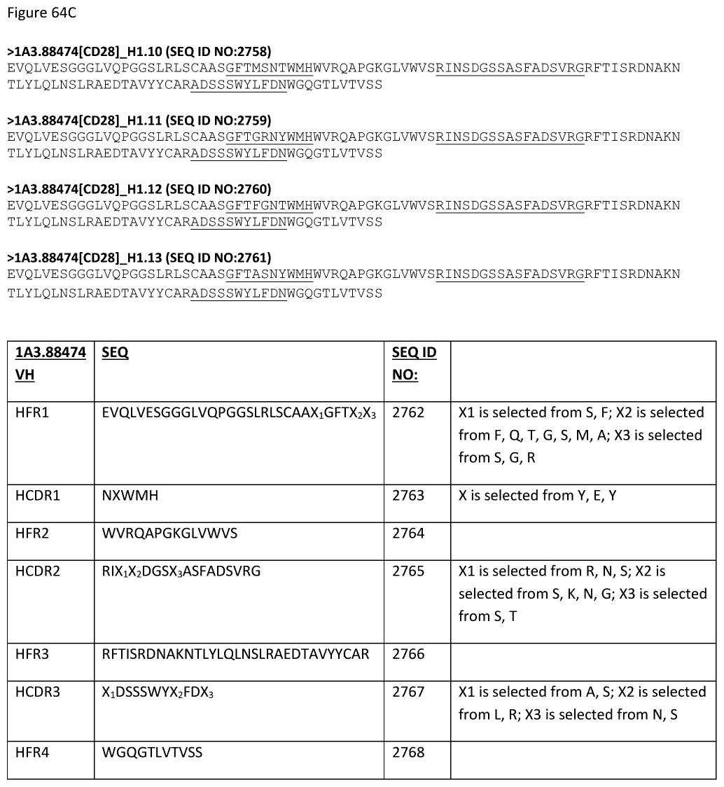

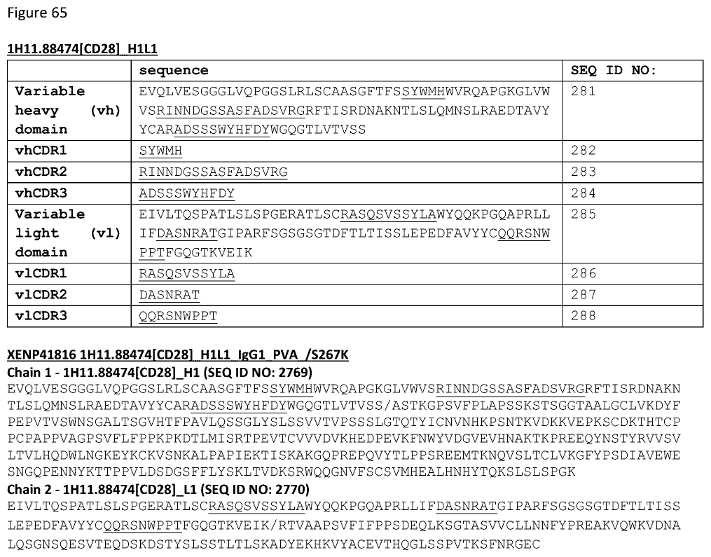

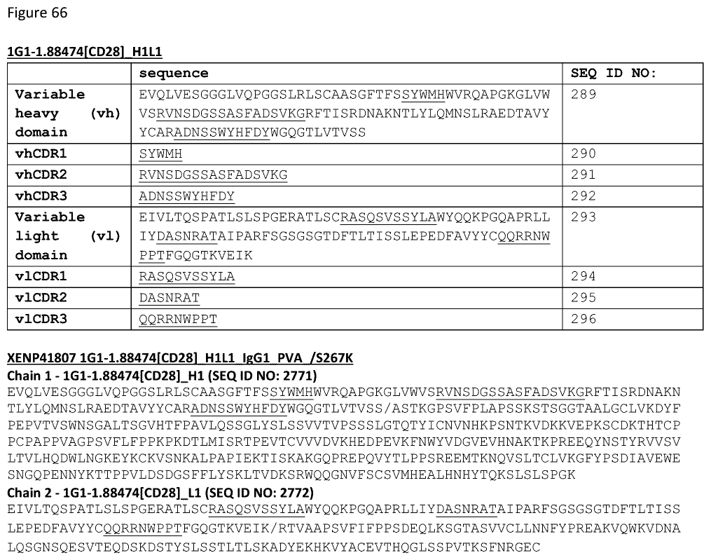

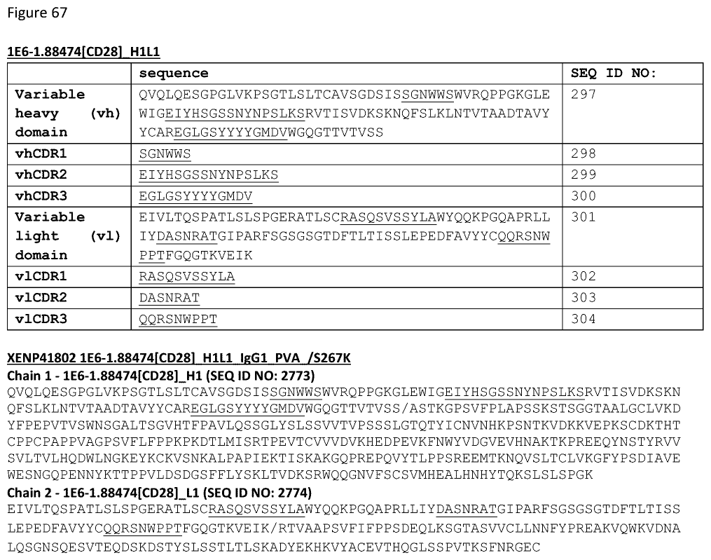

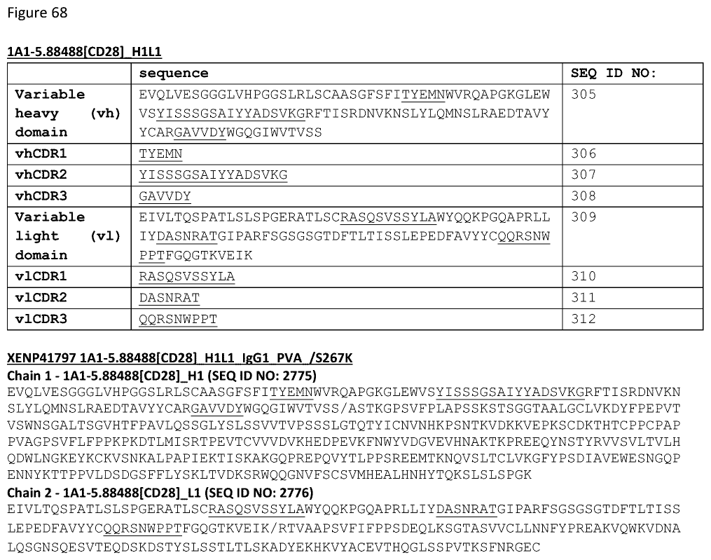

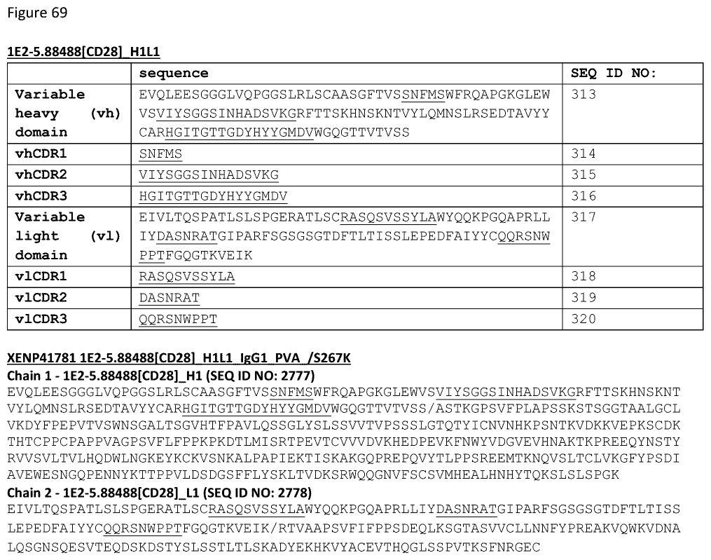

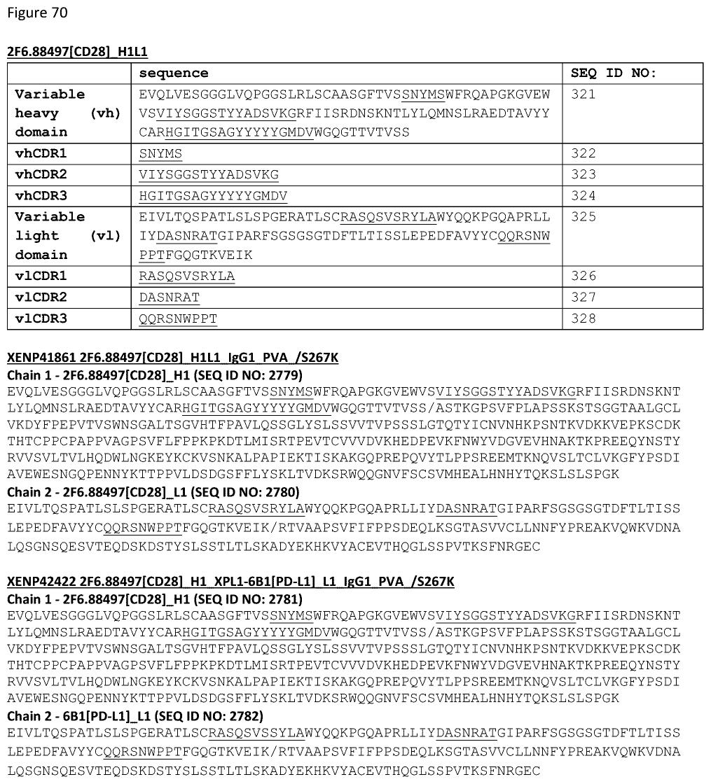

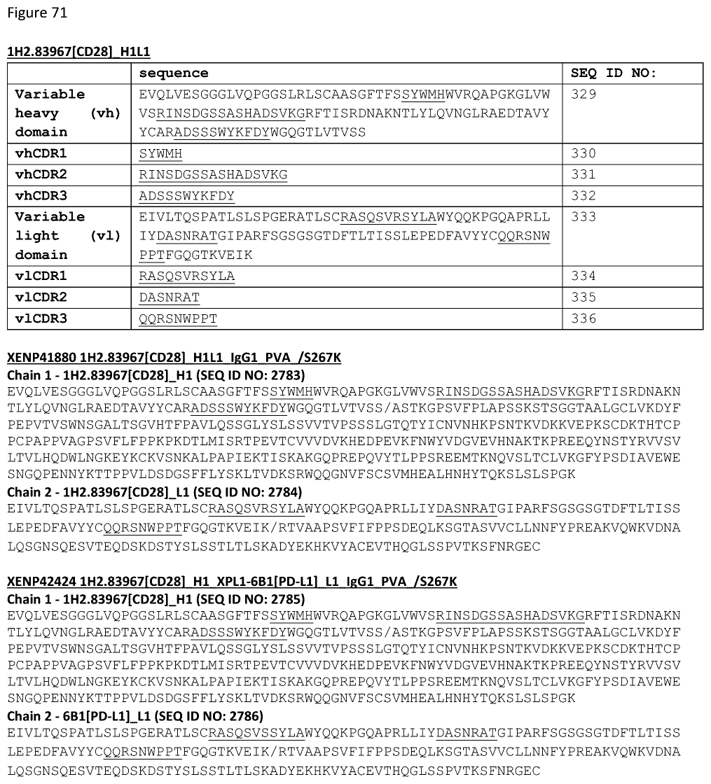

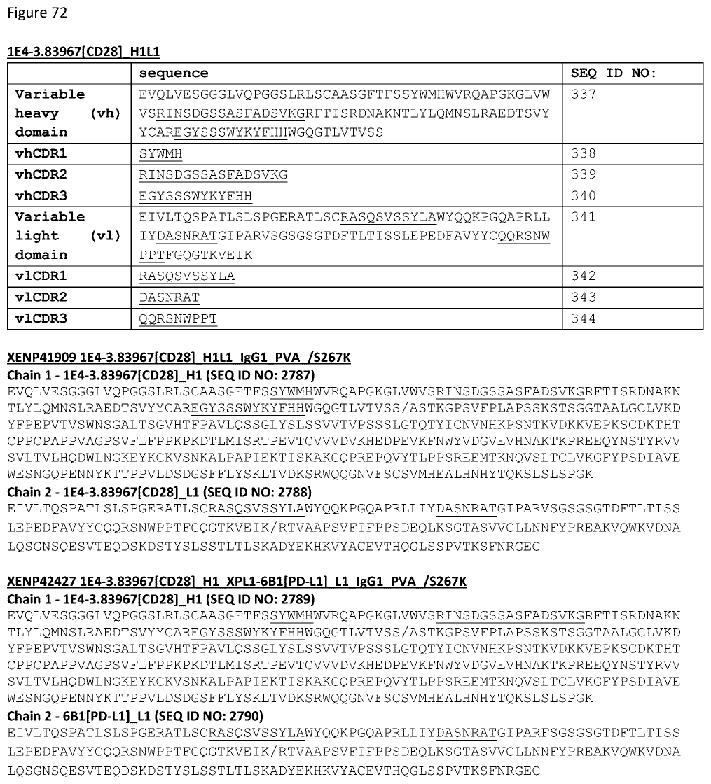

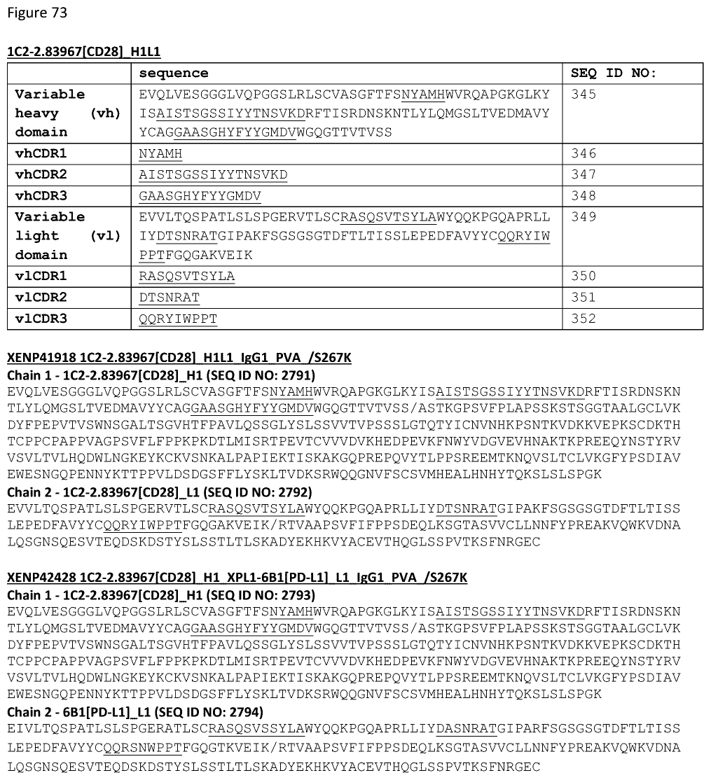

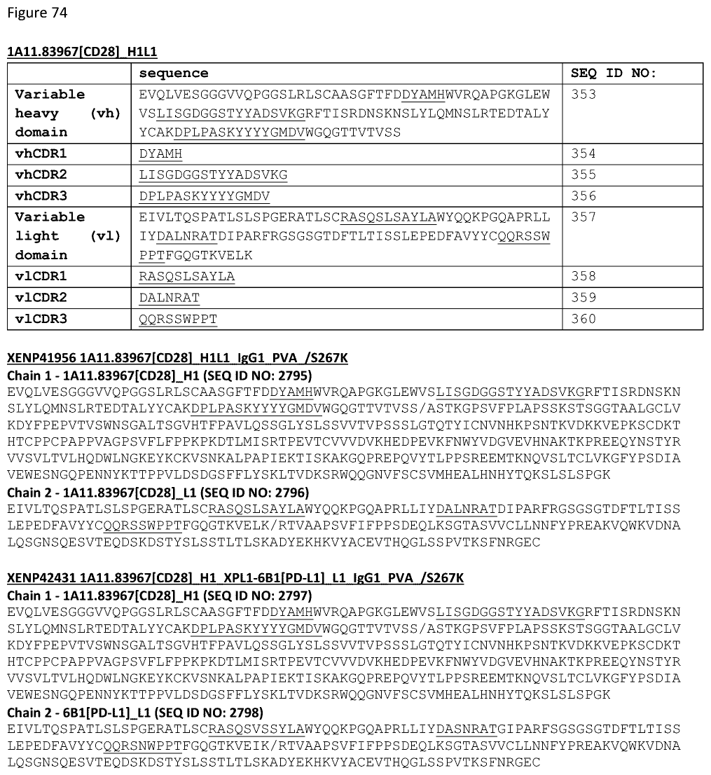

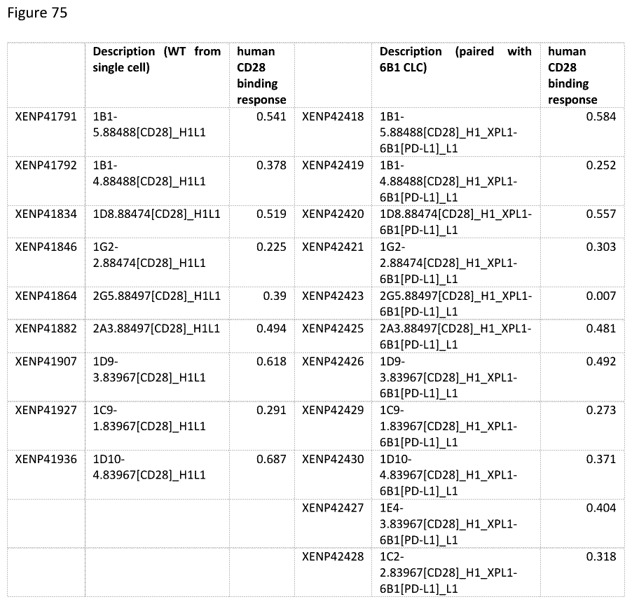

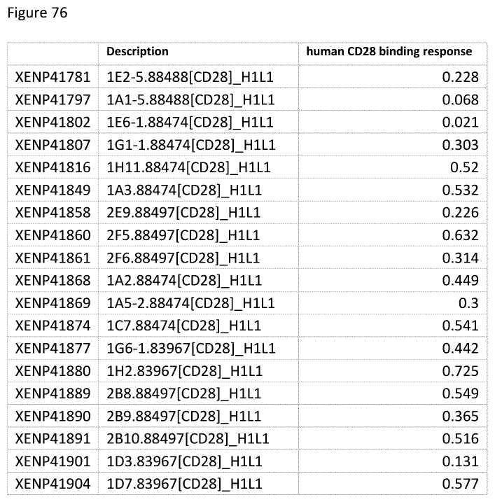

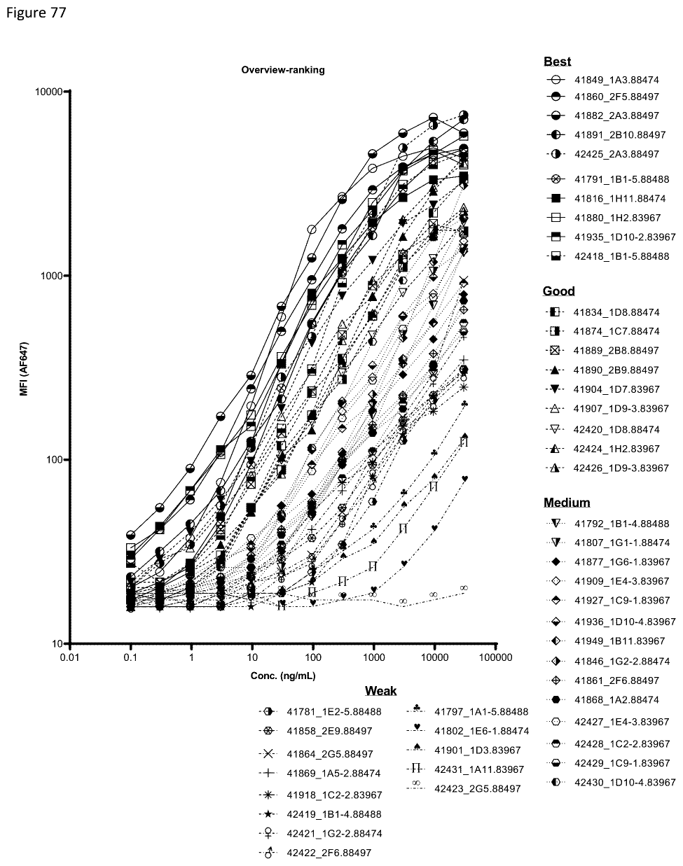

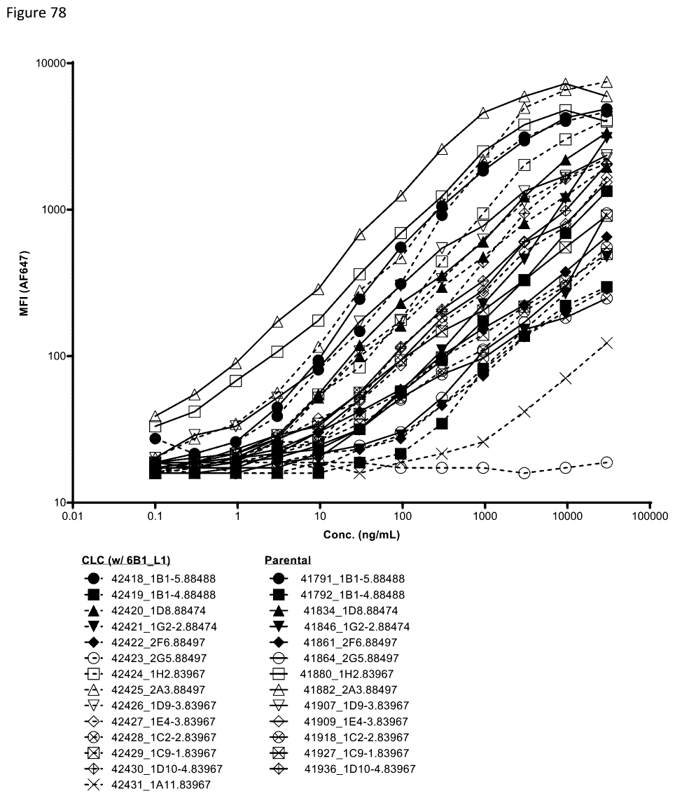

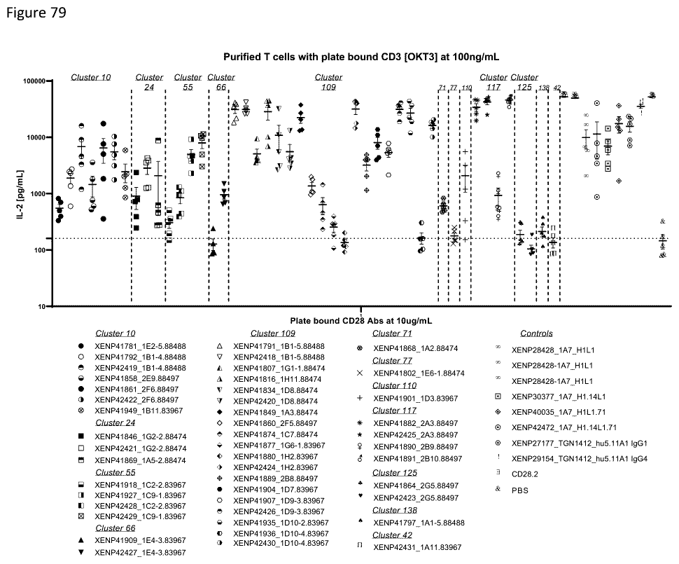

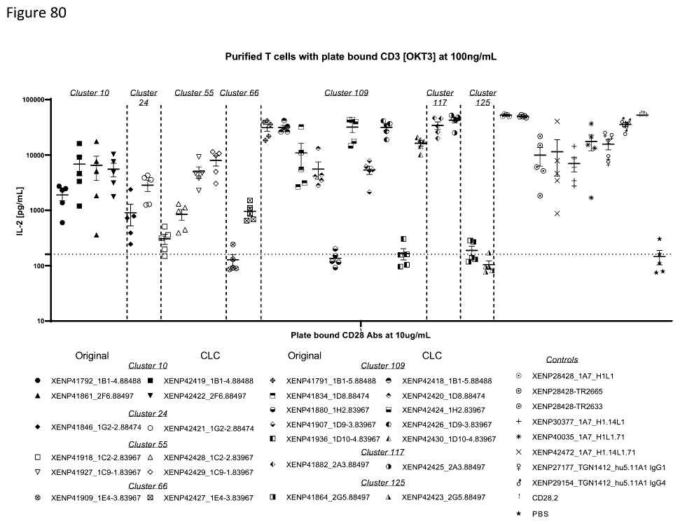

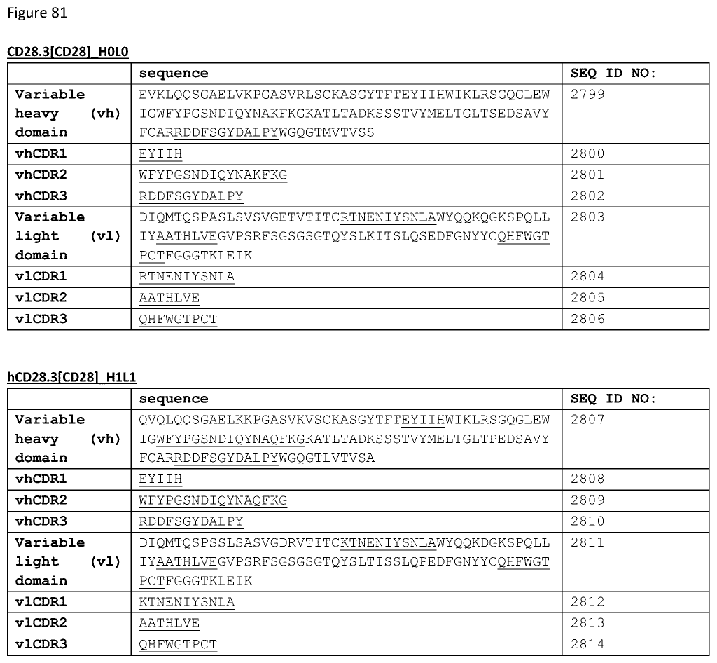

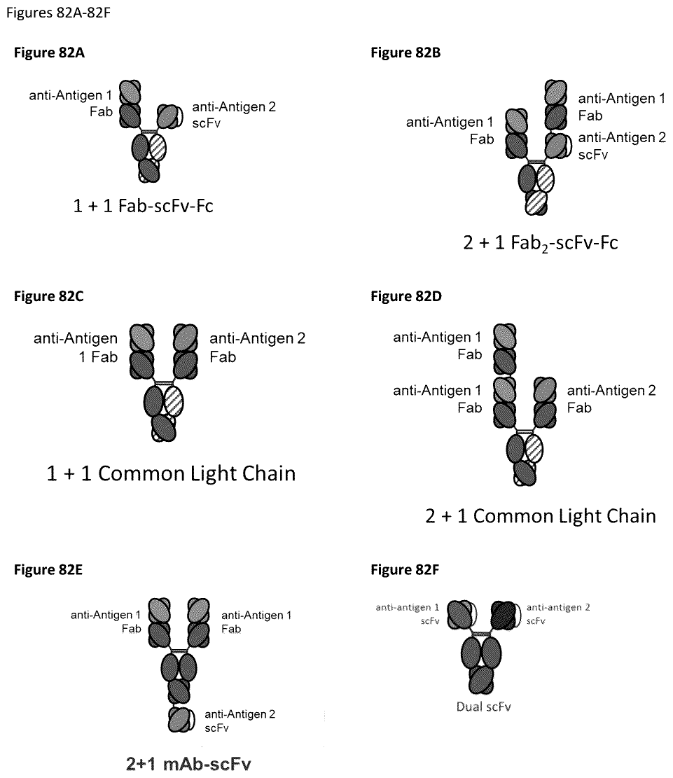

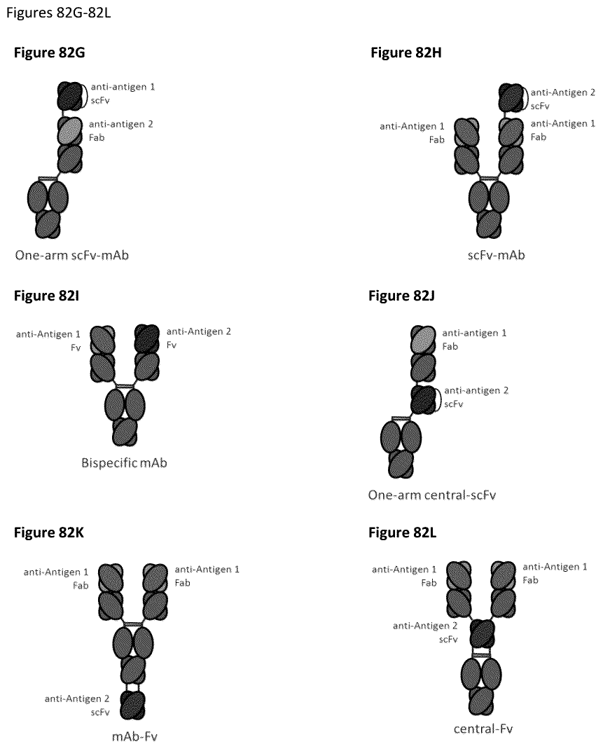

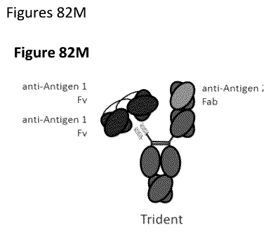

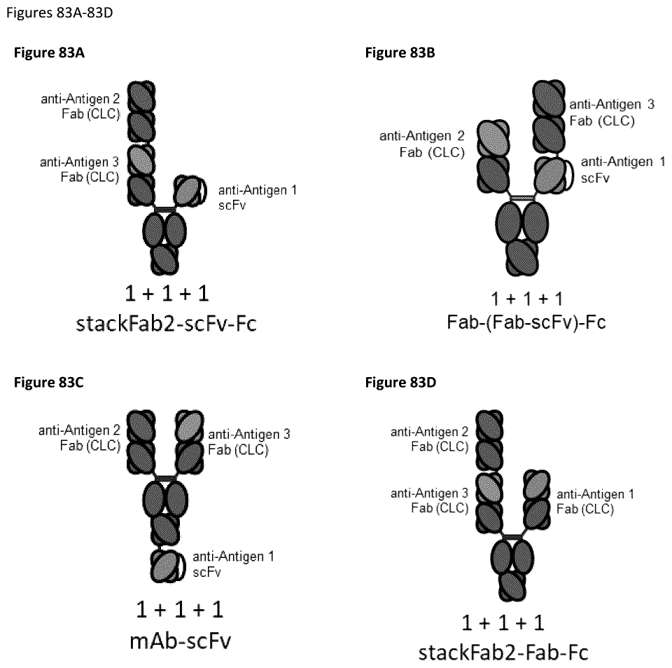

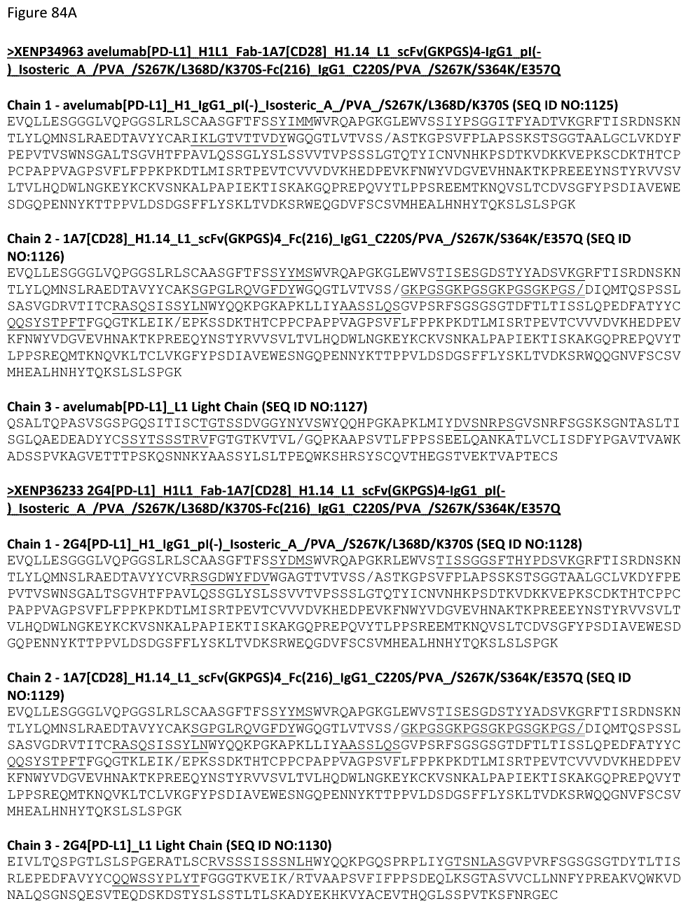

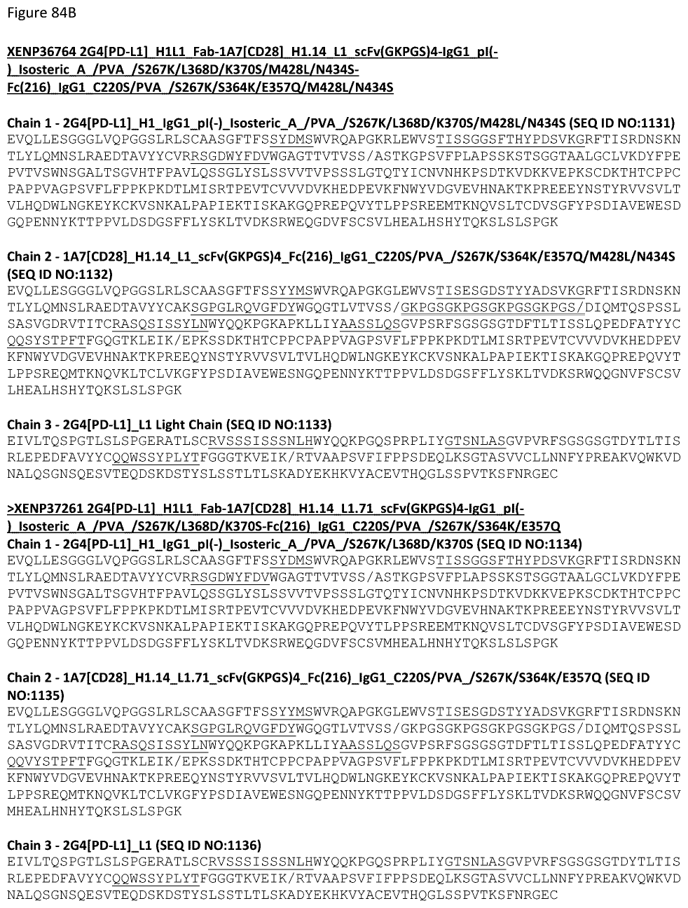

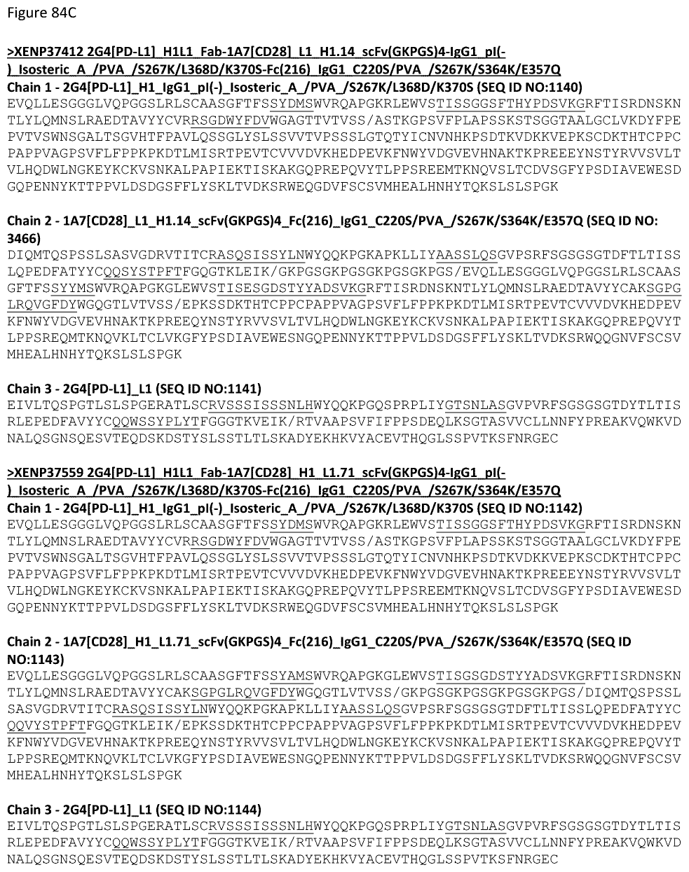

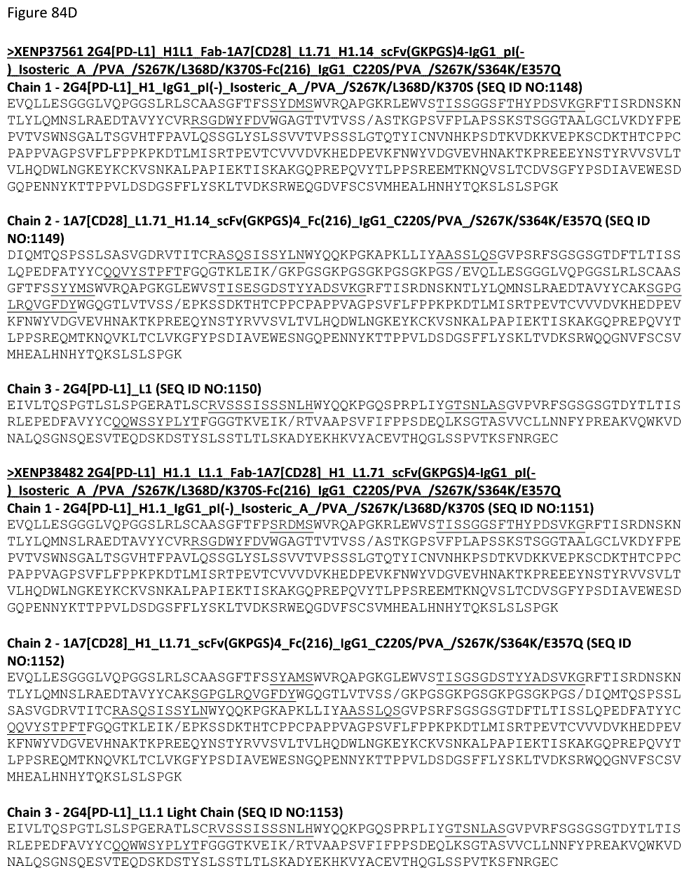

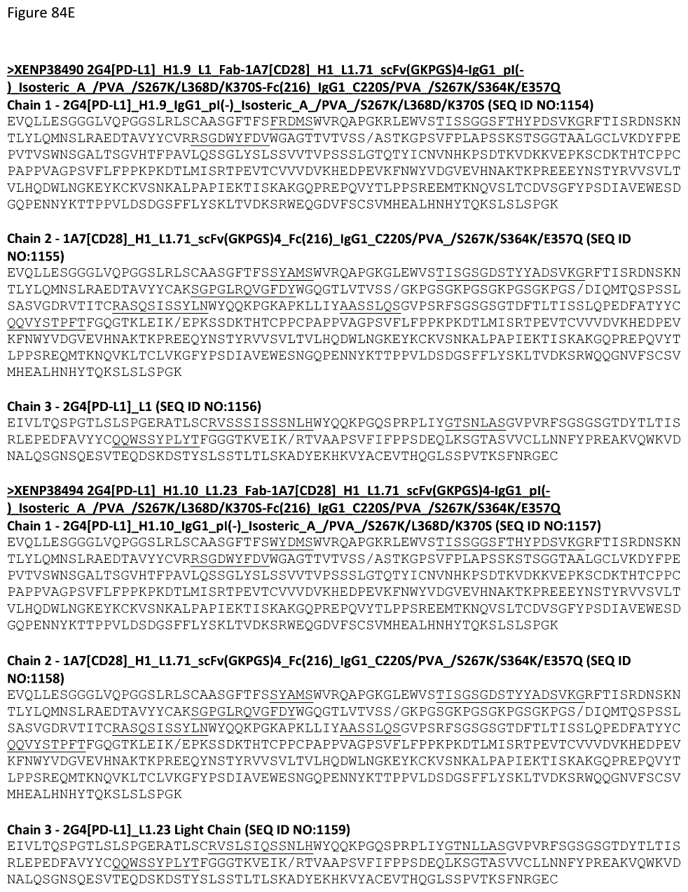

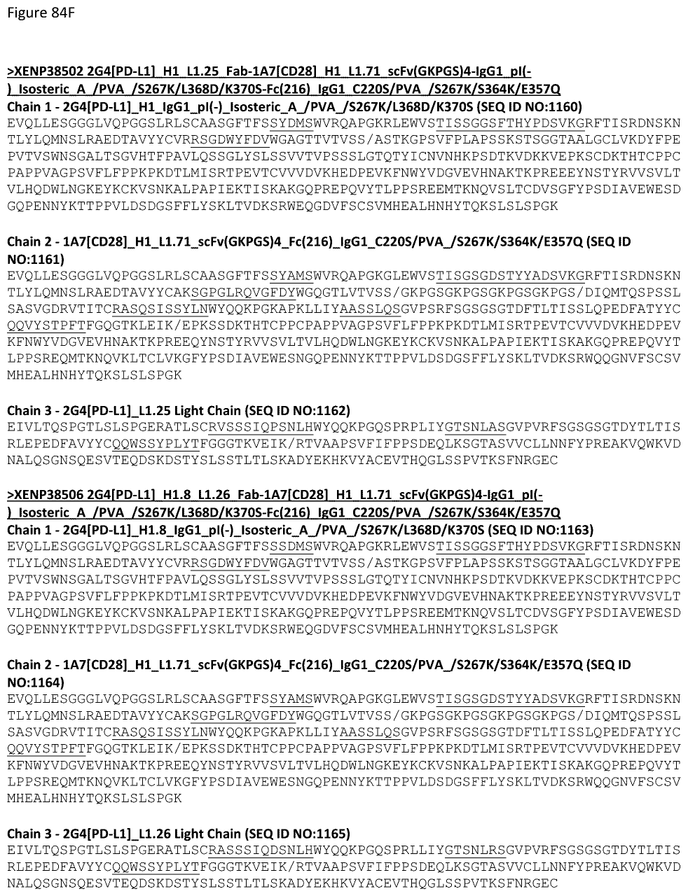

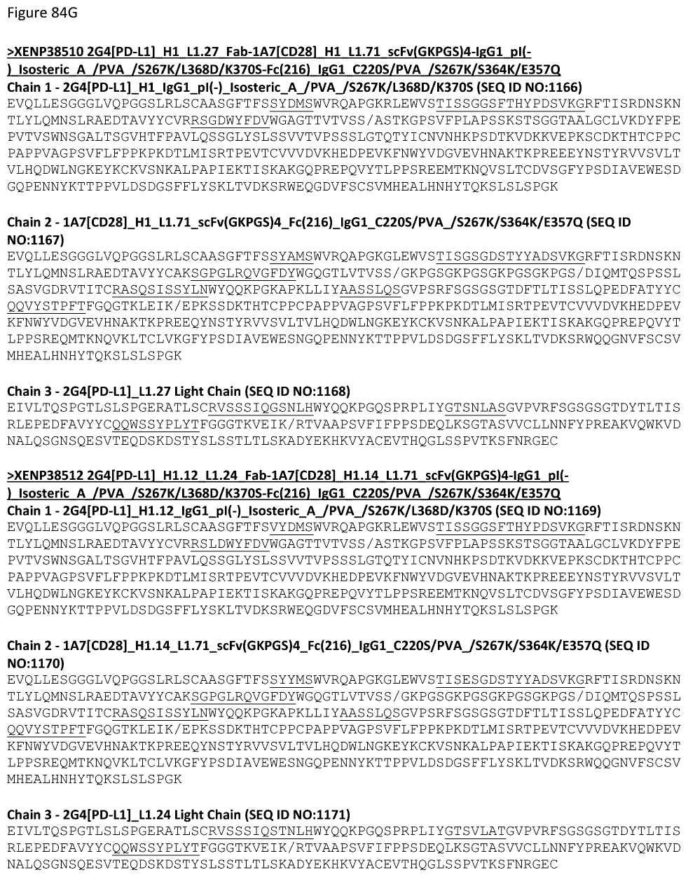

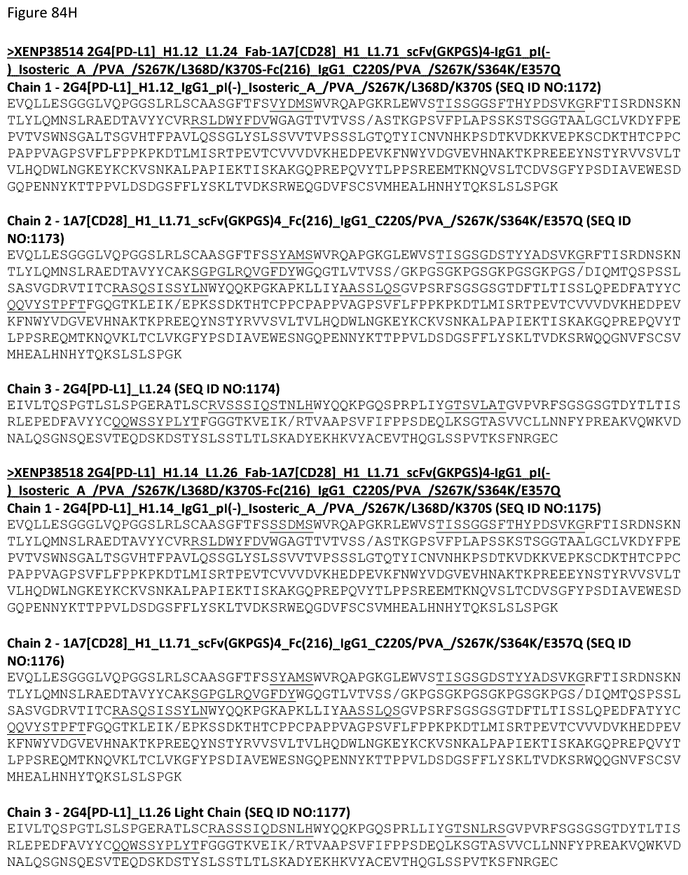

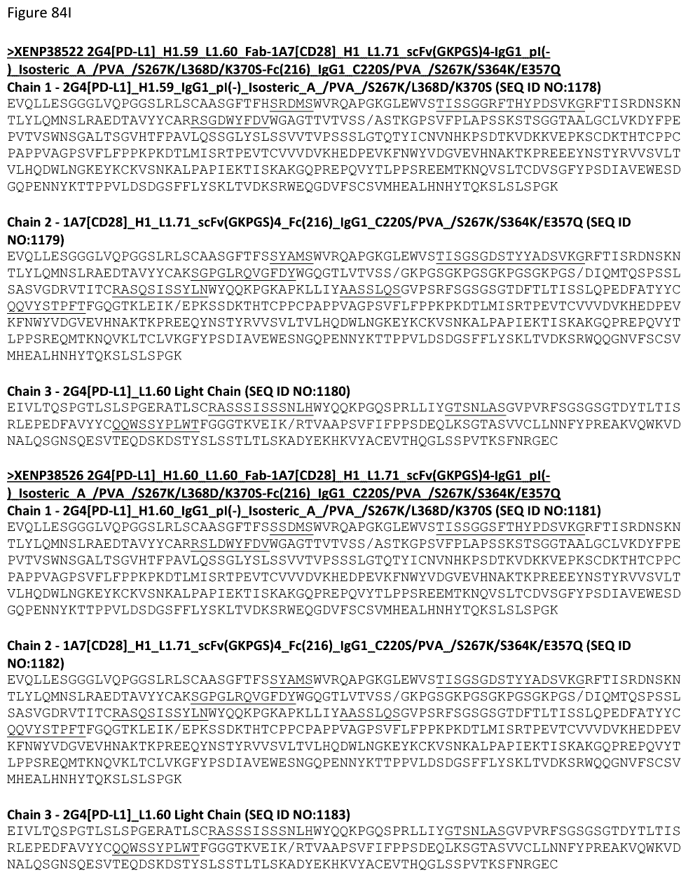

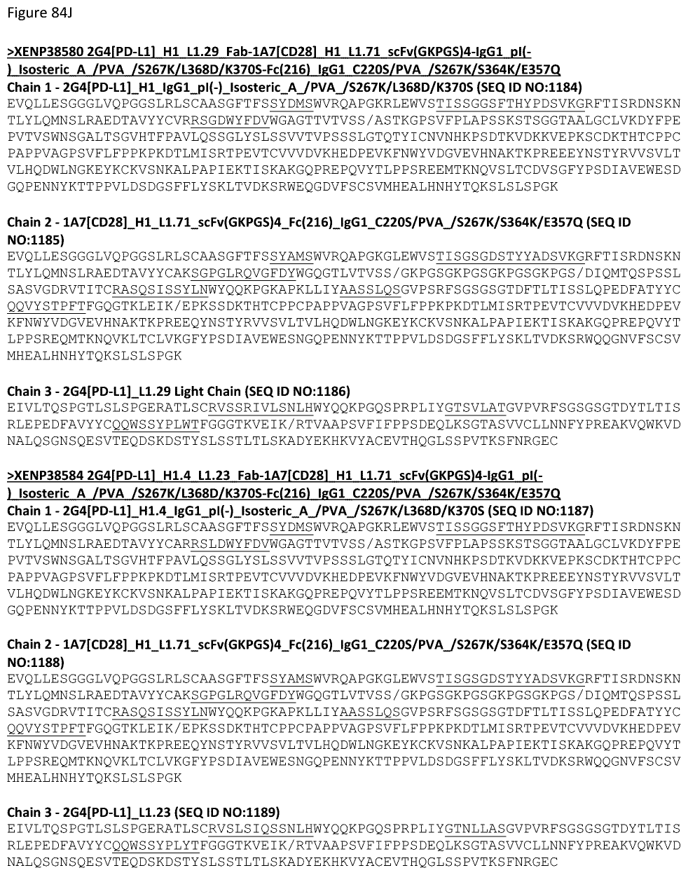

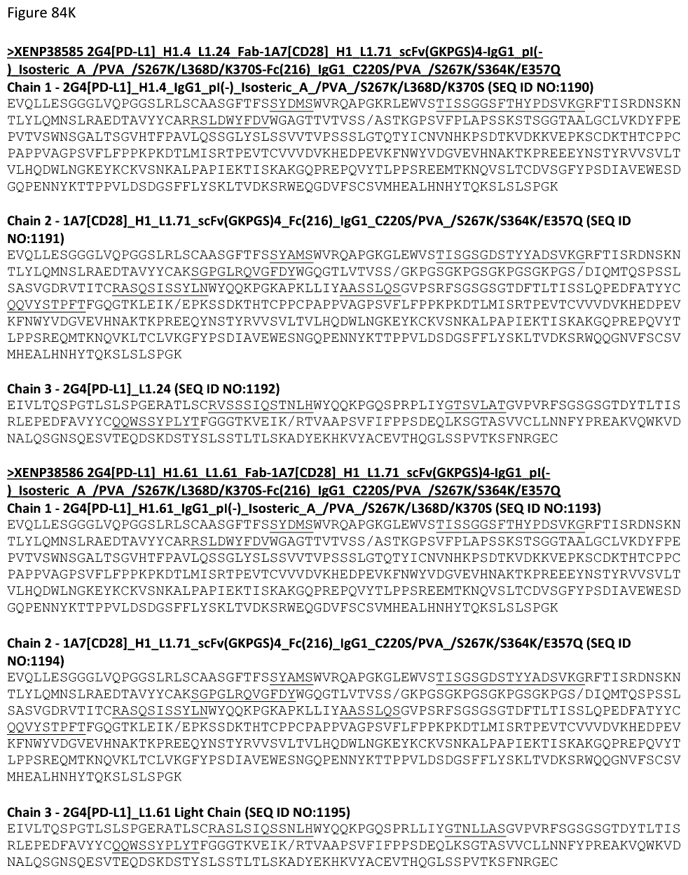

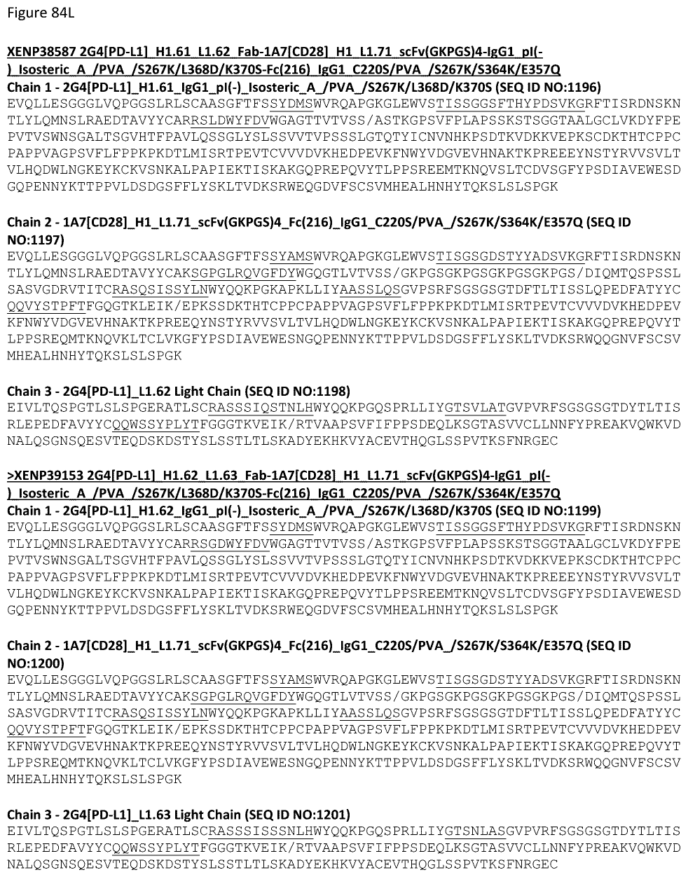

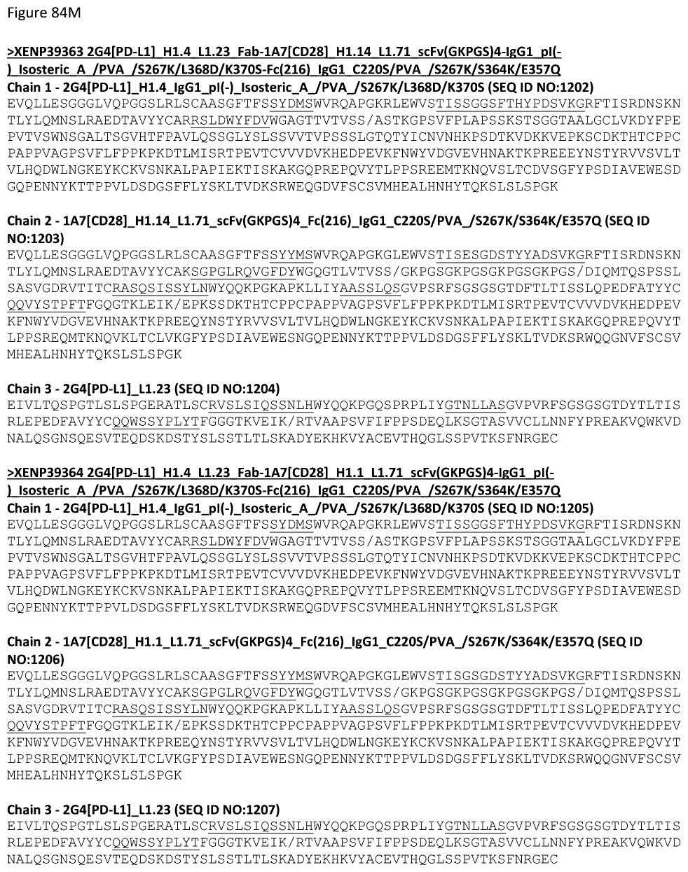

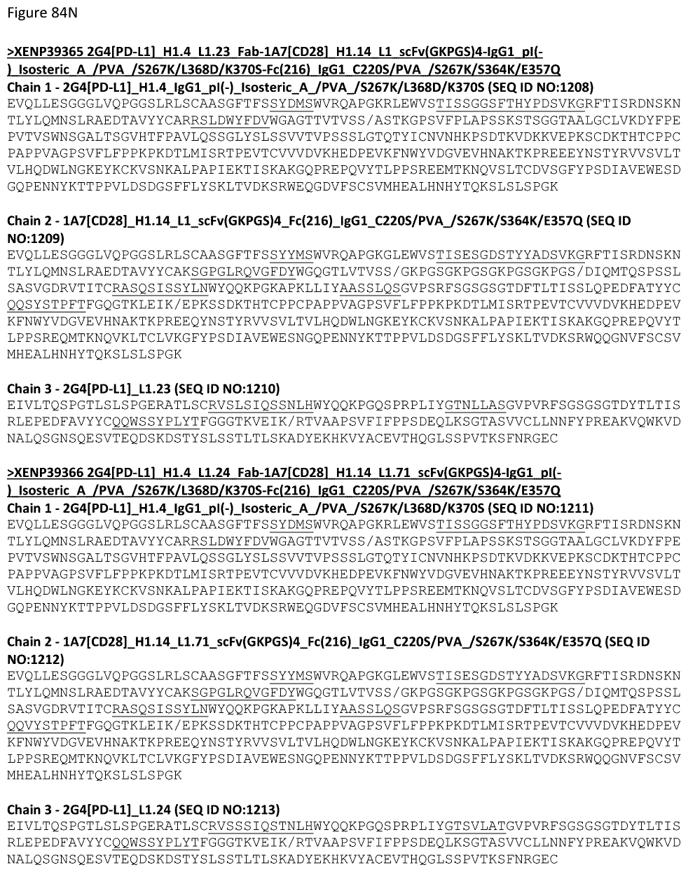

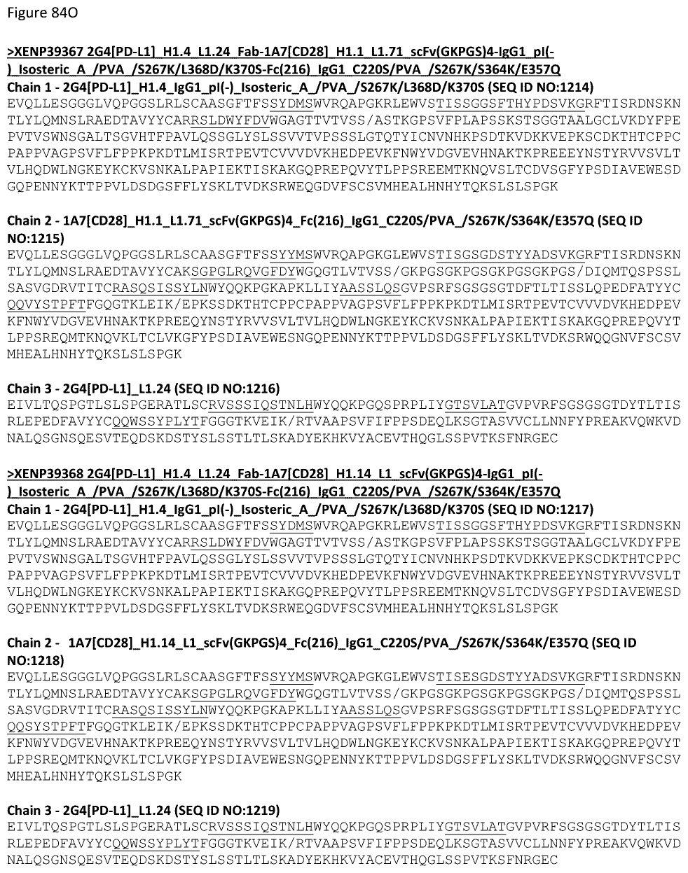

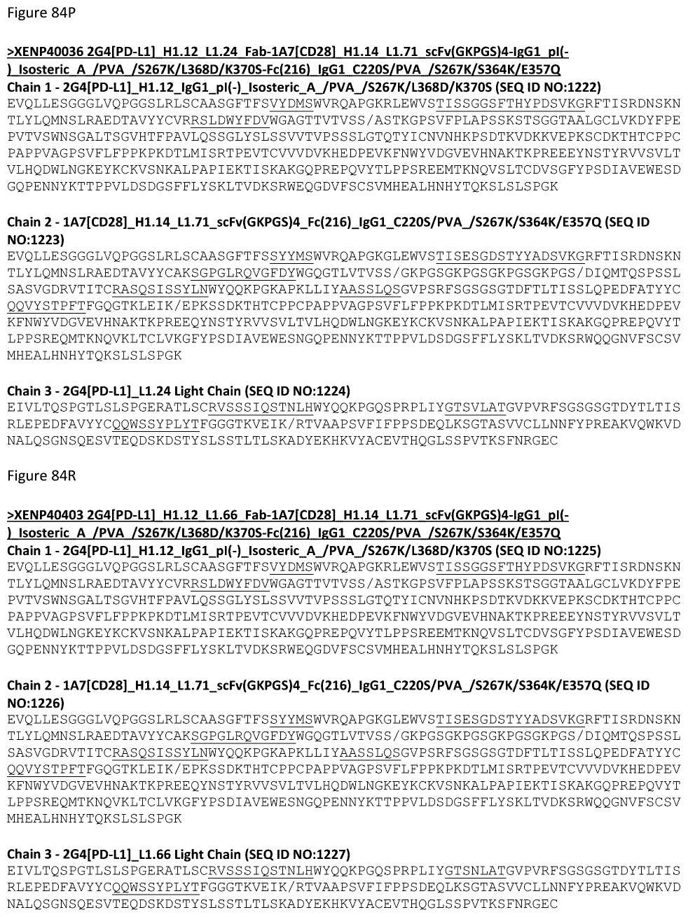

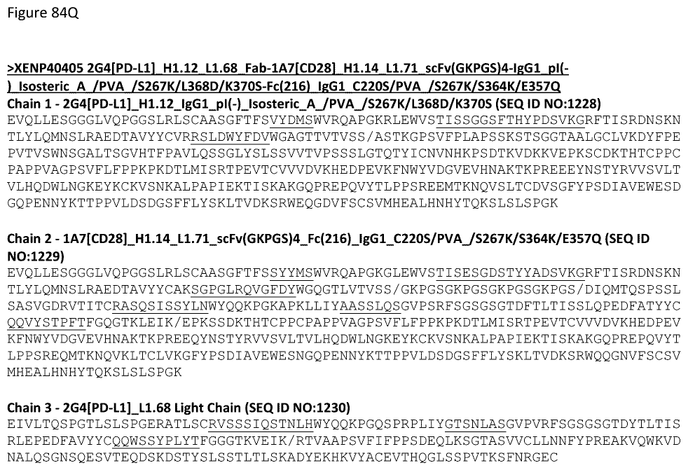

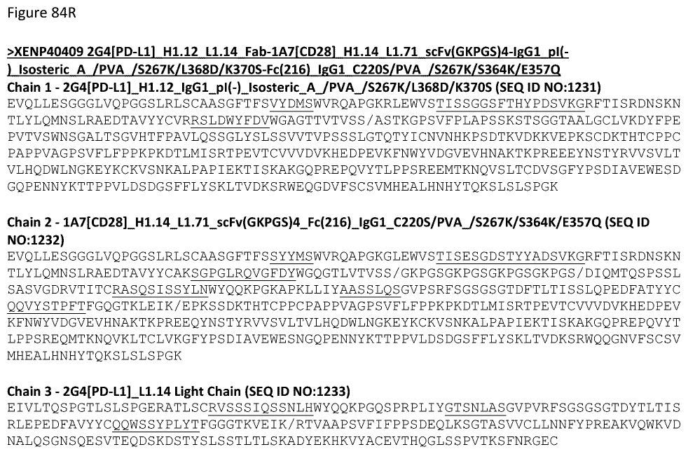

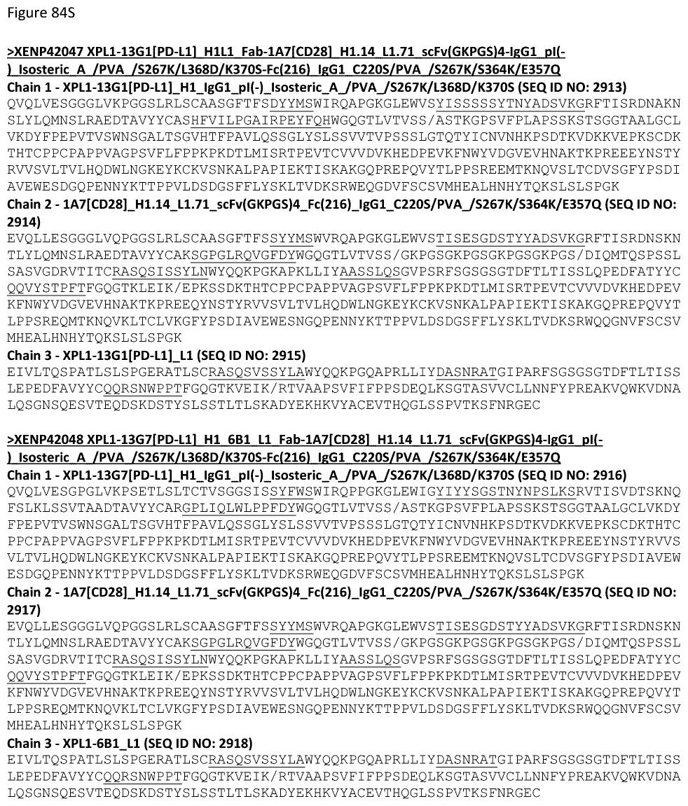

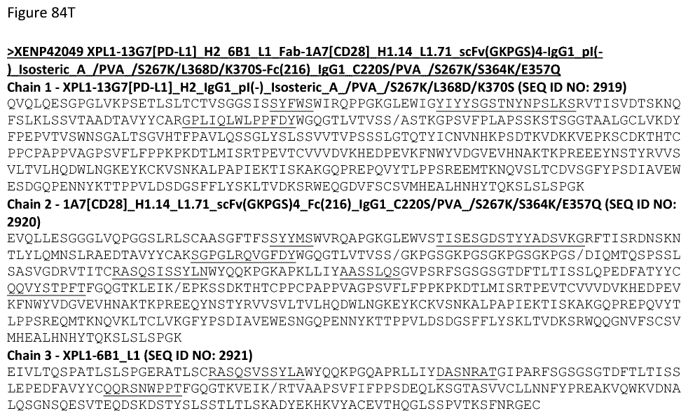

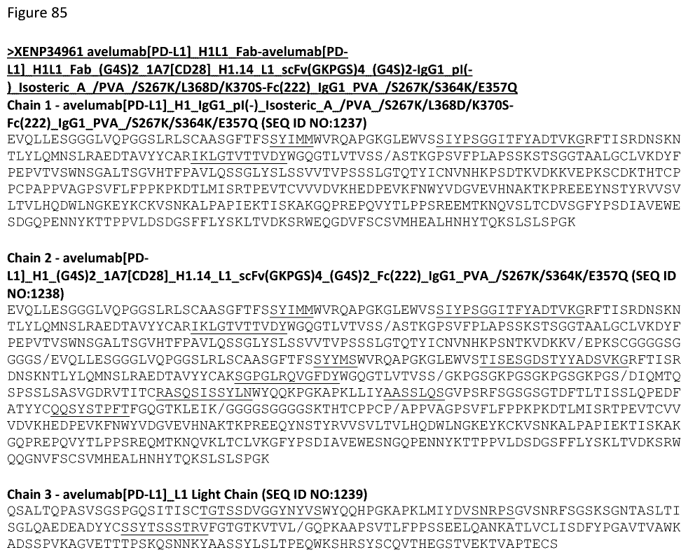

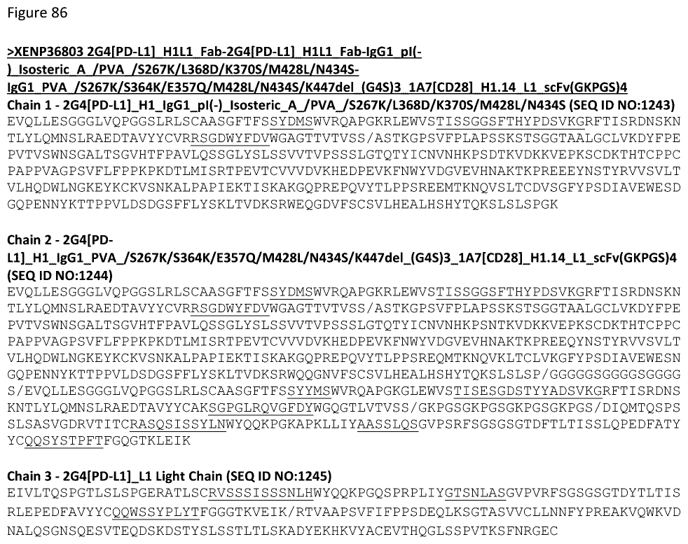

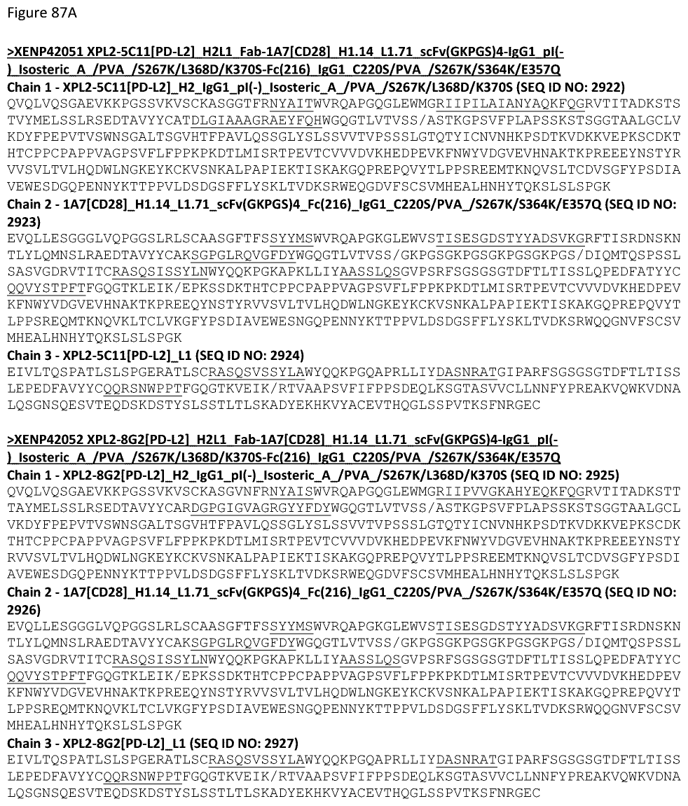

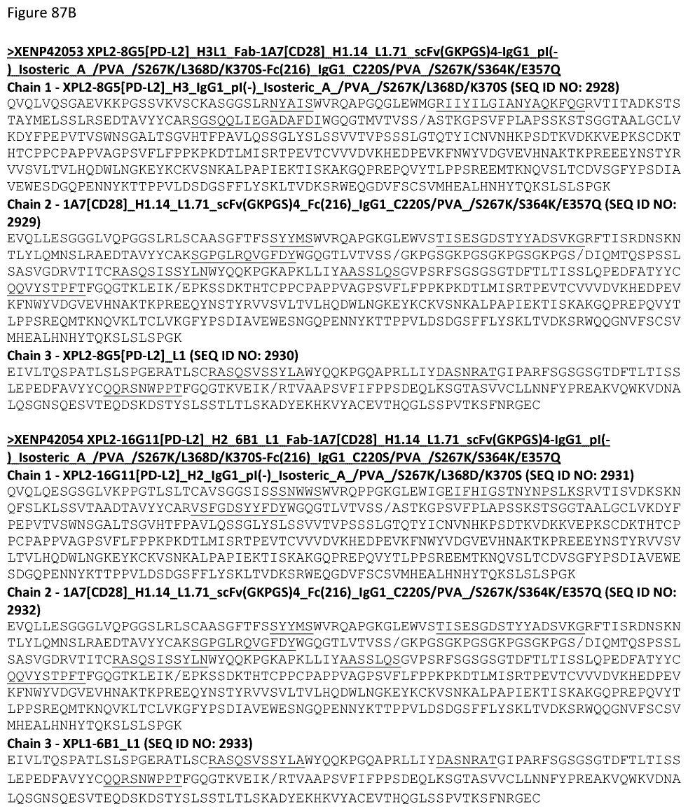

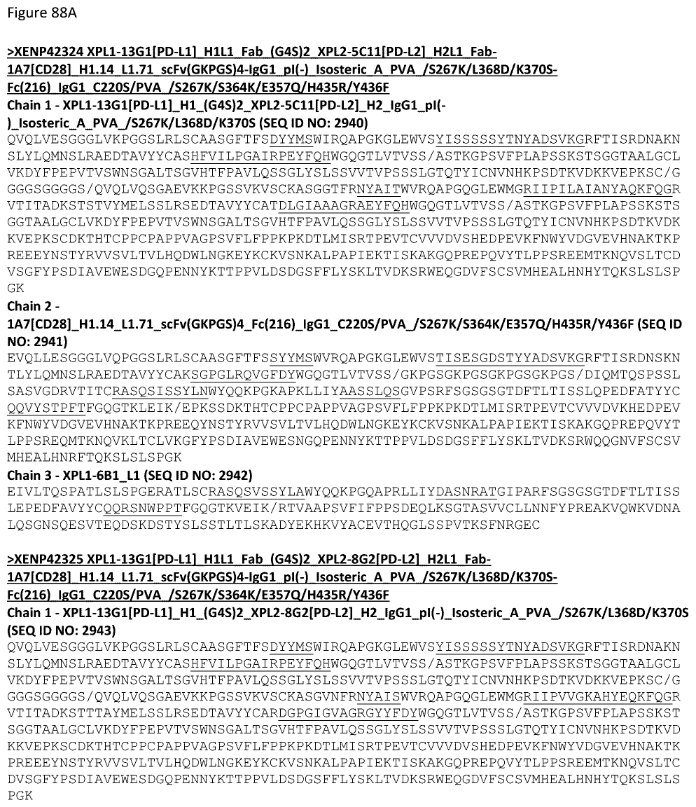

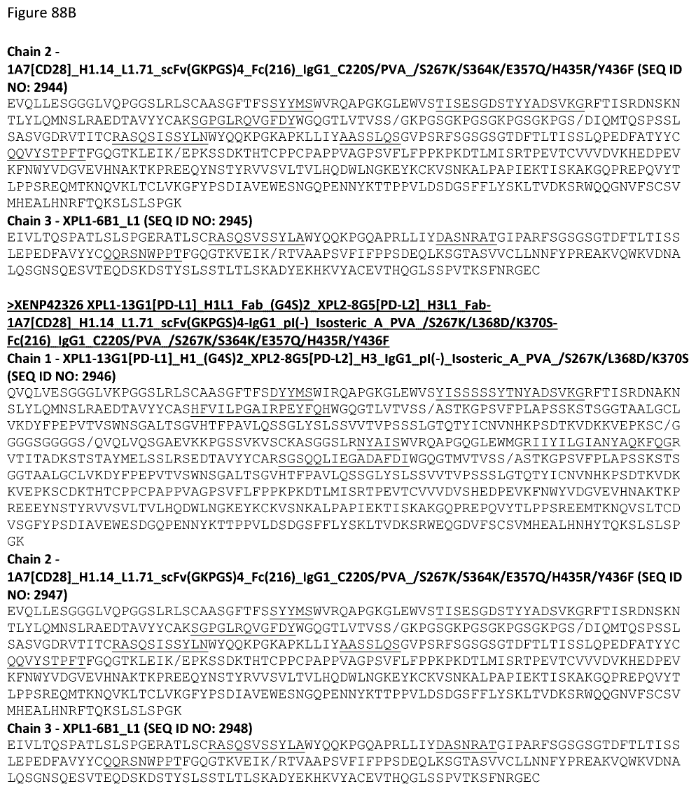

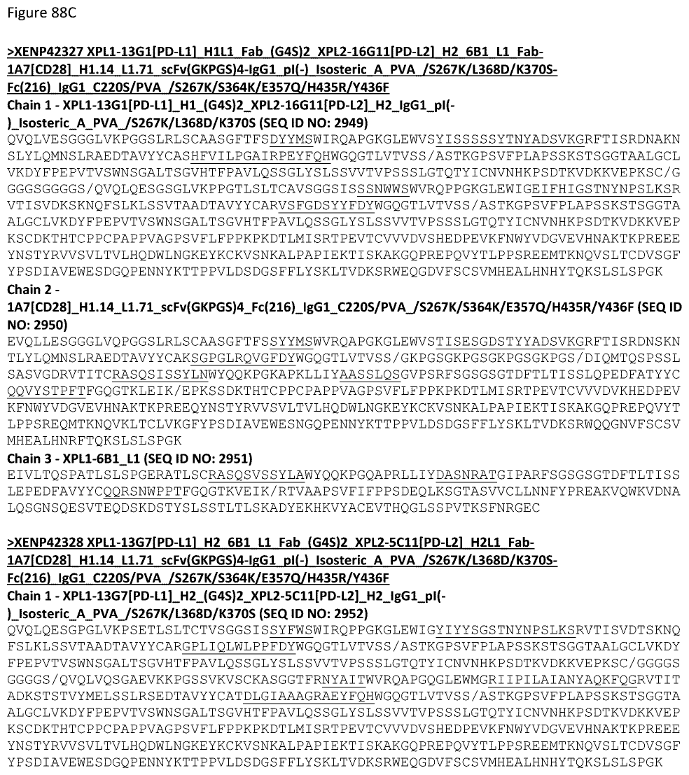

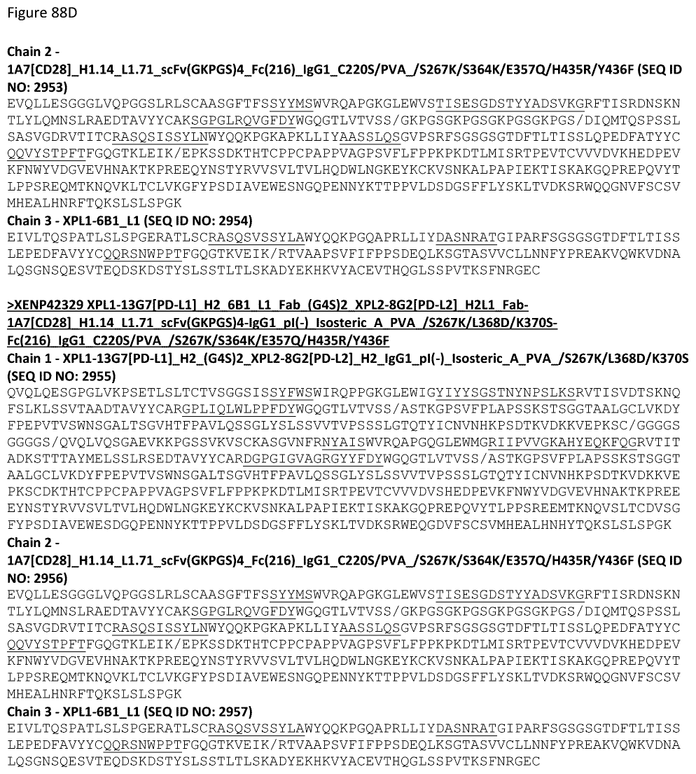

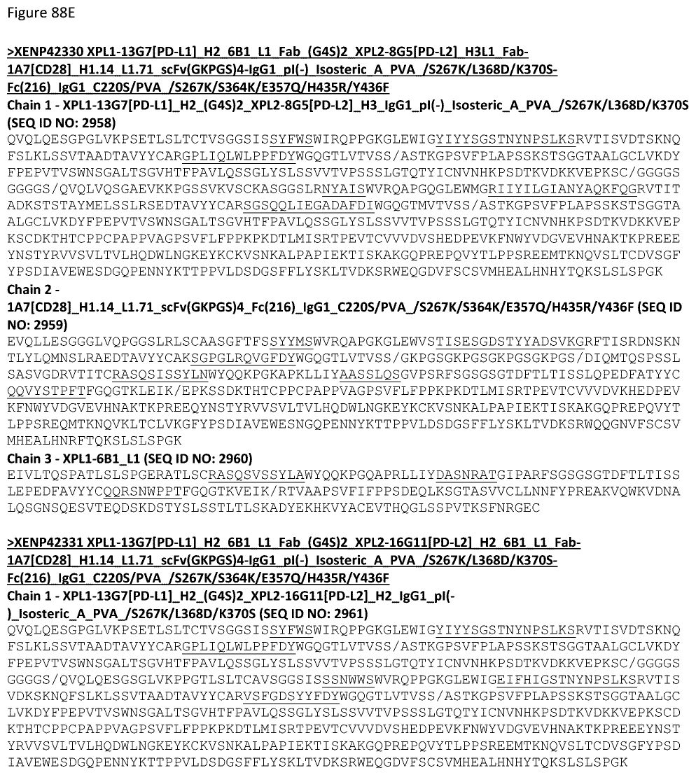

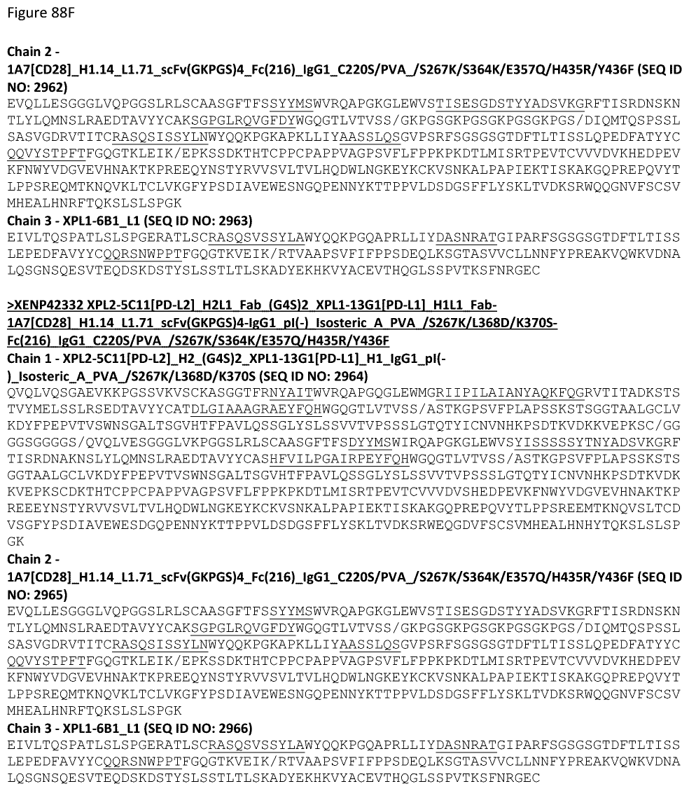

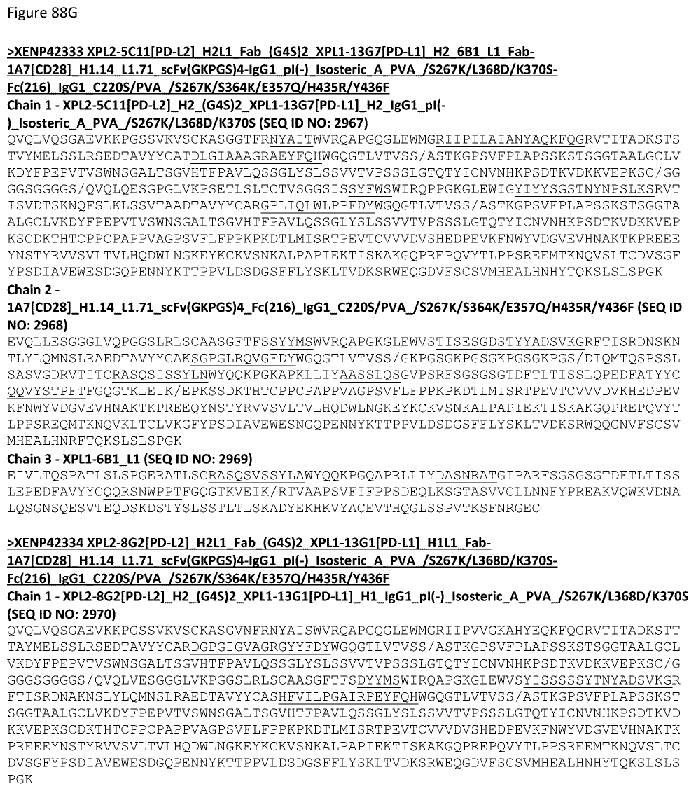

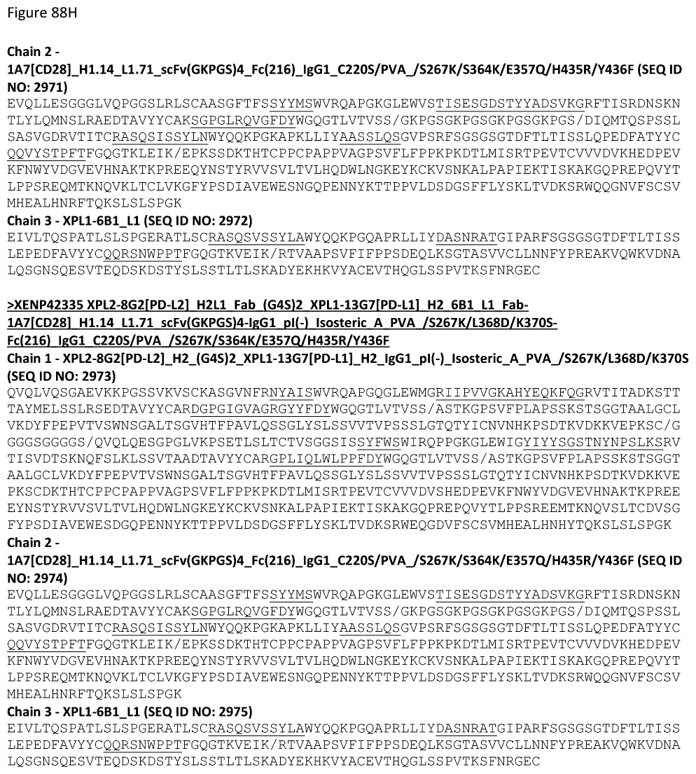

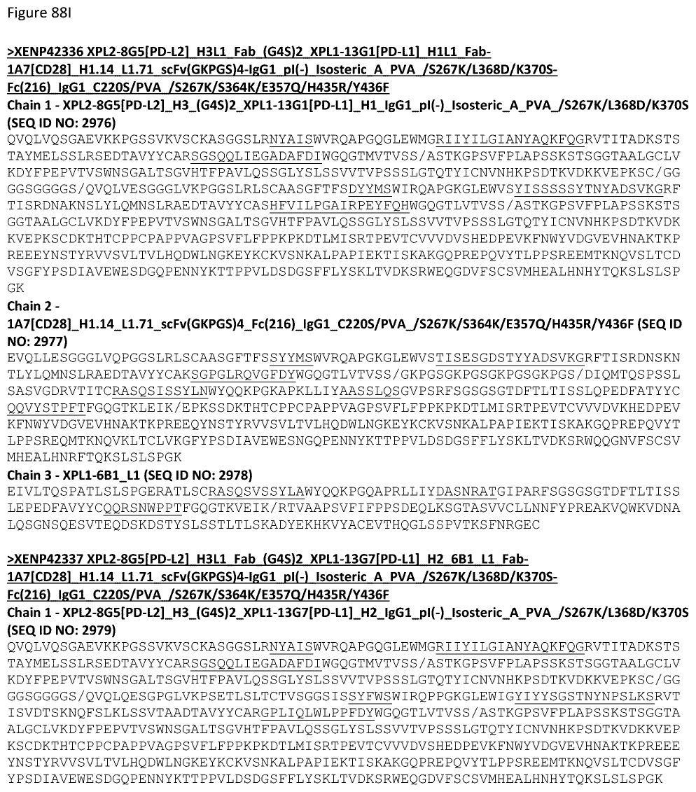

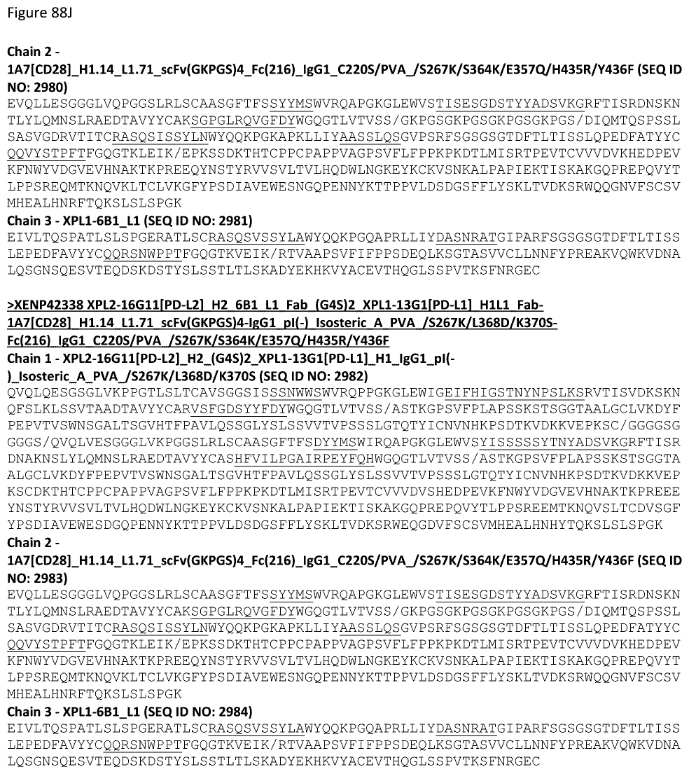

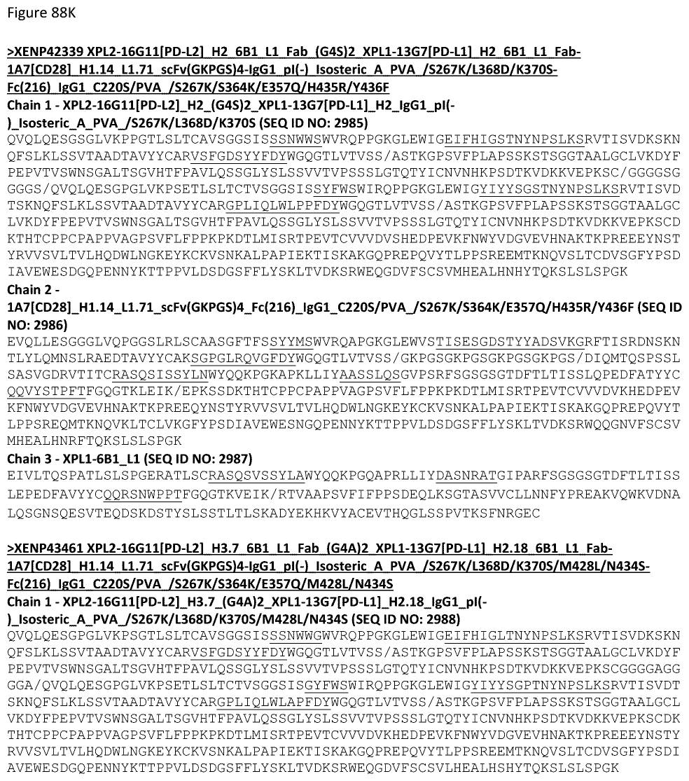

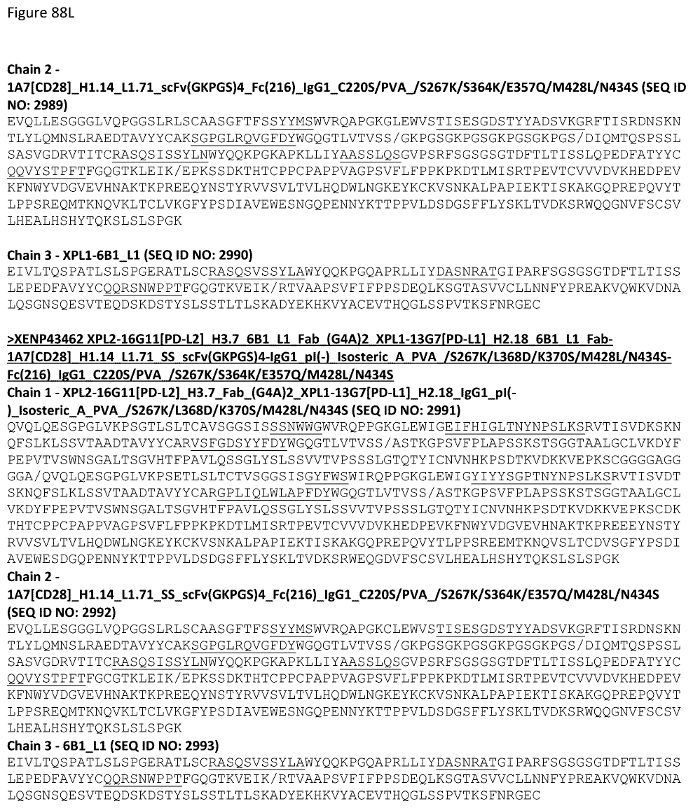

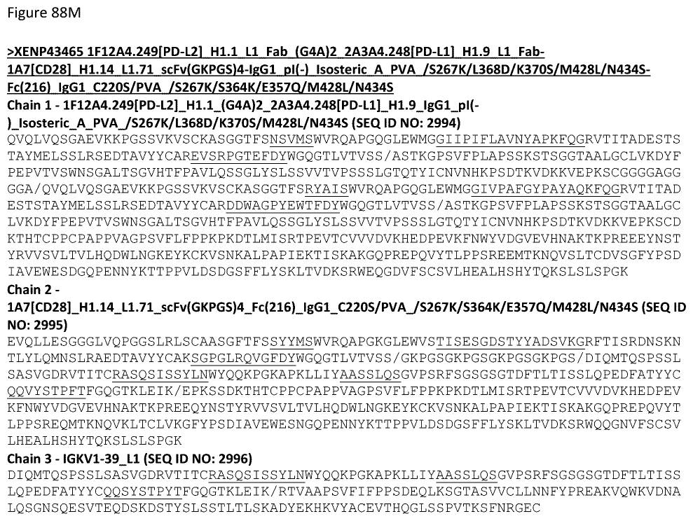

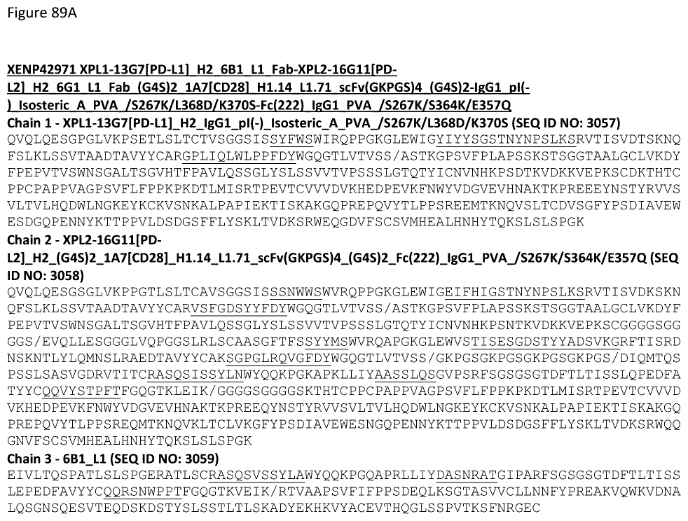

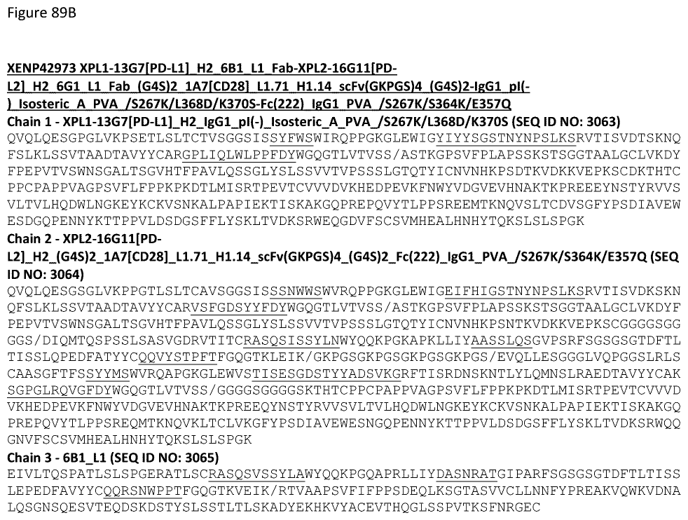

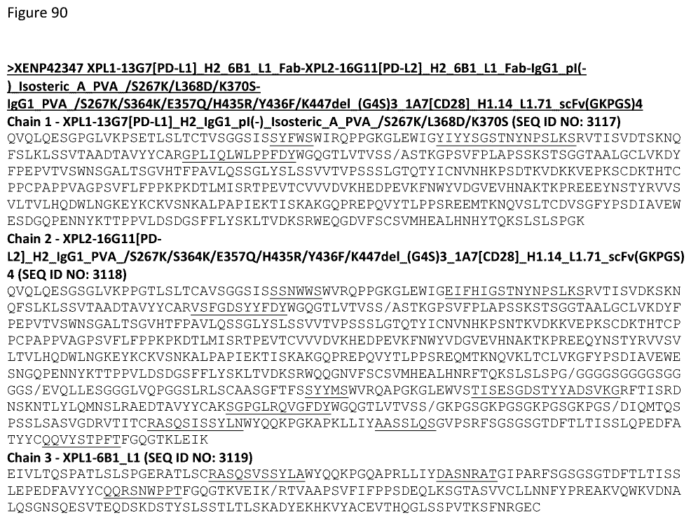

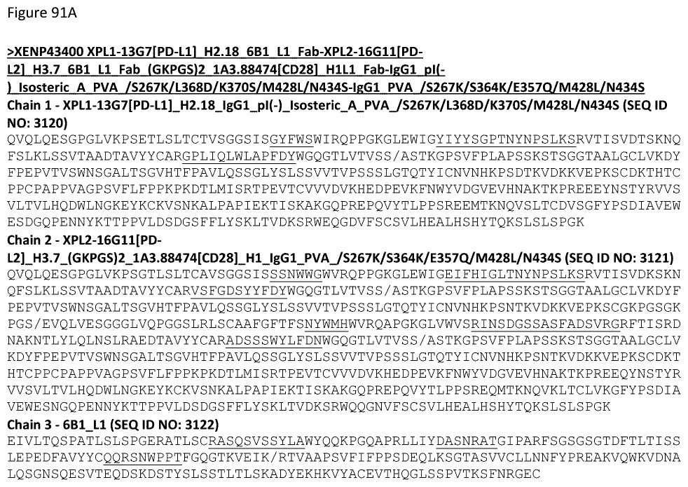

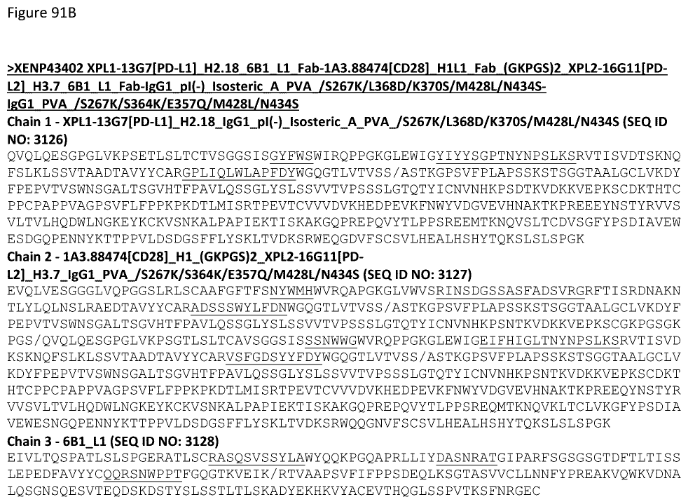

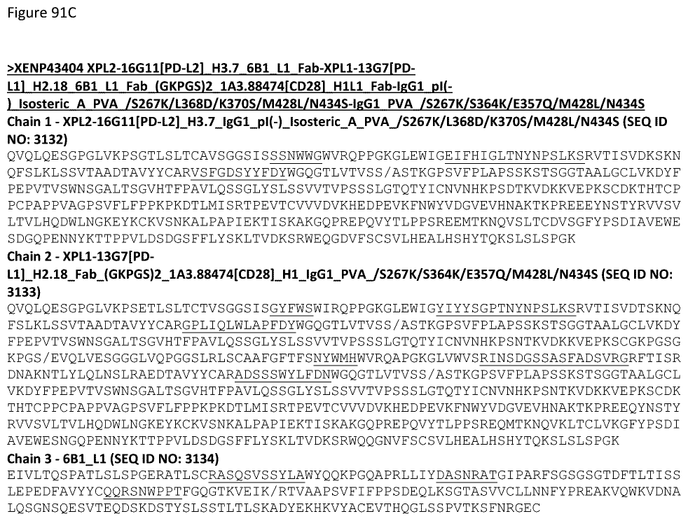

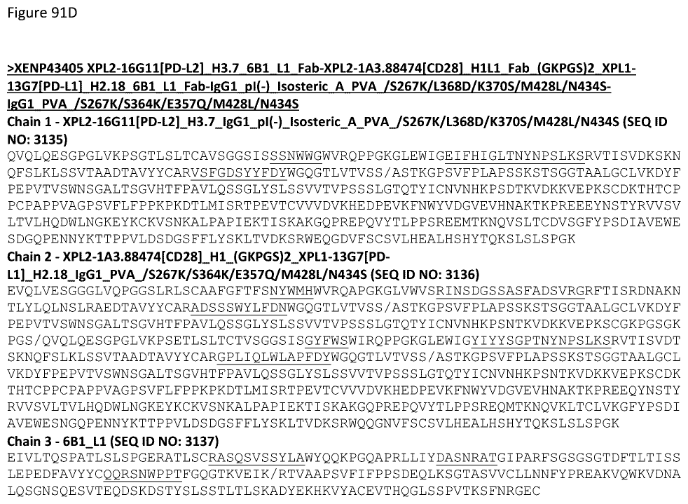

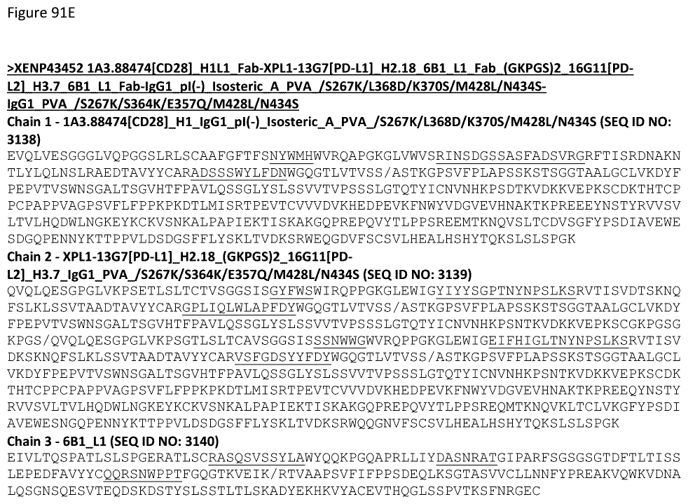

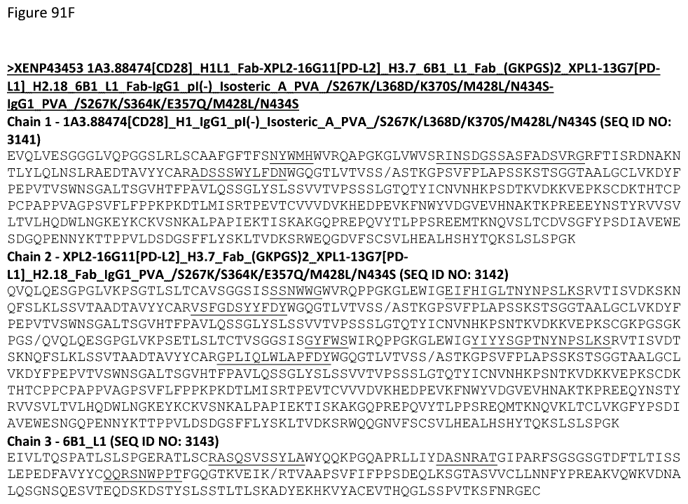

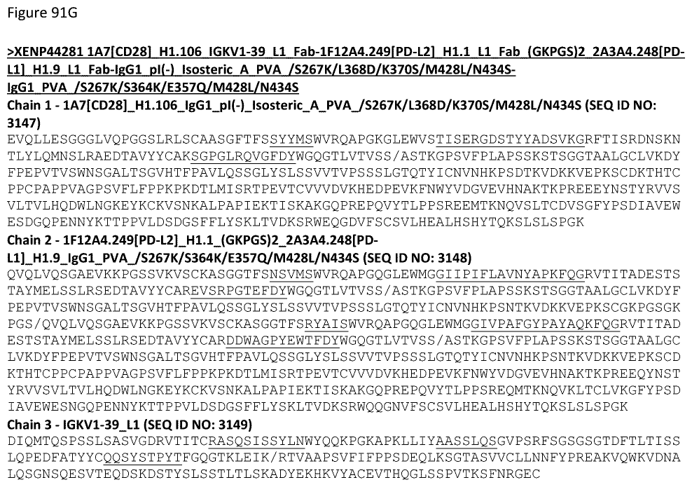

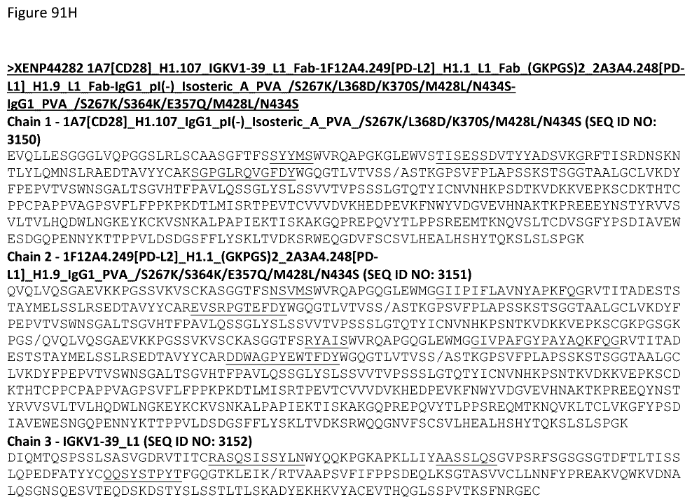

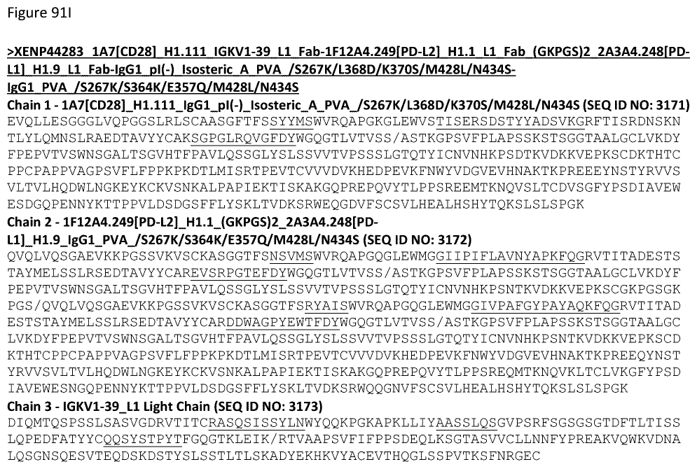

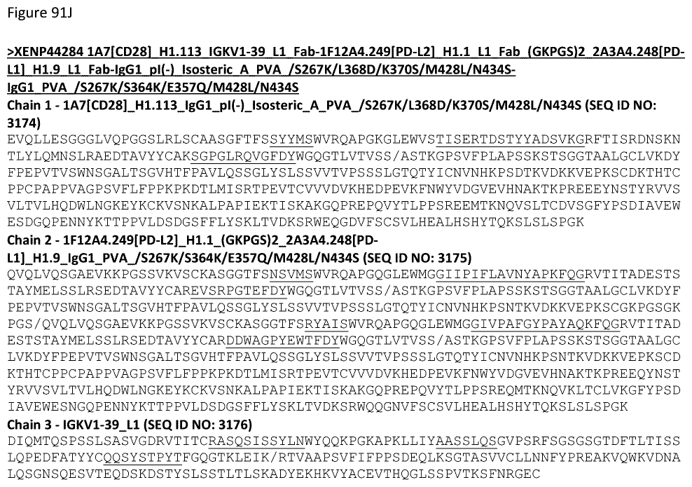

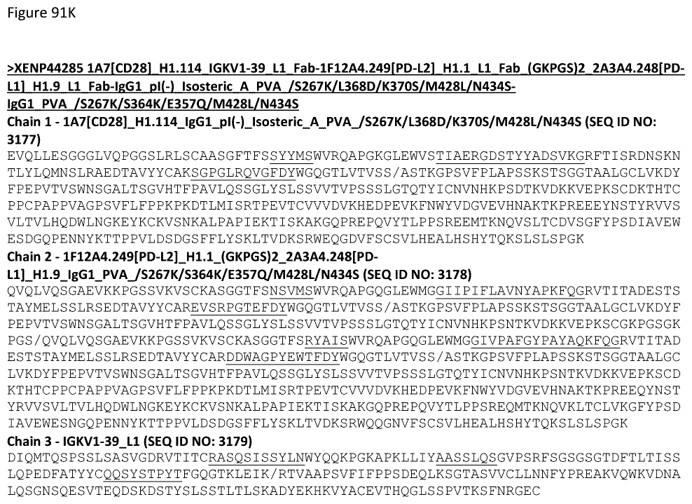

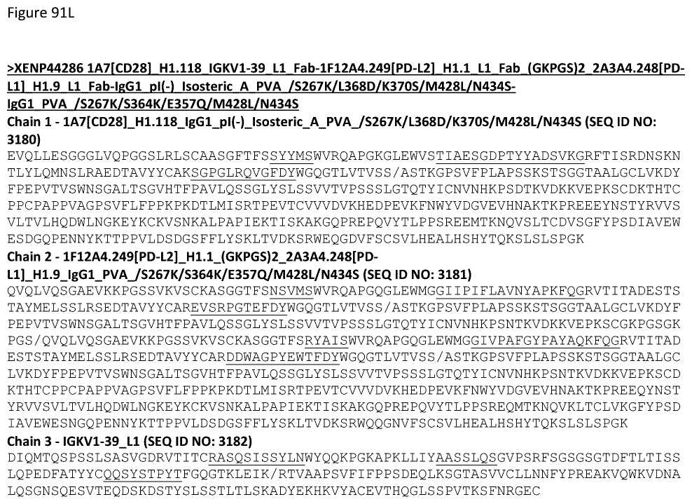

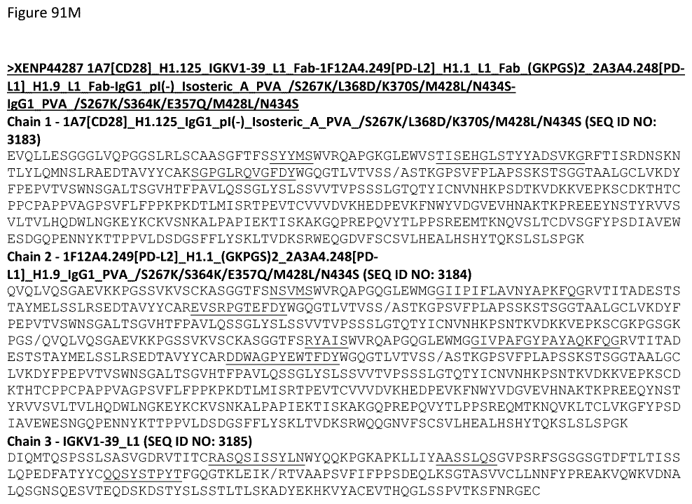

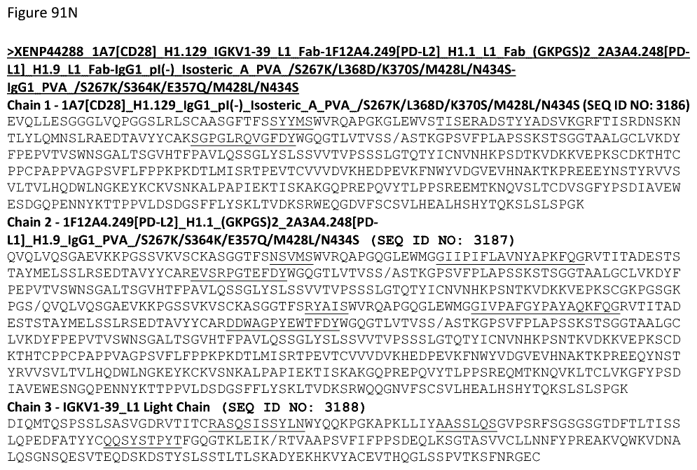

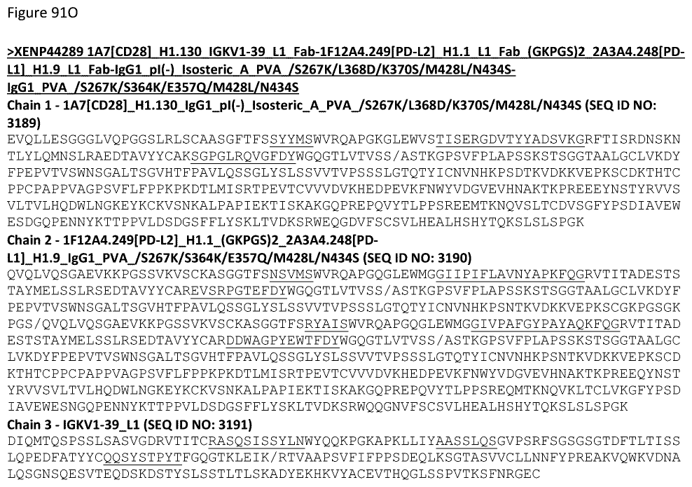

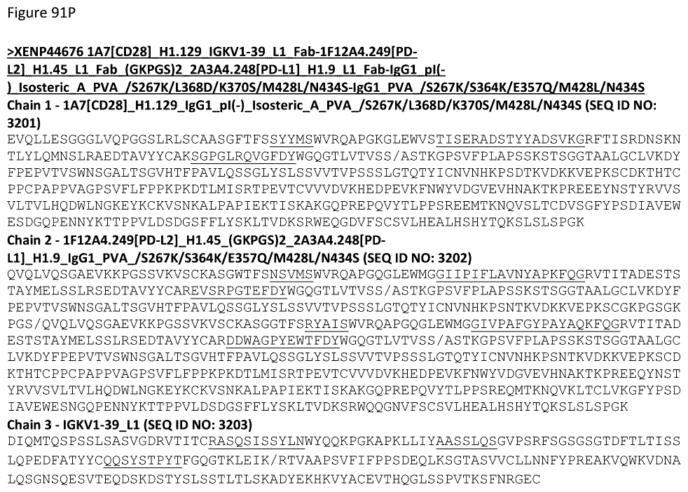

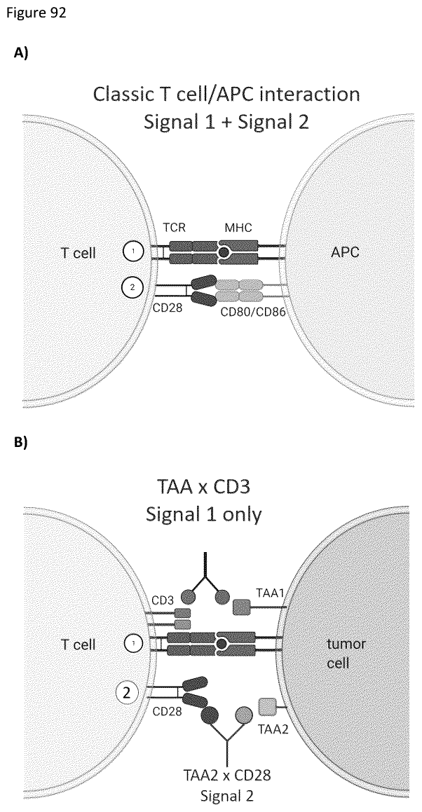

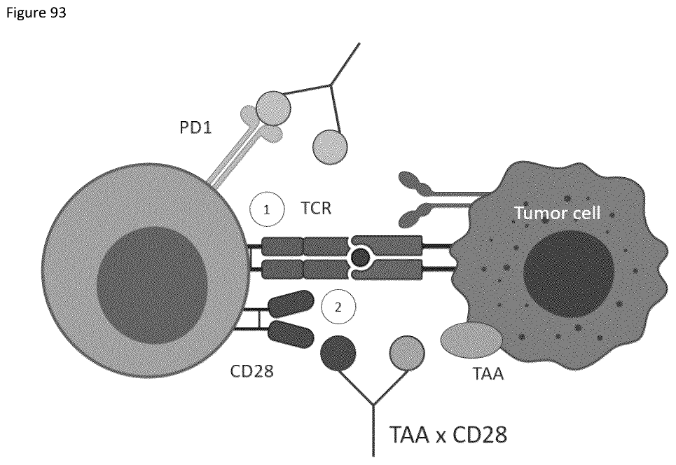

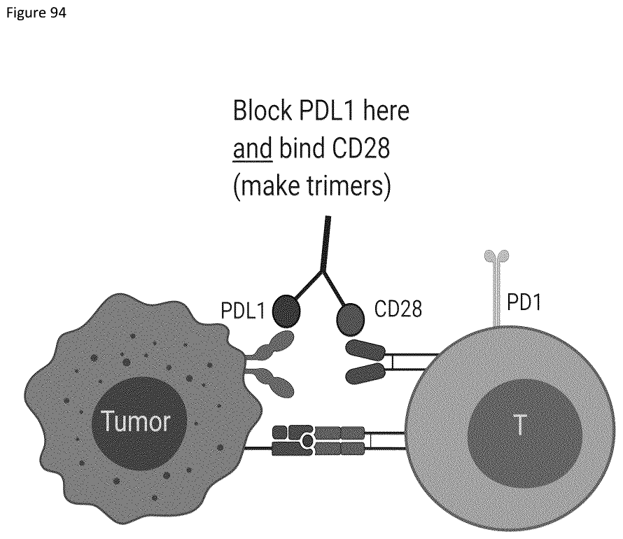

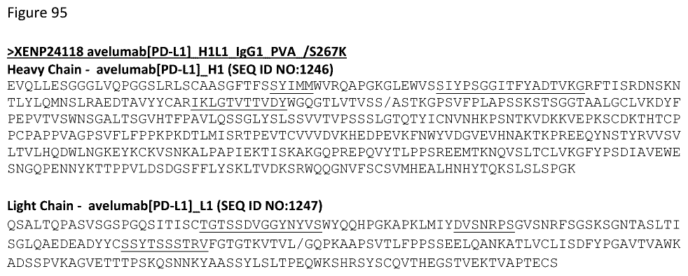

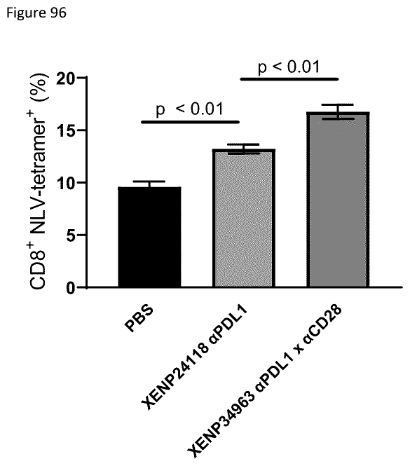

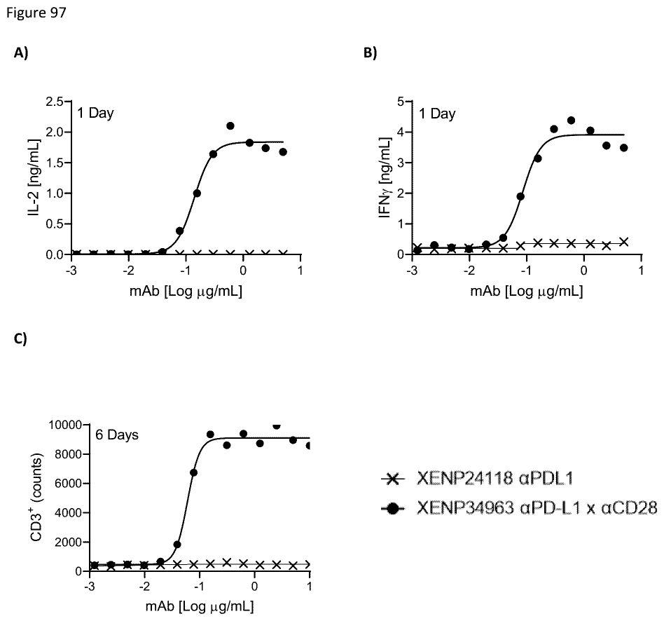

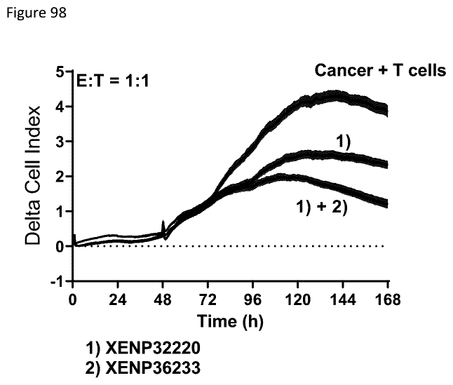

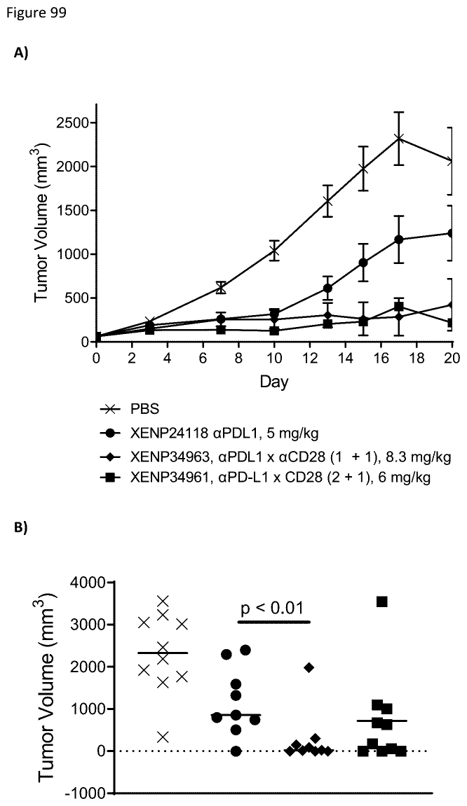

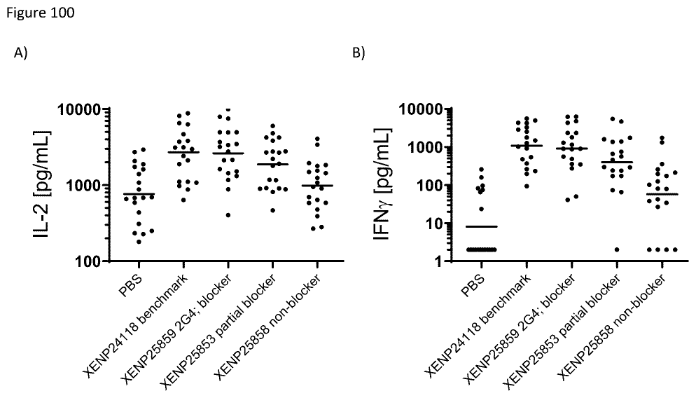

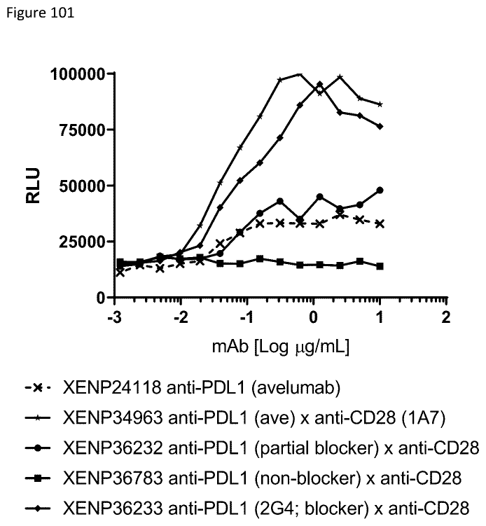

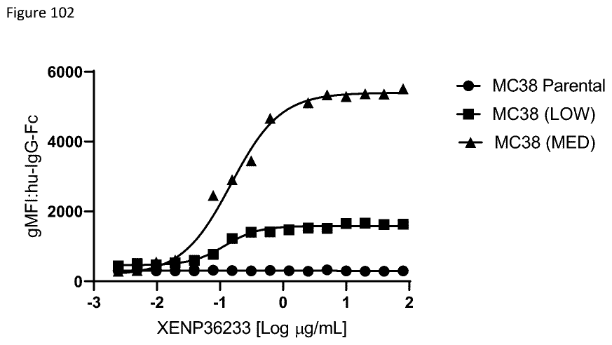

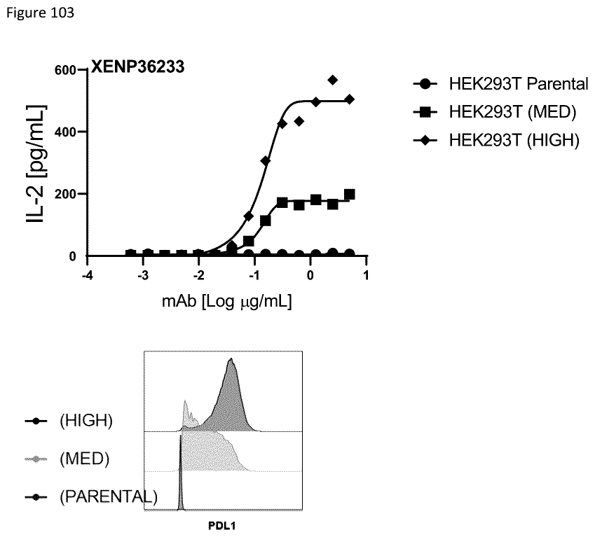

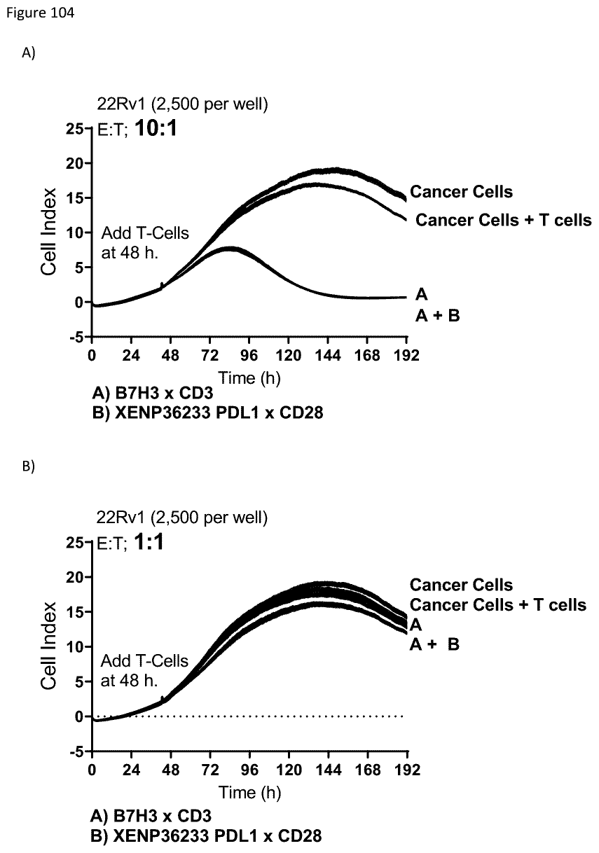

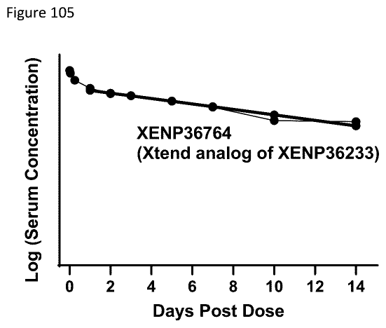

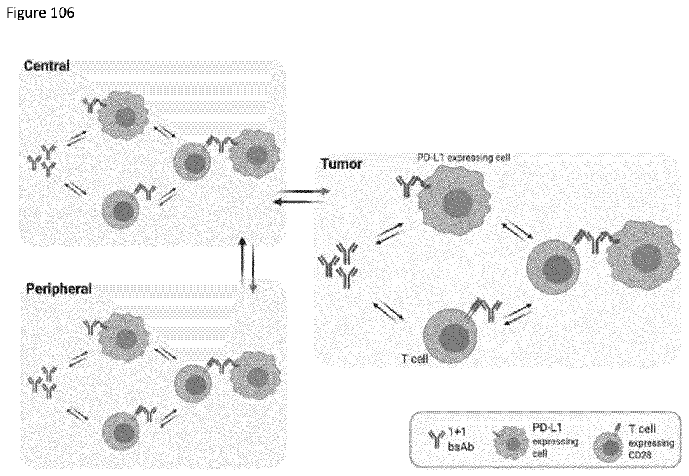

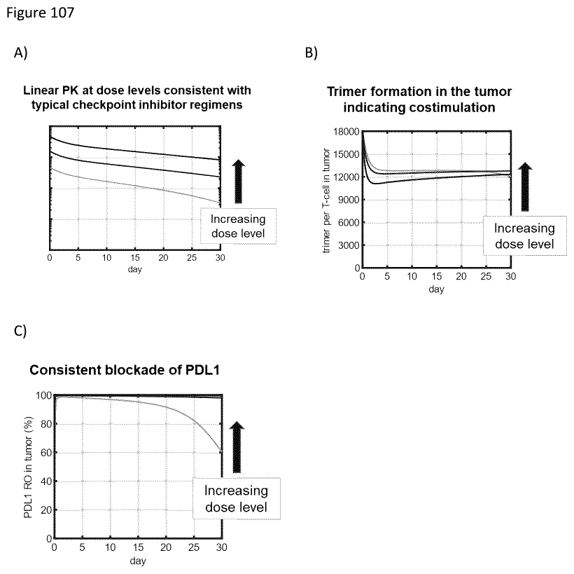

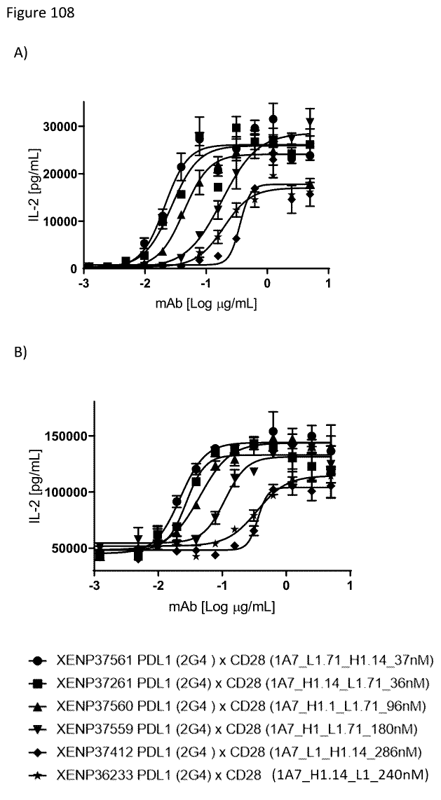

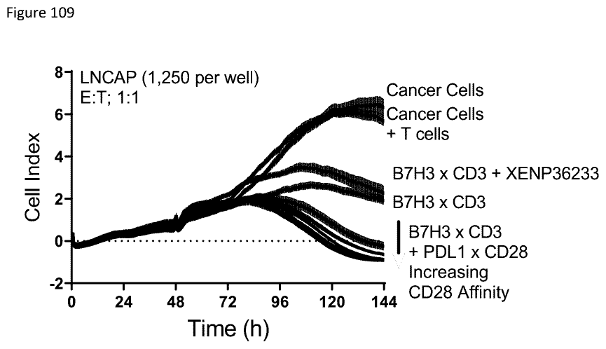

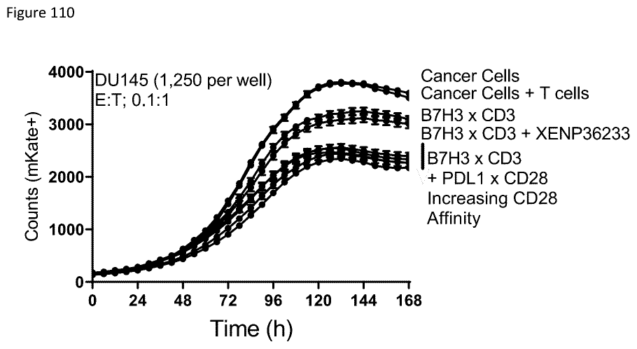

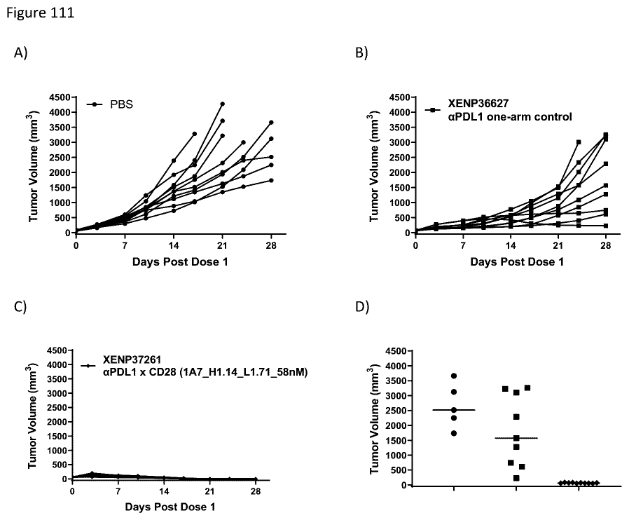

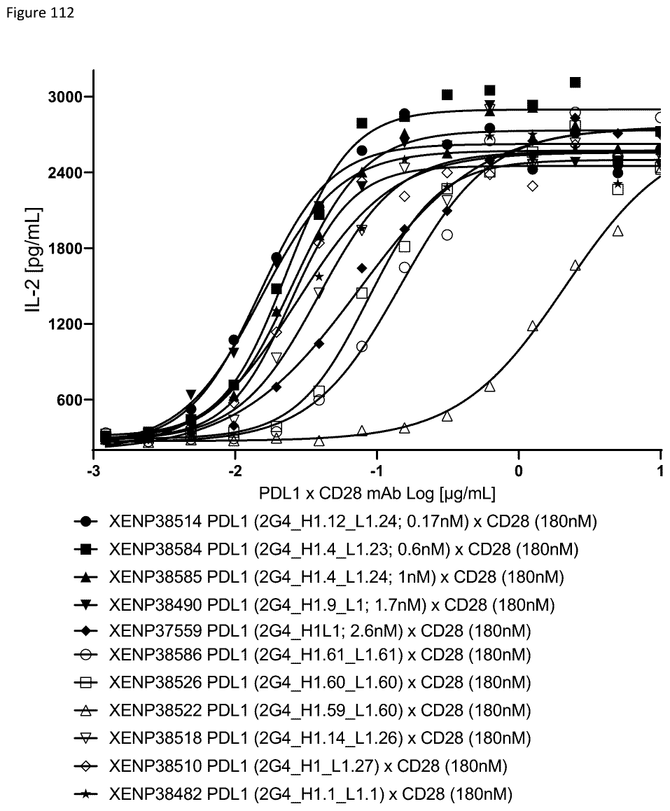

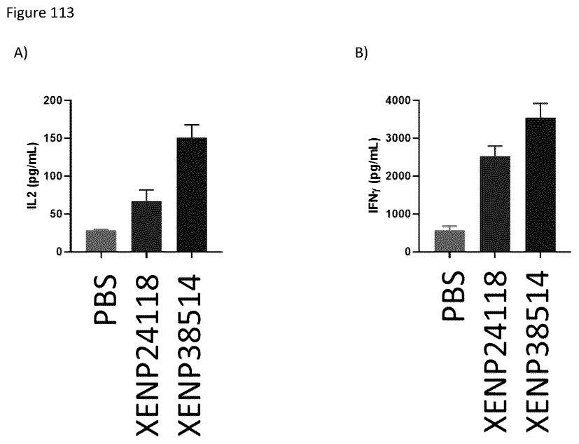

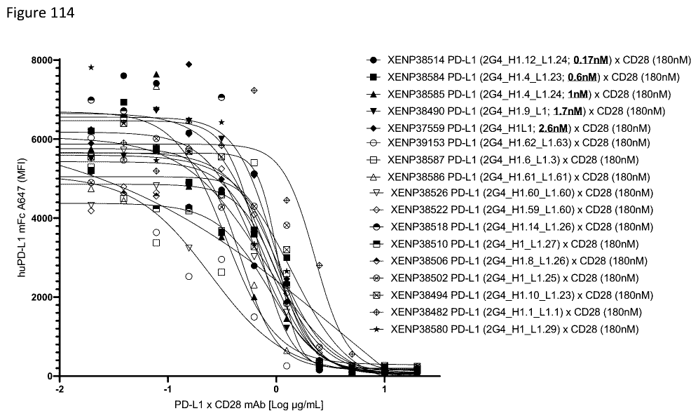

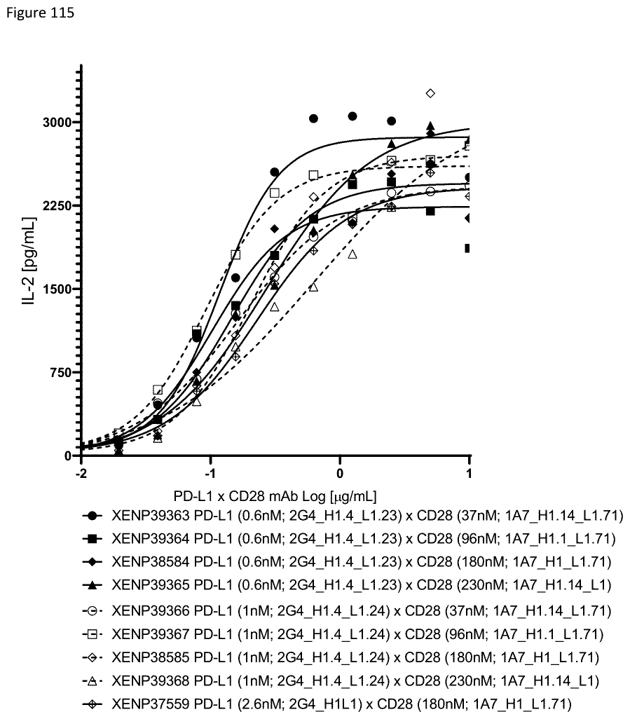

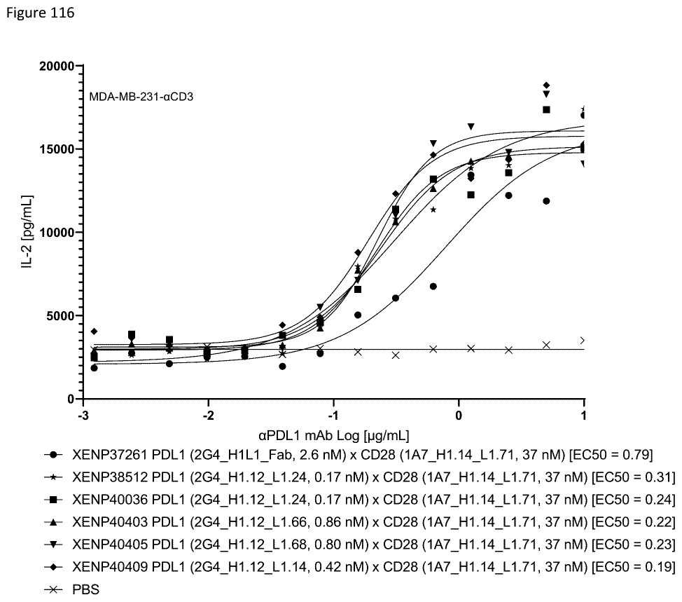

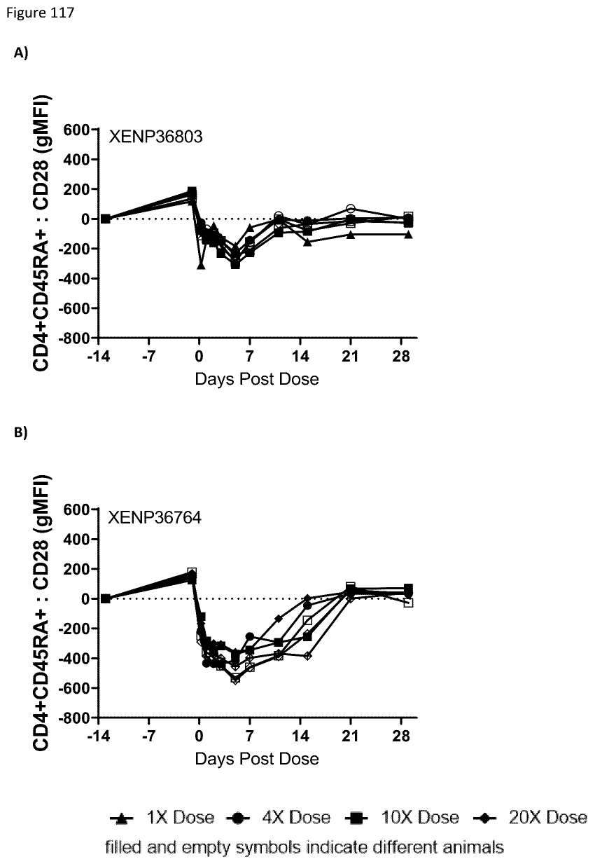

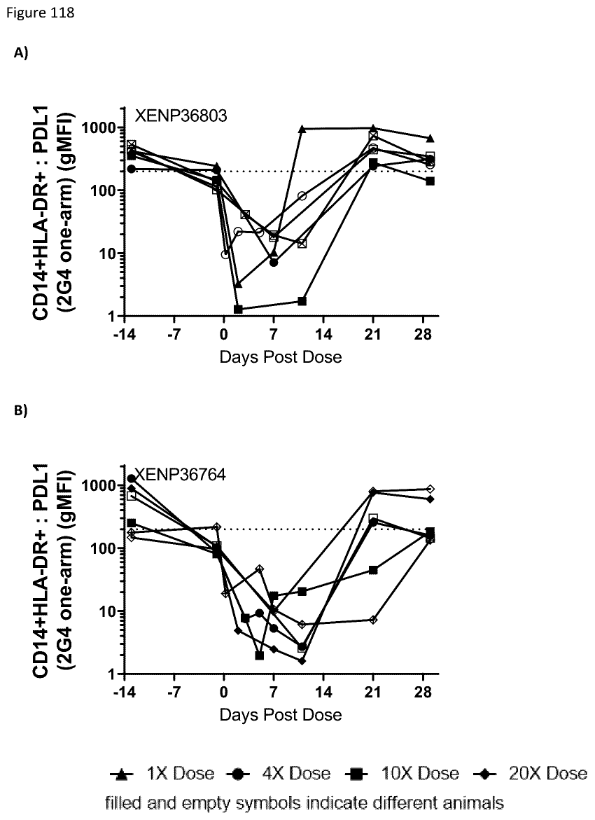

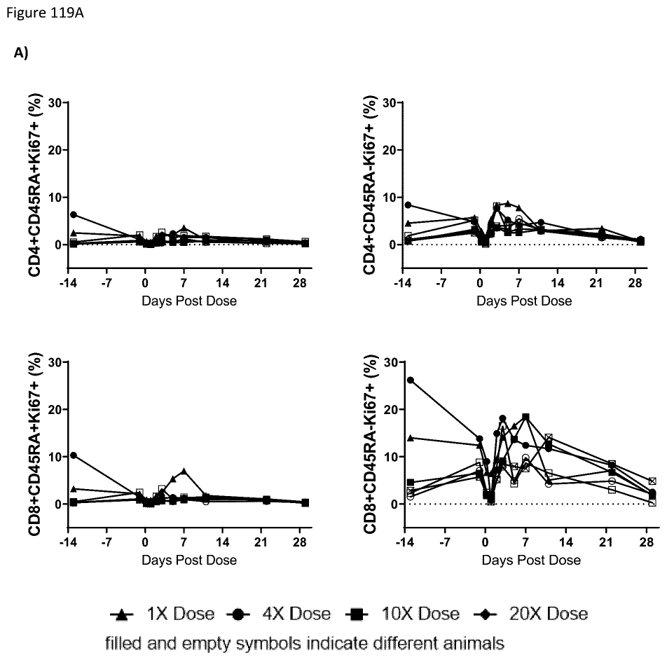

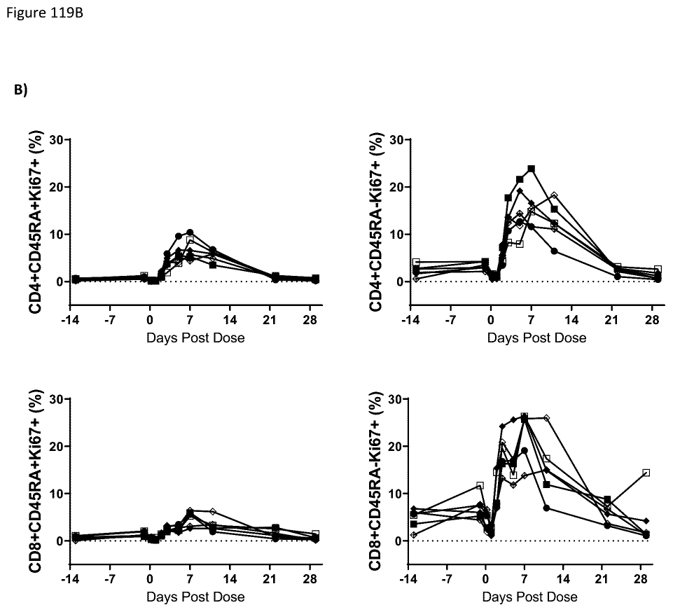

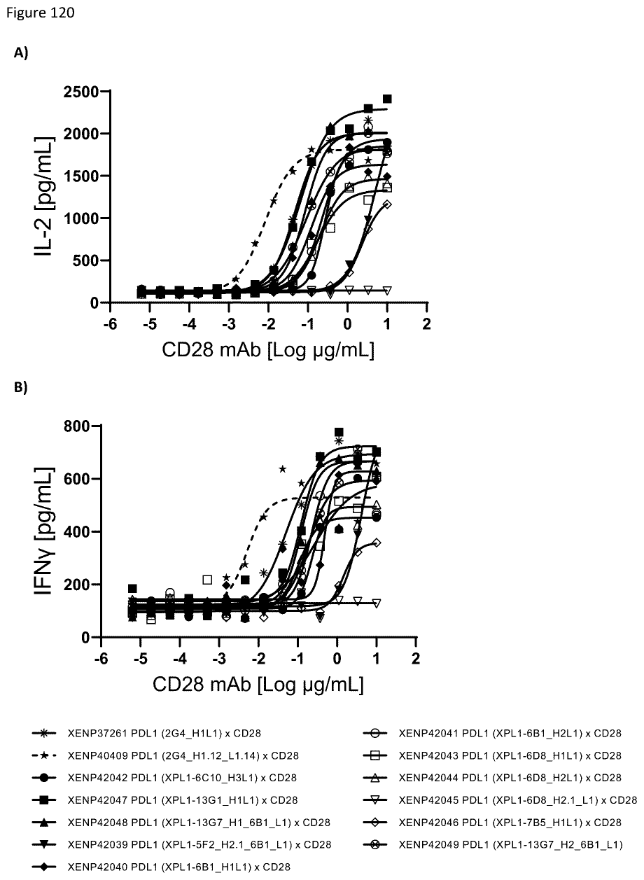

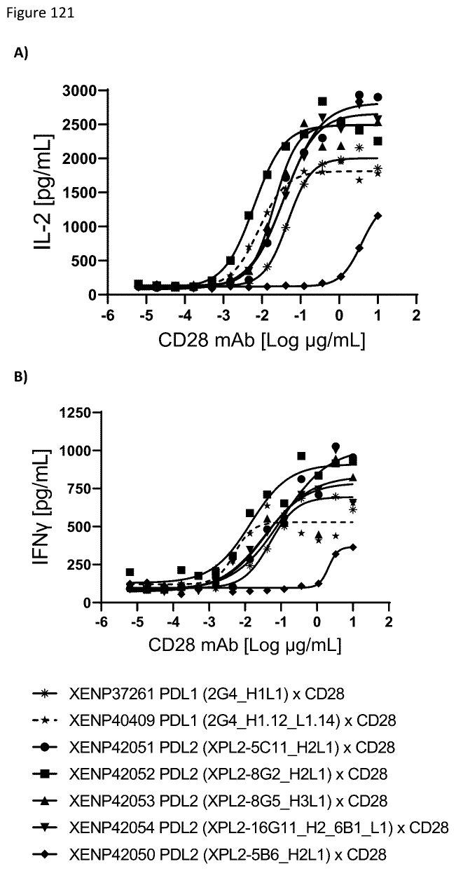

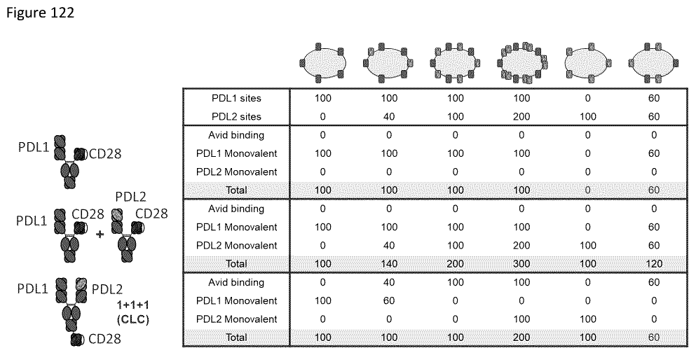

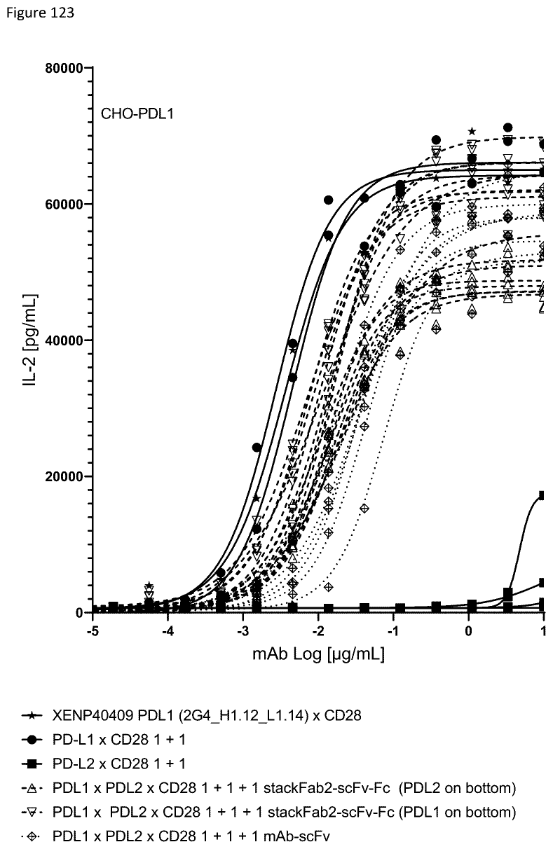

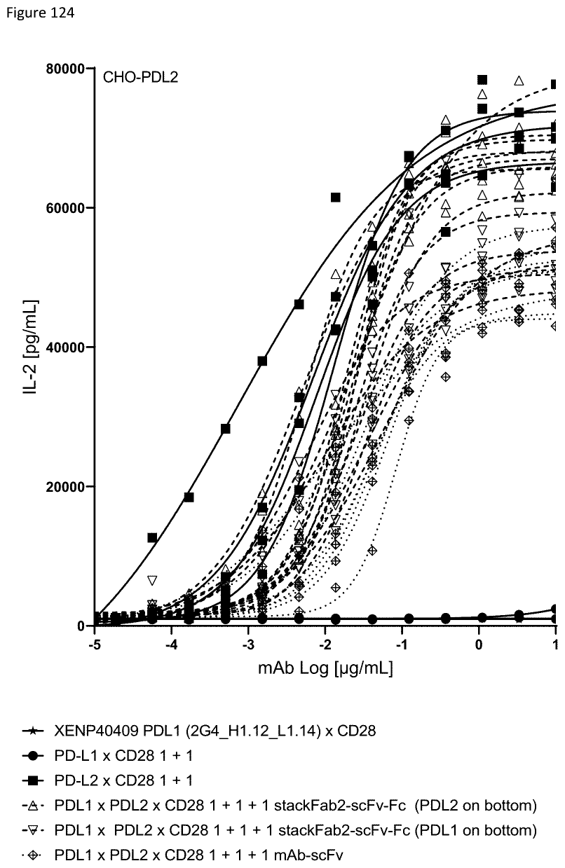

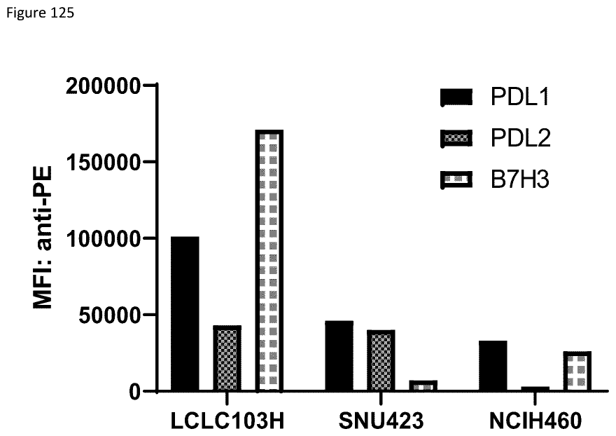

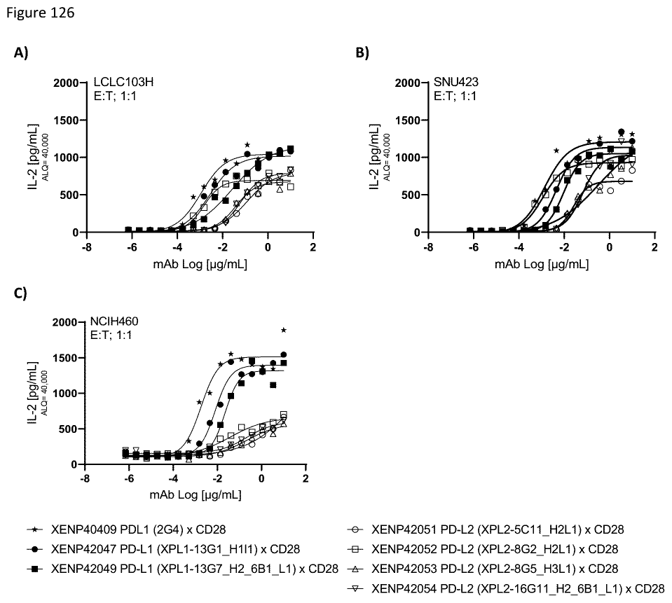

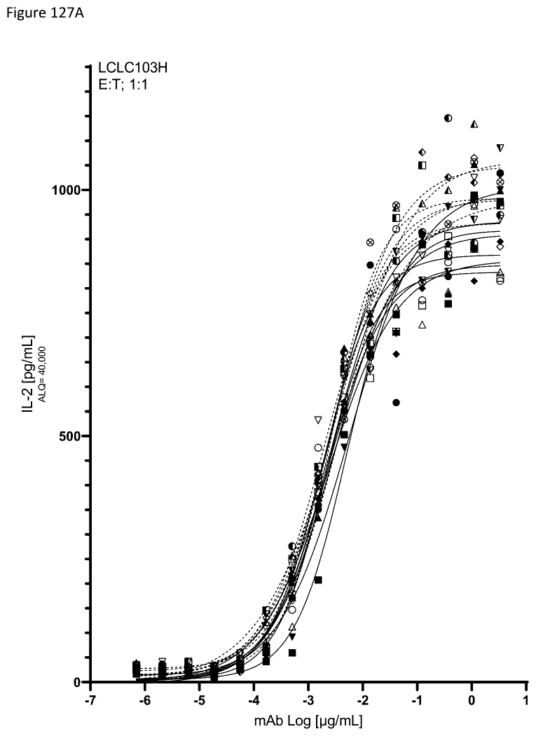

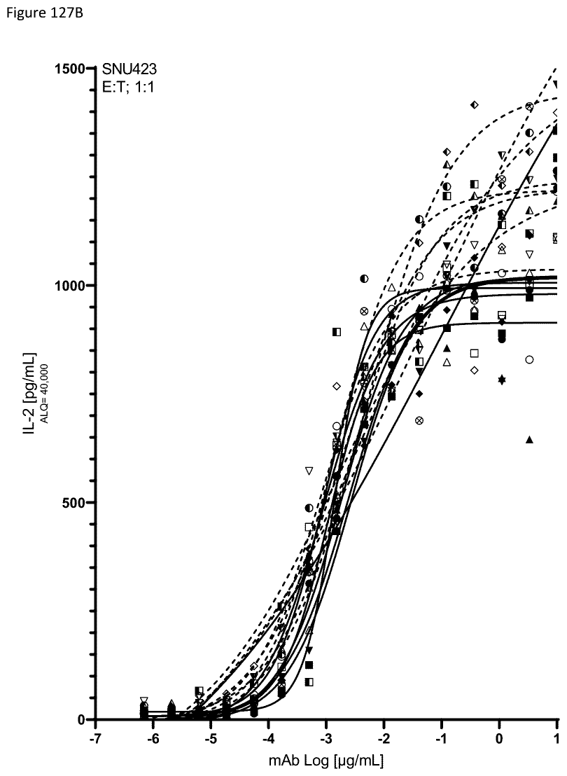

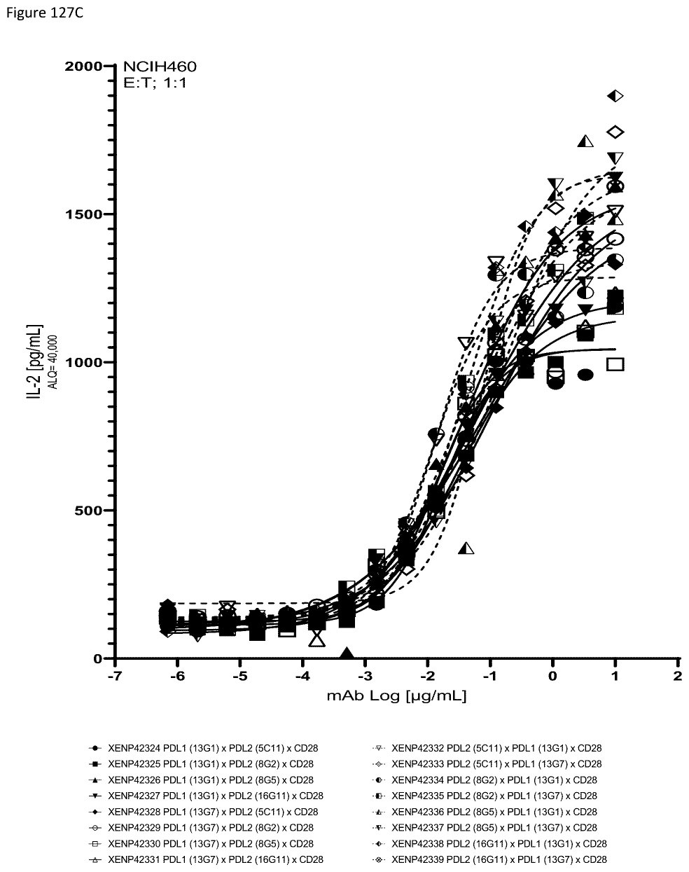

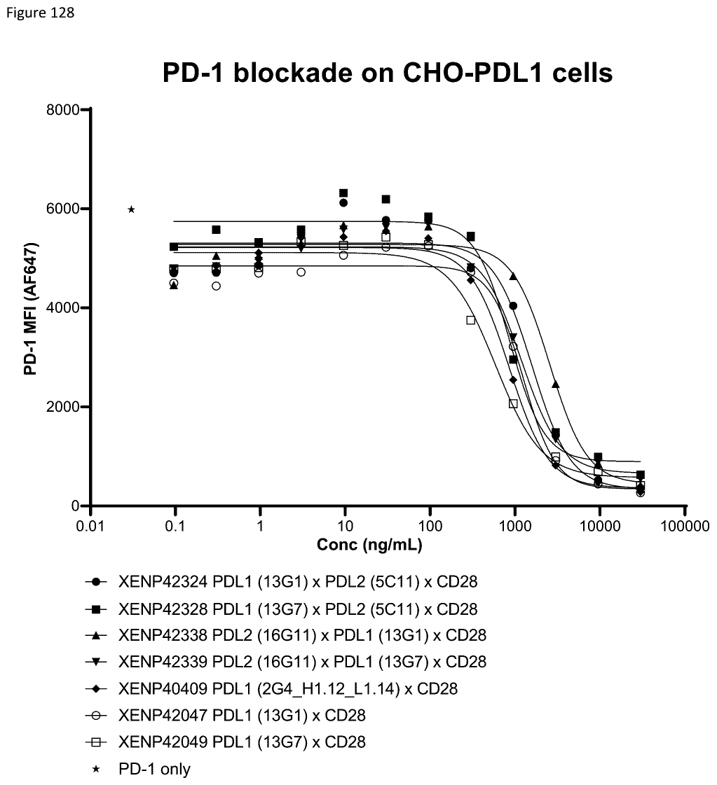

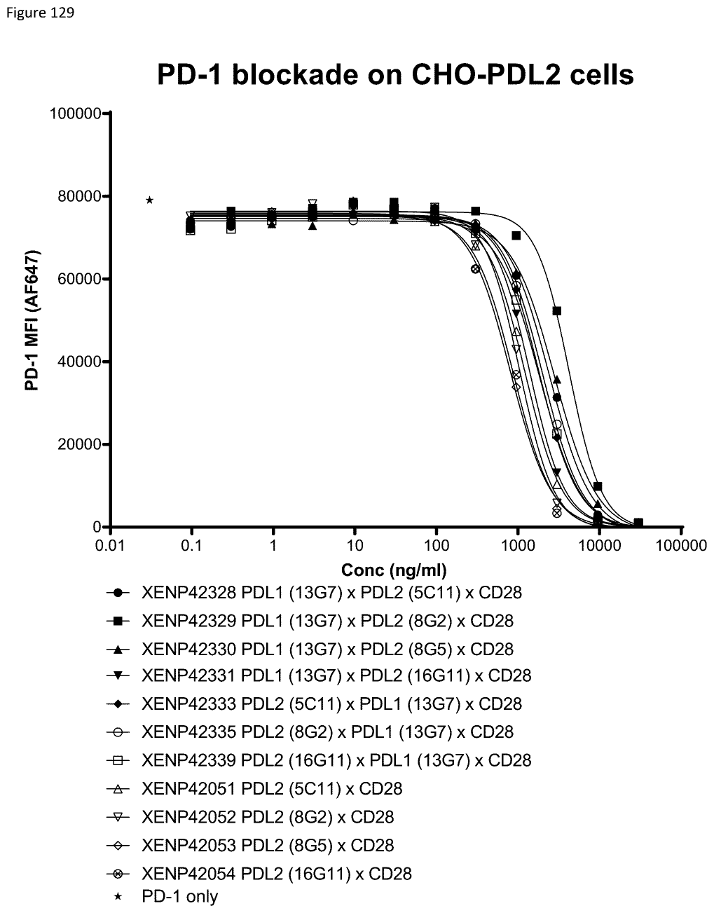

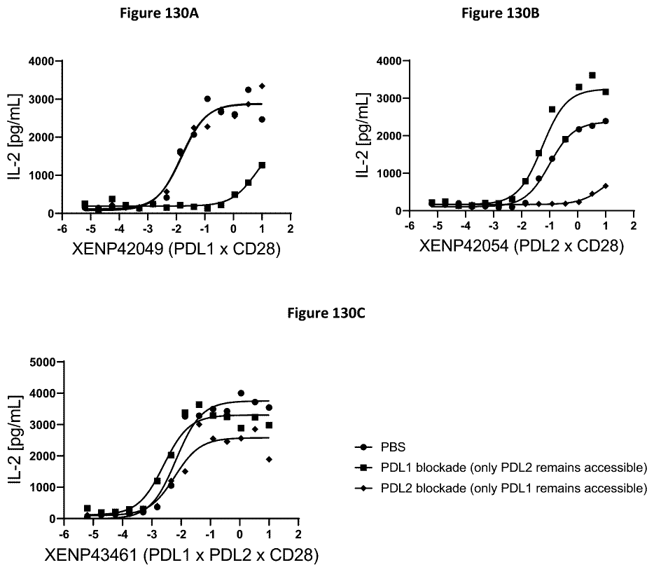

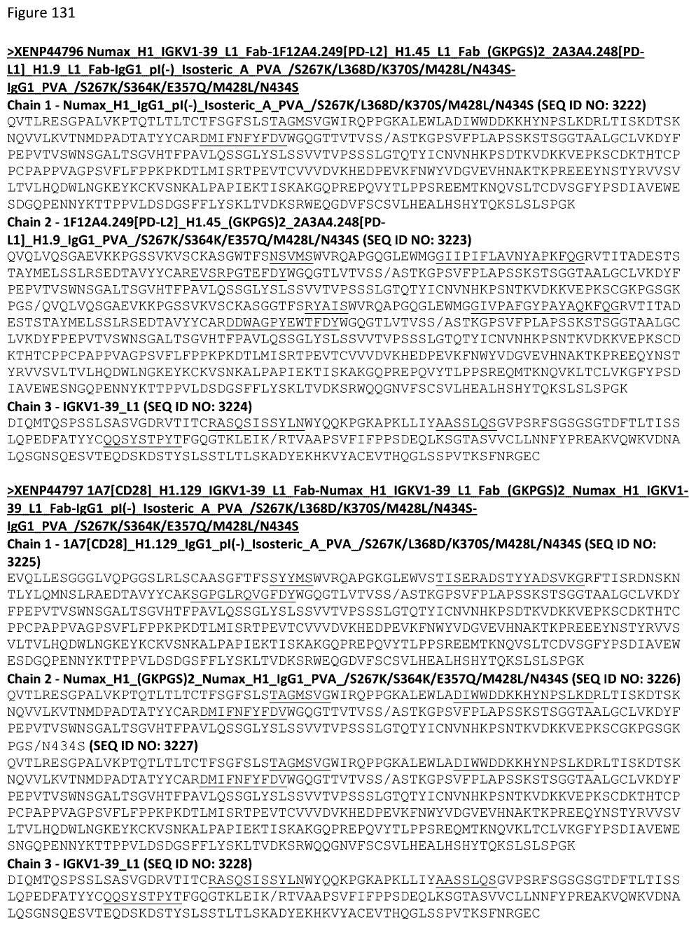

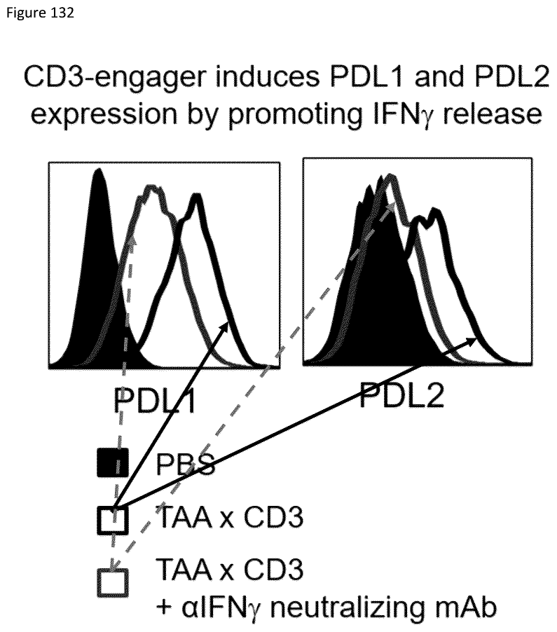

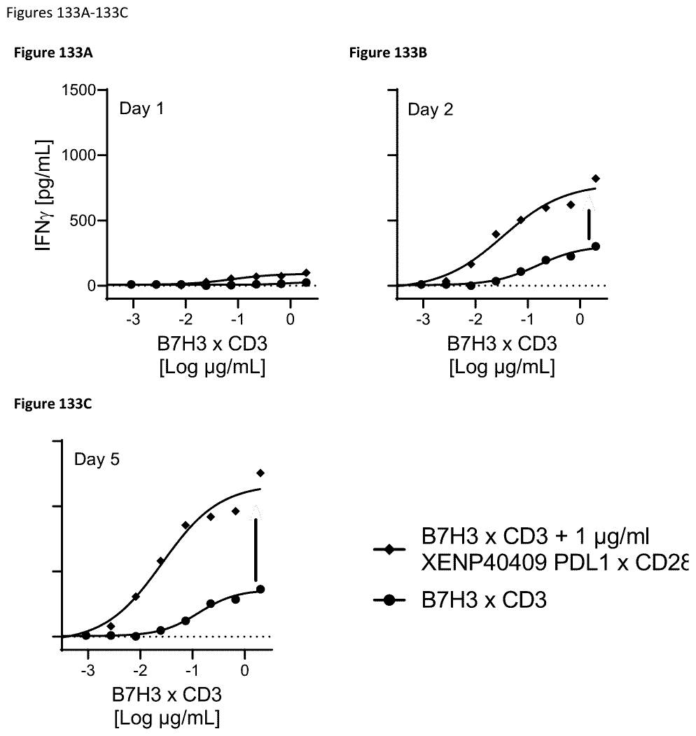

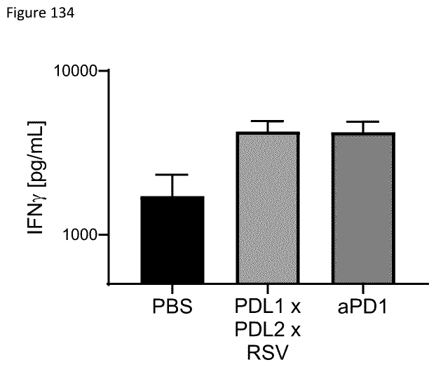

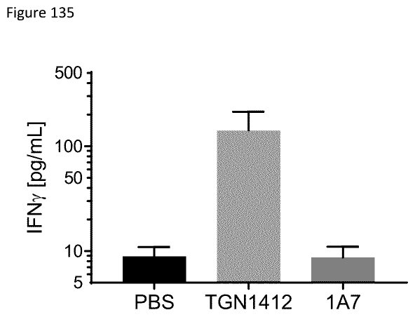

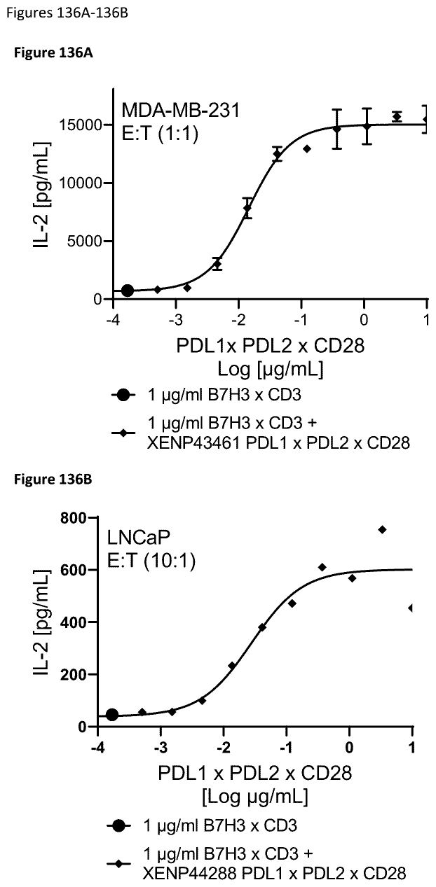

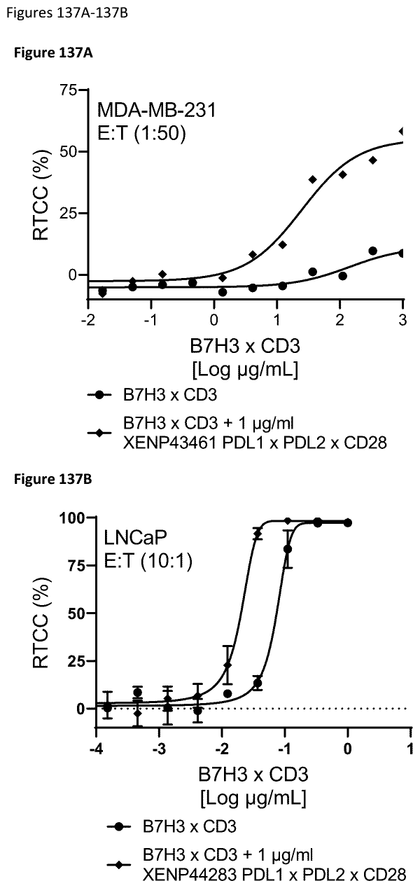

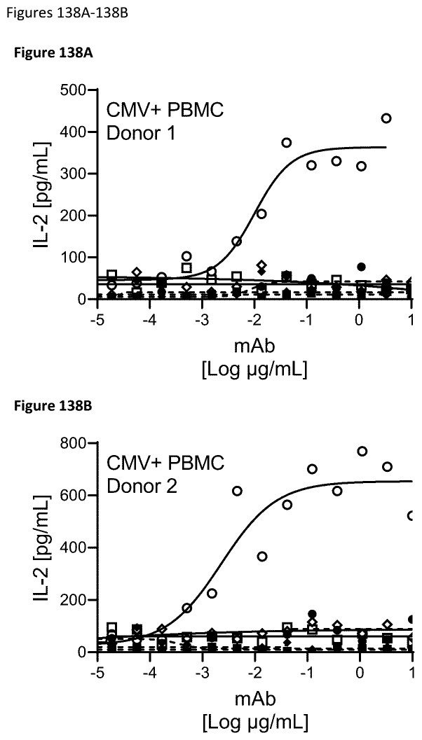

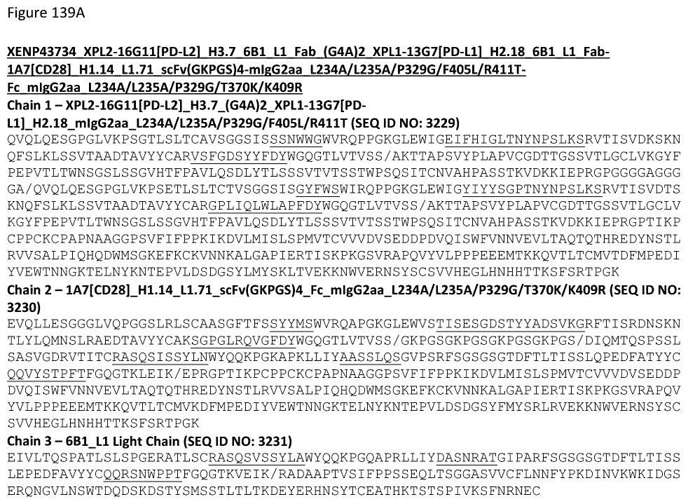

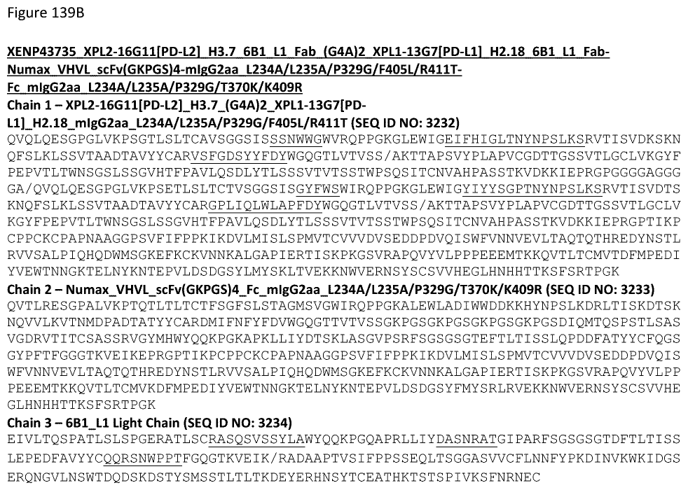

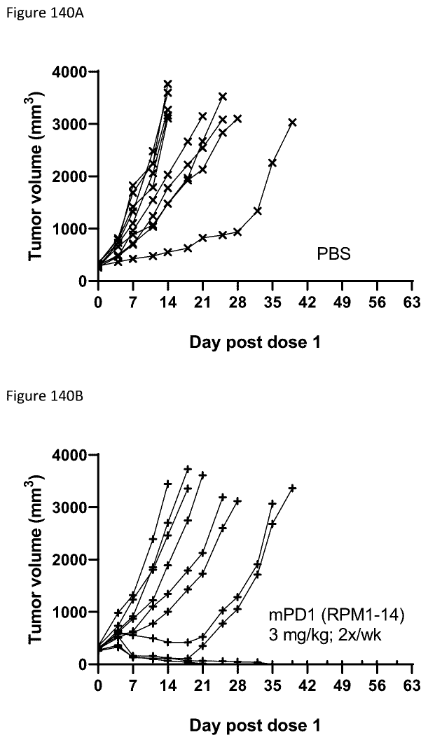

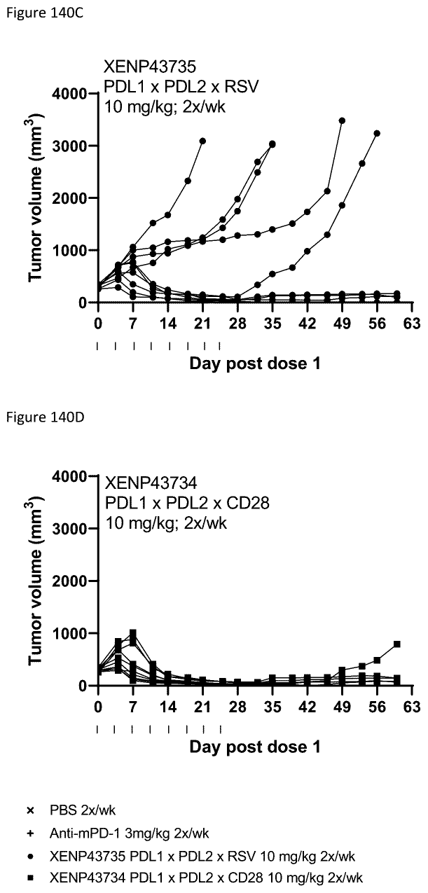

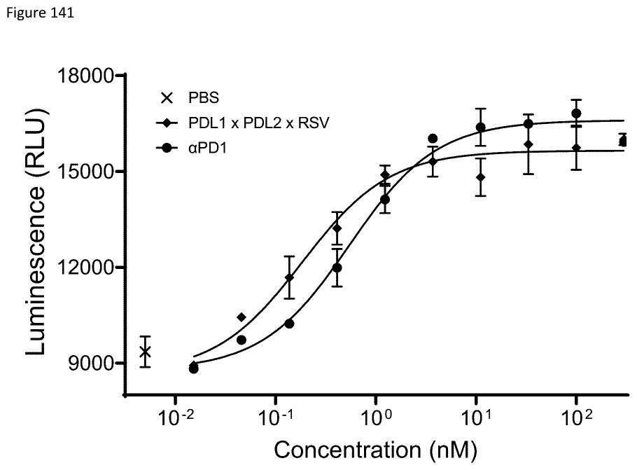

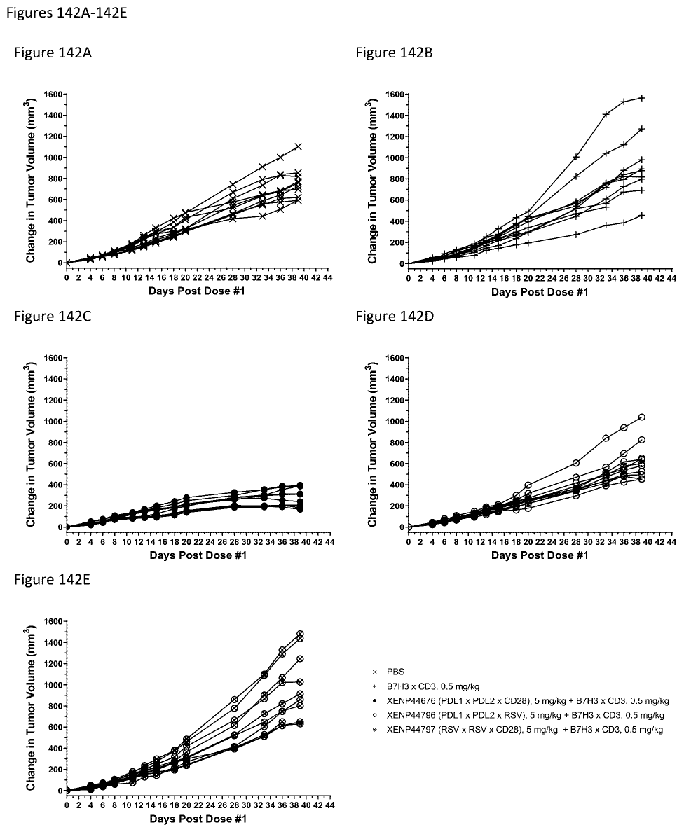

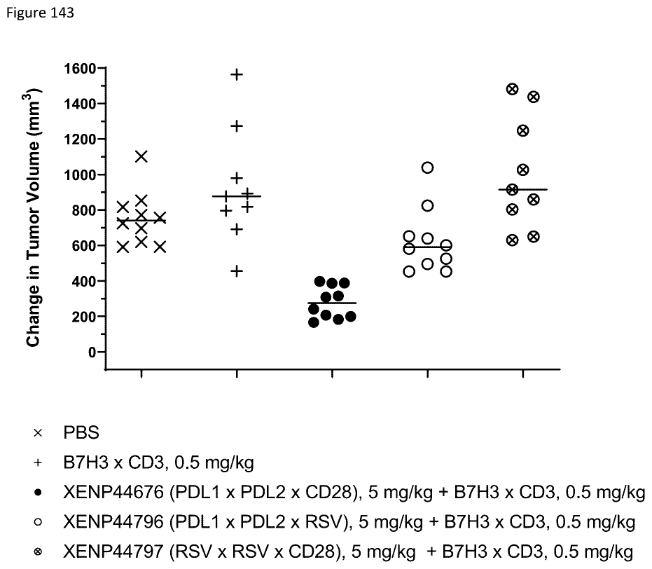

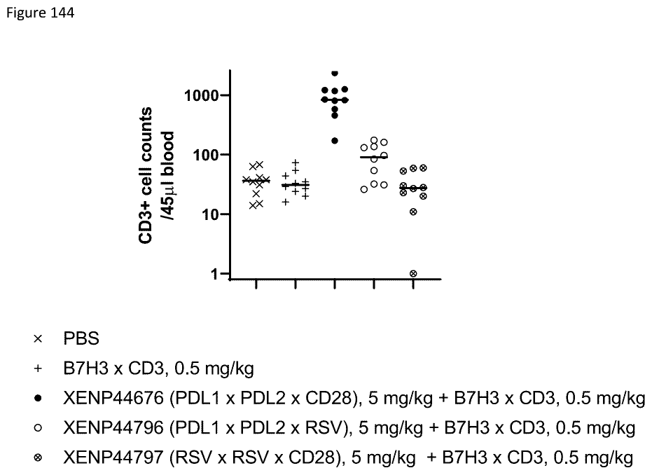

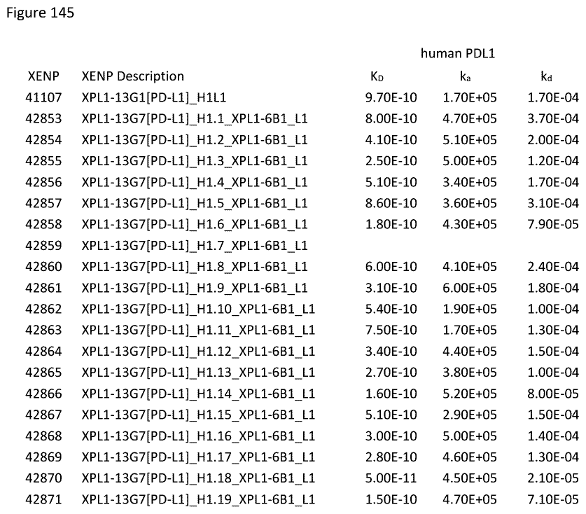

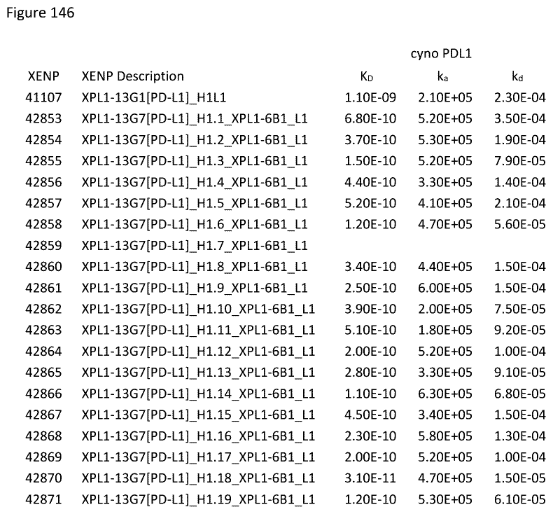

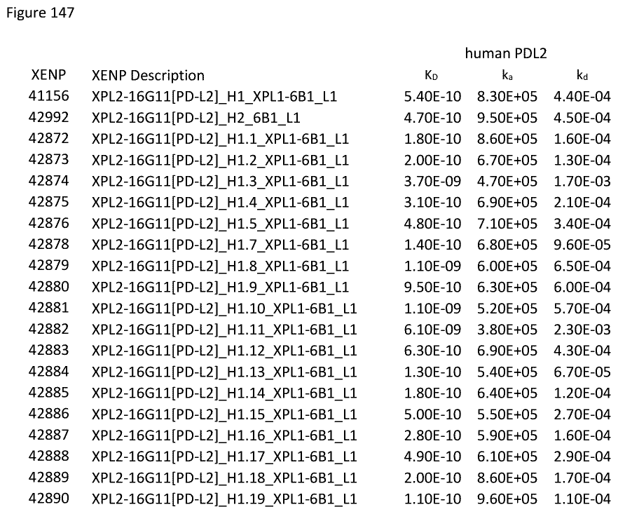

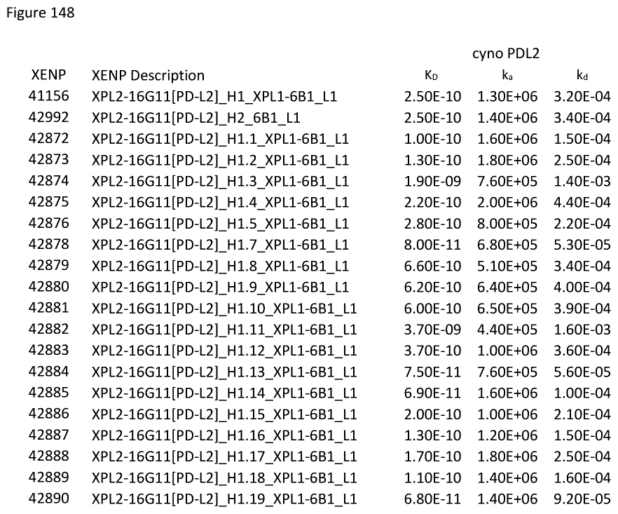

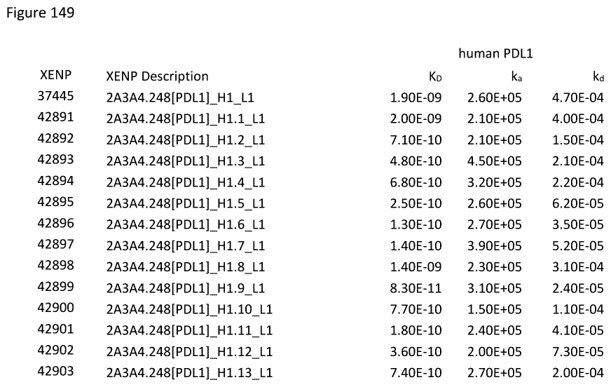

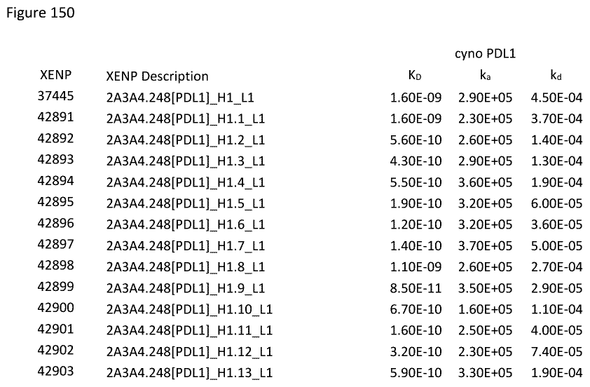

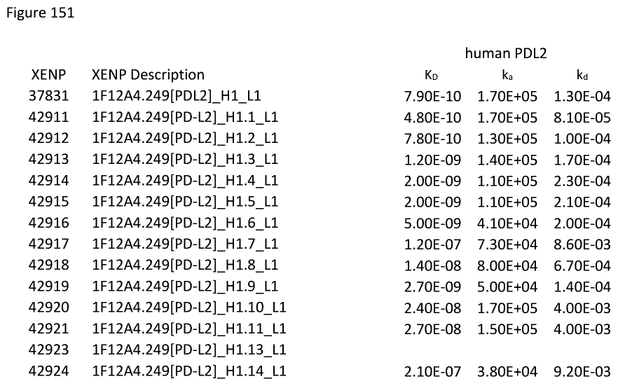

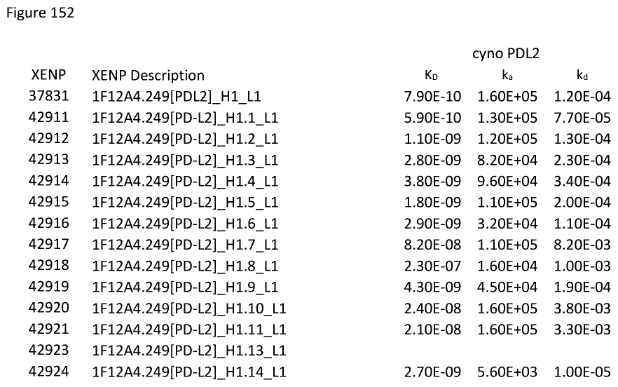

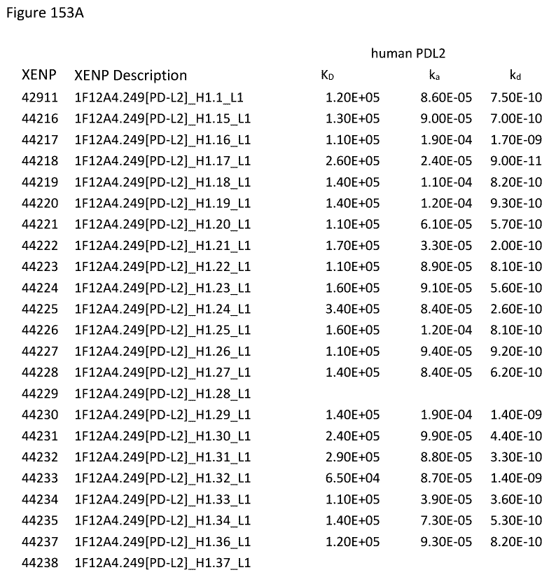

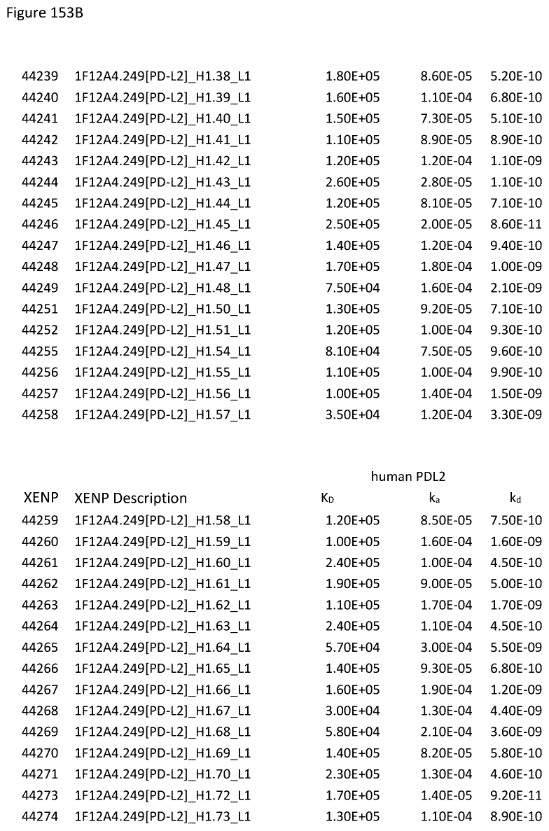

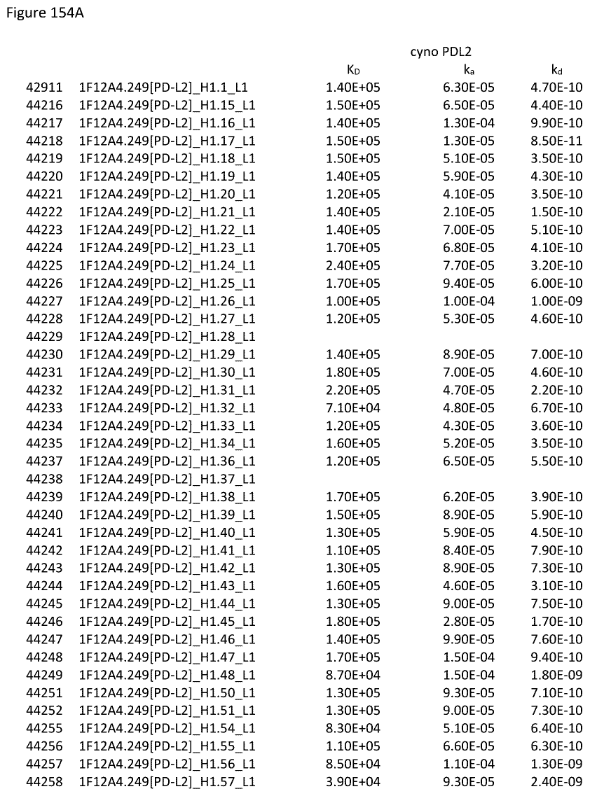

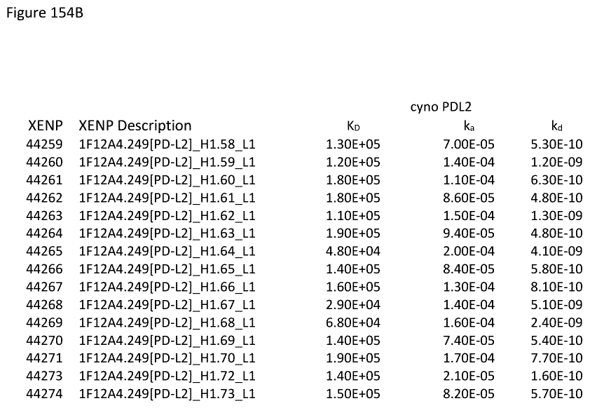

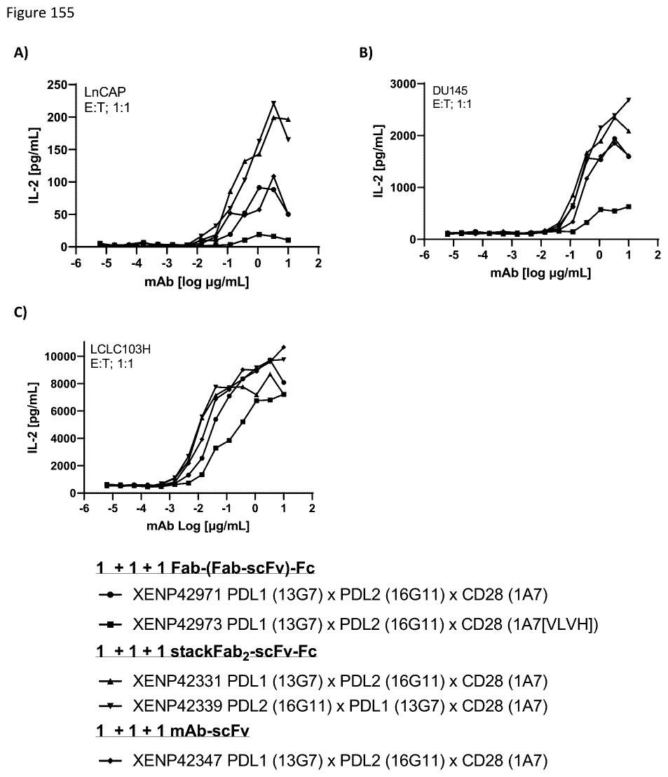

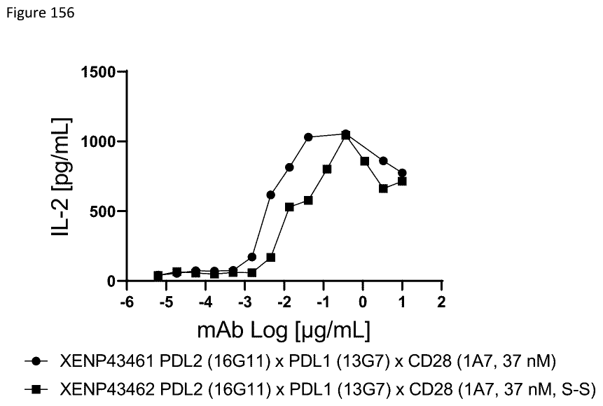

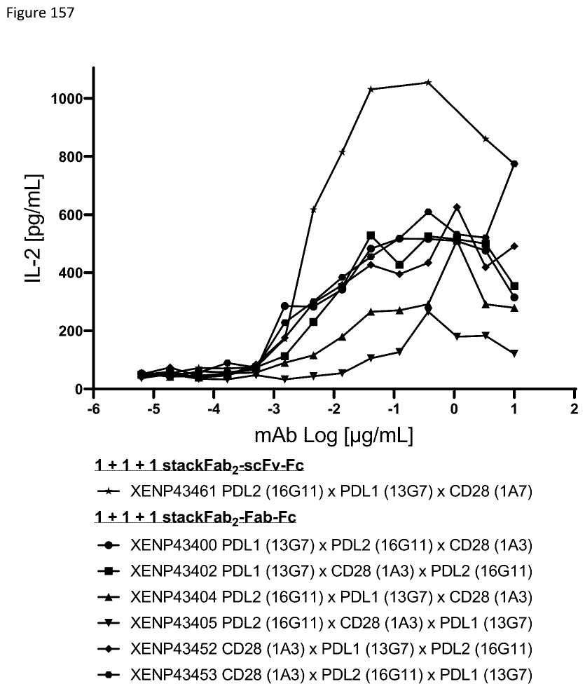

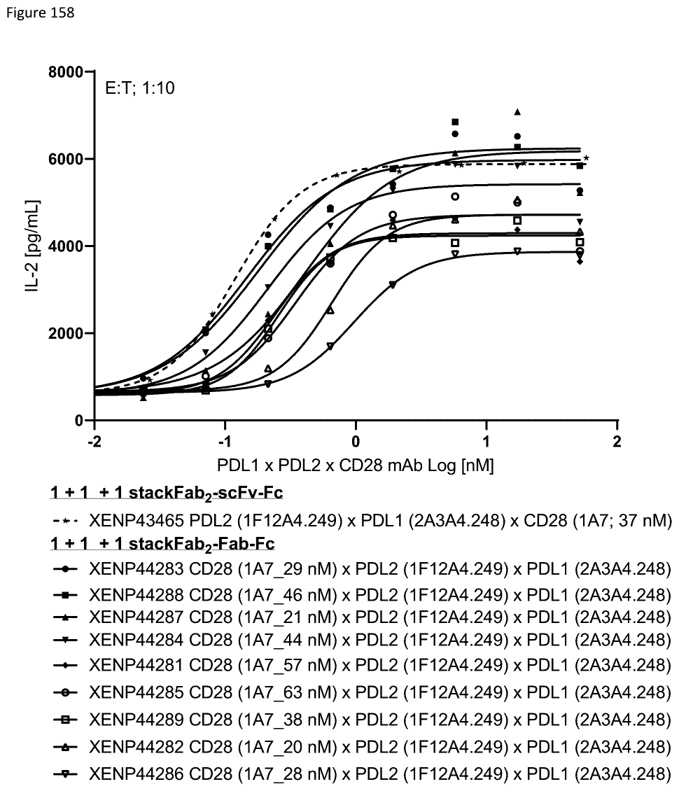

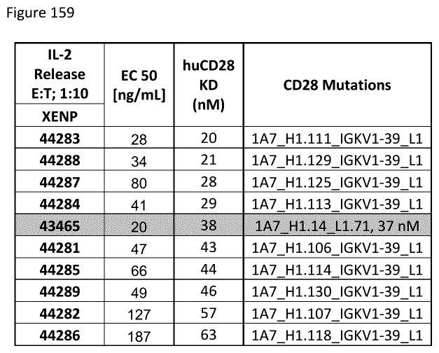

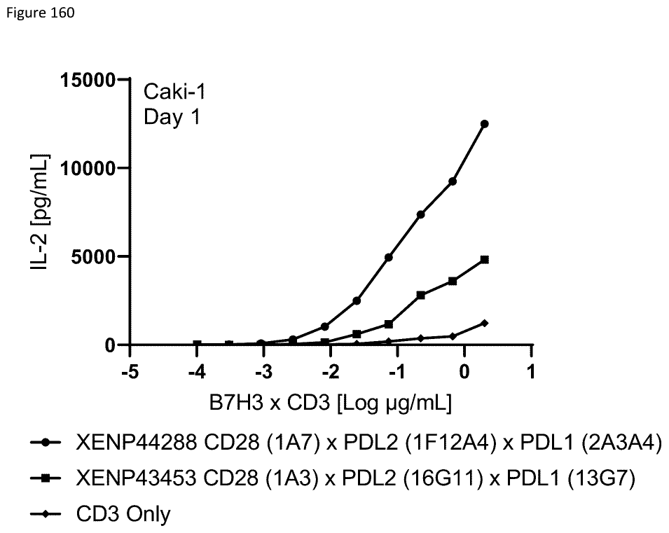

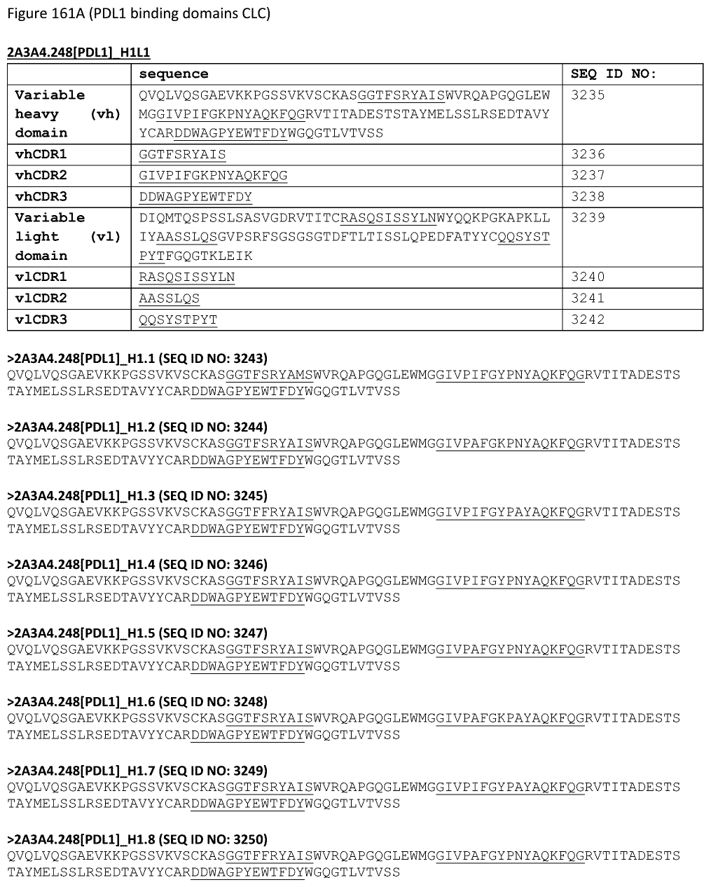

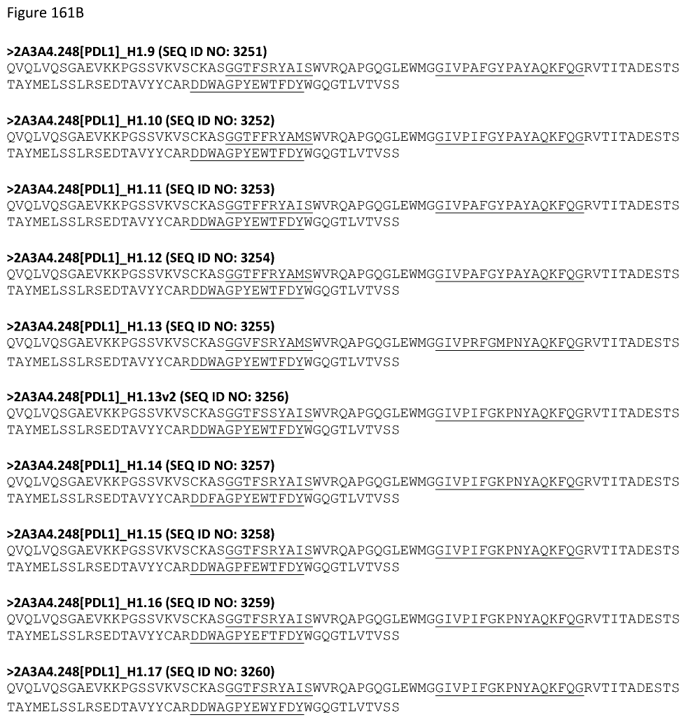

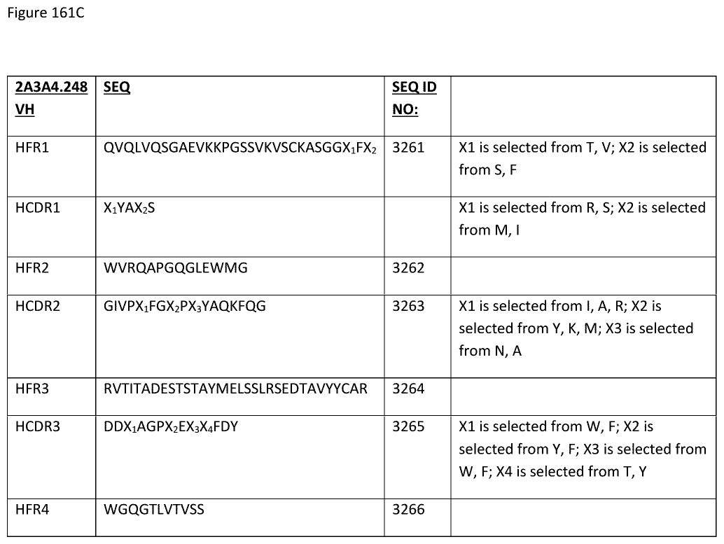

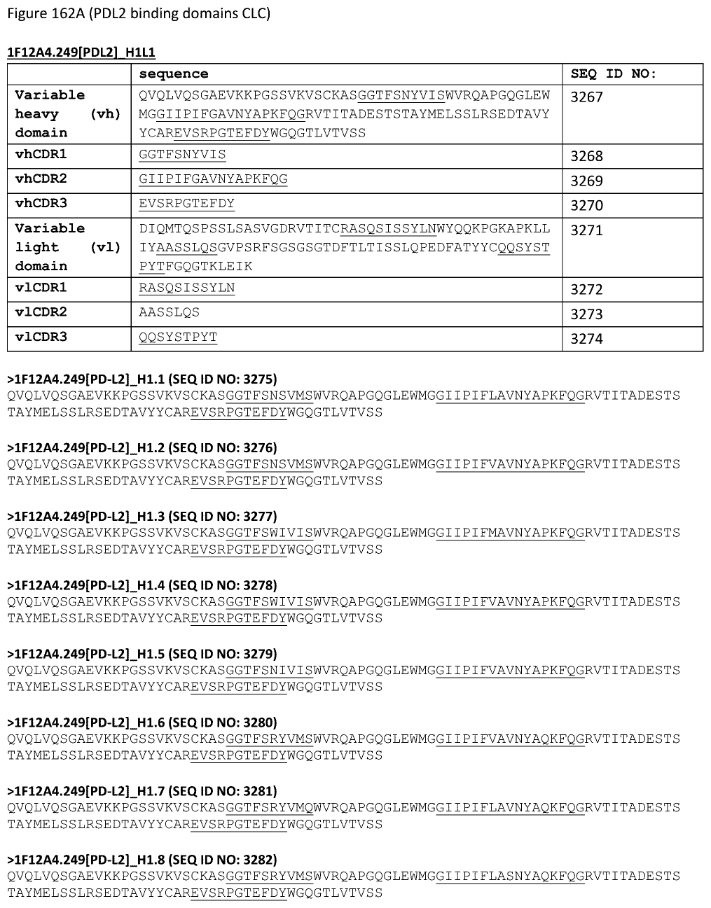

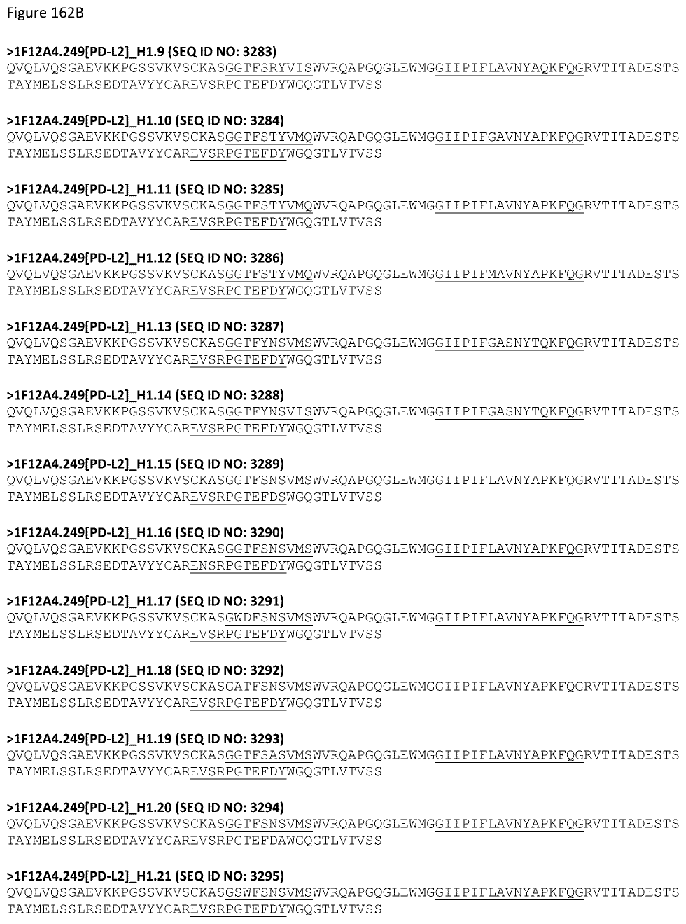

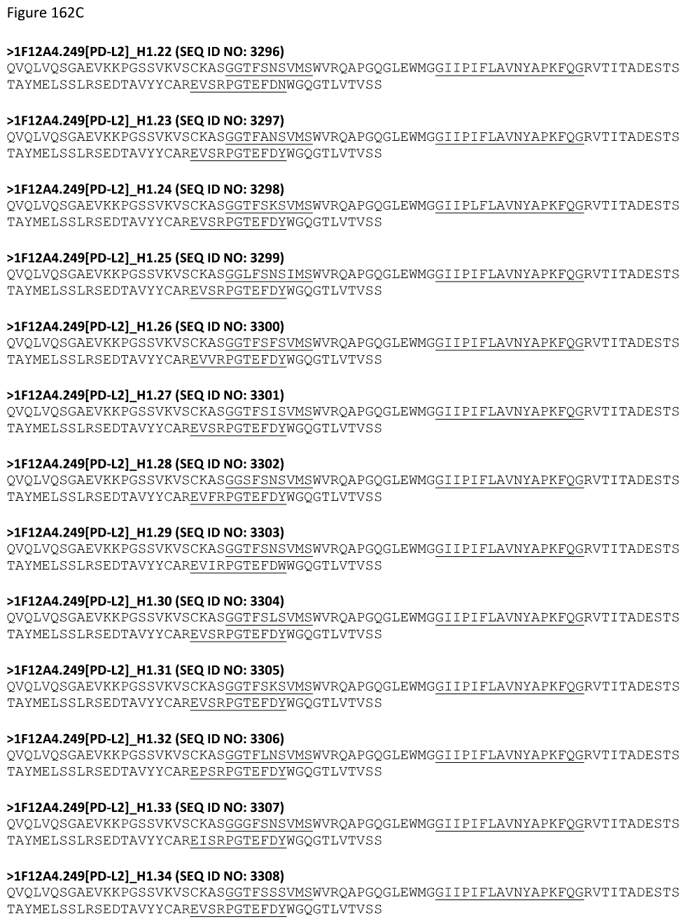

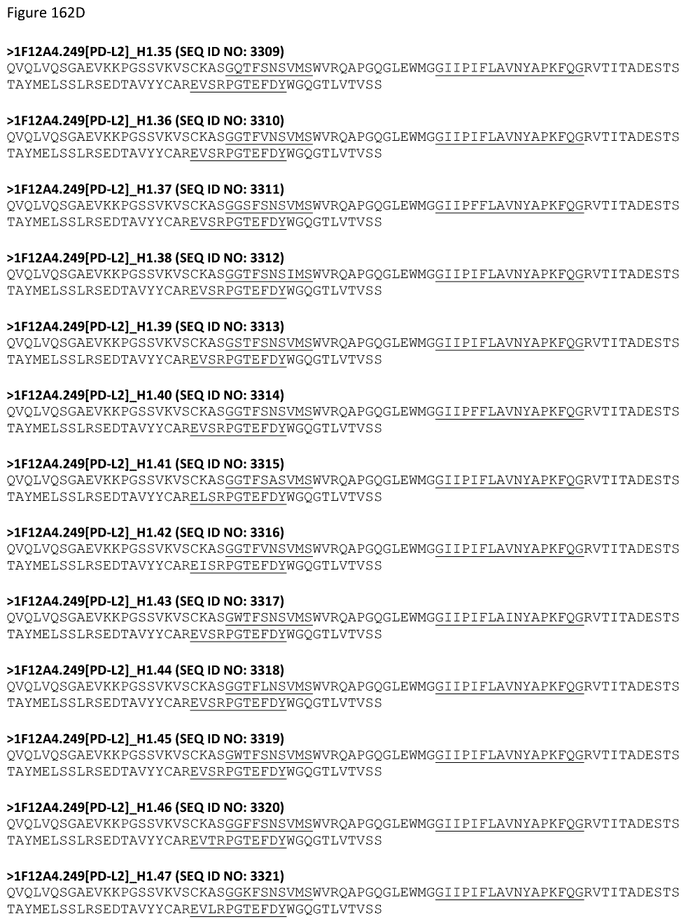

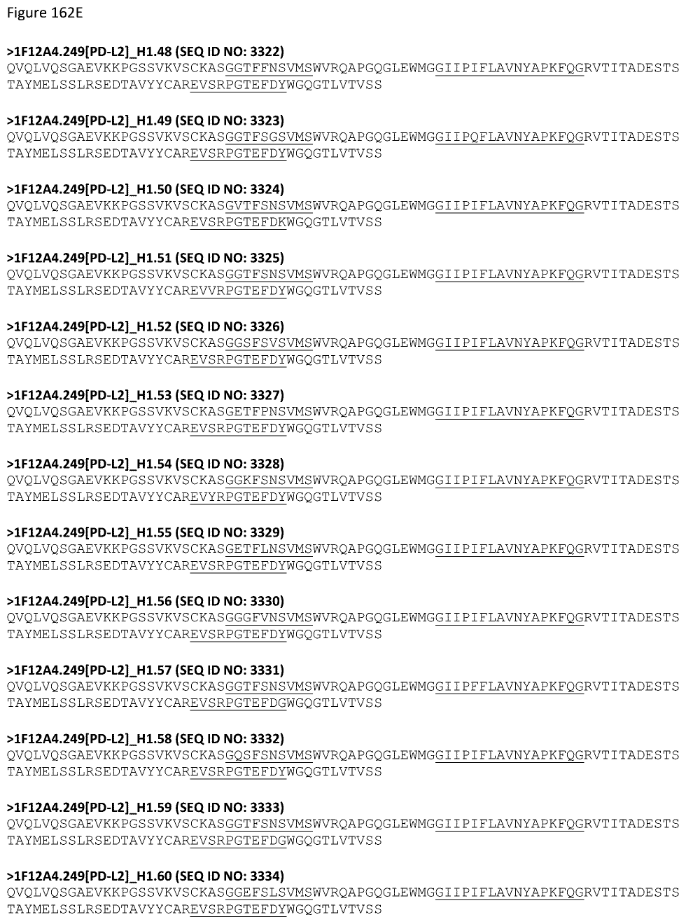

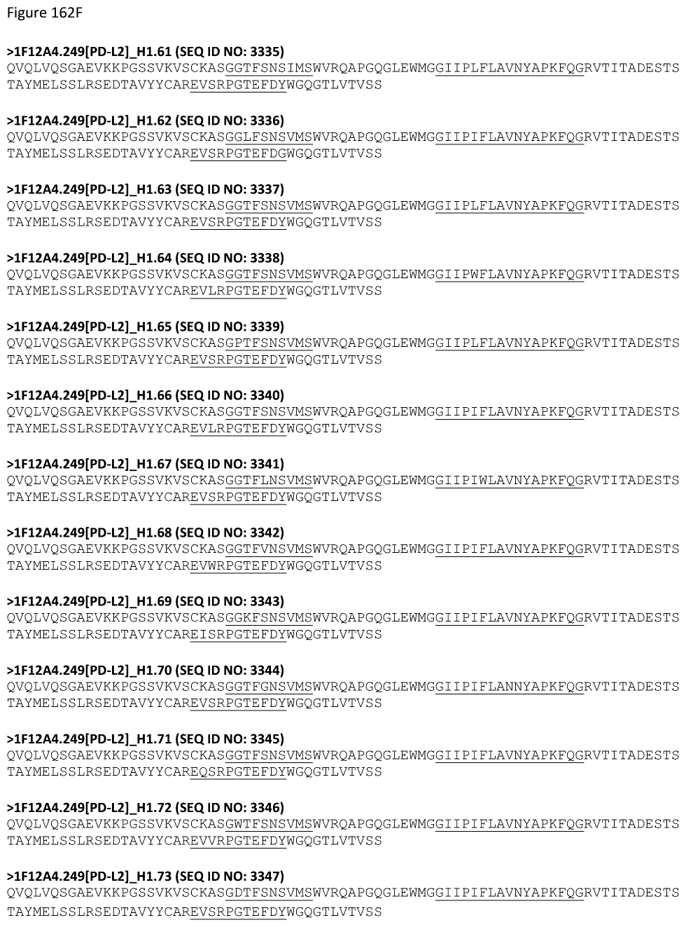

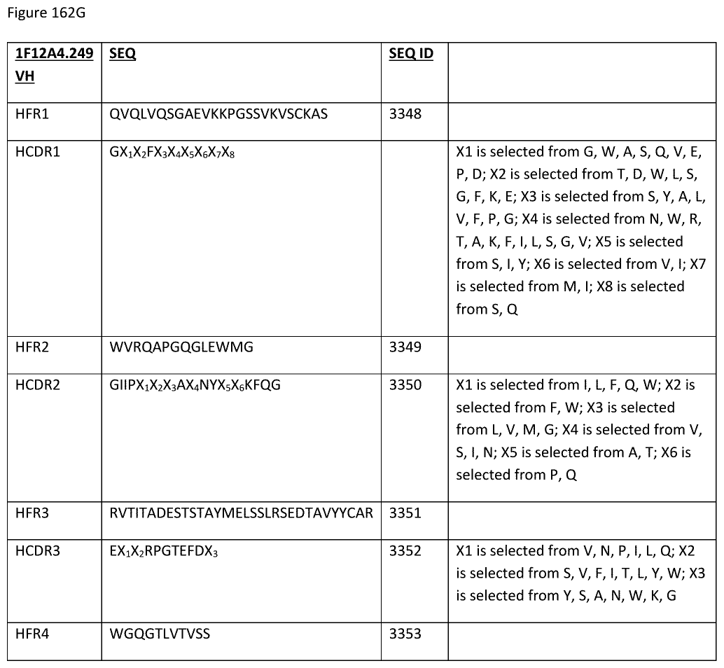

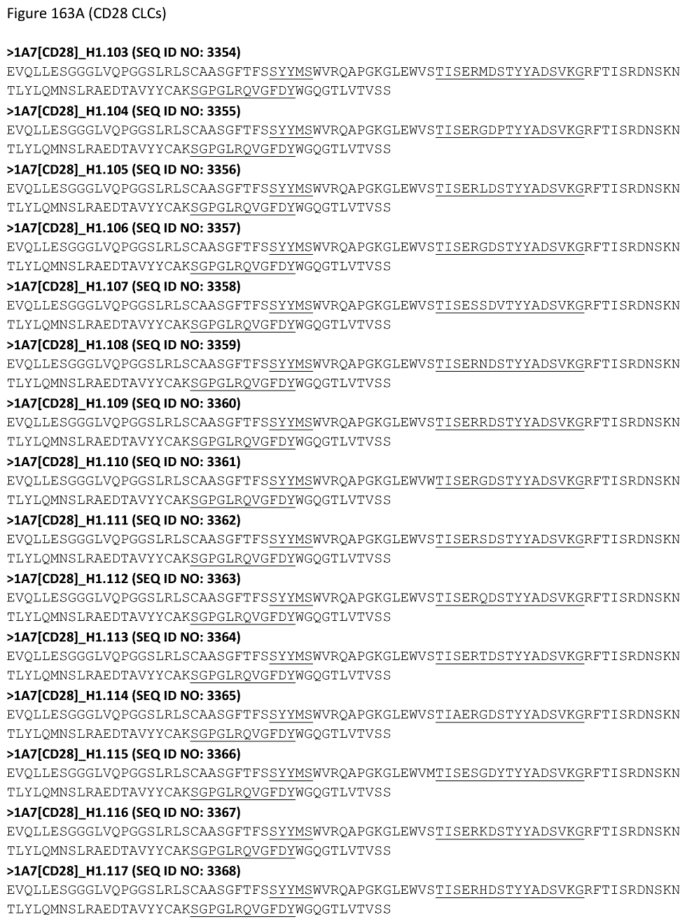

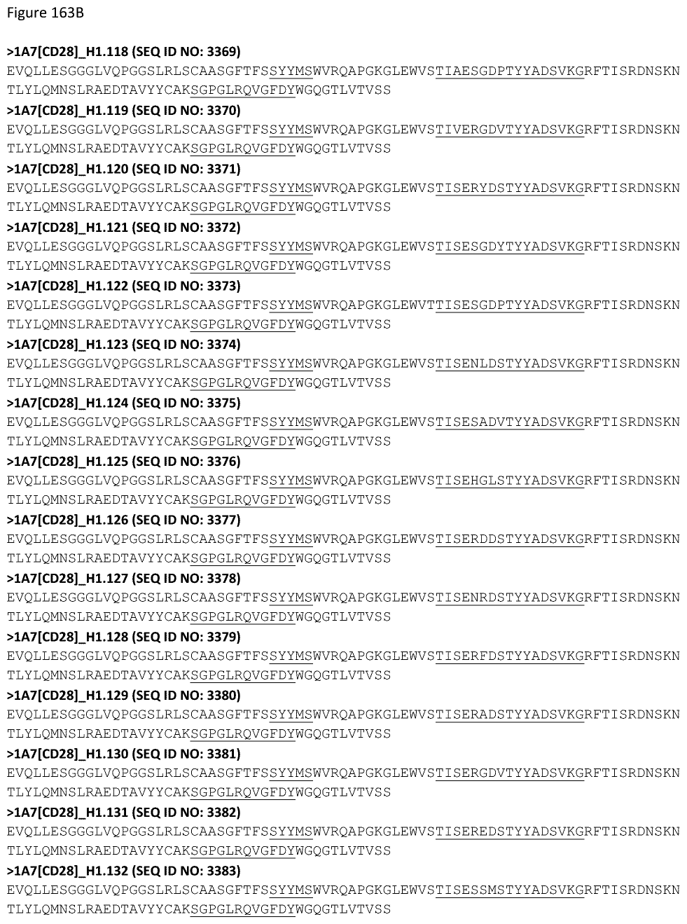

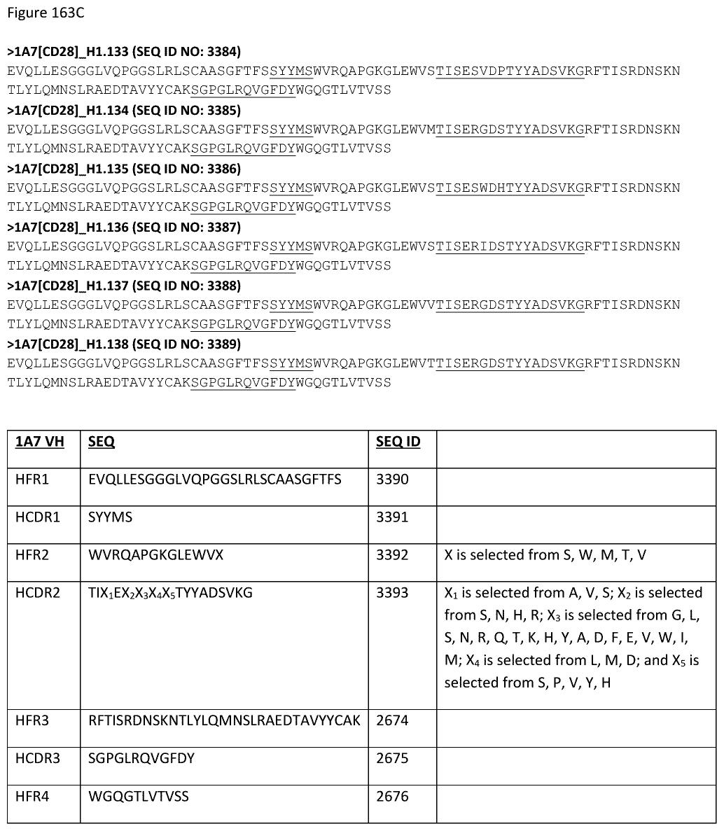

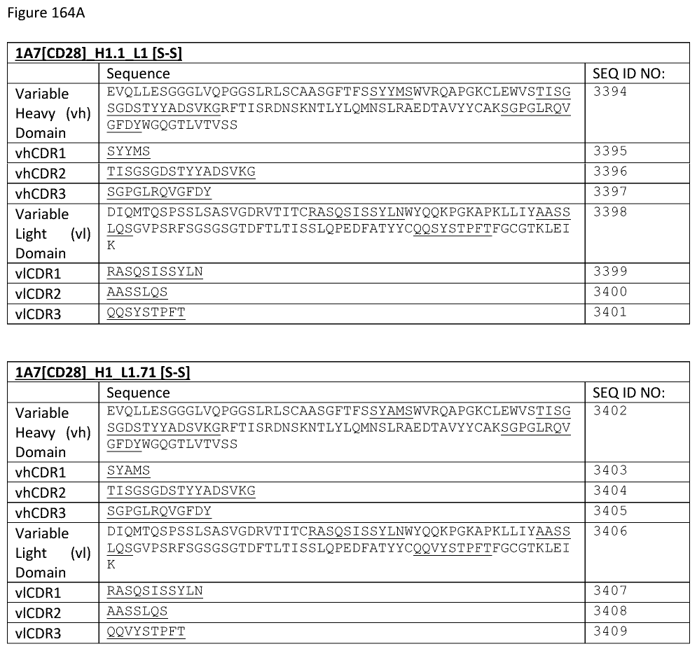

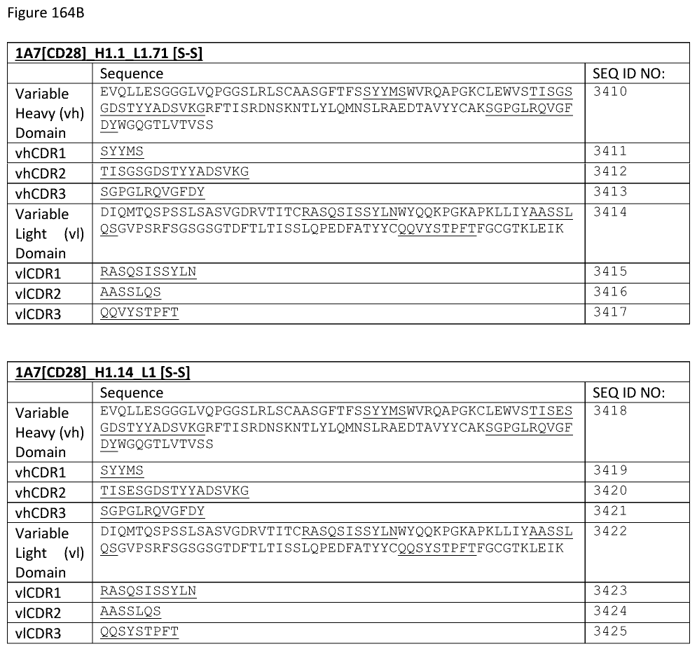

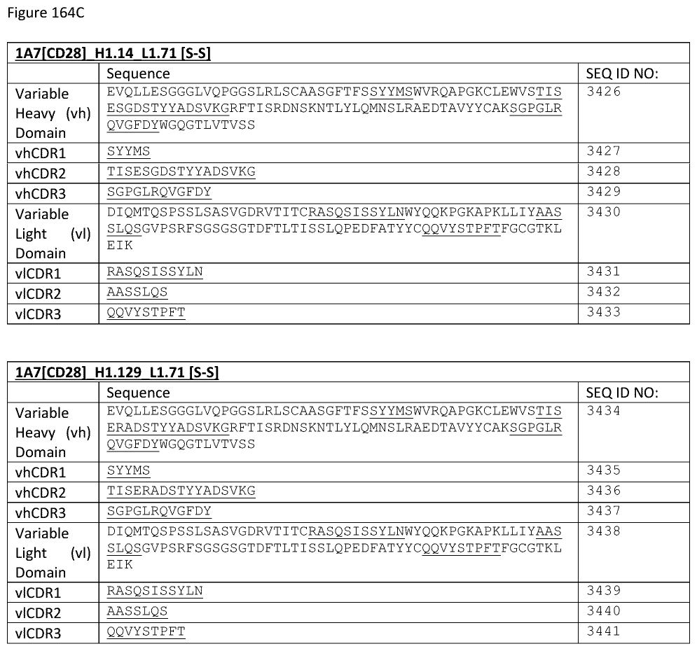

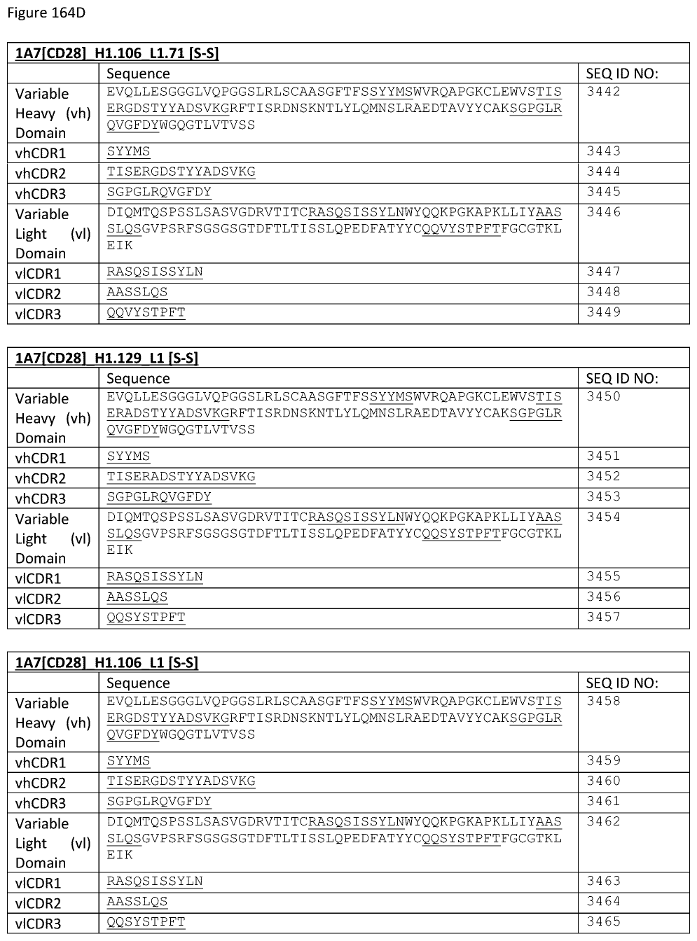

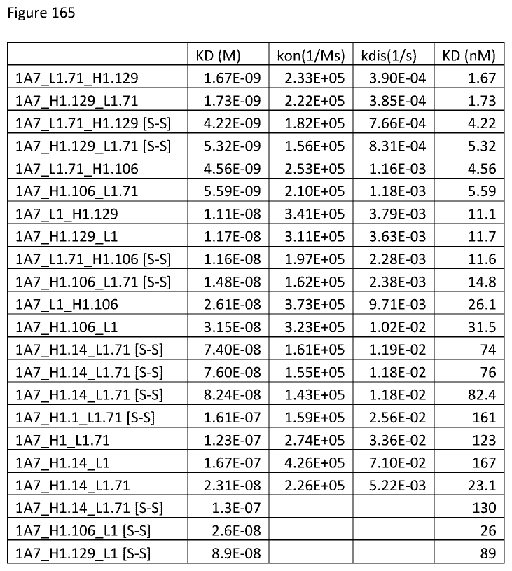

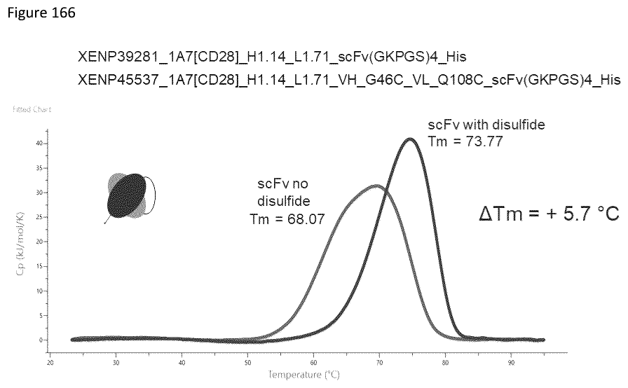

FIG. 10 shows some exemplary Fc domain sequences that are useful in the 1+1 Fab-scFv-Fc format antibodies. The “monomer 1” sequences depicted in FIG. 10 typically refer to the Fc domain of the “Fab-Fc heavy chain” and the “monomer 2” sequences refer to the Fc domain of the “scFv-Fc heavy chain.” In addition, FIGS. 12 and 13 provides exemplary CH1-hinge domains, CH1 domains, and hinge domains that can be included in the first or second monomer of the 1+1 Fab-scFv-Fc format. Further, FIG. 14 provides useful CL sequences that can be used with this format.

2. 2+1 Fab₂-scFv-Fc Format (Central-scFv Format)

One heterodimeric antibody format that finds particular use in the subject antibodies provided herein (e.g., anti-PD-L1×anti-CD28 and anti-PD-L1×anti-CD28 antibodies) is the 2+1 Fab₂-scFv-Fc format (also referred to as “central-scFv format”) shown in FIG. 82B. This antibody format includes three antigen binding domains: two Fab portions and an scFv that is inserted between the VH-CH1 and CH2-CH3 regions of one of the monomers. In some embodiments of this format, the two Fab portions each bind PD-L1.

In some embodiments, the “extra” scFv domain binds CD28. In some embodiments, the 2+1 Fab₂-scFv-Fc format antibody is a trivalent antibody.

In some embodiments of the 2+1 Fab₂-scFv-Fc format, a first monomer includes a standard heavy chain (i.e., VH1-CH1-hinge-CH2-CH3), wherein VH1 is a first variable heavy domain and CH2-CH3 is a first Fc domain. A second monomer includes another first variable heavy domain (VH1), a CH1 domain (and optional hinge), a second Fc domain, and an scFv that includes an scFv variable light domain (VL2), an scFv linker and a scFv variable heavy domain (VH2). The scFv is covalently attached between the C-terminus of the CH1 domain of the second monomer and the N-terminus of the second Fc domain using optional domain linkers (VH1-CH1-[optional linker]-VH2-scFv linker-VH2-[optional linker]-CH2-CH3, or the opposite orientation for the scFv, VH1-CH1-[optional linker]-VL2-scFv linker-VH2-[optional linker]-CH2-CH3). The optional linkers can be any suitable peptide linkers, including, for example, the domain linkers included in FIG. 8. This embodiment further utilizes a common light chain that includes a variable light domain (VL1) and a constant light domain (CL). The common light chain associates with the VH1-CH1 of the first and second monomers to form two identical Fabs. In some embodiments, the identical Fabs each bind PD-L1. As for many of the embodiments herein, these constructs can include skew variants, pI variants, ablation variants, additional Fc variants, etc. as desired and described herein.

In some embodiments, the first and second Fc domains of the 2+1 Fab₂-scFv-Fc format antibody are variant Fc domains that include heterodimerization skew variants (e.g., a set of amino acid substitutions as shown in FIGS. 4 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 Fab₂-scFv-Fc format antibody provided herein includes a charged scFv linker (including those shown in FIG. 7). In some embodiments, the 2+1 Fab₂-scFv-Fc format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 Fab₂-scFv-Fc format antibody provided herein includes a (GKPGS)₄ charged scFv linker. In some embodiments, the 2+1 Fab₂-scFv-Fc format antibody provided herein includes FcRn variants M428L/N434S, wherein numbering is according to EU numbering.

In some embodiments of the 2+1 Fab₂-scFv-Fc format antibody, the two Fab domains are PD-L1 or PD-L2 binding domains. In some embodiments, the scFv is the PD-L1 or PD-L2 binding domain.

Any suitable PD-L1 binding domain can be included in the subject 2+1 Fab₂-scFv-Fc format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20,
ii) a variable heavy domain and variable light domain of any of the PD-L1 binding domains in FIGS. 17 and 21,
iii) a variable heavy domain of any of the PD-L1 binding domains in FIGS. 25 and 26, and a variable light domain of any of the PD-L1 binding domains in FIGS. 25 and 26,
iv) a variable heavy domain and variable light domain of any of the PD-L1 binding domains in FIGS. 25 and 26,
v) a variable heavy domain of any of the PD-L1 binding domains in FIGS. 25 and 26, and a variable light domain having the amino acid sequence of SEQ ID NO:20 (FIG. 24), and
vi) a variable heavy domain and variable light domain of any of the PD-L1 binding domains in FIG. 28.

Any suitable PD-L2 binding domain can be included in the subject 2+1 Fab2-scFv-Fc format antibody including those provided herein (FIGS. 29-32 and 34 and the Sequence Listing) or a variant thereof. In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32,
ii) a variable heavy domain and variable light domain of any of the PD-L2 binding domains in FIGS. 29-32,
iii) a variable heavy domain of any of the PD-L2 binding domains in FIGS. 29-32, and a variable light domain having the amino acid sequence of SEQ ID NO:20 (FIG. 24), and
iv) a variable heavy domain and variable light domain of any of the PD-L2 binding domains in FIG. 34.

Any suitable CD28 binding domain can be included in subject 2+1 Fab2-scFv-Fc format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38,
ii) a variable heavy domain and a variable light domain of any of the CD28 binding domains in FIG. 38,
iii) a variable heavy domain of any of the CD28 binding domains in FIGS. 41-74, and a variable light domain of any of the CD28 binding domains in FIGS. 41-74,
iv) a variable heavy domain and variable light domain of any of the CD28 binding domains in FIGS. 41-74,
v) a variable heavy domain of any of the CD28 binding domains in FIGS. 41-74, and a variable light domain having the amino acid sequence of SEQ ID NO:20 (FIG. 24), and
vi) a variable heavy domain and variable light domain of any of the CD28 binding domains in FIG. 81.

FIG. 11 shows some exemplary Fc domain sequences that are useful with the 2+1 Fab2-scFv-Fc format. The “monomer 1” sequences depicted in FIG. 11 typically refer to the Fc domain of the “Fab-Fc heavy chain” and the “monomer 2” sequences refer to the Fc domain of the “Fab-scFv-Fc heavy chain.” In addition, FIGS. 12 and 13 provides exemplary CH1-hinge domains, CH1 domains, and hinge domains that can be included in the first or second monomer of the 2+1 Fab2-scFv-Fc format. Further, FIG. 14 provides useful CL sequences that can be used with this format.

Exemplary anti-PD-L1×anti-CD28×antibodies in the 2+1 Fab₂-scFv-Fc format are depicted in FIG. 85. In some embodiments, the anti-PD-L1×anti-CD28×antibody is an antibody depicted in FIG. 85.

3. 1+1 CLC Format

One heterodimeric antibody format that finds particular use in subject bispecific antibodies provided herein (e.g., anti-PD-L1×anti-CD28 and anti-PD-L1×anti-CD28 antibodies) is the “1+1 Common Light Chain” or “1+1 CLC” format, which is depicted in FIG. 82C. The 1+1 CLC format antibody includes a first monomer that includes a VH1-CH1-hinge-CH2-CH3, wherein VH1 is a first variable heavy domain and CH2-CH3 is a first Fc domain; a second monomer that includes a VH2-CH1-hinge-CH2-CH3, wherein VH2 is a second variable heavy domain and CH2-C3 is a second Fc domain; and a third monomer “common light chain” comprising VL-CL, wherein VL is a common variable light domain and CL is a constant light domain. In such embodiments, the VL pairs with the VH1 to form a first binding domain with a first antigen binding specificity; and the VL pairs with the VH2 to form a second binding domain with a second antigen binding specificity. In some embodiments, the 1+1 CLC format antibody is a bivalent antibody.

In some embodiments, the first and second Fc domains of the 1+1 CLC format are variant Fc domains that include heterodimerization skew variants (e.g., a set of amino acid substitutions as shown in FIGS. 4 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 6). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIG. 5). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 1+1 CLC format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the 1+1 CLC format antibody provided herein further includes FcRn variants M428L/N434S, wherein numbering is according to EU numbering.

In some embodiments of the 1+1 CLC format antibody, one of the first or second antigen binding domain is a PD-L1 binding domain. Any suitable PD-L1 binding domain can be included in the subject 1+1 CLC format antibody format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain of any of the PD-L1 binding domains in FIGS. 25 and 26, and a variable light domain of any of the PD-L1 binding domains in FIGS. 25 and 26,
ii) a variable heavy domain and variable light domain of any of the PD-L1 binding domains in FIGS. 25 and 26, and
iii) a variable heavy domain of any of the PD-L1 binding domains in FIGS. 25 and 26, and a variable light domain having the amino acid sequence of SEQ ID NO:20 (FIG. 24).

In some embodiments of the 1+1 CLC format antibody, one of the first or second antigen binding domain is a PD-L2 binding domain. Any suitable PD-L2 binding domain can be included in the subject 1+1 CLC format antibody format antibody including those provided herein (FIGS. 29-32 and the Sequence Listing) or a variant thereof. In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32,
ii) a variable heavy domain and variable light domain of any of the PD-L2 binding domains in FIGS. 29-32, and
iii) a variable heavy domain of any of the PD-L2 binding domains in FIGS. 29-32, and a variable light domain having the amino acid sequence of SEQ ID NO:20 (FIG. 24).

In some embodiments of the 1+1 CLC format antibody, one of the first or second antigen binding domain is a CD28 binding domain. Any suitable CD28 binding domain can be included in the subject 1+1 CLC format antibody format antibody including those provided herein (FIGS. 241-47 and the Sequence Listing) or a variant thereof. In some embodiments, the CD28 binding domain includes a variable heavy domain and variable light domain selected from the following:
i) a variable heavy domain of any of the CD28 binding domains in FIGS. 41-74, and a variable light domain of any of the CD28 binding domains in FIGS. 41-74,
ii) a variable heavy domain and variable light domain of any of the CD28 binding domains in FIGS. 41-74, and
iii) a variable heavy domain of any of the CD28 binding domains in FIGS. 41-74, and a variable light domain having the amino acid sequence of SEQ ID NO:20 (FIG. 24).

4. 2+1 CLC Format

Another heterodimeric antibody format that finds particular use in subject bispecific antibodies provided herein (e.g., anti-PD-L1×anti-CD28 and anti-PD-L1×anti-CD28 antibodies) is the “2+1 Common Light Chain” or “2+1 CLC” format, which is depicted in FIG. 82D. The 2+1 CLC format includes a first monomer that includes a VH1-CH1-linker-VH1-CH1-hinge-CH2-CH3, wherein the VH1s are each a first variable heavy domain and CH2-CH3 is a first Fc domain; a second monomer that includes a VH2-CH1-hinge-CH2-CH3, wherein VH2 is a second variable heavy domain and CH2-CH3 is a second Fc domain; and a third monomer that includes a “common light chain” VL-CL, wherein VL is a common variable light domain and CL is a constant light domain. The VL pairs with each of the VH1s of the first monomer to form two first binding domains, each with a first antigen binding specificity; and the VL pairs with the VH2 to form a second binding domain with a second antigen binding specificity. The linker of the first monomer can be any suitable linker, including any one of the domain linkers or combinations thereof described in FIG. 8. In some embodiments, the linker is EPKSCGKPGSGKPGS (SEQ ID NO:936). In some embodiments, the 2+1 CLC format antibody is a trivalent antibody.

In some embodiments, the first and second Fc domains of the 2+1 CLC format are variant Fc domains that include heterodimerization skew variants (e.g., a set of amino acid substitutions as shown in FIGS. 4 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 6). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first or second monomer includes pI variants (including those shown in FIG. 5). In exemplary embodiments, the constant domain (CH1-hinge-CH2-CH3) of the first or second monomer includes pI variants N208D/Q295E/N384D/Q418E/N421D, wherein numbering is according to EU numbering.

In some embodiments, the 2+1 CLC format antibody provided herein further includes FcRn variants M428L/N434S, wherein numbering is according to EU numbering.

In exemplary embodiments, the first variant Fe domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the 2+1 CLC format antibody provided herein further includes FcRn variants M428L/N434S, wherein numbering is according to EU numbering.

In some embodiments of the 2+1 CLC format antibody, each of the two first binding domains is a PD-L1 binding domain. In some embodiments, the second binding domain is a PD-L1 binding domain. Any suitable PD-L1 binding domain can be included in the subject 2+1 CLC format antibody format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain of any of the PD-L1 binding domains in FIGS. 25 and 26, and a variable light domain of any of the PD-L1 binding domains in FIGS. 25 and 26 or a variant thereof,
ii) a variable heavy domain and variable light domain of any of the PD-L1 binding domains in FIGS. 25 and 26 or a variant thereof, and
iii) a variable heavy domain of any of the PD-L1 binding domains in FIGS. 25 and 26, and a variable light domain having the amino acid sequence of SEQ ID NO:20 (FIG. 24) or a variant thereof.

In some embodiments of the 2+1 CLC format antibody, each of the two first binding domains is a PD-L2 binding domain. In some embodiments, the second binding domain is a PD-L1 binding domain. Any suitable PD-L2 binding domain can be included in the subject 1+1 CLC format antibody format antibody including those provided herein (FIGS. 29-32 and the Sequence Listing) or a variant thereof. In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32 or a variant thereof,
ii) a variable heavy domain and variable light domain of any of the PD-L2 binding domains in FIGS. 29-32 or a variant thereof, and
iii) a variable heavy domain of any of the PD-L2 binding domains in FIGS. 29-32, and a variable light domain having the amino acid sequence of SEQ ID NO:20 (FIG. 24) or a variant thereof.

In some embodiments of the 2+1 CLC format antibody, one of the antigen binding domain is a CD28 binding domain. Any suitable CD28 binding domain can be included in the subject 2+1 CLC format antibody format antibody including those provided herein (FIGS. 241-47 and the Sequence Listing) or a variant thereof. In some embodiments, the CD28 binding domain includes a variable heavy domain and variable light domain selected from the following:
i) a variable heavy domain of any of the CD28 binding domains in FIGS. 41-74, and a variable light domain of any of the CD28 binding domains in FIGS. 41-74 or a variant thereof,
ii) a variable heavy domain and variable light domain of any of the CD28 binding domains in FIGS. 41-74 or a variant thereof, and
iii) a variable heavy domain of any of the CD28 binding domains in FIGS. 41-74, and a variable light domain having the amino acid sequence of SEQ ID NO:20 (FIG. 24) or a variant thereof.

5. 2+1 mAb-scFv Format

One heterodimeric antibody format that finds particular use in the subject bispecific antibodies provided herein (e.g., anti-PD-L1×anti-CD28 and anti-PD-L1×anti-CD28 antibodies) is the 2+1 mAb-scFv format shown in FIG. 82E. This antibody format includes three antigen binding domains: two Fab portions and an scFv that is attached to the C-terminal of one of the heavy chains. In some embodiments of this format, the Fab portions each bind PD-L1. In some embodiments, the “extra” scFv domain binds CD28. That is, this mAb-scFv format is a trivalent antibody.

In these embodiments, the first chain or monomer comprises, from N- to C-terminal, VH1-CH1-hinge-CH2-CH3, the second monomer comprises, from N- to C-terminal, VH1-CH1-hinge-CH2-CH3-domain linker-scFv domain, where the scFv domain comprises a second VH (VH2), a second VL (VL2) and a scFv linker. As for all the scFv domains herein, the scFv domain can be in either orientation, from N- to C-terminal, VH2-scFv linker-VL2 or VL2-scFv linker-VH2. Accordingly, the second monomer may comprise, from N- to C-terminal, VH1-CH1-hinge-CH2-CH3-domain linker-VH2-scFv linker-VL2 or VH1-CH1-hinge-CH2-CH3-domain linker-VL2-scFv linker-VH2. The composition also comprises a light chain, VL1-CL. In these embodiments, the VH1-VL1 each form a first ABD and the VH2-VL2 form a second ABD. In some embodiments, the first ABD binds to a tumor target antigen, including human B7H3, and the second ABD binds human CD28.

In some embodiments, the first and second Fc domains of the 2+1 mAb-scFv format antibody are variant Fc domains that include heterodimerization skew variants (e.g., a set of amino acid substitutions as shown in FIGS. 4 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering.

In some embodiments, the variant Fc domains include ablation variants (including those shown in FIG. 5). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 4). In exemplary embodiments, the constant domain (CH1-hinge-CH2-CH3) of the first monomer includes pI variants N208D/Q295E/N384D/Q418E/N421D, wherein numbering is according to EU numbering.

In some embodiments, the scFv of the 2+1 mAb-scFv format antibody provided herein includes a charged scFv linker (including those shown in FIG. 7). In some embodiments, the 2+1 mAb-scFv format antibody provided herein includes FcRn variants M428L/N434S, wherein numbering is according to EU numbering.

In exemplary embodiments, the first variant Fc domain includes heterodimerization skew variants L368D/K370S and the second variant Fc domain includes heterodimerization skew variants S364K/E357Q; each of the first and second variant Fc domains include ablation variants E233P/L234V/L235A/G236_/S267K; and the constant domain (CH1-hinge-CH2-CH3) of the first monomer includes pI variants N208D/Q295E/N384D/Q418E/N421D, wherein numbering is according to EU numbering. In some embodiments, the scFv of the 2+1 mAb-scFv format antibody provided herein includes a (GKPGS)₄ charged scFv linker. In some embodiments, 2+1 mAb-scFv format antibody provided herein includes FcRn variants M428L/N434S, wherein numbering is according to EU numbering.

Any suitable PD-L1 binding domain can be included in the subject 2+1 mAb-scFv format antibody including those provided herein (FIGS. 17, 19, 20, 21, 24-26, and 28 and the Sequence Listing) or a variant thereof. In some embodiments of the 2+1 mAb-scFv, the VH1 of the first and second monomer and the VL1 of the common light chain each form a PD-L1 binding domain. In some embodiments, the scFv of the second monomer is a PD-L1 binding domain.

In some embodiments, the PD-L1 binding domain includes a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20 or a variant thereof,
ii) a variable heavy domain and variable light domain of any of the PD-L1 binding domains in FIGS. 17 and 21 or a variant thereof,
iii) a variable heavy domain of any of the PD-L1 binding domains in FIGS. 25 and 26, and a variable light domain of any of the PD-L1 binding domains in FIGS. 25 and 26 or a variant thereof,
iv) a variable heavy domain and variable light domain of any of the PD-L1 binding domains in FIGS. 25 and 26 or a variant thereof,
v) a variable heavy domain of any of the PD-L1 binding domains in FIGS. 25 and 26, and a variable light domain having the amino acid sequence of SEQ ID NO:20 (FIG. 24) or a variant thereof, and
vi) a variable heavy domain and variable light domain of any of the PD-L1 binding domains in FIG. 28 or a variant thereof.

Any suitable PD-L2 binding domain can be included in the subject 2+1 mAb-scFv format antibody including those provided herein (FIGS. 29-34 and the Sequence Listing) or a variant thereof. In some embodiments of the 2+1 mAb-scFv, the VH1 of the first and second monomer and the VL1 of the common light chain each form a PD-L2 binding domain. In some embodiments, the scFv of the second monomer is a PD-L2 binding domain.

In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32 or a variant thereof,
ii) a variable heavy domain and variable light domain of any of the PD-L2 binding domains in FIGS. 29-32 or a variant thereof,
iii) a variable heavy domain of any of the PD-L2 binding domains in FIGS. 29-32, and a variable light domain having the amino acid sequence of SEQ ID NO:20 (FIG. 24) or a variant thereof, and
iv) a variable heavy domain and variable light domain of any of the PD-L2 binding domains in FIG. 34 or a variant thereof.

In some embodiments, the 2+1 mAb-scFv format antibody is a bispecific antibody that binds CD28 and PD-L1 or PD-L2. In some embodiments, the scFv of the second monomer is a CD28 binding domain and the VH1 of the first and second monomer and the VL1 of the common light chain each form PD-L1 or PD-L2 binding domains. In some embodiments, the scFv of the second monomer is a PD-L1 or PD-L1 binding domain and the VH1 of the first and second monomer and the VL1 of the common light chain each form CD28 binding domains.

Any suitable CD28 binding domain can be included in subject 2+1 mAb-scFv format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38 or a variant thereof,
ii) a variable heavy domain and a variable light domain of any of the CD28 binding domains in FIG. 38 or a variant thereof,
iii) a variable heavy domain of any of the CD28 binding domains in FIGS. 41-74, and a variable light domain of any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain of any of the CD28 binding domains in FIGS. 41-74, and a variable light domain having the amino acid sequence of SEQ ID NO:20 (FIG. 24) or a variant thereof, and
vi) a variable heavy domain and variable light domain of any of the CD28 binding domains in FIG. 81 or a variant thereof.

FIGS. 10 and 11 show some exemplary Fe domain sequences that are useful with the 2+1 mAb-scFv format. The “monomer 1” sequences depicted in FIG. 10 typically refer to the Fc domain of the “Fab-Fc heavy chain” and the “monomer 2” sequences refer to the Fc domain of the “Fab-Fc-scFv” heavy chain.” In addition, FIG. 12 provides exemplary CH1 (optionally including hinge or half-hinge domains) that can be used in either the “Fab-Fc heavy chain” monomer or the “Fab-Fc-scFv” heavy chain.” FIG. 13 provides exemplary hinge domains that may be used in either the “Fab-Fc heavy chain” monomer or the “Fab-Fc-scFv” heavy chain.” Further, FIG. 14 provides useful CL sequences that can be used with this format.

An exemplary anti-PD-L1×anti-CD28×antibody in the 2+1 mAb-scFv format is depicted in FIG. 37.

6. Dual scFv Formats

One heterodimeric antibody format that finds particular use in the subject bispecific antibodies (e.g., anti-PD-L1×anti-CD28 and anti-PD-L1×anti-CD28 antibodies) is the dual scFv format, as are known in the art and shown in FIG. 82F. In this embodiment, the heterodimeric bispecific antibody is made up of two scFv-Fc monomers (both in either (vh-scFv linker-vl-[optional domain linker]-CH2-CH3) format or (vl-scFv linker-vh-[optional domain linker]-CH2-CH3) format, or with one monomer in one orientation and the other in the other orientation.

In this case, all ABDs are in the scFv format. Any suitable PD-L1 binding domain, PD-L2 binding domain and CD28 binding domain can be included in subject bispecific antibodies in the dual scFv format, including any of the PD-L1, PD-L2, and CD28 binding domains provided herein.

In addition, the Fc domains of the dual scFv format comprise skew variants (e.g. a set of amino acid substitutions as shown in FIGS. 4 and 9, with particularly useful skew variants being selected from the group consisting of S364K/E357Q:L368D/K370S; L368D/K370S:S364K; L368E/K370S:S364K; T411T/E360E/Q362E:D401K; L368D/K370S:S364K/E357L, K370S:S364K/E357Q, T366S/L368A/Y407V:T366W and T366S/L368A/Y407V/Y349C:T366W/S354C), optionally ablation variants (including those shown in FIG. 6), optionally charged scFv linkers (including those shown in FIG. 7) and the heavy chain comprises pI variants (including those shown in FIG. 5).

In some embodiments, the dual scFv format includes skew variants, pI variants, and ablation variants. Accordingly, some embodiments include formats that comprise: a) a first monomer that comprises the skew variants S364K/E357Q, the ablation variants E233P/L234V/L235A/G236del/S267K, and a scFv that binds a first antigen (VH1-scFv linker-VL1-[optional domain linker]-CH2-CH3 or VL1-scFv linker-VH1-[optional domain linker]-CH2-CH3) and b) a first monomer that comprises the skew variants L368D/K370S, the ablation variants E233P/L234V/L235A/G236del/S267K, and a scFv that binds a second antigen (VH1-scFv linker-VL1-[optional domain linker]-CH2-CH3 or VL1-scFv linker-VH1-[optional domain linker]-CH2-CH3). pI variants can be as outlined herein, but most common will be charged scFv linkers of opposite charge for each monomer. FcRn variants, particularly 428L/434S, can optionally be included.

Any suitable PD-L1 binding domain can be included in the subject dual scFv format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20 or a variant thereof,
ii) a variable heavy domain and variable light domain of any of the PD-L1 binding domains in FIGS. 17 and 21 or a variant thereof,
iii) a variable heavy domain of any of the PD-L1 binding domains in FIGS. 25 and 26, and a variable light domain of any of the PD-L1 binding domains in FIGS. 25 and 26 or a variant thereof,
iv) a variable heavy domain and variable light domain of any of the PD-L1 binding domains in FIGS. 25 and 26 or a variant thereof,
v) a variable heavy domain of any of the PD-L1 binding domains in FIGS. 25 and 26, and a variable light domain having the amino acid sequence of SEQ ID NO:20 (FIG. 24) or a variant thereof, and
vi) a variable heavy domain and variable light domain of any of the PD-L1 binding domains in FIG. 28 or a variant thereof.

Any suitable PD-L2 binding domain can be included in the subject dual scFv format antibody including those provided herein (FIGS. 29-34 and the Sequence Listing) or a variant thereof. In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32 or a variant thereof,
ii) a variable heavy domain and variable light domain of any of the PD-L2 binding domains in FIGS. 29-32 or a variant thereof,
iii) a variable heavy domain of any of the PD-L2 binding domains in FIGS. 29-32, and a variable light domain having the amino acid sequence of SEQ ID NO:20 (FIG. 24) or a variant thereof, and
iv) a variable heavy domain and variable light domain of any of the PD-L2 binding domains in FIG. 34 or a variant thereof.

Any suitable CD28 binding domain can be included in subject dual scFv format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38 or a variant thereof,
ii) a variable heavy domain and a variable light domain of any of the CD28 binding domains in FIG. 38 or a variant thereof,
iii) a variable heavy domain of any of the CD28 binding domains in FIGS. 41-74, and a variable light domain of any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain of any of the CD28 binding domains in FIGS. 41-74, and a variable light domain having the amino acid sequence of SEQ ID NO:20 (FIG. 24) or a variant thereof, and
vi) a variable heavy domain and variable light domain of any of the CD28 binding domains in FIG. 81 or a variant thereof.

7. One-Armed scFv-mAb Format

One heterodimeric antibody format that finds particular use in subject antibodies provided herein (e.g., anti-PD-L1×anti-CD28 and anti-PD-L1×anti-CD28 antibodies) is the one-armed mAb-scFv format shown in FIG. 82G. This format includes: 1) a first monomer that comprises an “empty” Fc domain; 2) a second monomer that includes a first variable heavy domain (VH), a scFv domain (a second antigen binding domain) and an Fc domain, where the scFv domain is attached to the N-terminus of the first variable heavy domain; and 3) a light chain that includes a first variable light domain and a constant light domain. The first variable heavy domain and the first variable light domain form a first antigen binding domain and the scFv is a second antigen binding domain. In this format, one of the first antigen binding domain and second binding domain binds CD28, and the other antigen binding domain binds PD-L1 or PD-L2. As for many of the embodiments herein, these constructs include skew variants, pI variants, ablation variants, additional Fc variants, etc. as desired and described herein.

Any suitable PD-L1 binding domain can be included in the subject one-armed mAb-scFv format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20 or a variant thereof,
ii) a variable heavy domain and variable light domain of any of the PD-L1 binding domains in FIGS. 17 and 21 or a variant thereof,
iii) a variable heavy domain of any of the PD-L1 binding domains in FIGS. 25 and 26, and a variable light domain of any of the PD-L1 binding domains in FIGS. 25 and 26 or a variant thereof,
iv) a variable heavy domain and variable light domain of any of the PD-L1 binding domains in FIGS. 25 and 26 or a variant thereof,
v) a variable heavy domain of any of the PD-L1 binding domains in FIGS. 25 and 26, and a variable light domain having the amino acid sequence of SEQ ID NO:20 (FIG. 24) or a variant thereof, and
vi) a variable heavy domain and variable light domain of any of the PD-L1 binding domains in FIG. 28 or a variant thereof.

Any suitable PD-L2 binding domain can be included in the subject one-armed mAb-scFv format antibody including those provided herein (FIGS. 29-34 and the Sequence Listing) or a variant thereof. In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32 or a variant thereof,
ii) a variable heavy domain and variable light domain of any of the PD-L2 binding domains in FIGS. 29-32 or a variant thereof,
iii) a variable heavy domain of any of the PD-L2 binding domains in FIGS. 29-32, and a variable light domain having the amino acid sequence of SEQ ID NO:20 (FIG. 24) or a variant thereof, and
iv) a variable heavy domain and variable light domain of any of the PD-L2 binding domains in FIG. 34 or a variant thereof.

Any suitable CD28 binding domain can be included in subject one-armed mAb-scFv format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38 or a variant thereof,
ii) a variable heavy domain and a variable light domain of any of the CD28 binding domains in FIG. 38 or a variant thereof,
iii) a variable heavy domain of any of the CD28 binding domains in FIGS. 41-74, and a variable light domain of any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain of any of the CD28 binding domains in FIGS. 41-74, and a variable light domain having the amino acid sequence of SEQ ID NO:20 (FIG. 24) or a variant thereof, and
vi) a variable heavy domain and variable light domain of any of the CD28 binding domains in FIG. 81 or a variant thereof.

8. scFv-mAb Format

One heterodimeric antibody format that finds particular use in subject antibodies provided herein (e.g., anti-PD-L1×anti-CD28 and anti-PD-L1×anti-CD28 antibodies) is the scFv-mAb format shown in FIG. 82H. In this embodiment, the format relies on the use of a N-terminal attachment of a scFv to one of the monomers, thus forming a third antigen binding domain, wherein the Fab portions of the two monomers each bind one target and the “extra” scFv domain binds a different target.

In this embodiment, the first monomer comprises a first heavy chain (comprising a variable heavy domain and a constant domain), with a N-terminally covalently attached scFv comprising a scFv variable light domain, an scFv linker and a scFv variable heavy domain in either orientation ((vh1-scFv linker-vl1-[optional domain linker]-vh2-CH1-hinge-CH2-CH3) or (with the scFv in the opposite orientation) ((vl1-scFv linker-vh1-[optional domain linker]-vh2-CH1-hinge-CH2-CH3)). The second monomer comprises a heavy chain VH2-CH1-hinge-CH2-CH3. This embodiment further utilizes a common light chain comprising a variable light domain and a constant light domain, that associates with the heavy chains to form two identical Fabs. As for many of the embodiments herein, these constructs include skew variants, pI variants, ablation variants, additional Fc variants, etc. as desired and described herein.

Any suitable PD-L1 binding domain can be included in the subject scFv-mAb format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20 or a variant thereof,
ii) a variable heavy domain and variable light domain of any of the PD-L1 binding domains in FIGS. 17 and 21 or a variant thereof,
iii) a variable heavy domain of any of the PD-L1 binding domains in FIGS. 25 and 26, and a variable light domain of any of the PD-L1 binding domains in FIGS. 25 and 26 or a variant thereof,
iv) a variable heavy domain and variable light domain of any of the PD-L1 binding domains in FIGS. 25 and 26 or a variant thereof,
v) a variable heavy domain of any of the PD-L1 binding domains in FIGS. 25 and 26, and a variable light domain having the amino acid sequence of SEQ ID NO:20 (FIG. 24) or a variant thereof, and
vi) a variable heavy domain and variable light domain of any of the PD-L1 binding domains in FIG. 28 or a variant thereof.

Any suitable PD-L2 binding domain can be included in the subject scFv-mAb format antibody including those provided herein (FIGS. 29-34 and the Sequence Listing) or a variant thereof. In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32 or a variant thereof,
ii) a variable heavy domain and variable light domain of any of the PD-L2 binding domains in FIGS. 29-32 or a variant thereof,
iii) a variable heavy domain of any of the PD-L2 binding domains in FIGS. 29-32, and a variable light domain having the amino acid sequence of SEQ ID NO:20 (FIG. 24) or a variant thereof, and
iv) a variable heavy domain and variable light domain of any of the PD-L2 binding domains in FIG. 34 or a variant thereof.

Any suitable CD28 binding domain can be included in subject scFv-mAb format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38 or a variant thereof,
ii) a variable heavy domain and a variable light domain of any of the CD28 binding domains in FIG. 38 or a variant thereof,
iii) a variable heavy domain of any of the CD28 binding domains in FIGS. 41-74, and a variable light domain of any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain of any of the CD28 binding domains in FIGS. 41-74, and a variable light domain having the amino acid sequence of SEQ ID NO:20 (FIG. 24) or a variant thereof, and
vi) a variable heavy domain and variable light domain of any of the CD28 binding domains in FIG. 81 or a variant thereof.

9. Non-Heterodimeric Bispecific Antibodies

As will be appreciated by those in the art, the subject antibodies provided herein (e.g., anti-PD-L1×anti-CD28 and anti-PD-L1×anti-CD28 antibodies) provided herein can also be included in non-heterodimeric bispecific formats (see FIG. 82I). In this format, the anti-CD28×anti-T anti-PD-L1×anti-CD28 and anti-PD-L1×anti-CD28 antibodies include: 1) a first monomer comprising a VH1-CH1-hinge-CH2-CH3; 2) a second monomer comprising a VH2-CH1-hinge-CH2-CH3; 3) a first light chain comprising a VL1-CL; and 4) a second light chain comprising a VL2-CL. In such embodiments, the VH1 and VL1 form a first antigen binding domain and VH2 and VL2 form a second antigen binding domain. One of the first or second antigen binding domains binds CD28 and the other antigen binding domain binds PD-L1 or PD-L2.

Any suitable PD-L1 binding domain can be included in the subject non-heterodimeric bispecific format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20 or a variant thereof,
ii) a variable heavy domain and variable light domain of any of the PD-L1 binding domains in FIGS. 17 and 21 or a variant thereof,
iii) a variable heavy domain of any of the PD-L1 binding domains in FIGS. 25 and 26, and a variable light domain of any of the PD-L1 binding domains in FIGS. 25 and 26 or a variant thereof,
iv) a variable heavy domain and variable light domain of any of the PD-L1 binding domains in FIGS. 25 and 26 or a variant thereof,
v) a variable heavy domain of any of the PD-L1 binding domains in FIGS. 25 and 26, and a variable light domain having the amino acid sequence of SEQ ID NO:20 (FIG. 24) or a variant thereof, and
vi) a variable heavy domain and variable light domain of any of the PD-L1 binding domains in FIG. 28 or a variant thereof.

Any suitable PD-L2 binding domain can be included in the subject non-heterodimeric bispecific antibody including those provided herein (FIGS. 29-34 and the Sequence Listing) or a variant thereof. In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32 or a variant thereof,
ii) a variable heavy domain and variable light domain of any of the PD-L2 binding domains in FIGS. 29-32 or a variant thereof,
iii) a variable heavy domain of any of the PD-L2 binding domains in FIGS. 29-32, and a variable light domain having the amino acid sequence of SEQ ID NO:20 (FIG. 24) or a variant thereof, and
iv) a variable heavy domain and variable light domain of any of the PD-L2 binding domains in FIG. 34 or a variant thereof.

Any suitable CD28 binding domain can be included in subject non-heterodimeric bispecific format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38 or a variant thereof,
ii) a variable heavy domain and a variable light domain of any of the CD28 binding domains in FIG. 38 or a variant thereof,
iii) a variable heavy domain of any of the CD28 binding domains in FIGS. 41-74, and a variable light domain of any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain of any of the CD28 binding domains in FIGS. 41-74, and a variable light domain having the amino acid sequence of SEQ ID NO:20 (FIG. 24) or a variant thereof, and
vi) a variable heavy domain and variable light domain of any of the CD28 binding domains in FIG. 81 or a variant thereof.

10. One-Armed Central-scFv

One heterodimeric antibody format that finds particular use in subject antibodies (e.g., anti-PD-L1×anti-CD28 and anti-PD-L1×anti-CD28 antibodies) provided herein is the one-armed central-scFv format shown in FIG. 82J. In this embodiment, one monomer comprises just an Fc domain, while the other monomer includes a Fab domain (a first antigen binding domain), a scFv domain (a second antigen binding domain) and an Fc domain, where the scFv domain is inserted between the Fc domain and the Fc domain.

In this embodiment, one monomer comprises a first heavy chain comprising a first variable heavy domain, a CH1 domain and Fc domain, with a scFv comprising a scFv variable light domain, an scFv linker and a scFv variable heavy domain. The scFv is covalently attached between the C-terminus of the CH1 domain of the heavy constant domain and the N-terminus of the first Fc domain using domain linkers, in either orientation, VH1-CH1-[optional domain linker]-VH2-scFv linker-VL2-[optional domain linker]-CH2-CH3 or VH1-CH1-[optional domain linker]-VL2-scFv linker-VH2-[optional domain linker]-CH2-CH3. The second monomer comprises an Fc domain (CH2-CH3). This embodiment further utilizes a light chain comprising a variable light domain and a constant light domain, that associates with the heavy chain to form a Fab. As for many of the embodiments herein, these constructs include skew variants, pI variants, ablation variants, additional Fc variants, etc. as desired and described herein.

Any suitable PD-L1 binding domain can be included in the subject one-armed central-scFv format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20 or a variant thereof,
ii) a variable heavy domain and variable light domain of any of the PD-L1 binding domains in FIGS. 17 and 21 or a variant thereof,
iii) a variable heavy domain of any of the PD-L1 binding domains in FIGS. 25 and 26, and a variable light domain of any of the PD-L1 binding domains in FIGS. 25 and 26 or a variant thereof,
iv) a variable heavy domain and variable light domain of any of the PD-L1 binding domains in FIGS. 25 and 26 or a variant thereof,
v) a variable heavy domain of any of the PD-L1 binding domains in FIGS. 25 and 26, and a variable light domain having the amino acid sequence of SEQ ID NO:20 (FIG. 24) or a variant thereof, and
vi) a variable heavy domain and variable light domain of any of the PD-L1 binding domains in FIG. 28 or a variant thereof.

Any suitable PD-L2 binding domain can be included in the subject one-armed central-scFv format antibody including those provided herein (FIGS. 29-34 and the Sequence Listing) or a variant thereof. In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32 or a variant thereof,
ii) a variable heavy domain and variable light domain of any of the PD-L2 binding domains in FIGS. 29-32 or a variant thereof,
iii) a variable heavy domain of any of the PD-L2 binding domains in FIGS. 29-32, and a variable light domain having the amino acid sequence of SEQ ID NO:20 (FIG. 24) or a variant thereof, and
iv) a variable heavy domain and variable light domain of any of the PD-L2 binding domains in FIG. 34 or a variant thereof.

Any suitable CD28 binding domain can be included in subject one-armed central-scFv format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38 or a variant thereof,
ii) a variable heavy domain and a variable light domain of any of the CD28 binding domains in FIG. 38 or a variant thereof,
iii) a variable heavy domain of any of the CD28 binding domains in FIGS. 41-74, and a variable light domain of any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain of any of the CD28 binding domains in FIGS. 41-74, and a variable light domain having the amino acid sequence of SEQ ID NO:20 (FIG. 24) or a variant thereof, and
vi) a variable heavy domain and variable light domain of any of the CD28 binding domains in FIG. 81 or a variant thereof.

11. mAb-Fv Format

One heterodimeric antibody format that finds particular use in subject antibodies provided herein (e.g., anti-PD-L1×anti-CD28 and anti-PD-L1×anti-CD28 antibodies) is the mAb-Fv format (FIG. 82K). In this embodiment, the format relies on the use of a C-terminal attachment of an “extra” variable heavy domain to one monomer and the C-terminal attachment of an “extra” variable light domain to the other monomer, thus forming a third antigen binding domain (i.e. an “extra” Fv domain), wherein the Fab portions of the two monomers bind PD-L1 or PD-L2 and the “extra” Fv domain binds CD28.

In this embodiment, the first monomer comprises a first heavy chain, comprising a first variable heavy domain and a first constant heavy domain comprising a first Fc domain, with a first variable light domain covalently attached to the C-terminus of the first Fc domain using a domain linker (vh1-CH1-hinge-CH2-CH3-[optional linker]-vl2). The second monomer comprises a second variable heavy domain, a second constant heavy domain comprising a second Fc domain, and a third variable heavy domain covalently attached to the C-terminus of the second Fc domain using a domain linker (vh1-CH1-hinge-CH2-CH3-[optional linker]-vh2. This embodiment further utilizes a common light chain comprising a variable light domain and a constant light domain, which associates with the heavy chains to form two identical Fabs that include two identical Fvs. The two C-terminally attached variable domains make up the “extra” third Fv. As for many of the embodiments herein, these constructs include skew variants, pI variants, ablation variants, additional Fc variants, etc. as desired and described herein.

Any suitable PD-L1 binding domain can be included in the subject mAb-Fv format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20 or a variant thereof,
ii) a variable heavy domain and variable light domain of any of the PD-L1 binding domains in FIGS. 17 and 21 or a variant thereof,
iii) a variable heavy domain of any of the PD-L1 binding domains in FIGS. 25 and 26, and a variable light domain of any of the PD-L1 binding domains in FIGS. 25 and 26 or a variant thereof,
iv) a variable heavy domain and variable light domain of any of the PD-L1 binding domains in FIGS. 25 and 26 or a variant thereof,
v) a variable heavy domain of any of the PD-L1 binding domains in FIGS. 25 and 26, and a variable light domain having the amino acid sequence of SEQ ID NO:20 (FIG. 24) or a variant thereof, and
vi) a variable heavy domain and variable light domain of any of the PD-L1 binding domains in FIG. 28 or a variant thereof.

Any suitable PD-L2 binding domain can be included in the subject mAb-Fv format antibody including those provided herein (FIGS. 29-34 and the Sequence Listing) or a variant thereof. In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32 or a variant thereof,
ii) a variable heavy domain and variable light domain of any of the PD-L2 binding domains in FIGS. 29-32 or a variant thereof,
iii) a variable heavy domain of any of the PD-L2 binding domains in FIGS. 29-32, and a variable light domain having the amino acid sequence of SEQ ID NO:20 (FIG. 24) or a variant thereof, and
iv) a variable heavy domain and variable light domain of any of the PD-L2 binding domains in FIG. 34 or a variant thereof.

Any suitable CD28 binding domain can be included in subject mAb-Fv format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38 or a variant thereof,
ii) a variable heavy domain and a variable light domain of any of the CD28 binding domains in FIG. 38 or a variant thereof,
iii) a variable heavy domain of any of the CD28 binding domains in FIGS. 41-74, and a variable light domain of any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain of any of the CD28 binding domains in FIGS. 41-74, and a variable light domain having the amino acid sequence of SEQ ID NO:20 (FIG. 24) or a variant thereof, and
vi) a variable heavy domain and variable light domain of any of the CD28 binding domains in FIG. 81 or a variant thereof.

12. Central-Fv format

One heterodimeric antibody format that finds particular use in subject antibodies provided herein (e.g., anti-PD-L1×anti-CD28 and anti-PD-L1×anti-CD28 antibodies) is the central-Fv format shown in FIG. 82L. In this embodiment, the format relies on the use of an inserted Fv domain thus forming an “extra” third antigen binding domain, wherein the Fab portions of the two monomers bind PD-L1 or PD-L2 and the “extra” central-Fv domain binds CD28. The Fv domain is inserted between the Fc domain and the CH1-Fv region of the monomers, thus providing a third antigen binding domain, wherein each monomer contains a component of the Fv (e.g. one monomer comprises a variable heavy domain and the other a variable light domain of the “extra” central Fv domain).

In this embodiment, one monomer comprises a first heavy chain comprising a first variable heavy domain, a CH1 domain, and Fc domain and an additional variable light domain. The additional variable light domain is covalently attached between the C-terminus of the CH1 domain of the heavy constant domain and the N-terminus of the first Fc domain using domain linkers (vh1-CH1-[optional linker]-vl2-hinge-CH2-CH3). The other monomer comprises a first heavy chain comprising a first variable heavy domain, a CH1 domain and Fc domain and an additional variable heavy domain (vh1-CH1-[optional linker]-vh2-hinge-CH2-CH3). The additional variable heavy domain is covalently attached between the C-terminus of the CH1 domain of the heavy constant domain and the N-terminus of the first Fc domain using domain linkers. This embodiment utilizes a common light chain comprising a variable light domain and a constant light domain, that associates with the heavy chains to form two identical Fabs that each bind TROP2. The additional variable heavy domain and additional variable light domain form an “extra” central Fv that binds CD28. As for many of the embodiments herein, these constructs include skew variants, pI variants, ablation variants, additional Fc variants, etc. as desired and described herein.

Any suitable PD-L1 binding domain can be included in the subject central-Fv format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20 or a variant thereof,
ii) a variable heavy domain and variable light domain of any of the PD-L1 binding domains in FIGS. 17 and 21 or a variant thereof,
iii) a variable heavy domain of any of the PD-L1 binding domains in FIGS. 25 and 26, and a variable light domain of any of the PD-L1 binding domains in FIGS. 25 and 26 or a variant thereof,
iv) a variable heavy domain and variable light domain of any of the PD-L1 binding domains in FIGS. 25 and 26 or a variant thereof,
v) a variable heavy domain of any of the PD-L1 binding domains in FIGS. 25 and 26, and a variable light domain having the amino acid sequence of SEQ ID NO:20 (FIG. 24) or a variant thereof, and
vi) a variable heavy domain and variable light domain of any of the PD-L1 binding domains in FIG. 28 or a variant thereof.

Any suitable PD-L2 binding domain can be included in the subject central-Fv format antibody including those provided herein (FIGS. 29-34 and the Sequence Listing) or a variant thereof. In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32 or a variant thereof,
ii) a variable heavy domain and variable light domain of any of the PD-L2 binding domains in FIGS. 29-32 or a variant thereof,
iii) a variable heavy domain of any of the PD-L2 binding domains in FIGS. 29-32, and a variable light domain having the amino acid sequence of SEQ ID NO:20 (FIG. 24) or a variant thereof, and
iv) a variable heavy domain and variable light domain of any of the PD-L2 binding domains in FIG. 34 or a variant thereof.

Any suitable CD28 binding domain can be included in subject central-Fv format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38 or a variant thereof,
ii) a variable heavy domain and a variable light domain of any of the CD28 binding domains in FIG. 38 or a variant thereof,
iii) a variable heavy domain of any of the CD28 binding domains in FIGS. 41-74, and a variable light domain of any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain of any of the CD28 binding domains in FIGS. 41-74, and a variable light domain having the amino acid sequence of SEQ ID NO:20 (FIG. 24) or a variant thereof, and
vi) a variable heavy domain and variable light domain of any of the CD28 binding domains in FIG. 81 or a variant thereof.

13. Trident Format

In some embodiments, the subject antibodies provided herein (e.g., anti-PD-L1×anti-CD28 and anti-PD-L1×anti-CD28 antibodies) are in the “Trident” format as generally described in WO2015/184203, hereby expressly incorporated by reference in its entirety and in particular for the Figures, Legends, definitions and sequences of “Heterodimer-Promoting Domains” or “HPDs”, including “K-coil” and “E-coil” sequences. Tridents rely on using two different HPDs that associate to form a heterodimeric structure as a component of the structure, see FIG. 82M. In this embodiment, the Trident format include a “traditional” heavy and light chain (e.g. VH1-CH1-hinge-CH2-CH3 and VL1-CL), a third chain comprising a first “diabody-type binding domain” or “DART®”, VH2-(linker)-VL3-HPD1 and a fourth chain comprising a second DART®, VH3-(linker)-(linker)-VL2-HPD2. The VH1 and VL1 form a first ABD, the VH2 and VL2 form a second ABD, and the VH3 and VL3 form a third ABD. In some cases, as is shown in FIG. 82M, the second and third ABDs bind the same antigen.

Any suitable PD-L1 binding domain can be included in the subject Trident format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain of any of the PD-L1 variable heavy domains in FIGS. 17 and 19, and a variable light domain of any of the PD-L1 variable light domains in FIGS. 17 and 20 or a variant thereof,
ii) a variable heavy domain and variable light domain of any of the PD-L1 binding domains in FIGS. 17 and 21 or a variant thereof,
iii) a variable heavy domain of any of the PD-L1 binding domains in FIGS. 25 and 26, and a variable light domain of any of the PD-L1 binding domains in FIGS. 25 and 26 or a variant thereof,
iv) a variable heavy domain and variable light domain of any of the PD-L1 binding domains in FIGS. 25 and 26 or a variant thereof,
v) a variable heavy domain of any of the PD-L1 binding domains in FIGS. 25 and 26, and a variable light domain having the amino acid sequence of SEQ ID NO:20 (FIG. 24) or a variant thereof, and
vi) a variable heavy domain and variable light domain of any of the PD-L1 binding domains in FIG. 28 or a variant thereof.

Any suitable PD-L2 binding domain can be included in the subject Trident format antibody including those provided herein (FIGS. 29-34 and the Sequence Listing) or a variant thereof. In some embodiments, the PD-L2 binding domain includes a variable heavy domain and variable light domain selected from the following:
i) a variable heavy domain of any of the PD-L2 binding domains in FIGS. 29-32, and a variable light domain of any of the PD-L2 binding domains in FIGS. 29-32 or a variant thereof,
ii) a variable heavy domain and variable light domain of any of the PD-L2 binding domains in FIGS. 29-32 or a variant thereof,
iii) a variable heavy domain of any of the PD-L2 binding domains in FIGS. 29-32, and a variable light domain having the amino acid sequence of SEQ ID NO:20 (FIG. 24) or a variant thereof, and
iv) a variable heavy domain and variable light domain of any of the PD-L2 binding domains in FIG. 34 or a variant thereof.

Any suitable CD28 binding domain can be included in subject Trident format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain of any of the CD28 binding domains in FIGS. 35, 36, and 38, and a variable light domain of any of the CD28 binding domains in FIGS. 35, 37, and 38 or a variant thereof,
ii) a variable heavy domain and a variable light domain of any of the CD28 binding domains in FIG. 38 or a variant thereof,
iii) a variable heavy domain of any of the CD28 binding domains in FIGS. 41-74, and a variable light domain of any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain of any of the CD28 binding domains in FIGS. 41-74, and a variable light domain having the amino acid sequence of SEQ ID NO:20 (FIG. 24) or a variant thereof, and
vi) a variable heavy domain and variable light domain of any of the CD28 binding domains in FIG. 81 or a variant thereof.

14. 1+1+1 stackFab₂-scFv-Fc

In one aspect, provided herein are novel 1+1+1 stackFab₂-scFv-Fc format antibodies (FIG. 83A). This antibody format includes three antigen binding domains: two Fab portions and an scFv binding domain. Each of the Fab portions include a common variable light chain. This heterodimeric antibody format that finds particular use in the subject trispecific antibodies provided herein (e.g., anti-PD-L1×anti-PD-L2×anti-CD28 antibodies).

The 1+1+1 stackFab₂-scFv-Fc format includes a first monomer, second monomer and a third monomer (common light chain). The first monomer includes from N-terminal to C-terminal, scFv-linker-CH2-CH3 wherein CH2-CH3 is a first Fc domain. The second monomer includes from N-terminal to C-terminal, VH1-CH1-linker-VH2-CH1-hinge-CH2-CH3 wherein VH1 is a first variable heavy domain, VH2 is a second variable heavy domain, and CH2-CH3 is a second Fc domain. The third monomer includes from N-terminal to C-terminal, VL1-CL, wherein VL1 is a first variable light domain and CL is a constant light domain. Further, the scFv comprises a third VH domain (VH3), a scFv linker, and a second variable light domain (VL2). In this format, the first variable heavy domain and the first variable light domain form a first antigen binding domain, the second variable heavy domain and the first variable light domain form a second antigen binding domain, and the third variable heavy domain and second variable light domain form a third antigen binding domain.

In some embodiments, this heterodimeric antibody is a trispecific antibody that binds PD-L1, PD-L2, and CD28, wherein each of the first antigen binding domain, second antigen binding domain, and third antigen binding domain is selected from the group consisting of a PD-L1 antigen binding domain, a PD-L2 antigen binding domain, and a CD 28 binding domain.

In exemplary embodiments, the first and second Fc domains are variant Fc domains. In some embodiments, the first and second Fc domains of the 1+1+1 stackFab₂-scFv-Fc format antibody are variant Fc domains that include heterodimerization skew variants (e.g., a set of amino acid substitutions as shown in FIGS. 4 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering. In exemplary embodiments, the first variant Fc domain includes heterodimerization skew variants S364K/E357Q and the second variant Fc domain includes heterodimerization skew variants L368D/K370S, wherein numbering is according to EU numbering.

In some embodiments, the variant Fc domains include ablation variants (including those shown in FIG. 6). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 5). In exemplary embodiments, the constant domain (CH1-hinge-CH2-CH3) of the second monomer includes pI variants N208D/Q295E/N384D/Q418E/N421D, wherein numbering is according to EU numbering.

In exemplary embodiments, the CH1-hinge-CH2-CH3 of the first monomer comprises amino acid variants S364K/E357Q/E233P/L234V/L235A/G236del/S267K, the second Fc domain comprises amino acid variants L368D/K370S/N208D/Q295E/N384D/Q418E/N421D/E233P/L234V/L235A/G236del/S267K, wherein numbering is according to EU numbering.

In some embodiments, the scFv of the 1+1+1 stackFab₂-scFv-Fc format antibody provided herein includes a charged scFv linker (including those shown in FIG. 7). In some embodiments, the 1+1 Fab-scFv-Fc format antibody provided herein includes FcRn variants M428L/N434S, wherein numbering is according to EU numbering.

Any suitable PD-L1 binding domain can be included in the subject 1+1+1 stackFab₂-scFv-Fc format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain selected from any of the PD-L1 variable heavy domain in FIGS. 17 and 19, and a variable light domain selected from any of the PD-L1 variable light domain in FIGS. 17 and 20 or a variant thereof,
ii) a variable heavy domain and variable light domain of any of the PD-L1 binding domains in FIGS. 17 and 21 or a variant thereof,
iii) a variable heavy domain selected from any of the PD-L1 binding domain in FIGS. 25 and 26, and a variable light domain selected from any of the PD-L1 binding domain in FIGS. 25 and 26 or a variant thereof,
iv) a variable heavy domain and variable light domain of any of the PD-L1 binding domains in FIGS. 25 and 26 or a variant thereof,
v) a variable heavy domain selected from any of the PD-L1 binding domains in FIGS. 25 and 26, and a variable light domain having the amino acid sequence of SEQ ID NO:20 (FIG. 24) or a variant thereof,
vi) a variable heavy domain and variable light domain of a PD-L1 binding domains in FIG. 28 or a variant thereof; and
vii) a variable heavy domain selected from any of the variable heavy domains in FIG. 161 or a variant thereof, and a variable light domain having the amino acid sequence of SEQ ID NO:3239 or a variant thereof.

Any suitable PD-L2 binding domain can be included in the subject 1+1+1 stackFab₂-scFv-Fc format antibody including those provided herein (FIGS. 29-34 and the Sequence Listing) or a variant thereof. In some embodiments, the PD-L2 binding domain includes a variable heavy domain and variable light domain selected from the following:
i) a variable heavy domain selected from any of the PD-L2 binding domain in FIGS. 29-32, and a variable light domain selected from any of the PD-L2 binding domain in FIGS. 29-32 or a variant thereof,
ii) a variable heavy domain and variable light domain of any of the PD-L2 binding domains in FIGS. 29-32 or a variant thereof,
iii) a variable heavy domain selected from any of the PD-L2 binding domain in FIGS. 29-32, and a variable light domain having the amino acid sequence of SEQ ID NO:20 (FIG. 24) or a variant thereof,
iv) a variable heavy domain and variable light domain of any of the PD-L2 binding domains in FIG. 34 or a variant thereof,
v) a variable heavy domain selected from any of the variable heavy domains in FIG. 162 or a variant thereof, and a variable light domain having the amino acid sequence of SEQ ID NO:3239 or a variant thereof.

Any suitable CD28 binding domain can be included in subject 1+1+1 stackFab₂-scFv-Fc format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain selected from any of the CD28 binding domains in FIGS. 35, 36, and 38, and a variable light domain selected from any of the CD28 binding domains in FIGS. 35, 37, and 38 or a variant thereof,
ii) a variable heavy domain and a variable light domain selected from any of the of the CD28 binding domains in FIG. 38 or a variant thereof,
iii) a variable heavy domain of any of the CD28 binding domains in FIGS. 41-74, and a variable light domain selected from any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain selected from any of the CD28 binding domains in FIGS. 41-74, and a variable light domain having the amino acid sequence of SEQ ID NO:20 (FIG. 24) or a variant thereof,
vi) a variable heavy domain and variable light domain of any of the CD28 binding domains in FIG. 81 or a variant thereof, and
vii) a variable heavy domain selected from any of the variable heavy domains in FIG. 163 or a variant thereof, and a variable light domain of selected from any of the variable light domains in FIGS. 35 and 37, or a variable light domain having the amino acid sequence of SEQ ID NO:3239 or a variant thereof.

FIG. 88 provides exemplary anti-PD-L1×anti-PD-L2×anti-CD28 trispecific antibodies in the 1+1+1 stackFab₂-scFv-Fc format.

15. 1+1+1 Fab-(Fab-scFv)-Fc

In one aspect, provided herein are novel 1+1+1 Fab-(Fab-scFv)-Fc format antibodies (FIG. 83B). This antibody format includes three antigen binding domains: two Fab portions and an scFv binding domain. Each of the Fab portions include a common variable light chain. This heterodimeric antibody format that finds particular use in the subject trispecific antibodies provided herein (e.g., anti-PD-L1×anti-PD-L2×anti-CD28 antibodies).

The 1+1+1 Fab-(Fab-scFv)-Fc format includes a first monomer, second monomer and a third monomer (common light chain). The first monomer includes from N-terminal to C-terminal, VH1-CH1-linker-scFv-linker-CH2-CH3 wherein VH1 is a first variable heavy domain, and CH2-CH3 is a first Fe domain. The second monomer includes from N-terminal to C-terminal, VH2-CH1-hinge-CH2-CH3 wherein VH2 is a second variable heavy domain, and CH2-CH3 is a second Fc domain. The common light chain comprising, from N-terminal to C-terminal, VL1-CL, wherein VL is a first variable light domain and CL is a constant light domain. In this format, the scFv comprises a third VH domain (VH3), a scFv linker, and a second variable light domain (VL2). Further, the first variable heavy domain and the first variable light domain form a first antigen binding domain, the second variable heavy domain and the first variable light domain form a second antigen binding domain, and the third variable heavy domain and second variable light domain form a third antigen binding domain.

In some embodiments, this heterodimeric antibody is a trispecific antibody that binds PD-L1, PD-L2, and CD28, wherein each of the first antigen binding domain, second antigen binding domain, and third antigen binding domain is selected from the group consisting of a PD-L1 antigen binding domain, a PD-L2 antigen binding domain, and a CD28 binding domain.

In exemplary embodiments, the first and second Fc domains are variant Fc domains. In some embodiments, the first and second Fc domains of the 1+1+1 Fab-(Fab-scFv)-Fc format antibody are variant Fc domains that include heterodimerization skew variants (e.g., a set of amino acid substitutions as shown in FIGS. 4 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering. In exemplary embodiments, the first variant Fc domain includes heterodimerization skew variants S364K/E357Q and the second variant Fc domain includes heterodimerization skew variants L368D/K370S, wherein numbering is according to EU numbering.

In some embodiments, the variant Fc domains include ablation variants (including those shown in FIG. 6). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the first monomer includes pI variants (including those shown in FIG. 5). In exemplary embodiments, the constant domain (CH1-hinge-CH2-CH3) of the second monomer includes pI variants N208D/Q295E/N384D/Q418E/N421D, wherein numbering is according to EU numbering.

In exemplary embodiments, the CH1-hinge-CH2-CH3 of the first monomer comprises amino acid variants S364K/E357Q/E233P/L234V/L235A/G236del/S267K, the second Fc domain comprises amino acid variants L368D/K370S/N208D/Q295E/N384D/Q418E/N421D/E233P/L234V/L235A/G236del/S267K, wherein numbering is according to EU numbering.

In some embodiments, the scFv of the 1+1+1 Fab-(Fab-scFv)-Fc format antibody provided herein includes a charged scFv linker (including those shown in FIG. 7). In some embodiments, the 1+1 Fab-scFv-Fc format antibody provided herein includes FcRn variants M428L/N434S, wherein numbering is according to EU numbering.

Any suitable PD-L1 binding domain can be included in the subject 1+1+1 Fab-(Fab-scFv)-Fc format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from the following:
i) a variable heavy domain selected from any of the PD-L1 variable heavy domain in FIGS. 17 and 19, and a variable light domain selected from any of the PD-L1 variable light domain in FIGS. 17 and 20 or a variant thereof,
ii) a variable heavy domain and variable light domain of any of the PD-L1 binding domains in FIGS. 17 and 21 or a variant thereof,
iii) a variable heavy domain selected from any of the PD-L1 binding domain in FIGS. 25 and 26, and a variable light domain selected from any of the PD-L1 binding domain in FIGS. 25 and 26 or a variant thereof,
iv) a variable heavy domain and variable light domain of any of the PD-L1 binding domains in FIGS. 25 and 26 or a variant thereof,
v) a variable heavy domain selected from any of the PD-L1 binding domains in FIGS. 25 and 26, and a variable light domain having the amino acid sequence of SEQ ID NO:20 (FIG. 24) or a variant thereof,
vi) a variable heavy domain and variable light domain of a PD-L1 binding domains in FIG. 28 or a variant thereof; and
vii) a variable heavy domain selected from any of the variable heavy domains in FIG. 161 or a variant thereof, and a variable light domain having the amino acid sequence of SEQ ID NO:3239 or a variant thereof.

Any suitable PD-L2 binding domain can be included in the subject 1+1+1 Fab-(Fab-scFv)-Fc format antibody including those provided herein (FIGS. 29-34 and the Sequence Listing) or a variant thereof. In some embodiments, the PD-L2 binding domain includes a variable heavy domain and variable light domain selected from the following:
i) a variable heavy domain selected from any of the PD-L2 binding domain in FIGS. 29-32, and a variable light domain selected from any of the PD-L2 binding domain in FIGS. 29-32 or a variant thereof,
ii) a variable heavy domain and variable light domain of any of the PD-L2 binding domains in FIGS. 29-32 or a variant thereof,
iii) a variable heavy domain selected from any of the PD-L2 binding domain in FIGS. 29-32, and a variable light domain having the amino acid sequence of SEQ ID NO:20 (FIG. 24) or a variant thereof,
iv) a variable heavy domain and variable light domain of any of the PD-L2 binding domains in FIG. 34 or a variant thereof,
v) a variable heavy domain selected from any of the variable heavy domains in FIG. 162 or a variant thereof, and a variable light domain having the amino acid sequence of SEQ ID NO:3239 or a variant thereof.

Any suitable CD28 binding domain can be included in subject 1+1+1 Fab-(Fab-scFv)-Fc format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain selected from any of the CD28 binding domains in FIGS. 35, 36, and 38, and a variable light domain selected from any of the CD28 binding domains in FIGS. 35, 37, and 38 or a variant thereof,
ii) a variable heavy domain and a variable light domain selected from any of the of the CD28 binding domains in FIG. 38 or a variant thereof,
iii) a variable heavy domain of any of the CD28 binding domains in FIGS. 41-74, and a variable light domain selected from any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain selected from any of the CD28 binding domains in FIGS. 41-74, and a variable light domain having the amino acid sequence of SEQ ID NO:20 (FIG. 24) or a variant thereof,
vi) a variable heavy domain and variable light domain of any of the CD28 binding domains in FIG. 81 or a variant thereof, and
vii) a variable heavy domain selected from any of the variable heavy domains in FIG. 163 or a variant thereof, and a variable light domain of selected from any of the variable light domains in FIGS. 35 and 37, or a variable light domain having the amino acid sequence of SEQ ID NO:3239 or a variant thereof.

FIG. 89 provides exemplary anti-PD-L1×anti-PD-L2×anti-CD28 trispecific antibodies in the 1+1+1 Fab-(Fab-scFv)-Fc format.

16. 1+1+1 mAb-scFv

In one aspect, provided herein are novel 1+1+1 mAb-scFv format antibodies (FIG. 83C). This antibody format includes three antigen binding domains: two Fab portions and an scFv that is attached to the C-terminal of one of the heavy chains. This heterodimeric antibody format that finds particular use in the subject trispecific antibodies provided herein (e.g., anti-PD-L1×anti-PD-L2×anti-CD28 antibodies). In some embodiments of this format, the Fab portions each bind PD-L1 and PD-L2. In some embodiments, the “extra” scFv domain binds CD28. That is, this mAb-scFv format is a trivalent antibody.

The 1+1+1 mAb-scFv format antibody generally includes a first monomer, a second monomer, and a third monomer (common light chain). The first monomer includes from N-terminal to C-terminal, VH1-CH1-hinge-CH2-CH3-domain linker-scFv, wherein VH1 is a first variable heavy domain, and CH2-CH3 is a first Fc domain. The second monomer includes from N-terminal to C-terminal, a VH2-CH1-hinge-CH2-CH3, wherein VH2 is a second variable heavy domain and CH2-CH3 is a second Fc domain. The common light chain includes from N-terminal to C-terminal, VL1-CL, wherein VL1 is a first variable light domain and CL is a constant light domain. In this format, the scFv comprises a second VH3 domain (VH3), a scFv linker, and a second variable light domain (VL2). Further, the VH1 and VL1 forms a first antigen binding domain (ABD), VH2 and VL1 form a second ABD, and VH3 and VL2 form a third antigen binding domain.

In some embodiments, this heterodimeric antibody is a trispecific antibody that binds PD-L1, PD-L2, and CD28, wherein each of the first antigen binding domain, second antigen binding domain, and third antigen binding domain is selected from the group consisting of a PD-L1 antigen binding domain, a PD-L2 antigen binding domain, and a CD 28 binding domain.

In exemplary embodiments, the first and second Fc domains are variant Fc domains. In some embodiments, the first and second Fc domains of the 1+1+1 mAb-scFv format antibody are variant Fc domains that include heterodimerization skew variants (e.g., a set of amino acid substitutions as shown in FIGS. 4 and 9). Particularly useful heterodimerization skew variants include S364K/E357Q:L368D/K370S; L368D/K370S: S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fc domain includes heterodimerization skew variants L368D/K370S and the second variant Fc domain includes heterodimerization skew variants S364K/E357Q, wherein numbering is according to EU numbering. In exemplary embodiments, the first variant Fc domain includes heterodimerization skew variants S364K/E357Q and the second variant Fc domain includes heterodimerization skew variants L368D/K370S, wherein numbering is according to EU numbering.

In some embodiments, the variant Fc domains include ablation variants (including those shown in FIG. 6). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the second monomer includes pI variants (including those shown in FIG. 5). In exemplary embodiments, the constant domain (CH1-hinge-CH2-CH3) of the second monomer includes pI variants N208D/Q295E/N384D/Q418E/N421D, wherein numbering is according to EU numbering.

In exemplary embodiments, the CH1-hinge-CH2-CH3 of the first monomer comprises amino acid variants S364K/E357Q/E233P/L234V/L235A/G236del/S267K, the second Fc domain comprises amino acid variants L368D/K370S/N208D/Q295E/N384D/Q418E/N421D/E233P/L234V/L235A/G236del/S267K, wherein numbering is according to EU numbering.

In some embodiments, the scFv of the 1+1+1 mAb-scFv format antibody provided herein includes a charged scFv linker (including those shown in FIG. 7). In some embodiments, the 1+1 Fab-scFv-Fc format antibody provided herein includes FcRn variants M428L/N434S, wherein numbering is according to EU numbering.

Any suitable PD-L1 binding domain can be included in the subject 1+1+1 mAb-scFv format antibody including those provided herein (FIGS. 17, 19, 20, 21, 24-26, and 28 and the Sequence Listing) or a variant thereof. In some embodiments of the 2+1 mAb-scFv, the VH1 of the first and second monomer and the VL1 of the common light chain each form a PD-L1 binding domain. In some embodiments, the scFv of the second monomer is a PD-L1 binding domain.

In some embodiments, the PD-L1 binding domain includes a variable heavy domain and variable light domain selected from the following:
i) a variable heavy domain selected from any of the PD-L1 variable heavy domain in FIGS. 17 and 19, and a variable light domain selected from any of the PD-L1 variable light domain in FIGS. 17 and 20 or a variant thereof,
ii) a variable heavy domain and variable light domain of any of the PD-L1 binding domains in FIGS. 17 and 21 or a variant thereof,
iii) a variable heavy domain selected from any of the PD-L1 binding domain in FIGS. 25 and 26, and a variable light domain selected from any of the PD-L1 binding domain in FIGS. 25 and 26 or a variant thereof,
iv) a variable heavy domain and variable light domain of any of the PD-L1 binding domains in FIGS. 25 and 26 or a variant thereof,
v) a variable heavy domain selected from any of the PD-L1 binding domains in FIGS. 25 and 26, and a variable light domain having the amino acid sequence of SEQ ID NO:20 (FIG. 24) or a variant thereof,
vi) a variable heavy domain and variable light domain of a PD-L1 binding domains in FIG. 28 or a variant thereof; and
vii) a variable heavy domain selected from any of the variable heavy domains in FIG. 161 or a variant thereof, and a variable light domain having the amino acid sequence of SEQ ID NO:3239 or a variant thereof.

Any suitable PD-L2 binding domain can be included in the subject 1+1+1 mAb-scFv format antibody including those provided herein (FIGS. 29-34 and the Sequence Listing) or a variant thereof. In some embodiments of the 2+1 mAb-scFv, the VH1 of the first and second monomer and the VL1 of the common light chain each form a PD-L2 binding domain. In some embodiments, the scFv of the second monomer is a PD-L2 binding domain.

In some embodiments, the PD-L2 binding domain includes a variable heavy domain and variable light domain selected from the following:
i) a variable heavy domain selected from any of the PD-L2 binding domain in FIGS. 29-32, and a variable light domain selected from any of the PD-L2 binding domain in FIGS. 29-32 or a variant thereof,
ii) a variable heavy domain and variable light domain of any of the PD-L2 binding domains in FIGS. 29-32 or a variant thereof,
iii) a variable heavy domain selected from any of the PD-L2 binding domain in FIGS. 29-32, and a variable light domain having the amino acid sequence of SEQ ID NO:20 (FIG. 24) or a variant thereof,
iv) a variable heavy domain and variable light domain of any of the PD-L2 binding domains in FIG. 34 or a variant thereof,
v) a variable heavy domain selected from any of the variable heavy domains in FIG. 162 or a variant thereof, and a variable light domain having the amino acid sequence of SEQ ID NO:3239 or a variant thereof.

Any suitable CD28 binding domain can be included in subject 1+1+1 mAb-scFv format antibody, including any of the CD28 binding domains provided herein (see, e.g., FIGS. 35, 37, 38, 41-74 and 81 and the Sequence Listing) or a variant thereof. In some embodiments, the CD28 binding domain includes a variable heavy domain and variable light domain selected from the following:
i) a variable heavy domain selected from any of the CD28 binding domains in FIGS. 35, 36, and 38, and a variable light domain selected from any of the CD28 binding domains in FIGS. 35, 37, and 38 or a variant thereof,
ii) a variable heavy domain and a variable light domain selected from any of the of the CD28 binding domains in FIG. 38 or a variant thereof,
iii) a variable heavy domain of any of the CD28 binding domains in FIGS. 41-74, and a variable light domain selected from any of the CD28 binding domains in FIGS. 41-74 or a variant thereof,
iv) a variable heavy domain and variable light domain of any of the CD28 binding domains in FIGS. 41-74 or a variant thereof,
v) a variable heavy domain selected from any of the CD28 binding domains in FIGS. 41-74, and a variable light domain having the amino acid sequence of SEQ ID NO:20 (FIG. 24) or a variant thereof,
vi) a variable heavy domain and variable light domain of any of the CD28 binding domains in FIG. 81 or a variant thereof, and
vii) a variable heavy domain selected from any of the variable heavy domains in FIG. 163 or a variant thereof, and a variable light domain of selected from any of the variable light domains in FIGS. 35 and 37, or a variable light domain having the amino acid sequence of SEQ ID NO:3239 or a variant thereof.

FIGS. 10 and 11 show some exemplary Fc domain sequences that are useful with the 1+1+1 mAb-scFv format antibody. The “monomer 1” sequences depicted in FIG. 10 typically refer to the Fc domain of the “Fab-Fc heavy chain” and the “monomer 2” sequences refer to the Fc domain of the “Fab-Fc-scFv” heavy chain.” In addition, FIG. 12 provides exemplary CH1 (optionally including hinge or half-hinge domains) that can be used in either the “Fab-Fc heavy chain” monomer or the “Fab-Fc-scFv” heavy chain.” FIG. 13 provides exemplary hinge domains that may be used in either the “Fab-Fc heavy chain” monomer or the “Fab-Fc-scFv” heavy chain.” Further, FIG. 14 provides useful CL sequences that can be used with this format.

An exemplary anti-PD-L1×anti-CD28×antibody in the 1+1+1 mAb-scFv format is depicted in FIG. 90.

17. 1+1+1 stackFab₂-Fab-Fc

In one aspect, provided herein are novel 1+1+1 stackFab₂-Fab-Fc format antibodies (FIG. 83D). This antibody format includes three antigen binding domains: three Fab portions that each share a common light chain (CLC). This heterodimeric antibody format finds particular use in the subject trispecific antibodies provided herein (e.g., anti-PD-L1×anti-PD-L2×anti-CD28 antibodies).

This antibody includes a first monomer, a second monomer, and three common light chains that have the same sequence (i.e., a first common light chain, a second common light chain, and a third common light chain). The first monomer includes from N-terminal to C-terminal, VH1-CH1-hinge-CH2-CH3, wherein VH1 is a first variable heavy domain and CH2-CH3 is a first Fc domain. The second monomer includes from N-terminal to C-terminal, VH2-CH1-linker-VH3-CH1-hinge-CH2-CH3 wherein VH2 is a second variable heavy domain, VH3 is a third variable heavy domain, and CH2-CH3 is a second Fc domain. The common light chains each include from N-terminal to C-terminal, VL-CL, wherein VL is a common variable light domain and CL is a constant light domain. In this format, the first variable heavy domain and the common variable light domain of the first common light chain form a first antigen binding domain, the second variable heavy domain and the common variable light domain of the second common light chain form a second antigen binding domain, and the third variable heavy domain and the common variable light domain of the third common light chain form a third antigen binding domain.

In some embodiments, this heterodimeric antibody is a trispecific antibody that binds PD-L1, PD-L2, and CD28, wherein each of the first antigen binding domain, second antigen binding domain, and third antigen binding domain is selected from the group consisting of a PD-L1 antigen binding domain, a PD-L2 antigen binding domain, and a CD28 binding domain.

In exemplary embodiments, the first and second Fc domains are variant Fc domains. In some embodiments, the first and second Fc domains of the 1+1+1 stackFab₂-Fab-Fc format antibody are variant Fc domains that include heterodimerization skew variants (e.g., a set of amino acid substitutions as shown in FIGS. 4 and 9). Particularly useful heterodimerization skew variants include S364K/E357Q:L368D/K370S; L368D/K370S:S364K; L368E/K370S:S364K; T411T/E360E/Q362E:D401K; L368D/K370S:S364K/E357L; K370S:S364K/E357Q; T366S/L368A/Y407V:T366W and T366S/L368A/Y407V/Y349C:T366W/S354C (EU numbering)). In exemplary embodiments, one of the first or second variant Fc domains includes heterodimerization skew variants L368D/K370S and the other of the first or second variant Fc domains includes heterodimerization skew variants S364K/E357Q, wherein numbering is according to EU numbering. In exemplary embodiments, the first variant Fe domain includes heterodimerization skew variants L368D/K370S and the second variant Fc domain includes heterodimerization skew variants S364K/E357Q, wherein numbering is according to EU numbering. In exemplary embodiments, the first variant Fc domain includes heterodimerization skew variants S364K/E357Q and the second variant Fc domain includes heterodimerization skew variants L368D/K370S, wherein numbering is according to EU numbering.

In some embodiments, the variant Fc domains include ablation variants (including those shown in FIG. 6). In some embodiments, each of the first and second variant Fc domains include ablation variants E233P/L234V/L235A/G236_/S267K, wherein numbering is according to EU numbering.

In some embodiments, the constant domain (CH1-hinge-CH2-CH3) of the second monomer includes pI variants (including those shown in FIG. 5). In exemplary embodiments, the constant domain (CH1-hinge-CH2-CH3) of the first monomer includes pI variants N208D/Q295E/N384D/Q418E/N421D, wherein numbering is according to EU numbering.

In exemplary embodiments, the CH1-hinge-CH2-CH3 of the first monomer comprises amino acid variants L368D/K370S/N208D/Q295E/N384D/Q418E/N421D/E233P/L234V/L235A/G236del/S267K, the second Fc domain comprises amino acid variants S364K/E357Q/E233P/L234V/L235A/G236del/S267K, wherein numbering is according to EU numbering.

In some embodiments, the 1+1 Fab-scFv-Fc format antibody provided herein includes FcRn variants M428L/N434S, wherein numbering is according to EU numbering.

Any suitable PD-L1 binding domain can be included in the subject 1+1+1 stackFab₂-Fab-Fc format antibody format antibody including those provided herein (FIGS. 17, 19, 20, 21, 24-26, and 28 and the Sequence Listing) or a variant thereof. In some embodiments, the PD-L1 binding domain includes a variable heavy domain and variable light domain selected from any of the variable heavy domains and variable light domains depicted in FIGS. 17, 19, 20, 21, 24-26 and 161 or a variant thereof.

In some embodiments, the PD-L1 binding domain includes a variable heavy domain and variable light domain selected from the following:
i) a variable heavy domain selected from any of the variable heavy domains in FIG. 161 or a variant thereof, and the “IGKV1-39_L1” variable light domain (SEQ ID NO:3239) or variant thereof,
ii) a variable heavy domain selected from any of the PD-L1 binding domains in FIGS. 25 and 26 or a variant thereof, and a variable light domain selected from any of the PD-L1 binding domains in FIGS. 25 and 26 or a variant thereof,
iii) a variable heavy domain and variable light domain of any of the PD-L1 binding domains in FIGS. 25 and 26 or a variant thereof, and
iv) a variable heavy domain selected from any of the PD-L1 binding domains in FIGS. 25 and 26 or a variant thereof, and a variable light domain having the amino acid sequence of SEQ ID NO:20 (FIG. 24) or a variant thereof.

In some embodiments, subject 1+1+1 stackFab₂-Fab-Fc format antibody includes a PD-L1 binding domain having a variable heavy domain having the amino acid sequence of SEQ ID NO:3251, and a variable light domain having the amino acid sequence of SEQ ID NO:3239.

Any suitable PD-L2 binding domain can be included in the subject 1+1+1 stackFab₂-Fab-Fc format antibody format antibody including those provided herein (FIGS. 29-32 and 162 and the Sequence Listing) or a variant thereof. In some embodiments, the PD-L2 binding domain includes a variable heavy domain and variable light domain selected from any of the variable heavy domains and variable light domains depicted in FIGS. 29-32 and 162 or a variant thereof.

In some embodiments, the PD-L2 binding domain includes a variable heavy domain and variable light domain selected from the following:
i) a variable heavy domain selected from any of the variable heavy domains in FIG. 162 or a variant thereof, and the “IGKV1-39_L1” variable light domain (SEQ ID NO:3239) or variant thereof,
ii) a variable heavy domain selected from any of the PD-L2 binding domains in FIGS. 29-32, and a variable light domain of any of the PD-L2 binding domains in FIGS. 29-32 or a variant thereof,
iii) a variable heavy domain and variable light domain of any of the PD-L2 binding domains in FIGS. 29-32 or a variant thereof, and
iv) a variable heavy domain selected from any of the PD-L2 binding domains in FIGS. 29-32, and a variable light domain having the amino acid sequence of SEQ ID NO:20 (FIG. 24) or a variant thereof.

In some embodiments, subject 1+1+1 stackFab₂-Fab-Fc format antibody includes a PD-L2 binding domain having a variable heavy domain having the amino acid sequence of SEQ ID NO:3319, and a variable light domain having the amino acid sequence of SEQ ID NO:3239.

In some embodiments of the 1+1+1 stackFab₂-Fab-Fc format antibody, one of the antigen binding domain is a CD28 binding domain. Any suitable CD28 binding domain can be included in the subject 2+1 CLC format antibody format antibody including those provided herein (FIGS. 241-47 and the Sequence Listing) or a variant thereof. In some embodiments, the CD28 binding domain includes a variable heavy domain and variable light domain selected from the following:
i) a variable heavy domain selected from a variable heavy domain in FIG. 163 or a variant thereof, and a variable light domain selected from a variable light domains in FIG. 35 or 37, or the “IGKV1-39_L1” variable light domain (SEQ ID NO:3239) or variant thereof,
ii) a variable heavy domain of a CD28 binding domain in FIGS. 41-74, and a variable light domain of a CD28 binding domain in FIGS. 41-74 or a variant thereof,
iii) a variable heavy domain and variable light domain of any of the CD28 binding domains in FIGS. 41-74 or a variant thereof, and
iv) a variable heavy domain of any of the CD28 binding domains in FIGS. 41-74, and a variable light domain having the amino acid sequence of SEQ ID NO:20 (FIG. 24) or a variant thereof.

In some embodiments, subject 1+1+1 stackFab₂-Fab-Fc format antibody includes a CD28 binding domain having a variable heavy domain having the amino acid sequence of SEQ ID NO: 3380, and a variable light domain having the amino acid sequence of SEQ ID NO:3239.

In one aspect, provided herein is an anti-PD-L1×anti-PD-L2×anti-CD28 trispecific antibody in the 1+1+1 stackFab₂-Fab-Fc format that includes the following:
a) a PD-L1 binding domain having a variable heavy domain having the amino acid sequence of SEQ ID NO:3251, and a variable light domain having the amino acid sequence of SEQ ID NO:3239;
b) a PD-L2 binding domain having a variable heavy domain having the amino acid sequence of SEQ ID NO:3319, and a variable light domain having the amino acid sequence of SEQ ID NO:3239; and
c) a CD28 binding domain having a variable heavy domain having the amino acid sequence of SEQ ID NO:3380, and a variable light domain having the amino acid sequence of SEQ ID NO:3239.

In some embodiments, the anti-PD-L1×anti-PD-L2×anti-CD28 trispecific antibody includes a first monomer having the amino acid sequence of SEQ ID NO:3201, a second monomer having the amino acid sequence of SEQ ID NO:3202; and a light chain having the amino acid sequence of SEQ ID NO:3203.

Exemplary anti-PD-L1×anti-PD-L2×anti-CD28 trispecific antibodies in the 1+1+1 stackFab₂-Fab-Fc format are depicted in FIG. 91.

18. Monospecific, Monoclonal Antibodies

As will be appreciated by those in the art, the novel CD28, PD-L1, and PD-L2 sequences outlined herein (see Section IV.A-C) can also be used in both monospecific antibodies (e.g., “traditional monoclonal antibodies”) or non-heterodimeric bispecific formats. Accordingly, the present invention provides monoclonal (monospecific) antibodies comprising the 6 CDRs and/or the vh and vl sequences from the figures, generally with IgG1, IgG2, IgG3 or IgG4 constant regions, with IgG1, IgG2 and IgG4 (including IgG4 constant regions comprising a S228P amino acid substitution) finding particular use in some embodiments. That is, any sequence herein with a “H_L” designation can be linked to the constant region of a human IgG1 antibody.

### VI. Nucleic Acids

In another aspect, provided herein are nucleic acid compositions encoding the antigen binding domains and antibodies provided herein (e.g., bispecific and trispecific antibodies).

As will be appreciated by those in the art, the nucleic acid compositions will depend on the format and scaffold of the heterodimeric protein. Thus, for example, when the format requires three amino acid sequences, such as for the 1+1 Fab-scFv-Fc, 2+1 Fab₂-scFv-Fc, and 2+1 mAb-scFv formats, three polynucleotides can be incorporated into one or more expression vectors for expression. In exemplary embodiments, each polynucleotide is incorporated into a different expression vector.

As is known in the art, the nucleic acids encoding the components of the binding domains and antibodies disclosed herein can be incorporated into expression vectors as is known in the art, and depending on the host cells used to produce the heterodimeric antibodies of the invention. Generally the nucleic acids are operably linked to any number of regulatory elements (promoters, origin of replication, selectable markers, ribosomal binding sites, inducers, etc.). The expression vectors can be extra-chromosomal or integrating vectors.

The polynucleotides and/or expression vectors of the invention are then transformed into any number of different types of host cells as is well known in the art, including mammalian, bacterial, yeast, insect and/or fungal cells, with mammalian cells (e.g., CHO cells), finding use in many embodiments.

In some embodiments, polynucleotides encoding each monomer are each contained within a single expression vector, generally under different or the same promoter controls. In embodiments of particular use in the present invention, each of these polynucleotides are contained on different expression vectors. As shown herein and in U.S. 62/025,931, hereby incorporated by reference, different vector ratios can be used to drive heterodimer formation. That is, surprisingly, while the proteins comprise first monomer:second monomer:light chains (in the case of many of the embodiments herein that have three polypeptides comprising the heterodimeric antibody) in a 1:1:2 ratio, these are not the ratios that give the best results.

The antibodies and ABDs provided herein are made by culturing host cells comprising the expression vector(s) as is well known in the art. Once produced, traditional antibody purification steps are done, including an ion exchange chromatography step. As discussed herein, having the pIs of the two monomers differ by at least 0.5 can allow separation by ion exchange chromatography or isoelectric focusing, or other methods sensitive to isoelectric point. That is, the inclusion of pI substitutions that alter the isoelectric point (pI) of each monomer so that such that each monomer has a different pI and the heterodimer also has a distinct pI, thus facilitating isoelectric purification of the “1+1 Fab-scFv-Fc” heterodimer (e.g., anionic exchange columns, cationic exchange columns). These substitutions also aid in the determination and monitoring of any contaminating dual scFv-Fc and mAb homodimers post-purification (e.g., IEF gels, cIEF, and analytical IEX columns).

### VII. Biological and Biochemical Functionality of the Bispecific and Trispecific Antibodies

Generally the bispecific and trispecific antibodies described herein are administered to patients with cancer (e.g., a PD-L1 or PD-L2 associated cancer or cancer that has been shown to benefit from anti-PD-1, anti-PD-L1, or anti-PD-L2 treatment), and efficacy is assessed, in a number of ways as described herein. Thus, while standard assays of efficacy can be run, such as cancer load, size of tumor, evaluation of presence or extent of metastasis, etc., immuno-oncology treatments can be assessed on the basis of immune status evaluations as well. This can be done in a number of ways, including both in vitro and in vivo assays.

### A. Antibody Compositions for In Vivo Administration

Formulations of the antibodies used in accordance with the present invention are prepared for storage by mixing an antibody having the desired degree of purity with optional pharmaceutically acceptable carriers, excipients or stabilizers (Remington's Pharmaceutical Sciences 16th edition, Osol, A. Ed. [1980]), in the form of lyophilized formulations or aqueous solutions.

### VIII. Treatments

Once made, the compositions of the invention find use in a number of oncology applications, by treating cancer, generally by enhancing immune responses (e.g., T cell activation and proliferation), particularly when used with anti-cancer therapies such as anti-PD1 and anti-tumor bispecific antibodies. In some embodiments, the antibodies provided herein enhance immune responses (e.g., T cell activation and proliferation) by providing agonistic co-stimulation of T cells in the microenvironment of tumors expressing PD-L1 and/or PD-L2.

### A. Anti-CD28×Anti-TAA/Anti-TAA Bispecific Antibody

In some embodiments, the subject bispecific and trispecific antibodies provided herein are administered with an anti-tumor bispecific antibody. In classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells (see FIG. 92A). In contrast, only the first signal is provided in treatment with CD3 bispecific antibodies that target a TAA (i.e., anti-CD3×anti-TAA bispecific antibodies)(see FIG. 92B).

Without being bound by any particular theory of operation, it is believed that the subject antibodies provided herein can enhance the anti-tumor response of an anti-CD3×anti-TAA bispecific antibody by providing CD28 costimulation and blockage of inhibitory PD-L1:PD-1 or PD-L2:PD-1 pathway interactions (see FIGS. 93 and 94 and Examples). Thus, in one aspect, provided herein are methods of methods of treating a cancer in a patient by administering the patient an anti-CD3×anti-TAA bispecific antibody and a subject bispecific or trispecific antibody provided herein. In some embodiments, the administration of the anti-CD3×anti-TAA bispecific antibody and subject bispecific or trispecific antibody enhances an immune response against the tumor in the patient. CD3 binding domains that can be included in the anti-CD3×anti-TAA bispecific antibodies are included in FIG. 15.

### B. Administrative Modalities

The antibodies provided herein administered to a subject, in accord with known methods, such as intravenous administration as a bolus or by continuous infusion over a period of time.

### C. Treatment Modalities

In the methods of the invention, therapy is used to provide a positive therapeutic response with respect to a disease or condition.

By “positive therapeutic response” is intended an improvement in the disease or condition, and/or an improvement in the symptoms associated with the disease or condition. For example, a positive therapeutic response would refer to one or more of the following improvements in the disease: (1) a reduction in the number of neoplastic cells; (2) an increase in neoplastic cell death; (3) inhibition of neoplastic cell survival; (5) inhibition (i.e., slowing to some extent, preferably halting) of tumor growth; (6) an increased patient survival rate; and (7) some relief from one or more symptoms associated with the disease or condition.

Positive therapeutic responses in any given disease or condition can be determined by standardized response criteria specific to that disease or condition. Tumor response can be assessed for changes in tumor morphology (i.e., overall tumor burden, tumor size, and the like) using screening techniques such as magnetic resonance imaging (MRI) scan, x-radiographic imaging, computed tomographic (CT) scan, bone scan imaging, endoscopy, and tumor biopsy sampling including bone marrow aspiration (BMA) and counting of tumor cells in the circulation.

In addition to these positive therapeutic responses, the subject undergoing therapy may experience the beneficial effect of an improvement in the symptoms associated with the disease.

Treatment according to the present invention includes a “therapeutically effective amount” of the medicaments used. A “therapeutically effective amount” refers to an amount effective, at dosages and for periods of time necessary, to achieve a desired therapeutic result.

A therapeutically effective amount may vary according to factors such as the disease state, age, sex, and weight of the individual, and the ability of the medicaments to elicit a desired response in the individual. A therapeutically effective amount is also one in which any toxic or detrimental effects of the antibody or antibody portion are outweighed by the therapeutically beneficial effects.

A “therapeutically effective amount” for tumor therapy may also be measured by its ability to stabilize the progression of disease. The ability of a compound to inhibit cancer may be evaluated in an animal model system predictive of efficacy in human tumors.

Alternatively, this property of a composition may be evaluated by examining the ability of the compound to inhibit cell growth or to induce apoptosis by in vitro assays known to the skilled practitioner. A therapeutically effective amount of a therapeutic compound may decrease tumor size, or otherwise ameliorate symptoms in a subject. One of ordinary skill in the art would be able to determine such amounts based on such factors as the subject's size, the severity of the subject's symptoms, and the particular composition or route of administration selected.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. Parenteral compositions may be formulated in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier.

The specification for the dosage unit forms of the present invention are dictated by and directly dependent on (a) the unique characteristics of the active compound and the particular therapeutic effect to be achieved, and (b) the limitations inherent in the art of compounding such an active compound for the treatment of sensitivity in individuals.

The efficient dosages and the dosage regimens for the bispecific antibodies used in the present invention depend on the disease or condition to be treated and may be determined by the persons skilled in the art.

All cited references are herein expressly incorporated by reference in their entirety.

Whereas particular embodiments of the invention have been described above for purposes of illustration, it will be appreciated by those skilled in the art that numerous variations of the details may be made without departing from the invention as described in the appended claims.

### EXAMPLES

Examples are provided below to illustrate the present invention. These examples are not meant to constrain the present invention to any particular application or theory of operation. For all constant region positions discussed in the present invention, numbering is according to the EU index as in Kabat (Kabat et al., 1991, Sequences of Proteins of Immunological Interest, 5th Ed., United States Public Health Service, National Institutes of Health, Bethesda, entirely incorporated by reference). Those skilled in the art of antibodies will appreciate that this convention consists of nonsequential numbering in specific regions of an immunoglobulin sequence, enabling a normalized reference to conserved positions in immunoglobulin families. Accordingly, the positions of any given immunoglobulin as defined by the EU index will not necessarily correspond to its sequential sequence.

General and specific scientific techniques are outlined in US Publications 2015/0307629, 2014/0288275 and WO2014/145806, all of which are expressly incorporated by reference in their entirety and particularly for the techniques outlined therein.

### Background

While checkpoint blockade immunotherapies have proven to be effective, many patients nonetheless fail to achieve a response. Engagement of T cell costimulatory receptors on TILs (e.g., CD80/CD86) with agonistic antibodies (e.g., anti-CD28 antibodies) could provide an additional positive signal capable of overcoming negative signals of immune checkpoints and may be a useful therapeutic modality to stack with checkpoint blockade.

The subject bispecific and trispecific antibodies provided herein provide a costimulatory signal for T-cell activation against tumor cells while advantageously also block inhibitory PD-L1:PD1 and/or PD-L2:PD1 pathway interactions.

### Example 1: PDL1 and PDL2 Binding Domains

Sequences for human, mouse, and cynomolgus PD-L1 and PD-L2 are depicted in FIGS. 1-2 and are useful for the development of cross-reactive PD-L1 and PD-L2 antigen binding domains for ease of clinical development.

1A: Novel PDL1 Binding Domain 2G4

In an initial campaign, numerous novel PDL1 binding domains were generated by hybridoma technology through IMMUNOPRECISE. The amino acid sequences for exemplary humanized hybridoma-derived clone 2G4 are depicted in FIG. 17. anti-PDL1 mAbs were incubated with indicated concentration of huPDL1-mFc fusions. The mixture was then combined with PD-1-expressing HEK293T cells. Cells were stained with anti-mouse Fc and binding was assessed by flow cytometry, data for which are depicted in FIG. 18. The data show that anti-PDL1 clone 2G4 blocks PD1:PDL1 interaction.

In a second experiment, CD3+ T cells from 11 unique donors were treated with a constant dose of the indicated test articles in the presence of 10,000 dendritic cells. Cytokine secretion was measured using MSD assay (Meso Scale Discovery, Rockville, Md.). The data depicted in FIG. 100 show that anti-PDL1 2G4 induced greater IL-2 and IFNγ release in comparison to the partial-blocking and the non-blocking anti-PDL1 clones.

Towards optimization of PDL1×CD28 bsAbs as described in Example 5B(a), numerous 2G4 affinity variants were developed by engineering VH variants (illustrative sequences as depicted in FIG. 19 and additional sequences depicted as SEQ ID NOs: 1467-1528), VL variants (illustrative sequences as depicted in FIG. 20 and additional sequences depicted as SEQ ID NOs: 1529-1599), and combinations thereof (illustrative sequences as depicted in FIG. 21). Consensus sequence of the variants are depicted in FIG. 22. Affinity of illustrative variants, in the context of PDL1×CD28 bsAbs having sequences as depicted in FIG. 84, was determined using Biacore. Experimental steps for Biacore generally included the following: Immobilization (capture of ligand onto a sensor chip); Association (flowing of various concentrations of analyte over sensor chip); and Dissociation (flowing buffer over the sensor chips) in order to determine the affinity of the test articles. A reference flow with buffer alone was also included in the method for background correction during data processing. Binding affinities and kinetic rate constants were obtained by analyzing the processed data using a 1:1 binding model. Binding affinity for human and cynomolgus PDL1 determined as such are depicted in FIG. 23.

1B: Novel Common Light Chain PDL1 and PDL2 Binding Domains

To engineer PDL1×PDL2×CD28 triAbs, common light chain formats (i.e. formats utilizing PDL1 and PDL2 binding domains having the same light chain; and in some formats, PDL1, PDL2, and CD28 binding domains having the same light chain) were explored, as multispecific antibody formats utilizing just Fab domains may be less immunogenic and more stable than those utilizing scFv domains.

1B(a): Single Cell Technology in Genetically Engineered Mice

In a first campaign, numerous novel PDL1 and PDL2 binding domains were generated by single cell technology in mouse genetically engineered with complete human heavy chain variable domain combined with a human common light chain substitution in situ (sequence for the common variable light domain is depicted in FIG. 24). A panel of 39 PDL1 mAbs and 33 PDL2 mAbs were identified and investigated for their binding affinity via Biacore, a surface plasmon resonance (SPR)-based technology. Experimental steps for Biacore generally included the following: Immobilization (capture of ligand onto a sensor chip); Association (flowing of various concentrations of analyte over sensor chip); and Dissociation (flowing buffer over the sensor chips) in order to determine the affinity of the test articles. Data depicted in FIGS. 27 and 33 show that there was a range of binding affinities. Sequences for illustrative PDL1 clones 13G1 and 13G7 are depicted in FIGS. 25-26; and sequences for illustrative PDL2 clones 5C11, 8G2, 8G5, and 16G11 are depicted in FIGS. 29-32. Although the mice were engineered with a common light chain, the mice were still capable of somatic hypermutation of the light chain, thus allowing the introduction of mutations in the light chains of many of these clones. Accordingly, the heavy chains were paired with the engineered germline light chain (hereon referred to as “6B1_L1”, sequences for which are depicted in FIG. 24). Additionally, further substitutions were made in the framework regions to revert non-germline amino acids back to germline when non-impact to antigen binding is expected. These sequences are also depicted alongside their parental sequences. To confirm that these novel binding domains were capable of PDL1 or PDL2 blockade, PDL1×CD28 bsAbs, PDL2×CD28 bsAbs, or PDL1×PDL2×CD28 triAbs based on these binding domains were incubated with CHO-PDL1 or CHO-PDL2 cells. After incubation with the bsAbs, the cells were incubated with AF647-labeled PD1. Data as depicted in FIGS. 128-129 show that the bsAbs based on these novel binding domains dose dependently blocked PD1 from binding cell surface PDL1 and PDL2.

To tune binding affinities for PDL1 and PDL2 (and potencies of the bispecific or trispecific molecules), an affinity ladder was engineered with the aim to achieve binding domains with K_{D} values ˜100 pM (0.1 nM) (which affinity range is optimal as determined by the modeling as will be described in Example 5D; illustrative sequences for which are depicted in FIGS. 26A-26D and SEQ ID NOs: 29-40 and 1764-1793 for affinity-engineered 13G7 and FIGS. 32A-32D and SEQ ID NOs: 77-88 and 2266-2295 for affinity-engineered 16G11; consensus sequences for FRs and CDRs of each clone and their affinity-engineered variants are depicted in FIGS. 26 and 32). While engineering was performed for 13G7 and 16G11 in the context of the H1 framework, the affinity-engineered CDRs may be grafted onto other parental frameworks. For example, CDRs of 13G7 H1.18 were grafted onto 13G7 H2 to generate 13G7 2.18. Similarly for example, CDRs of 16G11 H1.7 were grafted onto 16G11 H3 to generate 16G11 H3.7.

K_{D} of affinity-engineered 13G7 variants (formatted as bivalent IgG1 with PVA_/S267K variants) were determined via Carterra LSA Platform, another SPR-based technology. HC30M chip amine coupled with goat anti-human Fc antibody was used to capture the antibodies, and multiple concentrations of his-tagged was flowed over the antibodies. Data for which are depicted in FIGS. 145-146 show that 4 variants achieved K_{D} values ranging from 50 pM to 180 pM.

K_{D} of affinity-engineered 16G11 variants (formatted as bivalent IgG1 with PVA_/S267K variants) were determined via Carterra LSA Platform as described above, data for which are depicted in FIGS. 147-148. 5 variants achieved K_{D} values ranging from 110 pM to 180 pM.

1B(b): Phage

As will be described further in Example X, additional common light chain PDL1 and PDL2 binding domains that could be utilized with αCD28 clone 1A7 was desired. Therefore, in a second campaign, a phage library utilizing a constant human germline VL (utilizing the same human germline VL as in Example 2A, except without any diversity) and diversity in the VH was used. The amino acid sequences for exemplary phage-derived αPDL1 clone 2A3A4.248 and αPDL2 clone 1F12A4.249 are depicted in FIGS. 161 and 162.

As above, to tune binding affinities for PDL1 and PDL2 (and potencies of the bispecific or trispecific molecules) with the aim to achieve binding domains with K_{D} values ˜100 pM (0.1 nM), an affinity ladder was engineered (illustrative sequences for which are depicted in FIGS. 161A-161C and SEQ ID NOs: 3235-3266 for affinity-engineered 2A3A4.248 and FIGS. 162A-162G and SEQ ID NOs: 3267-3353 for affinity-engineered 1F12A4.249; consensus sequences for FRs and CDRs of each clone and their affinity-engineered variants are depicted in FIGS. 161 and 162).

K_{D} of affinity-engineered 2A3A4.248 variants (formatted as bivalent IgG1 with PVA_/S267K variants) were determined via Carterra LSA platform as described above, data for which are depicted in FIGS. 149-150. 4 variants achieved K_{D} values ranging from 83 pM to 180 pM.

K_{D} of affinity-engineered 1F12A4.249 variants (formatted as bivalent IgG1 with PVA_/S267K variants) were determined via Carterra LSA platform as described above, data for which are depicted in FIGS. 151-152 (round 1) and 153-154 (round 2). In the first round, no variants showed K_{D} in the desired range, with best clone having a K_{D} of 480 pM. In the second round, 3 variants achieved K_{D} values less than 100 pM. It should be noted that these PDL1 and PDL2 binding domains may be used in the context of CLC format PDL1×PDL2×CD28 triAbs, as well as in the context of PDL1×CD28 and PDL2×CD28 bsAbs.

1C: Additional PDL1 and PDL2 Binding Domains

Sequences for additional PDL1 and PDL2 binding domains which may find use in the PDL1×CD28 and PDL1×CD28 bsAbs and PDL2/L2×CD28 triAbs of the invention are depicted in FIGS. 28 and 34 as SEQ ID NOs: 1794-2265 and 2296-2447.

### Example 2: CD28 Binding Domains

Sequences for human, mouse, and cynomolgus CD28 are depicted in FIG. 3 and are useful for the development of cross-reactive CD28 antigen binding domains for ease of clinical development.

2A: CD28 Binding Domain 1A7

An approach considered to avoid the superagonism associated with TGN1412 was to generate novel CD28 binding domains having lower affinity binding to CD28 and/or binding to a different CD28 epitope than TGN1412. In one campaign to generate such novel CD28 binding domains, in-house de novo phage libraries were panned against CD28 which culminated in a phage-derived clone 1A7 which was determined to not be superagonistic (sequences depicted in FIG. 32). To enable tuning of CD28 bsAbs (e.g. to optimize potency and therapeutic index), numerous 1A7 affinity variants were developed by engineering VH variants (illustrative sequences as depicted in FIG. 35 and additional sequences depicted as SEQ ID NOs: 2448-2455), VL variants (illustrative sequences as depicted in FIG. 36 and additional sequences depicted as SEQ ID NOs: 2456-2524), and combinations thereof (illustrative sequences for which are depicted in FIG. 37). Consensus sequence of the variants are depicted in FIG. 39. Affinity of illustrative variants, in the context of PDL1×CD28 bsAbs having sequences as depicted in FIG. 38, was determined using Octet as generally described below, binding affinity as depicted in FIG. 40. Potential superagonism of clone 1A& was assessed by air-drying per the Stebbings protocol (Stebbings R. et al. 2007). Air-drying of test articles was achieved by drying in a SpeedVac™ for 2 hours at room temperature. Human PBMCs were treated for 24 hours with 10 pg of air-dried parental αCD28 mAb 1A7), and activity was compared to the superagonist TGN1412 or PBS control. Air-dried TGN1412 promoted IFNγ secretion from unstimulated human PBMC. In comparison, IFNγ level in PBMCs treated with air-dried αCD28 mAb 1A7 remained similar to the negative control of PBS (data shown in FIG. 135).

2B: Novel Common Light Chain CD28 Binding Domain

As described above, certain common light chain formats explored to develop PDL1×PDL2×CD28 triAbs utilized PDL1, PDL2, and CD28 binding domains having the same light chain.

2B(a): Single Cell Technology in Genetically Engineered Mice

Towards this, additional novel CD28 binding domains were generated using single cell technology in the above-described mouse genetically engineered with complete human heavy chain variable domain combined with a human common light chain substitution in situ, sequences for which are depicted in FIGS. 41-74. Antibody secreting plasma cells were isolated and enriched from lymph nodes harvested from immunized mice. Bulk plasma cells were imported onto a microfluidic chip and single cells were sequestered into individual wells for screening. Each plasma cell was individually screened for secretion of antigen-specific IgG that is detected using an antigen coated bead-based fluorescence assay. Individual cells of interest were lysed, their mRNA captured and used to generate cDNA encoding antibody VH an VL sequences. As above although the mice were engineered with a common light chain, the mice were still capable of somatic hypermutation of the light chain, thus allowing the introduction of mutations in the light chains of these clones. Accordingly, the heavy chains were paired with the expected common light chain (i.e. 6B1_L1). Binding to human CD28 antigen by the WT parental binding domains sequenced from the single plasma cells and binding domains pairing the VH with the 6B1_L1 VL (in the context of bivalent mAbs) was investigated using Octet, a BioLayer Interferometry (BLI)-based method. Experimental steps for Octet generally included the following: Immobilization (capture of ligand to a biosensor); Association (dipping of ligand-coated biosensors into wells containing serial dilutions of the analyte); and Dissociation (returning of biosensors to well containing buffer) in order to determine the affinity of the test articles. Resulting maximum binding responses are depicted in FIGS. 75-76 and show that that VH from most of the plasma cell-derived binding domains retain CD28 binding when paired with 6B1_L1 VL; however, VH from several of the clones (e.g. 1B1-4.88488, 2G5.88497 and 1D10-4.83967) demonstrated diminished CD28 binding response.

The novel CD28 binding domains were further investigated for binding to CD28⁺ Jurkat cells. CD28 mAbs were titrated on Jurkat cells with the top concentration of 30 μg/mL and binding was determined using AF647-labeled anti-Fc mAb, data for which are depicted in FIGS. 77-78. Next, agnostic properties of the novel CD28 binding domains were investigated. Purified T cells were added in the presence of plate bound CD28 mAbs (based on single-cell technology derived binding domains) and CD3 antibody OKT3 (100 ng/mL). One day after T cell seeding, TL-2 release was measured using MSD assay, data for which are shown in FIGS. 79-80. Notably, binding did not necessarily correlate to activity. XENP41858, XENP42419, XENP42422, XENP41869, XENP42421, and XENP41901 were weak binders but decent agonists. XENP41909, XENP41877, XENP41880, and XENP41936 were decent binders but did not show agonism.

2B(b): Enabling 1A7 to Utilize a Common Light Chain

As will be described in Example 7G, the 1A7 binding domain enabled stronger potency than the 1A3.88474 binding domain. Accordingly, there was a desire to utilize the 1A7 binding domain in common light chain formats. As described in Example 1B(b), the phage library for discovering CD28 clone 1A7, PDL1 clone 2A3A4.24, and PDL2 clone 1F12A4.249 binding domains utilized the same human germline VL, although the CD28 library included diversity in the LCDR3. Surprisingly, it was found that the variable light domain of clone 1A7 differed from the parental germline VL by only a single amino acid in the LCDR3; however, despite there being only a single amino acid difference, the VH of 1A7 does not pair well with the parental germline VL resulting in diminished binding. Accordingly, affinity-optimization library was generated with focus on substitutions only in the variable heavy domain of 1A7 (to ensure that ability of the germline VL to act as common light chain with the PDL1 and PDL2 binding domains from Example 1B(b)) was not perturbed. Amino acid sequences for exemplary affinity-optimized 1A7 variable domains are depicted in FIGS. 163A-163C and SEQ ID NOs: 3354-3393 and 2674-2676.

Monovalent K_{D} of affinity-engineered 1A7 Fab variants (in the context of PDL1×PDL2×CD28 triAbs in the 1+1+1 stackFab₂-Fab-Fc format) were determined via Octet as generally described above. In particular, his-tagged CD28 antigen was captured onto HIS1k sensors and dipped into the triAbs to determine affinity, data for which are depicted in FIG. 159.

After engineering this additional 1A7 VH variants, favorite variants were paired with the original 1A7_L1 and L1.71 VLs to generate further affinity variants (in the context of scFvs). KD of these additional variants are depicted in FIG. 165. Finally, to stabilize formats comprising scFvs, disulfide-stabilized scFvs may be used. Disulfide-stabilized scFvs may be achieved by including cysteine substitutions in the VH and VL so that a disulfide bond is formed between the two cysteines (see FIG. 166 demonstrating improved thermostability by incorporating disulfide stabilization). Illustrative such disulfide-stabilized scFvs are depicted in FIG. 164, and KD are depicted in FIG. 165.

2C: Additional CD28 Binding Domains

Sequences for additional CD28 binding domains which may find use in the PDL1×CD28 bsAbs of the invention are depicted in FIG. 81 and as SEQ ID NOs: 2799-2824 and 3468-3473.

### Example 3: Engineering PDL1×CD28 and PDL2×CD28 bsAbs

As described in Example 4, in classic T cell/APC interaction, there is a first signal provided by TCR reactivity with peptide-MHC (Signal 1) and a second signal provided by CD28 crosslinking by CD80/CD86 being expressed on APCs (Signal 2) which together fully activate T cells (see FIG. 92). Further, it may be useful to stack the CD28 signal with checkpoint blockade to mitigate any checkpoint mediated repression of the added CD28 signal (FIG. 93). PDL1×CD28 may provide the Signal 2 while advantageously further enabling blockade of PDL1:PD1 interaction (FIG. 94). Similarly, PDL2×CD28 may provide the Signal 2 while advantageously further enabling blockade of PDL2:PD1 interaction.

Accordingly, PDL1×CD28 and PDL2×CD28 bsAbs were engineered and produced. A number of formats were conceived, illustrative formats for which are outlined below and in FIG. 82.

3A: 1+1 Fab-scFv-Fc Format

An illustrative bivalent, bispecific format is the 1+1 Fab-scFv-Fc format (depicted schematically in FIG. 82A) which comprises: a first monomer that is a heavy chain comprising, from N-terminal to C-terminal VH1-CH1-hinge-CH2-CH3 wherein CH2-CH3 is a first heterodimeric Fc domain; a second monomer comprising, from N-terminal to C-terminal scFv-linker-CH2-CH3 wherein CH2-CH3 is a second heterodimeric Fc domain complementary to the first heterodimeric Fc domain and wherein the scFv has a first antigen binding specificity; and a third monomer that is a light chain comprising, from N-terminal to C-terminal VL-CL wherein the VL pairs with VH1 to form an antigen binding domain having a second antigen specificity. Sequences for illustrative PDL1×CD28 and PDL2×CD28 bsAbs (based on binding domains as described in Examples 1 and 2) in the 1+1 Fab-scFv-Fc format are respectively depicted in FIGS. 84 and 87.

3B: 2+1 Fab2-scFv-Fc Format

An illustrative trivalent, bispecific format is the 2+1 Fab2-scFv-Fc format (depicted schematically in FIG. 82B) which comprises: a first monomer that is a heavy chain comprising, from N-terminal to C-terminal VH1-CH1-hinge-CH2-CH3 wherein CH2-CH3 is a first heterodimeric Fc domain; a second monomer comprising, from N-terminal to C-terminal VH1-CH1-linker-scFv-linker-CH2-CH3 wherein CH2-CH3 is a second heterodimeric Fc domain complementary to the first heterodimeric Fc domain and wherein the scFv has a first antigen binding specificity; and a third monomer that is a light chain comprising, from N-terminal to C-terminal VL-CL wherein the VL pairs with the VH1 (in the first and second monomers) to form antigen binding domains having a second antigen binding specificity. Sequences for illustrative PDL1×CD28 bsAbs (based on binding domains as described in Examples 1 and 2) in the 2+1 Fab2-scFv-Fc format are depicted in FIG. 85.

3C: 2+1 MAB-SCFV Format

Another illustrative trivalent, bispecific format is the 2+1 mAb-scFv format (depicted schematically in FIG. 82E) which comprises: a first monomer that is a heavy chain comprising, from N-terminal to C-terminal VH1-CH1-hinge-CH2-CH3 wherein CH2-CH3 is a first heterodimeric Fc domain; a second monomer comprising, from N-terminal to C-terminal VH1-CH1-hinge-CH2-CH3-linker-scFv wherein CH2-CH3 is a second heterodimeric Fc domain complementary to the first heterodimeric Fc domain and wherein the scFv has a first antigen specificity; and a third monomer that is a common light chain comprising, from N-terminal to-C-terminal VL-CL wherein the VL pairs with VH1 to form an antigen binding domains having a second antigen specificity. Sequences for illustrative PDL1×CD28 bsAbs (based on binding domains as described here) in the 2+1 mAb-scFv format are depicted in FIG. 86.

Any number of heterodimerization approaches as is known in the art could find use in these (and other) bispecific formats, in combination with any number of approaches for purifying heterodimers from contaminating homodimers, including those depicted in FIGS. 4-5. For example, some of the sequences depicted herein include H435R/Y436F on one monomer to facilitate purification; however, the antibodies may instead utilize alternative variants to facilitate purification. Regardless of bsAb format, the CD28 bispecific antibodies are monovalent for CD28 and incorporate Fc variants engineered to ablate FcγR binding to avoid potential superagonism. Such Fc variants include those depicted in FIG. 6. Any of the number of linkers as is known in the art may find use in linking the various domains of the bispecific antibody including for example the VH and VL domains of the scFv. Finally, it may be useful to maximize serum half-life of the bsAbs, and any of the number of half-life extending variants as is known in the art may find use in these bsAbs.

### Example 4: Engineering PDL1×PDL2×CD28 triAbs

Although as will be described in Example 5, PDL1×CD28 bsAbs and PDL2×CD28 bsAbs are active, they may be less efficacious on tumors having low PDL1 or PDL2 expression levels. Accordingly, it was hypothesized that engineering binding to both PDL1 and PDL2 may enable binding to a wider breadth of tumor cells (e.g. PDL1^{high}PDL2^{low} or PDL1^{low} PDL2^{high}). It is expected that co-expression of PDL1 and PDL2 are not required for activity and CD28 agonism should be achieved when only one antigen is expressed.

Accordingly, PDL1×PDL2×CD28 triAbs were conceived, engineered and produced. A number of formats were conceived, illustrative formats for which are outlined below and in FIG. 83.

4A: 1+1+1 stackFab₂-scFv-Fc Format

An illustrative trivalent, trispecific format is the 1+1+1 stackFab2-scFv format (depicted schematically in FIG. 83A) which comprises: a first monomer comprising, from N-terminal to C-terminal VH1-CH1-linker-VH2-CH1-hinge-CH2-CH3 wherein CH2-CH3 is a first heterodimeric Fc domain; a second monomer comprising, from N-terminal to C-terminal scFv-linker-CH2-CH3 wherein CH2-CH3 is a second heterodimeric Fc domain complementary to the first heterodimeric Fc domain and wherein the scFv has a first antigen specificity; and a third monomer that is a common light chain comprising, from N-terminal to C-terminal VL-CL wherein the VL pairs with VH1 to form an antigen binding domain having a second antigen specificity and with VH2 to form an antigen binding domain having a third antigen specificity. Sequences for illustrative PDL1×PDL2×CD28 triAbs (based on binding domains as described in Examples 1 and 2) in the 1+1+1 stackFab2-scFv-Fc format are depicted in FIG. 88.

4B: 1+1+1 Fab-(Fab-scFv)-Fc Format

Another trivalent, trispecific format is the 1+1+1 Fab-(Fab-scFv)-Fc format (depicted schematically in FIG. 83B) which comprises: a first monomer that is a heavy chain comprising, from N-terminal to C-terminal VH1-CH1-hinge-CH2-CH3 wherein CH2-CH3 is a first heterodimeric Fc domain; a second monomer comprising, from N-terminal to C-terminal VH2-CH1-linker-scFv-linker-CH2-CH3 wherein CH2-CH3 is a second heterodimeric Fc domain complementary to the first heterodimeric Fc domain and wherein the scFv has a first antigen specificity; and a third monomer that is a common light chain comprising, from N-terminal to C-terminal VL-CL wherein the VL pairs with VH1 to form an antigen binding domain having a second antigen specificity and with VH2 to form an antigen binding domain having a third antigen specificity. Sequences for illustrative PDL1×PDL2×CD28 triAbs (based on binding domains as described in Examples 1 and 2) in the 1+1+1 Fab-(Fab-scFv)-Fc format are depicted in FIG. 89.

4C: 1+1+1 mAb-scFv

Another trivalent, trispecific format is the 1+1+1 mAb-scFv format (depicted schematically in FIG. 83C) which comprises: a first monomer that is a heavy chain comprising, from N-terminal to C-terminal VH1-CH1-hinge-CH2-CH3 wherein CH2-CH3 is a first heterodimeric Fc domain; a second monomer comprising, from N-terminal to C-terminal VH2-CH1-hinge-CH2-CH3-linker-scFv wherein CH2-CH3 is a second heterodimeric Fc domain complementary to the first heterodimeric Fc domain and wherein the scFv has a first antigen specificity; and a third monomer that is a common light chain comprising, from N-terminal to C-terminal VL-CL wherein the VL pairs with VH1 to form an antigen binding domain having a second antigen specificity and with VH2 to form an antigen binding domain having a third antigen specificity. Sequences for illustrative PDL1×PDL2×CD28 triAbs (based on binding domains as described in Examples 1 and 2) in the 1+1+1 mAb-scFv format are depicted in FIG. 90.

4D: 1+1+1 stackFab2-Fab-Fc Format

A further trivalent, trispecific format is the 1+1+1 stackFab2-Fab-Fc format (depicted schematically in FIG. 83D) which comprises: a first monomer that is a heavy chain comprising, from N-terminal to C-terminal VH1-CH1-hinge-CH2-CH3 wherein CH2-CH3 is a first heterodimeric Fc domain; a second monomer comprising, from N-terminal to C-terminal VH2-CH1-linker-VH3-CH1-hinge-CH2-CH3 wherein CH2-CH3 is a second heterodimeric Fc domain complementary to the first heterodimeric Fc domain; and a third monomer that is a common light chain comprising, from N-terminal to C-terminal VL-CL wherein the VL pairs with VH1 to form an antigen binding domain having a first antigen specificity, with VH2 to form an antigen binding domain having a second antigen specificity, and with VH3 to form an antigen binding domain having a third antigen specificity. Sequences for illustrative PDL1×PDL2×CD28 triAbs (based on binding domains as described in Examples 1 and 2) in the 1+1+1 Fab2-Fab-Fc format are depicted in FIG. 91.

Any number of heterodimerization approaches as is known in the art could find use in these (and other) trispecific formats, in combination with any number of approaches for purifying heterodimers from contaminating homodimers, including those depicted in FIGS. 4-5. For example, some of the sequences depicted herein include H435R/Y436F on one monomer to facilitate purification; however, the antibodies may instead utilize alternative variants to facilitate purification. As with the bsAbs, regardless of triAb format, the CD28 trispecific antibodies are monovalent for CD28 and incorporate Fc variants engineered to ablate FcγR binding to avoid potential superagonism. Such Fc variants include those depicted in FIG. 6. Any of the number of linkers as is known in the art may find use in linking the various domains of the trispecific antibody including for example the VH and VL domains of the scFv. Finally, it may be useful to maximize serum half-life of the triAbs, and any of the number of half-life extending variants as is known in the art may find use in these triAbs.

### Example 5: PDL1×CD28 bsAbs

5A: Initial Characterization of PDL1×CD28 bsAbs

5A(a): PDL1×CD28 bsAbs Induce Expansion of T Cells

MDA-MB-231 (PDL1+) cancer cells were ectopically loaded with pp65-derived NLV-peptide for 24 hours. The following day, T cells from a CMV+ donor were added along with a monovalent PDL1 antibody XENP24118 (based on avelumab; sequences depicted in FIG. 95) or PDL1×CD28 XENP34963. Expansion of CMV+ T cells was measured with an NLV-specific tetramer. The data as depicted in FIG. 96 show that the PDL1×CD28 bsAb XENP36233 significantly enhanced T cell expansion in comparison to PDL1 blockade alone.

5A(b): PDL1×CD28 bsAbs Block PD-1:PD-L1 Interaction During T-Cell:Cancer Cell Interactions

Jurkat-PD1 cells were treated with a dose titration of the indicated test articles (i.e. PDL1×CD28 bsAbs containing PDL1 clone 2G4, a partial-blocking PDL1 arm, or a non-blocking PDL1 arm) in the presence of CHO-PDL1-CD80-αCD3 and CHO-PDL1-αCD3 cells. 6 hours post Jurkat-PD1 seeding, luciferase activity was measured as an indicator of PD-1 blockade. The data as depicted in FIG. 101 show that XENP36233 having blocking PDL1 clone 2G4 induced strong luciferase activity in comparison to PDL1×CD28 bsAbs having partial blocking or non-blocking PDL1 arms.

5A(c): PDL1×CD28 bsAbs are More Active on Cells Having Higher PDL1 Antigen Density

PDL1^{null} MC38 cells were transfected to express low or medium levels of PDL1 antigen (respectively 40K and 59K antigen density). Parental, PDL1^{low}, and PDL1^{med} MC38 clones were treated with a dose titration of XENP36233. Secondary A647-conjugated anti-human Fc antibody was used to detect the bsAbs by flow cytometry, data for which are depicted in FIG. 102.

Next, PDL1^{null} HEK293T cells were transfected to express medium or high levels of PDL1 antigen. 10,000 CD3+ T cells were treated with a dose titration of XENP36233 and 1 μg/mL B7H3×CD3 in the presence of 10,000 PDL1-HEK293T density series. 1 day post T cell seeding, IL-2 secretion was measured using MSD assay, data as depicted in FIG. 103. Collectively, the data show that PDL1×CD28 bsAbs are more active on cells having higher PDL1 antigen density.

5A(d): PDL1×CD28 bsAbs Enhance Activity of CD3 Bispecific Antibodies, but Only on PDL1⁺ Cell Lines

In a first experiment, induction of cytokine (IL-2 and IFNγ) release and CD3⁺ T cell expansion by PDL1×CD28 bsAbs in combination with CD3 bispecific antibodies was assessed using Incucyte® Live-Cell Analysis system (Essen BioScience, Ann Arbor, MI). MDA-MB-231 cancer cells and T cells were mixed with 1 μg/ml of an illustrative B7H3×CD3 bispecific and indicated concentrations of PDL1 mAb XENP24118 or PDL1×CD28 bsAb XENP34963. Cancer cell growth was assayed over time with Incucyte, while cytokine secretion was measured using MSD assay (Meso Scale Discovery, Rockville, Md.) and T cell expansion was measured using flow cytometry. The data as depicted in FIG. 97 show that PDL1×CD28 bsAb synergizes with CD3 bispecific antibodies to induce T-cell activation.

In another experiment, cell kill at a 1:1 effector:target ratio was assessed using xCELLigence Real Time Cell Analysis instrument (ACEA Biosciences, San Diego, CA). 1,250 LNCaP cancer cells were first seeded. After 48 hours, freshly enriched CD3+ T cells at an effector:target of 1:1 were added along with antibodies (PSMA×CD3 XENP32220 alone or XENP32220 in combination with XENP36233; sequences for XENP32220 are depicted in FIG. 16) at a constant dose. Cell kill was recorded for 10 days post T cell seeding. In this experiment, the data as depicted in FIG. 98 show that XENP32220 alone was able to enhance cell kill in comparison to incubation of cancer and T cells alone; however, addition of PDL1×CD28 overcomes cancer cell resistance to the CD3 bispecific and further enhances cell kill.

However, CD3 bsAb activity is only enhanced on PDL1+ cells. CD3+ T cells were incubated with 22Rv1 (PDL1^{null}) at 10:1 and 1:1 E:T ratios with illustrative B7H3×CD3 alone or B7H3×CD3 in combination with XENP36233. Cell kill was recorded using xCELLigence for 7 days post T cell seeding. The data as depicted in FIG. 104 show that αPDL1×αCD28 bsAb XENP36233 did not synergize with the αB7H3×αCD3 to enhance cell kill of the PDL1 negative 22Rv1 cell line.

5A(e): PDL1×CD28 Synergizes with CD3 bsAb Over Time as PDL1 Expression Increases

PC3 cells were cocultured with T cells and then treated with illustrative B7H3×CD3 bsAb alone or with αIFNγ neutralizing mAb. PDL1 expression on PC3 cells was assessed by flow cytometry as depicted in FIG. 132. The data indicate that CD3 bsAbs induce PDL1 and PDL2 expression by promoting IFNγ release.

Next, PC3 cells were cocultured with T cells and then treated with indicated antibodies for 1 day, 2 days, or 5 days. IFNγ was measured in culture supernatants at indicated times after antibody treatment, data for which are depicted in FIG. 133 showing that CD3 bsAb synergizes with PDL1×CD28 over time as PDL1 expression increases.

5A(f): PDL1×CD28 Enhances Anti-Tumor Activity In Vivo

MC38 cancer cells stably expressing human PDL1 antigen were subcutaneously inoculated into human CD28 knock-in mice. When tumors were palpable (Day 0), mice were intraperitoneally treated weekly with either 5 mg/kg bivalent PDL1 antibody based on avelumab (XENP24118), 6 mg/kg PDL1×CD28 XENP34961 (2+1 mAb-scFv format), or 8.3 mg/kg PDL1×CD28 XENP34963 (1+1 Fab-scFv-Fc format). Mice were additionally dosed on Days 3, 7, 10, 14, and 17. Tumor volumes were monitored by caliper measurements, data for which are shown (days post 1^{st} dose) in FIG. 99. The data show that although PDL1 blockade inhibited tumor growth, the PDL1×CD28 bsAbs as a single-agents significantly enhanced anti-tumor activity.

5A(g): PDL1×CD28 was Well Tolerated in Cynomolgus Monkeys and Exhibited Favorable Pharmacokinetics

Cynomolgus monkeys were dosed with XENP36764 (sequences for which are depicted in FIG. 84; XENP36233 further engineered with M428L/N434S for enhanced half-life). The data as depicted in FIG. 105 show that the PDL1×CD28 exhibited favorable pharmacokinetics. Additionally, the bsAb was well tolerated (data not shown).

5B: Optimizing PDL1×CD28 bsAbs

5B(a): Mechanism-Based PK/PD Modeling Suggests Avenues for Tuning CD28 and PDL1 Binding Affinities

Surface expression of PD-L1 is low on tumors may limit synapse engagement and presents the problem of how to obtain optimal cross-linking of CD28 with a bispecific. With this background, computer modeling was used to assist engineering of optimized PDL1×CD28 bsAbs by finding the best balance of CD28 and PDL1 affinities to achieve low antigen density targeting. A mechanism-based model was developed using experimental data (from scientific literature and in-house generated) and the following assumptions (schematically depicted in FIG. 106): three well-mixed compartments (central, peripheral, and tumor); all the compartments contain both T cells and PDL1 expressing cells; free drug transport between all compartments; non-specific clearance of the drug occurs in all compartments with the same order rate; soluble target for CD28 and PDL1 are low affinity and therefore not included; drug-bound receptor has the same internalization rate as the free receptor; cross-linking CD28:drug_PD-L1 trimer is assumed to internalize with either target; and cross-linking CD28:drug_PD-L1 trimer is assumed to internalize at the rate of faster receptor. As depicted in FIG. 107, the model predicted intratumoral T cell costimulatory activity and consistent PDL1 blockade; linear PK at dose levels consistent with typical checkpoint inhibitor regimens; trimer formation in the tumor indicating costimulation; and consistent blockade of PDL1.

Notably in view of low surface expression of PDL1 on tumors, the simulations suggested that CD28 affinity must be enhanced (from 560 nM to 58 nM) and that PDL1 affinity must be enhanced (from 2.4 nM to 0.1 nM).

5B(b): Tuning CD28 Binding Affinity

Based on the above suggestion, CD28 clone 1A7 affinity variants were engineered as described in Example 1A(c) and paired with WT PDL1 clone 2G4.

5B(b)i: Increasing CD28 Affinity Leads to More Potent and Efficacious TL-2 Secretion

In a first experiment investigating the effect of enhanced CD28 binding affinity, MDA-MB-231 or DU145 cancer were incubated with CD3+ T cells at an E:T ratio of 10:1, 1 μg/mL B7H3×CD3, and titrated doses of the indicated PDL1×CD28 test articles having the following range of CD28 binding affinities: 37 nM (1A7_L1.71_H1.14 in XENP37561); 36 nM (1A7_H1.14_L1.71 in XENP37261); 96 nM (1A7_H1.1_L1.71 in XENP37560); 180 nM (1A7_H1_L1.71 in XENP37559); 286 nM (1A7_L1_H1.14 in XENP37412); and 240 nM (1A7_H1.14_L1 in XENP36233). IL-2 secretion was assessed after 24 hours, data for which are depicted in FIG. 108. The data show that increasing CD28 affinity leads to more potent and efficacious IL-2 secretion. Notably in this set, 180 nM binding affinity for CD28 is the minimum for maximum activity (with XENP37412 and XENP36233 respectively having 286 nM and 240 nM binding affinities for CD28 demonstrating not only less potent but also less efficacious activity).

5B(b)ii: Increasing CD28 Affinity Increases Cytotoxicity of PDL1^{low} Cancer Cells at an E:T of 1:1

In a second experiment investigating the effect of enhanced CD28 binding affinity, LnCAP cells (PDL1^{low}) were incubated with CD3+ T cells at a low E:T ratio of 1:1 with 1 μg/ml αB7H3×αCD3 bsAb alone or in combination with 1 μg/ml of PDL1×CD28 bsAbs having different CD28 binding affinities (same set as in Example 5B(a)). Cytotoxicity was recorded using xCELLigence for 6 days post T cell seeding, data for which are depicted in FIG. 109. The data show that increasing CD28 affinity increases cytotoxicity of low PDL1 expressing cancer cells at a low E:T ratio of 1:1.

5B(b)ii: Increasing CD28 Affinity Increases Cytotoxicity of PDL1^{med} Cancer Cells at an E:T of 0.1:1

In a second experiment investigating the effect of enhanced CD28 binding affinity, DU145 cells (PDL1^{med}) were incubated with CD3+ T cells at a low E:T ratio of 0.1:1 with 1 μg/ml αB7H3×αCD3 bsAb alone or in combination with 1 μg/ml of PDL1×CD28 bsAbs having different CD28 binding affinities (same set as in Example 5B(a)). Cytotoxicity was recorded using xCELLigence for 6 days post T cell seeding, data for which are depicted in FIG. 110. The data show that increasing CD28 affinity increases cytotoxicity of medium PDL1 expressing cancer cells at a very low E:T ratio of 0.1:1.

5B(b)iii: PDL1×CD28 bsAbs Tuned for Enhanced CD28 Binding Affinity are Active In Vivo

PDL1×CD28 bsAb XENP37261 having affinity-enhanced CD28 arm was investigated in an in vivo anti-tumor model similar to Example 4F. MC38 cancer cells stably expressing human PDL1 antigen were subcutaneously inoculated into human CD28 knock-in mice. When tumors were palpable (Day 0), mice were intraperitoneally treated weekly with either 6.6 mg/kg monovalent PDL1 antibody based on avelumab (XENP36627) or 8.3 mg/kg PDL1×CD28 XENP37261. Tumor volumes were monitored by caliper measurements, data for which are shown (days post 1^{st} dose) in FIG. 111. The data show that although PDL1 blockade inhibited tumor growth, the PDL1×CD28 bsAb XENP37261 as a single-agent significantly enhanced anti-tumor activity.

5B(c): Tuning PDL1 Binding Affinity

Next, PDL1 clone 2G4 affinity variants were engineered as described in Example 1A and paired with CD28 clone 1A7_H1_L1.71 (180 nM CD28 arm; lower CD28 activity allows for assessment of PDL1 blockade).

5B(c)i: Increasing PDL1 Affinity Promotes Cytokine Secretion

In a first experiment, DU145-NLR cells were incubated with CD3+ T cells at a E:T ratio of 1:1 and treated with 1 μg/mL B7H3×CD3 and dose response of the indicated PDL1×CD28 bsAbs having different PDL1 binding affinities. 1 day post T cell seeding, IL-2 secretion was assessed using MSD assay, data for which are depicted in FIG. 112. The data show that increased PDL1 affinity promotes IL-2 secretion.

In a second experiment, data for which are depicted in FIG. 113, XENP38514 having 0.17 nM PDL1 arm enhances T cell/APC interaction in DC:T cell mixed lymphocyte reaction as indicated by IL-2 and IFNγ release.

5B(c)ii: Increasing PDL1 Affinity Promotes PD1:PDL1 Blockade

In another experiment, indicated concentrations of the PDL1×CD28 bsAbs were incubated in the presence of huPD-L1-mFc fusion for 30 minutes at room temperature. The mixture was then combined with PD-1-expressing HEK293T cells and incubated for 1 hour at 4° C. Cells were stained with anti-mouse Fc and binding was assessed by flow cytometry, data for which are depicted in FIG. 114. The data show the PDL1×CD28 bsAbs can block interaction between PD1 and PDL1.

5B(d): Tuning Both CD28 and PDL1 Binding Affinities

As the mechanism-based modeling suggested that affinity-enhanced CD28 binding domains should be paired with affinity-enhanced PDL1 binding domains, bsAbs having affinity-enhanced CD28 clone 1A7 variants were paired with affinity-enhanced PDL1 clone 2G4.

In a first experiment, PDL1 binding domains having 0.6 nM, 1 nM, and 2.6 nM binding affinities were paired with CD28 binding domains having 37 nM, 96 nM, 180 nM, and 230 nM binding affinities and investigated in an SEB-stimulated PBMC assay. PBMCs were stimulated with 110 ng/mL SEB for 2 days. 200,000 PBMCs were then seeded in the presence of 10 ng/mL SEB and PDL1×CD28 bsAbs at the indicated doses. IL-2 secretion was assayed after 24 hours, data for which are depicted in FIG. 115. The data suggest that high affinity PD-L1 arms such as 0.6 nM arm is optimally paired with 37 nM CD28 arm; nonetheless, high affinity PD-L1 binding overcomes the requirement for increased CD28 affinity as it also enables good activity in combination with low affinity CD28 as in XENP39365 and XENP39368.

5C: Further Optimization of the PDL1×CD28 bsAbs

Additional optimization of other characteristics such as stability and expression are also important from a developability perspective. Although pairing high affinity PDL1 binding domain 2G4_H1.12_L1.24 with high affinity CD28 binding domain 1A7_H1.14_L1.71 as in XENP38512 enables a molecule having optimal biological activity, it turns out that XENP38512 suffered from less than ideal stability and antibody expression levels (data not shown). Accordingly, additional affinity variants were generated based on 2G4_H1.12_L1.24 by reverting or back mutating substitutions suspected to decrease stability and/or yield and/or to achieve a minimal necessary set of substitutions (relative to H1L1). Several binding domains including 2G4_H1.12_L1.14, 2G4_H1.12_L1.66, and 2G4_H1.12_L1.68 were identified which demonstrated 4.5° C. improved Tₘ in comparison to 2G4_H1.12_L1.24 as well as improved yield while maintaining high affinity PDL1 binding. Additionally, codon optimization was investigated.

In a further experiment, PDL1 binding domains 2G4_H1.12_L1.24 (0.17 nM), 2G4_H1.12_L1.66 (0.86 nM), 2G4_H1.12_L1.68 (0.80 nM), or 2G4_H1.12_L1.14 (0.42 nM) binding affinities were paired with CD28 binding domain having 37 nM binding affinity (1A7_H1.14_L1.71) and investigated in a reverse CAR-T experiment (wherein test articles were incubated with CD3+ enriched T cells, MDA-MB-231 transfected to express CD3 scFv (to act as Signal 1)), and 1 μg/mL of an illustrative B7H3×CD3 bsAb. It should be noted that XENP38512 and XENP40036 have the same amino acid but were expressed from different codons. The data as depicted in FIG. 116 show that the stability/production optimized variants enabled similar IL2 induction as compared to 2G4_H1.12_L1.24 albeit with XENP40409 (non-Xtend analog of XENP40706, containing 2G4_H1.12_L1.14) inducing IL2 production most potently.

5D: PDL1×CD28 bsAbs Selectively Induce Proliferation of Effector T Cells in Cynomolgus Monkeys

Cynomolgus monkeys were dosed with XENP36803 (1× dose, 4× dose, and 10× dose) or XENP36764 (4× dose, 10× dose, and 20× dose) to investigate pharmacodynamics of the PDL1×CD28 bsAbs of the invention. Data from the study show that CD28 receptor occupancy is detected up to day 14 on T cells (FIG. 117) and PDL1 receptor occupancy is detected in activated monocytes (FIG. 118). Finally as depicted in FIG. 119, it was found that the PDL1×CD28 bsAbs demonstrate cytokine-like activity in selectively inducing proliferation of effector CD4+ and CD8+ T cells, particularly CD45RA− subsets. Notably, XENP36764 in the 1+1 Fab-scFv-Fc format having monovalent binding for PDL1 induced proliferation more efficaciously than XENP36803 in the 2+1 mAb-scFv format having bivalent binding for PDL1.

5E: Characterizing PDL1×CD28 bsAbs Utilizing Additional PDL1 Binding Domains

Activity of the novel PDL1 binding domains from the common light chain single-cell campaign described in Example 2B were first investigated in the context of PDL1×CD28 bsAbs to confirm they are biologically active. Induction of cytokine (IL-2 and IFNγ) release was assessed as follows: MDA-MB-231 (PDL1+PDL2+) cancer cells and T cells (1:1 effector:target ratio) were mixed with 1 μg/ml of an illustrative B7H3×CD3 bispecific and indicated concentrations of PDL1×CD28 bsAbs. The data as shown in FIG. 120 show that XENP42047 and XENP42048 respectively utilizing 13G1 and 13G7 PDL1 CLC binding domains demonstrated similar potency and efficacy as XENP37261, while additional bsAbs utilizing other PDL1 CLC binding domains were less potent and/or efficacious.

### Example 6: PDL2×CD28 bsAbs

Activity of the novel PDL2 binding domains from the common light chain single-cell campaign described in Example 2B were first investigated in the context of PDL2×CD28 bsAbs. This is to confirm that the PDL2 domains are suitable for use in the PDL1×PDL2×CD28 triAbs of the invention but also demonstrates that PDL2×CD28 bsAbs can also be useful. Induction of cytokine (IL-2 and IFNγ) release was assessed as follows: MDA-MB-231 (PDL1+PDL2+) cancer cells and T cells (1:1 effector:target ratio) were mixed with 1 μg/ml of an illustrative B7H3×CD3 bispecific and indicated concentrations of PDL2×CD28 bsAbs. The data as depicted in FIG. 121 show that most of the PDL2×CD28 bsAbs (XENP42051, XENP42052, XENP42053, and XENP42054 respectively utilizing 5C11, 8G2, 8G5, and 16G11 PDL2 CLC binding domains) but not all (i.e. XENP42050 using another high affinity PDL2 CLC binding domain) were equally or more potent and/or efficacious than the PDL1×CD28 bsAbs in inducing cytokine release.

### Example 7: PDL1×PDL2×CD28 triAbs

As described in Example 5A(c), PDL1×CD28 bsAbs are more active on PDL1^{high} cells. In certain contexts (e.g. within certain tumor environments), there may be insufficient PDL1 on tumor cells which may attenuate the efficacy of PDL1×CD28 bsAbs. It was hypothesized that antibody binding capacity could be restored or enhanced by introducing a PDL2 binding domain to make PDL1×PDL2×CD28 triAbs in which the PDL1 and PDL2 arms work mutually exclusive of each other. FIG. 122 depicts a simplified depiction of the scenarios wherein tumor cells have different expression levels of PDL1 and PDL2 and relative binding capacities of a PDL1×CD28 bsAb, a combination of PDL1×CD28, and a PDL1×PDL2×CD28 triAb (in the 1+1+1 Fab-Fab-scFv format having PDL1 and PDL2 CLC binding domain and a CD28 scFv binding domain) and illustrates that the triAb targets PDL1⁻PDL2+, PDL1+PDL2⁻, and PDL1⁺PDL2⁺ tumors.

7A: 1+1+1 PDL1×PDL2×CD28 triAbs are Functional and Activity is Format Dependent

PDL1×PDL2×CD28 triAbs utilizing 13G1 and 13G7 CLC PDL1 binding domains, 5C11, 8G2, 8G5, and 16G11 CLC PDL2 binding domains, and 1A7-derived CD28 binding domain were produced in the 1+1+1 stackFab-scFv-Fc and the 1+1+1 mAb-scFv formats. Induction of cytokine (IL-2 and IFNγ) release by the triAbs was assessed as follows: PDL1-transfected CHO or PDL2-transfected CHO cells and T cells (1:1 effector:target ratio) were mixed with 1 μg/ml of an illustrative B7H3×CD3 bispecific (having murine B7H3 specificity) and indicated concentrations of bsAbs and triAbs. Data from these experiments are depicted in FIGS. 123-124. The PDL1×CD28 bsAbs did not induce any cytokine secretion in the presence of CHO-PDL2, and the PDL2×CD28 bsAbs did not induce any cytokine secretion in the presence of CHO-PDL1. However, the PDL1×PDL2×CD28 triAbs induced cytokine secretion in the presence of both the CHO-PDL1 and CHO-PDL2 cells (as hypothesized, mutually exclusive of each other so that only one target antigen is required). 1+1+1 mAb-scFv format bsAbs were functional and performed similarly on both CHO-PDL1 and CHO-PDL2 cells. Notably in the presence of CHO-PDL1 cells, stackFab-scFv-Fc format triAbs having PDL1 binding domain on the bottom was more potent than those triAbs having PDL2 binding domain on the bottom (although having PDL1 binding domain on the top was at least on par with the mAb-scFv format); while in the presence of CHO-PDL2 cells, stackFab-scFv-Fc format triAbs having PDL2 binding domain on the bottom was more potent that those triAbs having PDL1 binding domain on the bottom (although having PDL2 binding domain on top was at least on part with the mAb-scFv format).

In another experiment, PDL1×PDL2×CD28 triabs in 1+1+1 Fab-(Fab-scFv)-Fc (with the CD28 scFv in the VHVL or VLVH orientations), 1+1+1 stackFab₂-scFv-Fc, and 1+1+1 mAb-scFv formats were compared. Effector cells and LnCAP (PDL1^{low}PDL2^{null}), DU145 (PDL1^{med}PDL2^{null}), or LCLC103H (PDL1^{hi}PDL2^{med}) tumor cells were incubated at 1:1 effector:target with 1 μg/mL illustrative B7H3×CD3 bsAb and dose titration of the various PDL1×PDL2×CD28 triAbs, and resulting IL-2 secretion is depicted in FIG. 155. The data show that the triAbs in the stackFab₂-scFv-Fc format outperformed those in the Fab-(Fab-scFv)-Fc and mAb-scFv formats (which performed similarly). Notably, reversing the orientation of the CD28 scFv as in XENP42973 decreased activity.

In yet another experiment, PDL1×PDL2×CD28 triAbs in the 1+1+1 stackFab₂-scFv-Fc format with either WT scFv or stapled scFv. Effector cells and DU145 tumor cells were incubated at 1:1 effector:target with 1 μg/mL illustrative B7H3×CD3 bsAb and dose titration of the PDL1×PDL2×CD28 triAbs, and resulting IL-2 secretion is depicted in FIG. 156. The data show that activity was not affected by disulfide-stabilization of the scFv.

7B: 1+1+1 PDL1×PDL2×CD28 triAbs are Functional on Cells Having Various PDL1 and PDL2 Expression Densities

Next, the activity of PDL1×PDL2×CD28 triAbs (as well as PDL1×CD28 and PDL2×CD28 bsAbs) were investigated in the context of cancer cell lines endogenously expressing PDL1, PDL2, and B7H3. FIG. 125 depicts PDL1, PDL2, and B7H3 antigen densities on LCLC-103H, SNU-423, and NCI-H460 cell lines (as determined by FACS using fluorescently-labeled beads as advised by the QuickCal protocol (Bangs Laboratories, Inc., Fishers, IN). The data show that the three cell lines had varied PDL1:PDL2 ratios.

In an initial set of experiments, 10,000 LCLC-103H, SNU-423, or NCI-H460 cells were plated overnight, then treated with a dose titration of PDL1×CD28 or PDL2×CD28 bsAbs, constant 1 μg/mL dose of an illustrative murine B7H3×CD3 bsAb, and purified T cells (1:1 effector:target ratio). IL2 was measured 1 day post T cell seeding using MSD assay, data for which are depicted in FIG. 126. The data show that each of the bsAbs were active on LCLC-103H and SNU-423 with XPL1-13G1 as the most potent PDL1 binding domain and 8G2 as the most potent PDL2 binding domain. Consistent with the data in Example 6, the PDL2×CD28 bsAbs were less active on the PDL21^{low} NCI-H460 cells. Notably while most of the PDL2×CD28 bsAbs were weaker in activity on the LCLC-103H and SNU-423 cell lines, the PDL2×CD28 bsAb XENP45052 having XPL2-8G2 retains activity comparable to the PDL1×CD28 bsAbs.

Next, the triAbs were tested. As above, 10,000 LCLC-103H, SNU-423, or NCI-H460 cells were plated overnight, then treated with a dose titration of PDL1×CD28 or PDL2×CD28 bsAbs, constant 1 μg/mL dose of an illustrative murine B7H3×CD3 bsAb, and purified T cells (1:1 effector:target ratio). IL2 was measured 1 day post T cell seeding using MSD assay, data for which are depicted in FIG. 127.

In another experiment, the triAbs were tested on PDL1high MDA-MB-231 (˜130,000 PDL1 antigens) and PDL1low LNCaP (˜13,000 PDL1 antigens). T cells were co-cultured with (A & C) MDA-MB-231 (130,000 PDL1 antigens) or (B & D) LNCaP (13,000 PDL1 antigens) and treated with indicated concentrations of indicated antibodies (illustrative B7H3×CD3 bsAb alone or in combination with PDL1×PDL2×CD28 bsAbs XENP43461, XENP44283, or XENP44288, sequences for which are depicted in FIGS. 88 and 91). IL-2 activity was assayed 1 day following treatment. Redirected T cell cytotoxicity (RTCC) activity was measured by luminescence 5 days following treatment. Data as depicted in FIG. 136 show enhancement of IL-2 release on both high and low PDL1 density target cells. Data as depicted in FIG. 137A show enhancement of T cell-directed cytotoxicity at low effector to target ratios (1:50).

7C: 1+1+1 PDL1×PDL2×CD28 triAbs are Functional with Presence of Either PDL1 or PDL2

10,000 LCLC-103H (100,000 PDL1 surface antigens, 40,000 PDL2 surface antigens) were plated overnight and then pre-treated with either 5 μg/mL blocking bivalent PDL1 mAb, 5 μg/mL blocking bivalent PDL2 mAb, or PBS control. Cells were then treated with a dose titration of XENP42049 PDL1×CD28, XENP42054 PDL2×CD28, or XENP43461 PDL1×PDL2×CD28 triAb, constant 1 μg/mL does of an illustrative murine B7H3×CD28 bsAb, purified T cells (1:1 effector:target ratio). IL2 was measured 1 day post T cell seeding using MSD assay, data for which are depicted in FIG. 130. The data show that PDL1×CD28 bsAbs require PDL1 (as indicated by reduced potency of XENP42049 in the presence of PDL1 blockade) and PDL2×CD28 bsAbs require PDL2 (as indicated by reduced potency of XENP42054 in the presence of PDL2 blockade) while PDL1×PDL2×CD28 triAbs are active with presence of either PDL1 or PDL2 (as indicated by similar potency in the presence of either PDL1 or PDL2 blockade).

7D: PDL1 and PDL2 Blockade by PDL1×PDL2 triAbs are Functionally Equivalent to PD1 Blockade

To investigate effect of PDL1 and PDL2 blockade by PDL1×PDL2×CD28 triAbs without influence of the CD28 binding domain, PDL1×PDL2×RSV triAb XENP43456 (sequences as depicted in FIG. 131) was engineered and produced. Mixed lymphocyte reactions (n−14) were treated with indicated concentration of PBS, XENP43456 PDL1×PDL2×RSV triAb, or anti-PD1 mAb and IFNγ secretion was assayed after 5 days. Data depicted in FIG. 134 show that PDL1 and PDL2 blockade by PDL1×PDL2 triAbs are functionally equivalent to PD1 blockade.

In another experiment, Jurkat-PD1 cells were treated with a dose titration of PD-1 mAb or XENP43456 PDL1×PDL2×RSV triAb (sequences depicted in FIG. 131) in the presence of A431 cells (transfected to express αCD3 scFv). 6 hours post Jurkat-PD1 seeding, luciferase activity was measured as an indicator of PD-1 blockade. The data as depicted in FIG. 141 show that PDL1 and PDL2 blockade alone (absent CD28 agonism) is functionally equivalent to PD1 blockade.

7E: PDL1×PDL2×CD28 triAbs Enhance a T Cell Recall Response in a CD28- and HLA-Dependent Manner

A CMV recall assay derived with A431-02M-null cells (40,000 PDL1 antigen) stably expressing a fusion of HLA-A2, β2M and NLV-peptide, a peptide presented by CMV-infection (Carreno et. al., J Imm. 188:5839-5849, 2012). CD3+ enriched T cells from two unique CMV+ PBMC donors were mixed with indicated cell lines and antibodies (XENP43461 PDL2×PDL1×CD28 triAb, XENP43456 RSV×PDL2×PDL1 triAb control, and XENP39273 RSV×CD28 bsAb control; sequences depicted in FIGS. 88 and 131). IL-2 was measured in culture supernatants 24 h following treatment. Data as depicted in FIG. 138 show that PDL1×PDL2×CD28 triAbs enhance a T cell recall response in a CD28- and HLA-dependent manner.

7F: PDL1×PDL2×CD28 triAbs Reduces Tumor Growth in Syngeneic Mice MC38 Tumor Model

1×10⁶ MC38 cancer cells stably expressing high levels of human PDL1 antigen were subcutaneously inoculated into human PDL1/human CD28 double knock-in C57BL/6 mice. When tumors were at approximately 300 mm³ (Day 0), mice were intraperitoneally treated twice per week with either PBS control, bivalent anti-murine PD1 antibody (3 mg/kg), XENP43735 (mouse surrogate PDL1×PDL2×RSV triAb control, sequences as depicted in FIG. 139B; 10 mg/kg), or XENP43734 (mouse surrogate PDL1×PDL2×CD28 triAb analogous to XENP43461 (without Xtend), sequences as depicted in FIG. 139A; 10 mg/kg). Tumor volumes were monitored by caliper measurements twice per week, data for which are shown (days post 1^{st} dose) in FIG. 140. The data show that although PDL1/PDL2 blockade inhibited tumor growth, addition of the CD28 modality as in the PDL1×PDL2×CD28 triAb enhanced anti-tumor activity.

The data demonstrate that the PDL1×PDL2×CD28 trispecific antibody enhances the activity of a CD3 bispecific antibody through multiple mechanisms. Notably, the control antibody, PDL1×PDL2×RSV, demonstrates some improvement of anti-tumor activity by itself, consistent with the concept that blockade of the PD1 pathway can enhance the activity of a T cell engager. Moreover, when the CD28 binding arm is added in the PDL1×PDL2×CD28 trispecific antibody, additional antitumor activity and T cell expansion (data not shown) is observed, confirming that the targeted CD28 costimulation is also contributing to the potentiating effects of the molecule.

7G: Developing PDL1×PDL2×CD28 triAbs Utilizing Common Light Chain for all Three Binding Domains

While the triAbs described above utilized common light chain for the PDL1 and PDL2 binding domains, it was desired to develop triAbs that utilized a common light chain for all three binding domains (full CLC triAbs) as multispecific antibodies utilizing Fab domains only may be less immunogenic and more stable than those utilizing scFv domains.

Initial efforts to develop such trispecific molecules built on the triAbs described above already utilizing common light chain for the PDL1 and PDL2 binding domains by panning for novel CD28 binding domains having the same common light chain. As described in Example 2B(a) and depicted in FIGS. 77-80, a number of suitable and functional CD28 binding domains were identified, amongst which clone 1A3.88474 demonstrated favorable properties. Accordingly, full common light chain triAbs were engineered using this binding domain including XENPs 43400, 43402, 43404, 43405, 43452, and 43453 (each of which utilized the same affinity-optimized PDL1 and PDL2 binding domains and CD28 binding domain, albeit with the binding domains in different positions). These triAbs were compared to XENP43461 (matching PDL1 and PDL2 binding domains in combination with 1A7 scFv in 1+1+1 stackFab₂-scFv-Fc format). The triAbs were incubated with 1 μg/ml illustrative B7H3×CD3 bsAb and DU145 tumor cells (1:1 effector:target), and IL-2 secretion was measured after 24 hours, data for which are depicted in FIG. 157. The data show that while all the triAbs were functional, XENP43461 having the 1A7 binding domain outperformed all triAbs having the 1A3 binding domain.

Next, development efforts focused on engineering a triAb utilizing a common light chain for all three binding domains and including the 1A7 VH. As described in Example 2B(b), the 1A7 clone comprises a variable light domain that differs by only a single amino acid in the LCDR3 from the variable light domain in an in-house common light chain phage library. Accordingly, novel common light chain PDL1 and PDL2 binding domains were identified and affinity-matured as described in Example 1B(b) to combine with 1A7. Additionally as described in Example 2B(b), the VH of 1A7 does not pair well with the parental germline VL used in the phage library, and so the VH of 1A7 was affinity-matured with the goal to engineered a triAb that performs as well as a triAb comprising the 1A7 37 nM scFv. Exemplary such full CLC triAbs comprising 1A7 with germline VL and affinity-matured VH include XENPs 44281-44289.

In a first experiment, the activity of these full CLC triAbs (as well as XENP43465 which is a 1+1+1 stackFab-scFv-Fc triAb comprising same affinity-optimized PDL1 and PDL2 binding domains and the 1A7 37 nM scFv) were investigated by incubating with 1 μg/ml illustrative B7H3×CD3 bsAb and DU145 cancer cells at 1:10 effector:target ratio, and IL-2 secretion after 24 hour is depicted in FIGS. 158-159. The data show that several of the full CLC triAbs achieved potency comparable to that of XENP43465.

In a second experiment, the activity of XENP4288, an exemplary full CLC triAb comprising 1A7-derived CD28 binding domain, and XENP43453, an exemplary full CLC triAb comprising 1A3 CD28 binding domain, were compared. Dose titration of illustrative B7H3×CD3 bsAb was incubated with 1 μg/ml PDL1×PDL2×CD28 triAbs in the presence of T cells and Caki-1 cancer cells (1:1 effector:target ratio), and IL-2 secretion after 24 hour is depicted in FIG. 160. The data show that the 1A7-based full CLC triAb is more potent and efficacious that the 1A3-based full CLC triAb. Additionally, data in FIG. 137B show enhancement of T cell-directed cytotoxicity on low antigen density target cells by XENP44283, another 1A7-based full CLC triAb.

7H: PDL1×PDL2×CD28 triAbs Combines Productively with CD3 T Cell Engager to Reduce Tumor Growth in a Solid Tumor Xenograft Model

3×10⁶ pp65-MDA-MB231gfp cancer cells were intradermally inoculated into NSG mice that were MHC I/II-DKO (NSG-DKO; resistant to GVHD) on Day −21. On Day 0, mice were engrafted with 5×10⁶ human PBMCs. Mice were intraperitoneally treated on Days 0, 7, 14, 19, and 28 with the following test articles: PBS control, illustrative B7H3×CD3 bsAb (0.5 mg/kg), PDL1×PDL2×CD28 XENP44676 (sequences as depicted in FIG. 91) in combination with B7H3×CD3 bsAb (respectively 5 mg/kg and 0.5 mg/kg), control PDL1×PDL2×RSV XENP44796 (sequences as depicted in FIG. 131) in combination with B7H3×CD3 bsAb (respectively 5 mg/kg and 0.5 mg/kg), or control RSV×RSV×CD28 XENP44797 (sequences as depicted in FIG. 131) in combination with B7H3×CD3 bsAb (respectively 5 mg/kg and 0.5 mg/kg). Tumor volumes were monitored by caliper measurements (1-3 times per week), data for which are shown (days post 1^{st} dose) in FIG. 142 (and Day 39 in FIG. 143). Blood was drawn weekly for cell counts, and data depicted CD3+ cells on Day 7 are shown in FIG. 144.

1-110. (canceled)

111. A multispecific antibody comprising:
a) a CD28 antigen binding domain comprising a first variable heavy domain (VH1) having at least 90% identity to the amino acid sequence of SEQ ID NO:3380 and a first variable light domain (VL1) having at least 90% identity to the amino acid sequence of SEQ ID NO:2452.
b) a PD-L2 antigen binding domain comprising a second variable heavy domain (VH2) having at least 90% identity to the amino acid sequence of SEQ ID NO:3319 and a second variable light domain (VL2) having at least 90% identity to the amino acid sequence of SEQ ID NO:2452; and
c) a PD-L1 antigen binding domain comprising a third variable heavy domain (VH3) having at least 90% identity to the amino acid sequence of SEQ ID NO:3251 and a third variable light domain (VL3) having at least 90% identity to the amino acid sequence of SEQ ID NO:2452.

112. The multispecific antibody according to claim 111 comprising:
a) a first monomer comprising, from N-terminal to C-terminal, VH1-CH1-hinge-CH2-CH3, wherein CH2-CH3 is a first Fc domain;
b) a second monomer comprising, from N-terminal to C-terminal, VH2-CH1-linker-VH3-CH1-hinge-CH2-CH3 and CH2-CH3 is a second Fc domain;
c) a first common light chain comprising from N-terminal to C-terminal, VL1-CL, wherein CL is a constant light domain;
d) a second common light chain comprising from N-terminal to C-terminal, VL2-CL, wherein is a constant light domain; and
c) a third common light chain comprising, from N-terminal to C-terminal, VL3-CL, wherein CL is a constant light domain;
wherein VH1 and VL1 form said CD28 antigen binding domain, VH2 and VL2 form said PDL-2 antigen binding domain, and VH3 and VL3 form said PD-L3 antigen binding.

113. The multispecific antibody according to claim 112, wherein the first and second Fc domains are variant Fc domains.

114. The multispecific antibody according to claim 113, wherein the first and second Fc domains comprise a set of heterodimerization skew variants selected from the group consisting of S364K/E357Q:L368D/K370S; S364K:L368D/K370S; S364K:L368E/K370S; D401K:T411E/K360E/Q362E; and T366W:T366S/L368A/Y407V, wherein numbering is according to EU numbering.

115. The multispecific antibody according to claim 114, wherein the first and second Fc domains each comprise one or more ablation variants.

116. The multispecific antibody according to claim 115, wherein the one or more ablation variants are E233P/L234V/L235A/G236del/S267K, wherein numbering is according to EU numbering.

117. The multispecific antibody according to claim 116 wherein the CH1-hinge-CH2-CH3 of the first monomer comprises pI variants N208D/Q295E/N384D/Q418E/N421D, wherein numbering is according to EU numbering.

118. The multispecific antibody according to claim 117, wherein the first Fc domain comprises amino acid variants L368D/K370S/N208D/Q295E/N384D/Q418E/N421D/E233P/L234V/L235A/G236del/S267K,
wherein the CH1-hinge-CH2-CH3 of the second monomer comprises amino acid variants S364K/E357Q/E233P/L234V/L235A/G236del/S267K, and wherein numbering is according to EU numbering.

119. The multispecific antibody according to claim 113, wherein the first and second Fc domains each further comprise amino acid variants 428/434S.

120. The multispecific antibody according to claim 112 wherein:
a) the PD-L1 binding domain comprises a variable heavy domain having the amino acid sequence of SEQ ID NO:3251, and a variable light domain having the amino acid sequence of SEQ ID NO:3239;
b) the PD-L2 binding domain comprises a variable heavy domain having the amino acid sequence of SEQ ID NO:3319, and a variable light domain having the amino acid sequence of SEQ ID NO:3239; and
c) the CD28 binding domain comprises a variable heavy domain having the amino acid sequence of SEQ ID NO:3380, and a variable light domain having the amino acid sequence of SEQ ID NO:3239.

121. The multispecific antibody according to claim 112, wherein:
a) the first monomer has the amino acid sequence of SEQ ID NO:3201;
b) the second monomer has the amino acid sequence of SEQ ID NO:3202; and
c) the first, second, and third common light chains each have the amino acid sequence of SEQ ID NO:3203.

122. A nucleic acid composition comprising:
a) a first polynucleotide encoding the first monomer of claim 112;
b) a second polynucleotide encoding the second monomer of claim 2; and
c) a third polynucleotide encoding the first, second, and third common light chains of claim 2.

123. An expression vector composition comprising:
a) a first expression vector comprising the first polynucleotide of claim 122,
b) a second expression vector comprising the second polynucleotide of claim 122, and
c) a third expression vector comprising the third polynucleotide of claim 122.

124. A host cell comprising the nucleic acid composition of claim 122, or the expression vector composition or claim 123.

125. A multispecific antibody according to claim 111 comprising:
a) a first monomer comprising, from N-terminal to C-terminal, scFv-linker-CH2-CH3 wherein CH2-CH3 is a first Fc domain;
b) a second monomer comprising, from N-terminal to C-terminal, VH1-CH1-linker-VH2-CH1-hinge-CH2-CH3 wherein VH1 is a first variable heavy domain, VH2 is a second variable heavy domain, and CH2-CH3 is a second Fc domain,
c) a first common light chain and a second common light chain that each comprise, from N-terminal to C-terminal, VL1-CL, wherein VL1 is a first variable light domain and CL is a constant light domain;
wherein the scFv comprises a third VH domain (VH3), a scFv linker, and a second variable light domain (VL2)
wherein the first variable heavy domain and the first variable light domain of the first common light chain form a first antigen binding domain, the second variable heavy domain and the first variable light domain of the second common light chain form a second antigen binding domain, and the third variable heavy domain and second variable light domain form a third antigen binding domain,
wherein one of the first, second, and third antigen binding domains is the PD-L1 antigen binding domain,
wherein one of the first, second, and third antigen binding domains is the PD-L2 antigen binding domain, and
wherein one of the first, second and third antigen binding domain is the CD28 binding domain.

126. A multispecific antibody according to claim 111 comprising:
a) a first monomer comprising, from N-terminal to C-terminal, VH1-CH1-linker-scFv-linker-CH2-CH3 wherein VH1 is a first variable heavy domain, and CH2-CH3 is a first Fc domain;
b) a second monomer comprising, from N-terminal to C-terminal, VH2-CH1-hinge-CH2-CH3 wherein VH2 is a second variable heavy domain, and CH2-CH3 is a second Fc domain,
c) a first common light chain and a second common light chain that each comprise, from N-terminal to C-terminal, VL1-CL, wherein VL1 is a first variable light domain and CL is a constant light domain;
wherein the scFv comprises a third VH domain (VH3), a scFv linker, and a second variable light domain (VL2)
wherein the first variable heavy domain and the first variable light domain of the first common light chain form a first antigen binding domain, the second variable heavy domain and the first variable light domain of the second common light chain form a second antigen binding domain, and the third variable heavy domain and second variable light domain form a third antigen binding domain,
wherein one of the first, second, and third antigen binding domains is the PD-L1 antigen binding domain,
wherein one of the first, second, and third antigen binding domains is the PD-L2 antigen binding domain, and
wherein one of the first, second and third antigen binding domain is the CD28 binding domain.

127. A multispecific antibody according to claim 111 comprising:
a) a first monomer comprising, from N-terminal to C-terminal, VH1-CH1-hinge-CH2-CH3-domain linker-scFv,
wherein VH1 is a first variable heavy domain, and CH2-CH3 is a first Fc domain;
b) a second monomer comprising, from N-terminal to C-terminal, a VH2-CH1-hinge-CH2-CH3, wherein VH2 is a second variable heavy domain and CH2-CH3 is a second Fc domain; and
c) a first common light chain and a second common light chain that each comprise, from N-terminal to C-terminal, VL1-CL, wherein VL1 is a first variable light domain and CL is a constant light domain;
wherein the scFv comprises a third VH domain (VH3), a scFv linker, and a second variable light domain (VL2)
wherein the first variable heavy domain and the first variable light domain of the first common light chain form a first antigen binding domain, the second variable heavy domain and the first variable light domain of the second common light chain form a second antigen binding domain, and the third variable heavy domain and second variable light domain form a third antigen binding domain,
wherein one of the first, second, and third antigen binding domains is the PD-L1 antigen binding domain,
wherein one of the first, second, and third antigen binding domains is the PD-L2 antigen binding domain, and
wherein one of the first, second and third antigen binding domain is the CD28 binding domain.

128. A composition comprising a CD28 antigen binding domain, wherein the CD28 binding domain comprises:
a) a variable heavy domain having at least 90% sequence identity to a variable heavy domain in FIG. 163; and
b) a variable light domain having at least 90% sequence identity to a variable light domain in FIGS. 35 and 37, or a variable light domain having the amino acid sequence of SEQ ID NO:3239.

129. A composition comprising a PD-L1 antigen binding domain, wherein the PD-L1 binding domain comprises:
a) a variable heavy domain having at least 90% sequence identity to a variable heavy domain in FIG. 161; and
b) a variable light domain having at least 90% sequence identity to a variable light domain having the amino acid sequence of SEQ ID NO:3239.

130. A composition comprising a PD-L2 antigen binding domain, wherein the PD-L1 binding domain comprises:
a) a variable heavy domain having at least 90% sequence identity to a variable heavy domain in FIG. 162; and
b) a variable light domain having at least 90% sequence identity to a variable light domain having the amino acid sequence of SEQ ID NO:3239.
